SEQUENCE LISTING (24 sequences)

SEQ ID NO: 1 - DNA, 2220 bp, synthetic construct
  source 1..2220: mol_type = other DNA
atgaaatctcctgtcgatatggatcgctttattgatgatctgatgaagaagatgactctggaagagaaaatcggccagttgaacttgcctgttacgggtgaaataaccaccggacaagccaagagtagtaatgtggctaagcgtatccgtgccggtgaagtgggcggactctttaacttgaaaggcgtggagcgtattcgtgacgttcagaaacaggcagtagaagaaagtcgtctgggtattcctcttttatttggtatggatgtaattcatggatacgaaacggtatttcctattcctctgggattatcctgtacctggaacatgacagctattgaagaatctgcacgtattgctgctatcgaagccagtgctgatggtatttgctggacattcagtccgatggtggatgtttcccgtgatccccgttggggacgagtttccgaagggaatggtgaagatcccttcttgggagcggagattgcgcgtgctatggtacgtggttatcaagggaaagatatgagtagtaatgatgaaattatggcttgcgtgaagcactttgcgttatatggggcatcagaagccggacgcgactataatacagtggatatgagtcatcaacgtatgttcaacgaatatatgttaccttatcaggctgccgtggaagaaggtgtgggtagtgtgatggcttcattcaatgaagtggatggtgtaccggctaccggaaataagtggctgatgaccgatgtacttcgtaagcagtggaattttgatgggttcgttgtgacggactataccggtatcactgaaatgaccgatcatggtatgggtgatacacaaacagttgcagccctggctctgaatgcaggtgtcgatatggatatggtgagcgatgcttttacaagcacacttaaaaaatctctggaagaaggaaaagtttcagtaaaggctgttgatgctgcttgtcgccgtattctggaagctaagtataagctggggctttttgataatccctataaatattgtgatataacccgtcctaaaaaacaaatctttacaaaagaacaccgcgctatagcccgtaagacagcttcggaaagctttgttctcttgaagaatgagaatagtgtactccctctggcaaagaaaggtaccattgctgtagtaggtcctttggccgatagccgtagcaatatgccgggcacgtggagtgtggccgctgtgatgaacaaatatccttctttgattgaaggcttgaaagaagtagtgggaggcaaggctaaaattcttacggctaaaggaagtaatctgatgagtgatgccgaatacgaagaacgtgctactatgtttggccgtactctgcatcgtgacaatcgtacagataaggaactgctggatgaggcgcttgctgtagctgccaagtctgacgtgattgttgctgctttgggtgagtcttccgagatgagcggtgaaagtagttgccgtacagacctcgaaatgccggatacgcaacgtgtacttttgcaggaattgttgaaaaccggcaaaccggtggtattggtgttgtttaccggtcgtccgttagtattgaattgggagcaggaaaatgtacctgctattctgaatgtgtggtttggtggtagtgaagctgctcttgccattggtgatgtactgtttggaaatgtaaatccgagtggcaaacttactactacttttccgaagagtgtaggacagattcctttgttctataaccataagaatactggtcgtcctttgcctcaaggggcctggttccagaagttccgtagcaattatctggatgtagataacgaaccgctttatccgtttggatatggcttgagctatactactttctcttatagtgatattacattggataaatcgtccatgaatatcaatggagagattatggcaactgtaacggtaaccaatacaggtaagtatgacggttcggaagtagtgcagctatatatccgcgatcttataggcagtgtaacacgtccggtgaaagaactgaaaggctttgaaaaaatcttcttgaaagccggtgaatccaaacaagtgtctttcaagttaacagctgatatgttgaagttctacaattacaatctggattttgtgtgcgaaccgggtgactttgaagtaatgataggtggtgatagccgtgatgtgaataaggccttattttcgcttcaataa

SEQ ID NO: 2 - DNA, 2274 bp, synthetic construct
  source 1..2274: mol_type = other DNA
aagtcaccgcaagacatggatcgcttcatcgacgcactgatgaagaagatgaccgtggaagagaaaatcggacaattgaacctacccgtcacgggagacatcaccacgggacaggccaaaagtagcgacgtggcacaaaagattgaaaaaggattggtgggcggactcttcaacctaaaaggtgtagaccgtattcttgaagtgcaaaagctggcagtagagaaatcacgcctcggtattcccctgctgttcggcatggatgtgatacatggctacgaaaccatcttccccattccattgggattgtcctgcacctgggatatggcggctatcgagaaatccgcccgtattgcagccatcgaagcaagtgccgatggcatttcctggacattcagtccgatggtagacatcagtcgcgacccacgttggggacgtgtcagcgagggctcgggagaagatccgtttctgggtggagctatcgcacaggcaatggtatacggataccagggtgccaatctgcaagaccaattgcaccgcaacgatgaaatcatggcttgcgtaaaacactttgcattgtatggagccggagaagccggacgcgactataatacagtagatatgagccgcaaccggatgttcaatgaattcatgtacccgtatgaggctgccgtagaggccggagtgggtagtgtgatggcgtcattcaatgaaatagacggtattcccgccaccggaaacaaatggctgctgagcgatttgctgcgtggccagtggggcttcgaagggtttgtggtaacggacttcacaggcatttcagagatgatagagcatggtgtcggcgacttgcaaaccgtcagtgcactcgctcttaatgcaggggtggacatggatatggtaagtgagggcttcgtcggtacactgatgaaatcaattaaagaaggaaaagtaagaatgggcacgttgaatacagcctgccgccggatattggaagcgaaatacaagctgggactgtttgacaatccttataaatactgcgacgtgaaccgtccgaagcgggatatcttcacaaaagagcatcgtgacgccgcccgcaagattgccggcgaaagttttgttcttctgaagaatgcccccgccaccgcacagccactcgcagctcatagctcgtcacccgtaactgcttcccccgtgcttccgttgaagaaacaaggtacagttgccgtcatcggccctctcggaaatacccgcagcaacatgccgggcacctggagcgtagccgcacgcctcaacgattatccttctttatacgaaggcttgaaagaaatgatggcaggcaaggtgaacatcacctatgccaaaggtagtaacctcatcggcgatgcagcttacgaagaacgtgccaccatgttcggccgttcattgaaccgcgataatcgcacggaccaggagttactggacgaagcactgaaaattgcagccggcgccgatgttatcgtagctgccctgggagaatcttctgaaatgagcggtgaaagttcaagccgcaccgaactcggcttgcccgatgtacaacataccctgttggaagccttactgaaaacgggtaaacccgtagtactaaccctctttaccggtcggccgttgacgctgaactgggaacaggagcatgtacctgccatcttgaatgtatggttcggaggcagtgaagcggcttatgccattggcgatgttctgttcggtgacgtcaatccgagtggaaaactaaccatgactttcccgaagaatgtaggccagatacctttgttctacaatcataagaataccggtcggccactggcggcaggcaaatggttcgaaaagttccgttcaaactatctggatgtggataacgaaccgctgtatcccttcggttatggattgtcgtataccactttccagtacagtgacattgcattgagcacaccgacattgggaaaagatggttccgttacagccgtagtcaccgtcaccaatactggtaaacatgacggtgcggaagtagttcaactctatatccgcgacctcgtaggaagtatcacccgccctgtacgcgagttgaaaggtttcaataaaatcttccttcgcgccggagaaagcaaaacggtatcattcactatcacgcgtgatctgcttcgcttctatgattacgacctgaactacgtagccgaaccgggtgactttgacatcatgatcggtggaaacagccaggctgtgaagacggcgaagttgacactt

SEQ ID NO: 3 - DNA, 2229 bp, synthetic construct
  source 1..2229: mol_type = other DNA
aagagtggggatgcgtcgatgaacaaatttattgataaactgatggacaggatgaccttggaagagaagattggtcagcttaatcttcccagctcgggagatataaccaccggacaggcacgcagcaacaatattgcagacaaaatcagagcaggtgcagtgggtggcttattcaatataaaaggagttgagaagatacaggaagtacaacgtattgctgtagaggagagtcgcctgaaaattcctttactctttggcatggatgttattcatgggtatgaaactgttttccctattcctttgggtatggctgccacatgggatatgaaggctatagaacaatctgctcgtatagcggcgatagaagccagtgccgatggcatctgctggacatttagtccgatggttgatatcagccgtgatccacgttggggacgtgtatccgaaggtagcggagaagatccttttttaggtggtgaaattgctaaggcgatggtatatggctatcagggtaaaggtgatagcgcatatcgtgaaaagactaatattatggcttgtgtgaagcactatgccttgtatggggcagcagaagccggtttggactataatacaactgacatgagccgtattcgtatgtttaatgaatatatgtatccttatcaggcggctgtggatgcgggtgccggcagtgtcatgtcttctttcaacgaggtcgatggaattcctgcaacagccaacaaatggttgataactgatgtcctgcgtaaacagtggggattcggtggttttgtcgttacggactataccggtatcatggaaatggtaaatcatggtattggagatatgcgagaagtctctgcccgtgctttgagtgcaggagtggatatggatatggtgagcgaaggttatctttctacacttcaacaatcattgaaggagggtaagataacagagaaagagatagatcaagcttgccgtcgtattttggaggcaaaatataagctgggattatttgataatccttataagtattgtgatactgaacgtgccaaaacggatatctacactgatgaacatcggagtattgcacgccggatctctgctgaaagctttgttcttttaaagaatgataaacagacactgcctataaagaaaaaaggtaagattgctgtagttgggccgttggcgaatacgagttctaatatgcccggaacgtggagtgtagcggtcaatatggaagctccagctacgcttgtggagggtttgaaagaagtggcaggtgataaagttgaaattgtgtatgctaagggtagccatctgatgagtgatgcggcttatgaggaacgtgcaacactctttggacgtacattataccgggataaggaaaaacgttccgatatccagatgctgaatgaagcattaaatgttgctcatggtgccgatgttgttgttgcggcattaggtgaatcttctgaaatgagtggtgaatcgagtagtcgaacagatttgaatattcctgatgttcaaaaaacattattggaagaattagtgaaaacaggtaaacctgtcgttctggtattattcactgggcgtccgttgaccctgacatgggaagacaaaaatgtatctgctattctgaatgtttggtttggaggtaccgaagccgcttatgctataggagatgtcctattcggaaatgtaaatcctggaggtaagctgcctgtaacatttcctcagaatgtagggcagattcctttattctataaccataaaaatactggacgtccgctggctgagggcggttggtttgagaagttccgggcaaattatctggatgtaacgaatgaacctctttatccatttggctatggactaagttatgcacaatttgattatagcgatgtgagattaagtacggatcaaatagaccggaatggcatgttaaccgcaagtgtgactgtaaccaataacagtgagtgtgatggagatgaaattgttcagttgtatattcgcgatttggtcggtagtgttactcgtccggtgaaagaattgaaaggatttgaaaaagtaacaattagagcaggggagtcaaaagatatttcttttaagatcactccggaaatgcttaagttctacaattcggatatccagtttgtgaatgaagttggtgaattcgaagtaatgatcggaacgaacagcagggatgtgaaaaaagcaacgtttagcttg

SEQ ID NO: 4 - DNA, 2136 bp, synthetic construct
  source 1..2136: mol_type = other DNA
gtagaatctctcctgtctaagatgacccttgaggagaaaatcggtcagatgaaccagatttcctcttacggtaatatcgaggatatgagtgctttgattaagaaaggtgaaatcggttccatcttgaatgaggtggatccggtgcgtattaatgcgctacagcgcgtggcaatggaagaatcccgtttgggtattcctttattgatagcgcgtgatgtcattcacgggtttaaaacaattttccctattcccttgggacaagcggcttcgttcaatccgcaggtagcgaaagacggtgcacggatagcagctattgaagcttcgtctgtaggtatccggtggacttttgcgccaatgattgatattgcccgcgatcctcgctggggacgtattgccgaagggtgtggtgaagatacgtaccttacttccgtaatgggagcagctatggtagaaggttttcagggagattcgctgaatagtcctacttcaattgcagcttgccctaaacattttgtaggttacggtgcagccgaaggaggacgtgattataattccacgttcattcccgaacgtcgtctgcgcaatgtttatttgccaccttttgaagctgccaccaaagcgggtgcagccacgtttatgacttcatttaatgataatgatggaatcccttctaccgggaatgcttttattttgaagaatgtactccgtgacgagtggggattcgatggttttgttgtgacggactgggcttctgccagcgaaatgataagccatggttttgccgccggttcaaaagaagtggcaatgaaatctgtgaatgcaggagtagatatggaaatggtgagttacacttttgtgaaggaactgccggaattagtgaaagagggaaaggtgaaggaaagcactatcgatgaggctgttcgtaatattttgcgtataaagtatcgtttaggattgtttgatacaccttatgtagatgaacaacaaacatctgtcatgtatgctccttctcatttggaagcagctaagcaagccgctgttgaatcggctattctgttgaagaatgataaggaagtgttgccgttacagccatctgtgaaaactgttgcagtggtaggacctatggctaatgcaccttatgaacagttaggtacttggatatttgatggtgagaaagctcgtactcagactccgttgaacgctattaaagaaatggttggcgataaagtacaggtgatttatgaaccgggactagcatatagtcgtgagaaaaatccggcaagtgtggctaaagcagctgccgccgctgcacgtgcagatgtcattcttgcttttgtgggtgaagaatctattctttcgggtgaagctcactgtttggctgatctggatttgcagggtgatcagggagctttgattacagctttggctaagacgggtaaacctgtagtgactattgtgatggcgggtcgtccgttgactatcggtaaagaagtcgaagagtcgactgctgttctctattcattccatccgggcacaatgggcggtcctgcattggctgatttgctttgggggaaggctgtgccgagtggaaaggcgccggtcactttcccgaggatggtgggacaaattcctgtgtactacgctcataataataccggacgtccggctacacggaatgaagtgttgctgaatgatattgctgttgaggcaggacagacttcactgggctgtacttccttctatatggatgcgggttttgatcccttgtttccgtttggttatggcttgtcgtacaccacatttaagtatagcaacatcaaactggcgtctgatgtactgaaaaaagatgatgtgctgacagtgacattcgatctggaaaataccgggaaatatgaaggaacggaagtagctcaattgtatatacaagataagattggttccgtgactcgtccggtgaaagaactgaaacgcttcactcgtgtgacattgaagccgggtgagaaaaaaagcgtttcgtttgaactccctgttagtgaacttgcattttggaacatagatatggctaaagttgtggaacccggagactttgggctttgggtggcaacggatagtcagtccggagaagaagttttcttc

SEQ ID NO: 5 - DNA, 2241 bp, synthetic construct
  source 1..2241: mol_type = other DNA
aagtctccgcaggacatggatcgcttcatcgatgcattgatgaagaagatgactgtagaggaaaagatcggtcagctgaacctacccgtttccggcgagatcgtcaccgggcaggcacaaaacagcgatgtggcaaaaaagattgaacaagggctcgtgggcggactcctcaacctgaaaggggtggagaagatacgcgatgtacaaaaactggccatagagaagtcacgcctgggcatccccctgatattcggcatggacgtagtgcatggttacgaaaccattttccctattccattaggcctctcctgttcctgggatatggaagccatcaggaaatctgcccgcgttgcagccatcgaggccagtgctgatggtatttcctggacattcagcccgatggtagacatcagccgtgatccgcgctggggacgcgtcagcgagggtaacggcgaagacccattcttgggtggagccatcgctaaagcaatggtatcgggttatcagggtatcgacctcaacaaccaactgaagcgcaacgatgaaattatggcatgtgtaaagcacttcgcactgtatggtgccggagaagccggacgtgattacaataccgtagatatgagtcgtaaccgtatgttcaacgaatacatgtatccctaccaagctgccgtagatgcaggtgtaggcagcgtaatggcgtctttcaacgaaatagacggcataccagccacggccaataaatggctgatgaccgacgtactgcgcaagcaatggggcttcgacggctttgtggtgacagactttaccggtatctccgaaatgatagcgcacggcatcggtgacttgcagactgtttccgcacgtgcactcaatgcaggcgtggatatggacatggtaagtgaaggcttcacgggtacaatcaagaaatccatagacgaaggcaagatcagtatggaaaccctggacaaagcctgtcgccgcatccttgaagccaaatacaaactgggattattcgacaatccttataagtactgcgacctgaaacgcccgaagcgtgacatcttcaccaaggaacatcgcgacgctgctcgtaagattgcgggagagagctttgtactcctgaaaaacgacaagtcaggttcctctgcaaacccaacacttcctttgaaaaaagaaggtacggtggctgtcatcggcccactggcaaatacccgcagtaacatgccgggtacctggagtgtagccgcacgcctcaacgactatccttctgtgtacgaaggattgaaagagatgatgaaaggcaaggtaaacatcacttatgccaaaggtagtaacctcatcagtgatgcagcctacgaagaacgtgccacaatgttcggccgttcattaaatcgtgataatcgtacagacaaagagatgctggatgaggcgctgaaagtggccgctaatgcagatgtaataatagccgcattgggagaatcatctgaaatgagtggtgaaagttcaagccgcactaacctggctcttcccgatgtacagcgcactctattggaagctttgctgaaaactggaaagcctgttgtactgacgctctttacaggtcgcccactaacgttgacttgggaacaggagcatgtgcccgccatcctgaatgtatggttcggtggaagtgaggcagcatacgccattggcgatgtattgttcggcgatgtaaatcccagcggcaaactaacgatgacattccccaaaaacgtaggccaaatacctttgttttacaatcataaaaataccggtcgtcctttacttgaaggcaaatggttcgaaaaattccgtagtaattacctggatgtagacaacgatccattgtatccattcggctatggtttgtcgtataccaactttcaatacagcgacataactctgagcgccccgactatgggacaggatggttctgttactgctatggtcacggtaaccaataccggtaagtacgatggtgcagaagtagtgcaactttatatccgtgaccttgtaggaagcatcacccgtccggtaaaagaactgaaagggtttgataaaattttcctcaaagcgggtgaaagtaagactgtatctttcaaaatcactccggaattactgcgcttctacgactatgaactcaactacgtagccgaaccgggagacttcgacataatgatcggggggaacagccaaagtgtaaaaacgactcatctgagtttg

SEQ ID NO: 6 - DNA, 2247 bp, synthetic construct
  source 1..2247: mol_type = other DNA
aagtccccccaagacatggaccgcttcatcgatgcgctgatgaagaaaatgactgtggaagagaaaatcggacagttgaacctacccgtcacgggagacatcaccacaggacaggccaagagcagcgacgtagccgcaaagattgaaaaaggattggtaggcggactcttcaacctgaaaggggtagaccgcattcttgaagtgcaaaagctggcagtagagaaatcacgtctcggtattcccctgttattcggcatggacgtgatacatggatacgaaaccatcttccccatcccattggggctgtcctgcacttgggatatggccgccatcgagaagtctgcccgtatcgcagccatcgaagcaagtgccgatggcatctcctggacattcagtccgatggtagacatcagccgtgatccacgttggggacgtgtcagcgaaggttcgggagaagaccctttcctgggtggagctatcgcacaggcaatggtatacggataccagggtgccaatctgcaagaccagttgcgccgtaatgatgaaatcatggcctgcgttaaacatttcgccctgtatggagccggagaggccggacgcgattataacacagtggacatgagccgcaaccggatgttcaatgaatttatgtatccgtacgaagctgccgtagaggcaggtgtaggtagcgtaatggcttcattcaatgaaatagacgggataccggctaccgggaacaaatggctattgagcgacttgctgcgtggccaatgggggtttgaagggtttgtggtaacagactttacaggtattgcggagatgatagaacatggtgtcggcgacttacaaaccgtcagtgcacttgccctgaatgcaggtgtggatatggatatggtaagtgaaggttttgtcggcacgctgatgaaatccattaaagaaggaaaagtgagaatgggtacgctaaatacggcttgccgccggatattggaagcaaaatataaattgggcctgttcgacaatccttataaatattgtgatgtgaaccgtccgaagcgggacatctttacaaaagaacatcgggatgccgcccgtaagattgccagtgaaagttttgtacttttaaagaacgctcccttagcagcacagaaaaatgccgcccccgtgcttccattaaagaagcaaggcaccgttgcagtaatcggtcctctcggcaatacgcgtagcaatatgccgggcacttggagtgtagctgcacgcctcaacgattatccttctttgtacgaaggactgaaagagatgatggcaggcaaagtcaacatcacctacgccaagggcagcaaccttatcggtgatgctgcttacgaagaacgtgccaccatgttcggtcgctcactgaaccgcgacaaccgtacggatcaggaattattggacgaagcgctgaaagtggcagccggagccgatgtcatcgtagccgcactgggggaatcttctgaaatgagtggtgaaagttcaagccgcacagaactcggcttacccgatgtgcagcatactttactggaagccttactaaaaacaggcaagcctgtagtacttactctgtttaccggtcgcccgttgacactgaactgggaacaggaacatgtacctgctatcctcaatgtatggttcggaggtagcgaggcagcttatgccattggcgatgtattgttcggcgacgtaaatccaagtggaaagctgacgatgacgttcccgaagaatgtaggccagatacctttgttctacaatcataagaataccggtcgcccgttggcagaaggtaaatggttcgaaaagttccgttcaaattatctggatgtggataatgaaccattgtaccccttcggttatggattatcatataccaacttccagtatagtgacattgcactgagcacgcctacactgggaaaagacggttctgttaccgccgtagttactgtaaccaatacgggtaaatacgatggtgcggaagtagtacaactctatatccgtgatcttgtaggaagcatcacccgtccggtgcgcgagctgaaggggttcaataagatcttccttcgtgccggagaaagtaaaacagtatcattcaccatcacgcgcgacctgctccggttctatgattatgatatgaattacgtagccgaacccggtgatttcaatattatgatcggtggaaacagccagacggtgaagacggcaaaattaacactt

SEQ ID NO: 7 - DNA, 2142 bp, synthetic construct
  source 1..2142: mol_type = other DNA
cgggaacaatctttcgatgaggcatggttatttcatcgtggagatattgccgaaggagaaaagcaatctttagatgactcacaatggcgtcagataaatcttcctcatgattggagtattgaagatattcctggaaccaattctccttttacagcggatgctgcaacggaagttgcaggtggttttactgtaggtggtacgggatggtatagaaagcacttctacatagatgcggctgaaaaaggtaaatgtattgctgtctctttcgatggaatttatatgaatgcagatatctgggtgaatgatcgccatgtagccaatcatgtttatggatatactgcatttgaactggatataaccgattatgtacgtttcggagctgaaaatctgatagctgtccgtgtgaagaatgaaggtatgaattgccgttggtatacaggttcgggtatttacaggcatactttcttgaagataaccaatccgcttcattttgaaacttggggaacgtttgtcacgactcccgttgcaacggcggataaagcagaggtacatgtacagagtgttctggcaaatactgaaaaagtaaccggaaaagtgattctggaaacgcggattgtagataagaataaccatactgtagctcggaaagagcaactggtaacattggataacaaagaaaaaacagaggttggccatgcgttggaagtgcttgctccgcaattatggtctatagacaatccttacttatatcaggttgtaaaccgtcttctgcaagatgataaagttatagatgaggaatatatttcaataggtatacgcaatattgcatttagtgcggagaatggtttccagctgaatggtaaatccatgaaactaaaaggcggatgtatccatcatgacaatggtcttttgggtgcaaaggcttttgaccgggcagaggaaaggaaaatagaactactgaaagcggctggtttcaatgcgctgcgcttgtctcataatcctccctcaatcgctttactcaatgcctgcgaccgcttaggtatgctggtcatagatgaggcttttgatatgtggcgctatggtcattatcagtatgattatgcacaatactttgataaattgtggaaagaagatttgcatagtatggttgcacgggataggaatcatcctagtgttatcatgtggagtattggtaatgaaatcaagaacaaagaaactgctgaaattgtggatatatgcagggagttgacaggttttgtgaagacgcttgatacaacgcggcctgttacggcgggagttaattctattgttgatgcaacggatgattttctggctcctctggatgtttgtggttataattactgtttaaaccgttatgaatcggatgccaaacgtcatccggaccgtattatctatgcttcggagtcctacgcatcccaggcttatgattattggaaaggagtagaagatcattcatgggtgatcggtgattttatctggactgcttttgactatattggtgaggcaagtatcggctggtgtgggtatccgcttgataaacgtattttcccttggaatcatgccaattgtggtgatttgaatctttcgggcgaacgtcgtccccagtcctatttgcgtgaaacgttatggagtgatgcaccggtatcccatattgttgtgacgcctcctgttccttcttttcctctgaatccggataaggcggattggagtgtatgggattttccggatgttgtggatcattggaatttcccgggatatgaggggaaaaagatgacagtatctgtatactccaattgtgaacaggttgaactgttcttgaatggggaatctttaggaaaacaagaaaatactgccgataagaaaaatacgcttgtctgggaagtaccttatgctcatggaatattgaaagccgtaagttataataaaggcggtgaagtgggcactgcaacgttggaaagtgctggtaaggttgaaaagatcagattatctgcggacagaacggaaatcgtagctgatggtaatgatctaagctatatcacattagaattggtagatagtaaaggcattagaaatcagttggctgaagaattggtagcattttctatagaaggagatgctacg

SEQ ID NO: 8 - DNA, 2343 bp, synthetic construct
  source 1..2343: mol_type = other DNA
ggggaaaaagattccacacttcgggaacaatctttcgatgaggcatggttatttcatcgtggagatattgccgaaggagaaaagcaatctttagatgactcacaatggcgtcagataaatcttcctcatgattggagtattgaagatattcctggaaccaattctccttttacagcggatgctgcaacggaagttgcaggtggttttactgtaggtggtacgggatggtatagaaagcacttctacatagatgcggctgaaaaaggtaaatgtattgctgtctctttcgatggaatttatatgaatgcagatatctgggtgaatgatcgccatgtagccaatcatgtttatggatatactgcatttgaactggatataaccgattatgtacgtttcggagctgaaaatctgatagctgtccgtgtgaagaatgaaggtatgaattgccgttggtatacaggttcgggtatttacaggcatactttcttgaagataaccaatccgcttcattttgaaacttggggaacgtttgtcacgactcccgttgcaacggcggataaagcagaggtacatgtacagagtgttctggcaaatactgaaaaagtaaccggaaaagtgattctggaaacgcggattgtagataagaataaccatactgtagctcggaaagagcaactggtaacattggataacaaagaaaaaacagaggttggccatgcgttggaagtgcttgctccgcaattatggtctatagacaatccttacttatatcaggttgtaaaccgtcttctgcaagatgataaagttatagatgaggaatatatttcaataggtatacgcaatattgcatttagtgcggagaatggtttccagctgaatggtaaatccatgaaactaaaaggcggatgtatccatcatgacaatggtcttttgggtgcaaaggcttttgaccgggcagaggaaaggaaaatagaactactgaaagcggctggtttcaatgcgctgcgcttgtctcataatcctccctcaatcgctttactcaatgcctgcgaccgcttaggtatgctggtcatagatgaggcttttgatatgtggcgctatggtcattatcagtatgattatgcacaatactttgataaattgtggaaagaagatttgcatagtatggttgcacgggataggaatcatcctagtgttatcatgtggagtattggtaatgaaatcaagaacaaagaaactgctgaaattgtggatatatgcagggagttgacaggttttgtgaagacgcttgatacaacgcggcctgttacggcgggagttaattctattgttgatgcaacggatgattttctggctcctctggatgtttgtggttataattactgtttaaaccgttatgaatcggatgccaaacgtcatccggaccgtattatctatgcttcggagtcctacgcatcccaggcttatgattattggaaaggagtagaagatcattcatgggtgatcggtgattttatctggactgcttttgactatattggtgaggcaagtatcggctggtgtgggtatccgcttgataaacgtattttcccttggaatcatgccaattgtggtgatttgaatctttcgggcgaacgtcgtccccagtcctatttgcgtgaaacgttatggagtgatgcaccggtatcccatattgttgtgacgcctcctgttccttcttttcctctgaatccggataaggcggattggagtgtatgggattttccggatgttgtggatcattggaatttcccgggatatgaggggaaaaagatgacagtatctgtatactccaattgtgaacaggttgaactgttcttgaatggggaatctttaggaaaacaagaaaatactgccgataagaaaaatacgcttgtctgggaagtaccttatgctcatggaatattgaaagccgtaagttataataaaggcggtgaagtgggcactgcaacgttggaaagtgctggtaaggttgaaaagatcagattatctgcggacagaacggaaatcgtagctgatggtaatgatctaagctatatcacattagaattggtagatagtaaaggcattagaaatcagttggctgaagaattggtagcattttctatagaaggagatgctacgattgaaggagtaggtaatgccaaccctatgagcatagaaagtttcgttgctaatagtcggaagacgtggcgcggaagtaacttattggttgttcgttccgggaaatcttcaggacggattattgtaacagcaaaggtaaaggcacttccggttgcgagtattactataactcagaaaaaa

SEQ ID NO: 9 - DNA, 2214 bp, synthetic construct
  source 1..2214: mol_type = other DNA
aaatctcctgtcgatatggatcgctttattgatgatctgatgaagaagatgactctggaagagaaaatcggccagttgaacttgcctgttacgggtgaaataaccaccggacaagccaagagtagtaatgtggctaagcgtatccgtgccggtgaagtgggcggactctttaacttgaaaggcgtggagcgtattcgtgacgttcagaaacaggcagtagaagaaagtcgtctgggtattcctcttttatttggtatggatgtaattcatggatacgaaacggtatttcctattcctctgggattatcctgtacctggaacatgacagctattgaagaatctgcacgtattgctgctatcgaagccagtgctgatggtatttgctggacattcagtccgatggtggatgtttcccgtgatccccgttggggacgagtttccgaagggaatggtgaagatcccttcttgggagcggagattgcgcgtgctatggtacgtggttatcaagggaaagatatgagtagtaatgatgaaattatggcttgcgtgaagcactttgcgttatatggggcatcagaagccggacgcgactataatacagtggatatgagtcatcaacgtatgttcaacgaatatatgttaccttatcaggctgccgtggaagaaggtgtgggtagtgtgatggcttcattcaatgaagtggatggtgtaccggctaccggaaataagtggctgatgaccgatgtacttcgtaagcagtggaattttgatgggttcgttgtgacggactataccggtatcactgaaatgaccgatcatggtatgggtgatacacaaacagttgcagccctggctctgaatgcaggtgtcgatatggatatggtgagcgatgcttttacaagcacacttaaaaaatctctggaagaaggaaaagtttcagtaaaggctgttgatgctgcttgtcgccgtattctggaagctaagtataagctggggctttttgataatccctataaatattgtgatataacccgtcctaaaaaacaaatctttacaaaagaacaccgcgctatagcccgtaagacagcttcggaaagctttgttctcttgaagaatgagaatagtgtactccctctggcaaagaaaggtaccattgctgtagtaggtcctttggccgatagccgtagcaatatgccgggcacgtggagtgtggccgctgtgatgaacaaatatccttctttgattgaaggcttgaaagaagtagtgggaggcaaggctaaaattcttacggctaaaggaagtaatctgatgagtgatgccgaatacgaagaacgtgctactatgtttggccgtactctgcatcgtgacaatcgtacagataaggaactgctggatgaggcgcttgctgtagctgccaagtctgacgtgattgttgctgctttgggtgagtcttccgagatgagcggtgaaagtagttgccgtacagacctcgaaatgccggatacgcaacgtgtacttttgcaggaattgttgaaaaccggcaaaccggtggtattggtgttgtttaccggtcgtccgttagtattgaattgggagcaggaaaatgtacctgctattctgaatgtgtggtttggtggtagtgaagctgctcttgccattggtgatgtactgtttggaaatgtaaatccgagtggcaaacttactactacttttccgaagagtgtaggacagattcctttgttctataaccataagaatactggtcgtcctttgcctcaaggggcctggttccagaagttccgtagcaattatctggatgtagataacgaaccgctttatccgtttggatatggcttgagctatactactttctcttatagtgatattacattggataaatcgtccatgaatatcaatggagagattatggcaactgtaacggtaaccaatacaggtaagtatgacggttcggaagtagtgcagctatatatccgcgatcttataggcagtgtaacacgtccggtgaaagaactgaaaggctttgaaaaaatcttcttgaaagccggtgaatccaaacaagtgtctttcaagttaacagctgatatgttgaagttctacaattacaatctggattttgtgtgcgaaccgggtgactttgaagtaatgataggtggtgatagccgtgatgtgaataaggccttattttcgcttcaa

SEQ ID NO: 10 - DNA, 1755 bp, synthetic construct
  source 1..1755: mol_type = other DNA
caatggaaaccggccggagatagaataaagacaaagtgggcagaacagatcaatccttccgatgtattgcccgagtatccaaggcccatcatgcagcgtaatgactggaaaaacctgaatggtttgtgggattatgctattattgataaaggtggacgcattccaacggattttgaaggccaaattctcgtaccttttgctgtagaatcgtctttgtccggagtaggaaaaagagtgaacgaaaatcaggaagtaatctatcagcggagctttgagataccttcagcctggagaggaaaacaggttttgctacattttggtgccgttgactggaaaaccgatgtatgggtgaacgatattaaggttggaagtcataccggaggatttactccattctcctttgatataactcctgccttgtcggctaaaggtaacaaccgtctggttgtaaaggtttgggaccctacggacagaggccctcaaccacgtggtaagcaagtcagcagaccggaaggtatctggtacactcctgtaacaggtatctggcaaactgtatggctggaacctgttgctggtaaacatattgagaatcttcgtattactcctgatattgaccgtcatctgttaacggtaaaagctgaactgaacaccaacagcacatcagacttcgtggaggtgaatgtgtatgatggtaatcaattaattgctgccggtaagagtattaatggggaacctgtagaagtggcaatgcctgaaaatgcaaaactgtggagccctgattctccttttctctatactttgaaagttactttaaaagaggggaataagattgtggataaggtggatagctatgcggccatgcgtaaatattccactcgcagggatgccaatggtatcgtacgtttggaactgaataatgaagcgctgttccagtttggcccgcttgatcaaggttggtggcctgacggtctgtatacggctcctacggatgaagctttgctgtacgacattcagaagacaaaagattttggttataatatgatccgtaaacatattaaagtagagcctgcccgttggtatacatattgcgaccagcttggaattattgtgtggcaagacatgccgagtggtgaccgcaacccgcaatggcagaaccggaagtactttgatggtacggaaatgaagcgttcagccgaatcagaagcttattatcgcaaagaatggaaagaaataatggactgtctgtattcttatccttgcattggtacctgggtgccatttaatgaggcttggggacagtttaagaccgttgaaattgctgaatggacgaaacaatatgatccgacccgtttggtgaatccagcaagtggcggtaatcattatacttgtggtgatatgcttgacctgcataattatccggcacctgagatgtacttgtatgatgctcagcgtgcaactgttttgggtgaatacggtggtatcggtcttgttctgaaggatcatatctgggagccgaaccgtaactggggttatgttcaatttaattcttccaaagaagctacggatgaatatgtgaagtatgccgatatgctgtataagatggtagacagaggattctccgcagctgtctatacacagactactgacgtggaagtggaagtgaatggcctgatgacctatgaccgtaaggttattaaactggatgaaaagcgtgctaaagaaataaatacacgtatctgtaattcgttgaaaaag

SEQ ID NO: 11 - DNA, 2790 bp, synthetic construct
  source 1..2790: mol_type = other DNA
cagacacttccgcagacagagcggcaatacctctccggccacggatgcgacgacacagtagaatgggactttttctgtaccgacggacgtaactccggtcgatggacgaaaataggcgtcccctcttgctgggagttgcagggttttggtacctatcagtatggaattagtttttatggtaaagcctttcccgaaggcattgccggtgagaaaggaatgtataaatatgagtttgaagttcccgaggaatttcgtggcaagcaggtcagccttgtgttcgaagcatccatgaccgatacggaagttaaggttaacggacgtaaggcaggatcgaaacaccagggagccttctattgcttttcatataatgtcacggatttactgaaatatggcaagaagaatcagctggaagtaacagtttccaaggagagtgagaatgccagtgtgaatcttgccgaacggcgcgccgattattggaactttggcggtatcttccgcccggtatttctggaagtaaaacctgccgtcaatctccgtcatattgctattgatgcacaaatggacggatcattccgtgccaattgctacacgaatatctccggtgacggaatgagtatccgtgcacagattttggacggtaaagggaagaaactggcagataccaccgtacccctaaaagccggaagcgactggactactttacaattgaacgtttctgcccctgccttatggacggcagaaactccgaatctttataaagctcaattttcactgttggataaaggaggtaaagtcctgcatcatgagaccgagacattcggtttccgtactatcgaagttcgtgaaagtgacggattgtacgtgaacggggtgcgtatcaacgtgcgtggtgtcaaccgtcatagtttccgtcccgaaagcggtcgtaccctaagtaaagcgaagaatattgaagatgtacttctgatgaagggcatgaatatgaattctgtccgtctgagccactatccggcggacccggaatttctggaagcatgcgactctcttggactctatgttatggatgaactgggtggctggcatggcaagtacgacacccctacgggagtacgtctgattgaaggcatgatagaacgtgatgtgaaccatccgtccattatctggtggagcaatggtaatgaaaaaggctggaacattgaactggacggagaattccataaatacgatctgcagaaacgcccggtcatccatccgcaaggtaacttctccggtttcgaaaccatgcactatcgttcgtatggagaaagccagaactacatgcgcctgccggaaatctttatgcctactgaattcctgcatggtttgtacgacggaggtcatggtgccggcctgtatgattactgggaaatgatgcgtaaacatccgcgttgtatcggtggtttcctgtgggtattggcggatgaaggcgtgaagcgcgtggatatggacgggttcatagacaatcagggaaatttcggagctgacggaattgtaggccctcaccatgaaaaggaaggcagctattacactatcaagcagctatggagcccggtgcaggttatgaataccgctatcgaccggaatttcgacggtaaactctctgtggagaaccgttatgattatctgaacctgaacacctgtcgttttatctggcagcaagtgaagttcccgtcggtaacggatgcttccaatacaactacacggattctgaaacaaggtgaagtgcaaggaagcgatgtagcagcccatggagtgggagtggtggatatcaagacttctattcttcccgaagcggatgctcttttcctgacagttatagataaatatgggtatgaactttggcgctggactttccccgtagataaactgaatcgggaaacagaacagttttctgcatcatccggccgtgtatcctatacggaaacagaaaaaggtattacggtaaaagcaaacgggcgtacttttgtcttttcaaagaaagacgggcagctgaaagatgtatccgtcaataaccgtaagattagttttgctaacggtccccgttttatcggtgcacgtcgtgcagaccgttccctagatcagttctataatcatgatgacgaaaaagccaaggcaaaggaccgtacttacagtgaatttaccgatgcggcagtcttcacgaaactggatgtgaaagaagagggggggaatctgatcctcaccgctaattataaactgggtaatttagataaagctcagtggacaattcatccggacggcatggctactcttgattatacctacaacttctccggtgtggtagacctgatgggtatttgctttgattaccctgaagaacaagtgctcagcaagcgttggttgggagcaggtccgtatcgtgtatggcagaatcgtattcatggcacgcagtatgatatctgggagaatgattataacgatcctattccgggtgagacattcacctatcctgaattcaagggatattttggcagtgtctcttggatgagtattcgcacgaaagagggaaccatcagcctgacgaatgaaacacctgattcctatatcggagtatatcaaccccgtgatggtcgtgaccggttactgtatacacttcccgaaagcggaatttctgttttgaatgtaattcctccggtgcgtaataaagtaaattccacggacttgtgcggtccttcttcacaaccaaaatgggtggatggctcgcaaacgggacgccttgttatccggtttgaa

SEQ ID NO: 12 - DNA, 2415 bp, synthetic construct
  source 1..2415: mol_type = other DNA
cagcgcagtgagtatctacttgaaaagaactggaagttcatgaagggggaagctccggaagccatgaagccggaatttgacgaccggaagtgggaaaccgtaaccgtgcctcacgactgggccatttttggtcccttcgatcgcagcaacgatttgcaggaagtggcggtaacgcagaacttcgagaagaaagcttccgtcaagaccggacgtaccggtggacttccttatgttggcatcggatggtatcgtactaggttcgatgcccccgtcaatcaacagacgacacttgtctttgatggtgccatgagcgaagcccgtgtatatgtcaatggacaagaagcatgcttctggccatttggttataattctttccattgtgatgtcaccggacttttgaataaagacggtaaaaacaatacgcttgccgtgcgtttggaaaataaaccacaatcttcccgttggtatcctggcgcaggactttatcgcaatgtgcgtgtagtgagtaccgataaagtacatgttcctgtatggggtactcagctgactactcctcatgtttctgatgagtatgcttcagtacgtctgttgaccactattgccaatgatgaagaaagagatatccgtatcgtgacagagataatctctcccgatgggaaagtcgttgcaacgaaggataatacccgtaagattaatcatggtcagccttttgaacaaaacttcctggtgaatgctccttgcttgtggtcgccggagacaccttatttatataaagctgtttctaaaatctatgccgatggcaagcaaacggatgaatacactactcgtttcggcatccgcagcatagaaatcattgccgacaaaggatttttcctgaacggtaagcatcgcaagttccagggggtgtgcaatcaccacgatcttggtccgttaggcgctgccatcaatgttgctgcattgcgccgtcaacttacgatgctgaaagatatgggttgtgatgccatccgcaccgctcacaatatgccggcaccggagttagtgcaactttgtgatgaaatgggttttatgatgatgctggaacctttcgacgaatgggacattgccaaatgtgagaatggctatcaccgttatttcaacgagtgggcagaacgtgatatgataaatatgttgcatcagttccgcaacaatccttgtgtcgtaatgtggagtatcggtaatgaagttcctacccaatgtagtcccgtaggctataaagtcgcttctttcttgcaggatatctgtcatcgtgaagatccgacacgtcctgttacttgcggcatggatcaggtgacttgtgttcttgctaatggttttgccgccatgattgatgtgcccggttttaattatcgcgcacaccgttatctggaagcttatgaactgttgccgcagaatatagtacttggttctgaaacatcctctaccgttagttctcgtggcgtatataaatttcctgtagagaaacgcggggatgcgaagtacgatgatcaccagtcttccggatatgacttggagcattgtgcctggtctaatgttccagatgaagattttgctttagcggatgattatgactggactatcggtcaattcgtttggacaggattcgattatctgggtgagccttctccttatgatacggatgcatggccaagtcatagctctttgtttggtatcattgaccttgccagtttgccaaaagaccgctactatctgtaccgtagtctttggaataagaatgtgaatacactccatatacttcctcactggacatggccgggtagggaaggagagaatactcctgtctttgtttacacaaactatcctgctgccgaacttttcgttaatggaaaaagctatggtaaacagcataaactgacagccgaagagagtaaagctattcaggacaaagatacacttgccctccagcgtcgttaccgcctgatgtggatggacgttccttatgagccgggtgaagtgaaagtggtggcttacgatgcttccggcaaacctgctgaagaaaaagtagttcgtacttccggcaaacctcatcatctggaagtcattgctgaccgtgaccaactcactgccgatggtaaagatttggcatacatcactgttcgtgtggttgataaagacggaaacctttgtcctgctgataatcgtcttgtaaactttacggtgaaaggcgcggggcgttatcgtgctgccgctaatggagatgcaacttcacttgatttattccacttgccgaagatgcccgctttcagtggtcagctgacagccattgttcaaatgaccgaacagcccggtgaaattattttcgaggctaaggctaaaggggtgaaatctggtaagcttgtgctgaggtctgttagagag

SEQ ID NO: 13 - DNA, 1182 bp, synthetic construct
  source 1..1182: mol_type = other DNA
ggtgaaaaggcagaaaaaatacaggattttgctgagtttataaccattcaggggcaagacctgataaaacctgatggtacgaaactctttatcatgggtaccaatctgggcaattggctgaatccggaagggtatatgtttaagtttaacaaaacgaattctccccggtttatcaatgaaatgttctgccaattggtaggacccgactttactgctgagttttggaaagctttcaaagacaattatatcattcgtgaagatattcagtttattaagaatacaggtgcgaataccattcgtcttccattccattataagcttttcacggatgaggactttatggggttgactgccggtcaggatggttttgcccgtgtagacagtgttgtggaatggtgccgtgaagccgatctttatctgattcttgatatgcatgatgctccgggtggacaaacgggtgataatatagatgatagctacggatatccttggttgtttgaaagtgaagccagccagcaattgtattgcgatatctggcgcaagattgcagaccggtataagaatgaaccggtgattctcggttatgagcttttcaatgaacctatcgctccgtattttccgaatatggaagaattgaacggtaaactggaagatatttataagaaaggggtagctgctatccgcgaggtggacaataaccatattattctgttgggtggcgctcagtggaacggtaacttcaagccgttcaaggattctaagtttgatgataaaataatgtatacttgccatcgttatggaggtgatcctactaaagatgatattcaaactataatagacttccgcgacagtgtgaacttaccaatgtatatgggtgagataggacataacacggacgaatggcaagctgctttttgccagacgatgcgtgagaataatatcggttataccttctggccgtataagaagatggatggttccagctttgtaggtattactccgccggaaaattgggcgaatatcctttatttctccgaatctccacgcacatcttataaagaaatccgggatgcccgtcccgaccagatgatggtacgcaaggcaatgatggatttcattgaggcttgcaaactgaagaactgtgtggtgcaggaagggtatattcagtcgttaggtatgaaa

SEQ ID NO: 14 - DNA, 2193 bp, synthetic construct
  source 1..2193: mol_type = other DNA
acacaagtggcaaataaaggtagcgatgcggcaaccgagaaaaaagtagagtctcttttatccagaatgacccttgaagagaaaatcggtcagatgaaccagattacctcttacgggaatattgaggatatgagtagtttaattaagaaaggtgaagtcgggtctatcctgaatgaggtggatccggtacgtattaatgcgttgcaacgcgtagcgatggaggagtcccggttgggaatccctttgttgatagctcgcgatgttattcacgggtttaaaaccatttttcccatcccattgggacaagcggcttcgttcaatccgcagattgcgaaagacggtgcacgggtagcggctattgaggcttcttccgtaggtatccgttggacttttgcaccgatgatcgacattgcccgtgatcctcgctgggggcgcattgccgaaggatgtggtgaagacacttacctgacttctgtaatgggagctgccatggtagaaggttttcagggagattctttgaatagtcccacttccatagctgcctgtcctaaacattttgtgggctatggtgcagctgaaggcggacgtgactataattcgacatttattcctgaacgtcgcctgcgtaatgtttacttgccaccgtttgaagcggcaacgaaagcgggtgcagctacgtttatgacttcctttaatgataatgatgggataccctctaccggaaatgctttcatattgaaagatgtgcttcgtggcgagtggggatttgatggtttggtagtgacagactgggcttctgccagcgaaatgataagtcatggttttgctgccgattctaaagaggtagccatgaaatcagtgaatgctggggtggatatggaaatggtaagttatacctttgtaaaagaattgcctgcattgataaaagaaggaaaggtgaaagaaagcaccattgatgaagccgttcgtaatatattgcgcgtcaagtatcgtctgggattgtttgatgttccttatgtagatgaaaagcaaccctctgtcatgtatgatccttctcatctgaaagtagctaagcaggctgctgtagaatcggctatcctgttgaagaatgataaagaagtactgccgttacaggagtctctgaaaaccattgctgtggtaggacctatggccaatgcgccttatgaacaattgggtacctggatctttgatggtgagaaagctcatactcagacaccactgaatgctattaaggaaatagttggcgacaaagtacaggtgatttatgaacccggattagcttatagccgtgagaaaaatccggcaggcgtagcaaaagctgctgctgttgctgcacgtgcagatgtcattcttgcttttgtgggtgaagaagccattctttcgggtgaagcacactgtctggcagatttgaatcttcagggtgatcaaagtgctttgattacggctttggctaagacaggtaaacctgtagtaaccattgtgatggcaggtcgtccgttgactatcggtcaggaagtggaagaatcaacagctgttctttattcattccatccgggtacgatgggtggaccggcattggccgatctgctgtggggtaaggcggttccaagtggaaaaacaccggttactttcccgaagatggtaggacaaattccggtatattatgctcataacaataccgggcggccggctacacgtaatgaggtgttgctggatgatattgctgttgaggctggacaaacttcattgggatgtacttctttctatatggatgccggttttgatcctttattcccatttggctatggcttgtcgtatacaacgttcaagtatagtaatgtcaaactttcatcagcgtcattgaagaaagatgatgtattgactgtgacatttgatctggaaaatacaggtaaatataaggggacggaagttgctcaattgtatatacaagataaggttggttctgtaactcgtccggtgaaagaactgaaacgttttactcgggtaaccttgaaaccgggcgagaaaaagaatgtttcgtttgaactacccgttagtgaacttgcattttggaacatcgatatggtgaaagttgtggaacccggagactttggactttgggtggcaacagacagccaatcgggagaagaagttttctttaaggtggtagat

SEQ ID NO: 15 - DNA, 2460 bp, synthetic construct
  source 1..2460: mol_type = other DNA
tctgattcaaatgttgatttcaataaagattggaaattcgtactgaaagattctgctcattattcatatacttcttatgtccctggtgatgaatggaagaaagtgaacctgccacacgactggagtgttggtctgccttacgactccatctctggcgaagggtgtgtagctttccttcagggaggaataggatggtatagcaaatcatttcccacaacaatcagcgcaaatcagaaatgctatatagtgttcgatggagtatataataattctgagtattggataaatggcaaaaaacttggatatcatctttcgggatatgctcctttttattttgatgtcacagactatctcaatcccaatgaggataaccgcatgactgtaagggtcgaccacagccattatgccgacagcagatggtacaccggttcaggtatatacagggatgtgaaaatgattgtaaccgacagactgcatattccggtttggggaacatttgtcactactcccgtggttactgataaatatgctaaagtaaacaaccaaattaccgtgcgcaacagttactctgaacccagaacagctgttgttgagatagtgtataaagataataaaggcaatatcgcagcctttgaggtcttcagtataaaactgaatgctggtgaggagaaaattatcgacatcgtatcggagataaaacagccggatttgtggagcgtcgagataccagtcctctatacagccgagacccgtattaagaatggcgatgaagtcatttctgaaaacactgtcaggttcggtatacgaacattccactttgatgcagacaaaggtttcttccttaacggaaaaaatatgaagataaaaggagtatgcctgcatcatgatgccggtatagttggcacagcaatgatacgcgatgtgtggtaccgacgtctgaaaacccttaaggaaggaggatgtaacgccatccgcctttcgcacaatccgggagcggatgagtttctgtctttgtgcgatgagataggtcttctggtccaggaagagttcttcgatgagtgggattatcccaaagataaaaggctcaatatgaaggaaacggtagaagactatcctactcatggttattgtgagcatttccaggaatgggctgaaagggatttgaaaaacgtaatgaggagaagccgtaatcatgcctgtatcttccagtggagtataggtaatgaaatagaatggacttataccggatgccgtgaggcaacaggtttctttggagccgattccaacggtaattacttctggaaccagcctccatactctaaagaaaaaatcagagaaatgtggaaaatccagcctaaacaagcatacgacattggtcgtacagcgcaaaaattagcagcatggacacgccagatggatactacacgagtggttaccgccaactgcatcctgccttccataagttttgagacaggatatatcgatgcacttgatgtggctggtttcagctacagacgcgtgatgtatgattatgctaagaagaattatcctgacaaacctataatgggtacagaaaatcttggtcagtggcacgaatggaaggcggtgattgaaagagatttcgttccgggtatgtttatatggacaggagtcgattatctgggagaaagtggaagccgcctttcaagatggcctcaaaagtcaataggatgtggtctcctggatatgtgcggctatgtgaagccttcgtacgacatgatgaaatcattgtggactgacaagccttttattgctatatattcacagactccagacaaatcttcgtatctccaggtaaaagatggctttactgataagaaaggacatgaatgggatagaagattatgggtttgggatgatgtaaactctcactggaattatcagaaaggtgactcggtaatagtagaaatatattccaattgtgatgaagtggaacttttcgttaacggcaagtcgatgggaaagaagtatatagacgattttgaggatcatatctataaatgggcagttcagtacaagcctggcactattaccgcaaaaggaaaaaataagttaggtaataccactacagctataaggacttcaggcaaagaacattcgatattgctagcggttgacaaacaaagtatcgcagcaaatggaaaggatgttctgcatgtcacagcccagcttacagacaaaaaaggtaatcctgtaaagacaacagaacagatgcttaagttcaacatcgatggagagtaccgtctgttgggtatagacaatggaaatgtaaagaacgtatctccatatcaaagcaaggagattatgacatatcagggaagatgtatgctgatgcttcagtcaacagaaaaaacatcggtactgaatatcagtgcagaaacaagtgaattacagtcgaataaactaacaattaatataaaa

SEQ ID NO: 16 - DNA, 2373 bp, synthetic construct
  source 1..2373: mol_type = other DNA
cagcgacatgaacaactcttggaaaccggctggaaattccacaaaggagaaaccaatggagctgaaactgtttcatttaatgattctcaatgggaatctgtctgtattccacacgactgggccatttatggaccgtttgaccgtaataatgatttacaaaatgtagccattactcagaacttggagaaacaggcatctgtcaagaccggacgtaccggaggacttccttatgtgggagtaggatggtatcgcacccgtttcgatgcagaccctgacaaaaagacaacactggtttttgatggagccatgagtgaagcccgcgtgtatgtcaatggaaaagaagcctgcttctggcctttcggttacaattccttccattgtgacattactgagcttctgcacaaagaaggaaaagacaatgtattggctgtacgtctggaaaaccgtcctcaatcttcccgctggtatccgggagccggactttaccggaatgtccatctgattactgcagaaaaaatacatgtacctgtatggggaacacaggttaccaccccacacgtagctaatgactatgcttctgtttgccttcgtacctctttacagaatgtgggaaaagaagaaattaccatagaaacagaaatactggacccgaacgggaaaaaagtttctttcaagaagaacagcggacgcatcaatcacgggcaaccgtttacacaaaatttcattgtggaaaacccgcaattgtggtcacctgaaacaccgttcttatatcaggccgtatctaaaatctatgccaacggaaaacttacagatacttataccacccgctttggtatccgttccatcgaatttgtagccgacaagggctttttcctgaacggccagcaccgtaaattccagggggtatgcaaccaccacgacttaggtcctttaggagctgccatcaacgtatcggctctacgccaccagcttacattattaaaagacatgggctgcgatgccattcgtaccgcacacaacatgccggcacccgagcttgtcagactctgcgatgaaatgggattcatgatgatgattgagcctttcgatgaatgggacattgccaagtgtgaaaacggataccaccgctatttcaacgaatgggccgaaaaagacatggtaaacatgctacggcaataccggaataatccctgtgtggtgatgtggagtatcggtaatgaagtacccacccaatgcagcagtgaaggatacaaagtagccaagttcctgcaagacatttgccatcgggaagaccctacccgtccggttacctgcggcatggaccaggttagttgtgtactcgacaacggatttgcggccatgctcgacattccgggattcaattatcgcgcacaccgctatgaagaagcttaccaacgcctgcctcaaaatcttgtattaggctcagaaacctcttctaccgtcagttcacgcggtgtatacaaattcccggcagagcgtaaagccgatgcaaaatacgaagaccatcagtcttcttcttacgacttggaatactgctcctggtctaacattcccgatatagactttgctctggctgatgaccaccaatggactttggggcagtttgtctggacaggttttgattatctgggtgaacccagtccatacgatacggatgcatggcccaaccacagctctatgttcggtattatcgacctggcttccttacccaaagaccggtactatttataccgcagcatatggaacaagcaagctgaaacacttcatattcttcctcattggaactgggagggcagagaaggaaaagaagtacctgtattcgtctataccaactatccgacagccgaacttttcatcaacggaaaaagttatgggaaacagacgaagaacaaccaaagcgtagagaaccgttaccgcctgatgtggcacaacgccatttacgaaccgggagaagtaaaagtcgtggcatacgatgaacacggtacggctaaagcagaaaagataatccgcacggcaggcaaacctcaccatattgaattggtttcttcacgccagtcgctcacagccgatggaaaagatttggcttacgtaaccgtacgtgttgtggacaaagacggaaatctctgccccacagatatgcgcttggtgaaatttaaagtaaaaggagctggaagctacaaagcctcagccaatggagatccaacttgtctggatttgttccacctgcctcagatgcacgccttcaacggcatgctgactgcaattgtgcaatcaggaaaagaagcaggtacccttgagttacaagtcaccgcaaaagggctgaaatcaggaaagatacaaatcgaagtaaaa

SEQ ID NO: 17 - DNA, 2739 bp, synthetic construct
  source 1..2739: mol_type = other DNA
cagactgataagattgacctggccggctcgtggacattttctacggacagcatggactggagccgggtgattgaactgccgggttcaatggcttccaatggttttggggaagatattgccgtgggtactgattggacgggcggtattgtggattcttcttatttctttaaaccttcgtatgccaaataccgtgaggcaggaaatatcaaggtacctttctggcttcagccggtaaaatattacaagggtaaggcgtggtatcagaaagaggtggtgattccggacagttgggaaggaaaggacatttctctctttttggaacgatgccattgggagagccgtttgtatatagacggaaaggaaatcggcatgcaaaatgctttgggggcgccccatcgttatgacctgacaggcaagctttcagcagggaaacatgtgttgatgctgtgtgtagacaatcgggtgaaaaacattgatccgggggagaactcacatagtatttccgaccatacacaaggaaactggaacggggtggtaggcgatatgttcctggaagtaaagccggaagtgaatgtgtcttccgtcaagattatgccggagcgtctggctaagaaagtcagtgtgtcggcttccttgatgaaccgttatgaaaaagatgccaatgtggtactggagatgacggtaggtaatgaaaaagtacagcaacaatgtacgttgaagccgggcgaaaatcaagtgatgatgtcgctggccatgaagggagacattaagtgctgggatgagttttctccatccttatatgatttgaagctgagtgtgaaggatgcggatagcggtgaaacggatgtctatgcggaacgttttggtttccgtgatgtgaaggtgaaagacggcaaactcaccatcaacgaccgccgtttgttcctgcgtggtacgctggattgtgccgtatttccgaagaccggtttcccgcccacggatgtagaatcctggaaaaagatttataccacctgtcggcagcacggactgaaccatgtgcgcttccattcctggtgtccgcccgaagctgcttttgcagctgccgatgggatgggtatgtacctggagatagaatgttcttcctgggctaaccagtcgactaccattggcgatggaggcgatctggaccgctttatctgggaggaaagtgaacgcatcgtccgtgagtttggtaaccatccttctttctgcatgatgatgtacggtaacgaaccggctggtgagggaagtaatgcctatctgactaattttgttactacctggaaagagcgcgatgcccgccgtttatattgttcgggtgccggatggcccaatttgccggttaacgacttcttgagcgattccaatcctcgtattcaggcgtggggacaaggtgtgaagagtattatcaacgcacaggctccgcgtaccgactatgactggtcagaatacatcggacgtttccagcagccgatggtgagccacgaaatcgggcagtggtgtgtatatcccaacttcaaggaaatggccaaatacgacggggtgatgcgcccgcgtaattttgagatattccaggaaacactggctgaaaacggtatggcacatttggctgacagcttcctgctggcttccggaaaattgcaggcgttgtgttataaggccgatatcgaagctgctttgcgtacaaaagacttcggtggattccagttactgggcttgtctgatttcccggggcagggtacggctttggtaggagtgctcgatgcgttctgggaagaaaaaggctacatccgtccggaagaataccgtcgtttctgtaatagtacggtaccattactgcgcttgccgaagttgatttataccaaccaggaaacggtgaaaggaagtctggaagtggcacatttcggagctgctccgctggaggtgacttctactgtctggaccctgaaaacaaaagaaggaaagacaattgcttcgggcacgctggcacaccagccggtaggtatcggcaattgtattccgttggggcagctggagattccattggataaggtggacgtcccttcatgtctgacactggaagctacattgggagattacgccaacagctggcacatctgggtatatcctgctgcggtacagaaagtagctgatgaagcacaattgctgatgaccgaccgtctggatgcaaaagctttgcaacgtcttcaggaaggtggcaacgtactgctttctttacggaaaggctccttgcctgccgaagcgggaggcgaagtagtgataggtttctctagcatcttctggaacacggcctggacgctgggacaagcaccgcacacactgggtatcctgtgtaaccccgctcatccggcactttcagagttccctacagagtattacagtgattatcagtggtgggatgccatgagccattccggtgccatcgaagtggtcaagattgataaaaacttgcagccgattgtacgagttatcgacgactggtttacgaaccgtccgctggctttgttgttcgaagtgaaggtgggtaagggtaaattgcttgtgtcaggaattgatttctggcaggatatggacaagcgtacggaagcccgtcagttactctacagcttgaagaaatatatgtgcggtaatcgcttcaatccctcttctgaagtcgatgcgaaagatttaagtattttgttttccattaaaaatcaaaaa

SEQ ID NO: 18 - DNA, 2202 bp, synthetic construct
  source 1..2202: mol_type = other DNA
aaggatgcggagatggaccgctttatcagtgacctgatgggaaggatgaccttgcaggaaaagttaggacagttgaatctgccggctgggaatgacctggtgtcgggagcagtgaagaacagcaagatggcagaagctatccgagctggtgaggtcggcggctttttcaatgtgaagggagtggataagatttaccagatgcagcgtatggcggtggaggaaactcgtctgggaattcctttgatagtgggtgccgatgtgattcacgggtacgaaacaatcttcccgattccgttggccctgtcttgtagctgggatacggcggcggtgacacgtatggcacgtatttctgccacggaagccagtgccgatggaatcagctggaccttcagtccgatggtagacatctgtcgggatgcccgctggggacgtattgcagaaggaagtggagaggacccgtacctcggggcgttgatggctggagcctatgtgcgcggttatcagggtgacggcatgaagcagaacaatgaaatcatggcctgtgtgaagcactttgcgctgtatggagcttcggaatcgggacgtgactacaattcggtggatatgagtcgaaacctgatgtataatgtgtacctggctccttataaaggggcggtggaagccggagtgggttcggtgatgagctcgttcaataccatcaacggggtacctgctacagctgacaaatggctgctgacggatttgctccgcaatgagtgggggttcacggggtttgtggtgaccgactacaattcgattggtgagatgaagactcatggggtggccgacttgaaggaggcttctgcacgggcgttgaatgcaggaacggacatggatatggtggcacatggtttcttgcatacgctggaagcttcattgaaggagaaggccgtgacgcaggagcggattgacgaggcttgtcgtcgggtattggaagccaagtataagttaggattgtttgaaaatccttataagtattgtgatacgcttcggggacgcaaggaattgtttacggaggcgaatcgtaaagcggcacgtgagattgcggctgaaacgtttgtgctgttgaagaacgagggtaagttgttgcctttgcagaaaaaaggacgcattgcattgattgggccgatggctgatgcgcagaacaatatgtgcggcacgtggaacatggattgtcagacagaccgtcatgtgacgatgtacgaagctttccgtcgtgcggtaggtgataaggctacggtttcttatgccaagggaagtaatgtgtattatagtgagcatattgagaaaggggcggtcgaacctcgtccgctgacacgtggcgatgaccgtcagttgcgggctgaggctttgcgcgtggcggcttctgccgatgtgattgtggccgcattaggtgagagtgctgagatgagcggagagtcttcttctcgtacagatattcagattccggatgcgcagaaagatttgttgaaggcattgatagctaccggaaagccggtggtactggctttgtttaccggtcgtccgctggatttatgctgggagtctgagcatgttccggctatcctgaacgtgtggtttgccggcagtgaagcgggtgatgccattgccgatgtgatgtttggagaagtatctccttcgggtaagctgactacgagtttcccacgtgcggtgggacagttgccgctttattataatcacctgaatacgggtcgtccggatacggatgacactactttcaatcgttatggcagcaattacatcgaccagagtaatgaaccgctttatccttttggctatggtttgagttataccactttccgttacggtaatttgcagttgagtgcggagcgtatggccaagggtgggcagttgaaggtaaccgtgcctgtaaccaattccggcgagtgtgacggagtagagattgtgcagttgtatcttcacgatgtgtatgcagaaatctcccgtccggtgaaggagctgaaagctttccgccgtgtggcccttaaaaagggagagacacagaatgtagagtttgtactcgatgaggatgatttgaagtattataattctcgtctggaatatggatatgaaccgggagagtttgaagtgatggtgggtccggacagccggaatgtgcagcacgcgacttttgtggctgaa

SEQ ID NO: 19 - DNA, 912 bp, synthetic construct
  source 1..912: mol_type = other DNA
atggaactaagacacctcagatattttgtgaccgtggttgaagagcaaagcatttccaaagctgctgaaaagttgtgtattgcccagccgcccctcagccgacaaattcaaaaactcgaagaagaattgggaattcagctatttgaacgcggcttcagaccggctaaagtgactgaagcaggcatgtttttttatcagcatgctgtgcagattttgactcatactgcacaagcgtcctcaatggcaaaacggattgcaacggtcagtcaaaccttgagaattggttacgtcagctccttactgtatggtttgttacctgaaattatttatctgtttcgtcaacaaaatcctgaaattcacatcgaactcatcgaatgcggcaccaaagatcaaattaatgcccttaagcagggaaaaatcgatctgggttttggtcggctcaaaattaccgatcctgcaattcgacgtatcgtgttgcataaagaacagctcaaacttgcaatccataagcatcatcacctcaatcagtttgcagcaacaggggttcatctctctcaaattattgatgaaccgatgctgctgtacccagtctctcaaaagcccaattttgcgacctttattcagtcactctttaccgaactaggcctagtaccatccaaactcaccgaaattcgagaaattcaactggcactcggcttggtggcagcaggtgaaggcgtctgcatcgtaccggcgtctgccatggatattggggtgaagaatctactttatattccaattttagatgatgatgcctatagcccaatttcactcgcggtgcgaaatatggaccacagtaattacattcctaaaattctcgcctgtgtacaggaggtgtttgcaacgcaccatatcaggccactcatcgaataa

SEQ ID NO: 20 - DNA, 18 bp, synthetic construct
  source 1..18: mol_type = other DNA
taatacgactcactatag

SEQ ID NO: 21 - DNA, 111 bp, synthetic construct
  source 1..111: mol_type = other DNA
ttttcaataaatactatttacataccttaaattaatgtaataataaaaaccaacaccaatttggtatttttgcatactaaaaaggtatataaaaccaattagggcgtataa

SEQ ID NO: 22 - DNA, 48 bp, synthetic construct
  source 1..48: mol_type = other DNA
ctagcataaccccttggggcctctaaacgggtcttgaggggttttttg

SEQ ID NO: 23 - DNA, 3256 bp, synthetic construct
  source 1..3256: mol_type = other DNA
ttattgaagcgaaaataaggccttattcacatcacggctatcaccacctatcattacttcaaagtcacccggttcgcacacaaaatccagattgtaattgtagaacttcaacatatcagctgttaacttgaaagacacttgtttggattcaccggctttcaagaagattttttcaaagcctttcagttctttcaccggacgtgttacactgcctataagatcgcggatatatagctgcactacttccgaaccgtcatacttacctgtattggttaccgttacagttgccataatctctccattgatattcatggacgatttatccaatgtaatatcactataagagaaagtagtatagctcaagccatatccaaacggataaagcggttcgttatctacatccagataattgctacggaacttctggaaccaggccccttgaggcaaaggacgaccagtattcttatggttatagaacaaaggaatctgtcctacactcttcggaaaagtagtagtaagtttgccactcggatttacatttccaaacagtacatcaccaatggcaagagcagcttcactaccaccaaaccacacattcagaatagcaggtacattttcctgctcccaattcaatactaacggacgaccggtaaacaacaccaataccaccggtttgccggttttcaacaattcctgcaaaagtacacgttgcgtatccggcatttcgaggtctgtacggcaactactttcaccgctcatctcggaagactcacccaaagcagcaacaatcacgtcagacttggcagctacagcaagcgcctcatccagcagttccttatctgtacgattgtcacgatgcagagtacggccaaacatagtagcacgttcttcgtattcggcatcactcatcagattacttcctttagccgtaagaattttagccttgcctcccactacttctttcaagccttcaatcaaagaaggatatttgttcatcacagcggccacactccacgtgcccggcatattgctacggctatcggccaaaggacctactacagcaatggtacctttctttgccagagggagtacactattctcattcttcaagagaacaaagctttccgaagctgtcttacgggctatagcgcggtgttcttttgtaaagatttgttttttaggacgggttatatcacaatatttatagggattatcaaaaagccccagcttatacttagcttccagaatacggcgacaagcagcatcaacagcctttactgaaacttttccttcttccagagattttttaagtgtgcttgtaaaagcatcgctcaccatatccatatcgacacctgcattcagagccagggctgcaactgtttgtgtatcacccataccatgatcggtcatttcagtgataccggtatagtccgtcacaacgaacccatcaaaattccactgcttacgaagtacatcggtcatcagccacttatttccggtagccggtacaccatccacttcattgaatgaagccatcacactacccacaccttcttccacggcagcctgataaggtaacatatattcgttgaacatacgttgatgactcatatccactgtattatagtcgcgtccggcttctgatgccccatataacgcaaagtgcttcacgcaagccataatttcatcattactactcatatctttcccttgataaccacgtaccatagcacgcgcaatctccgctcccaagaagggatcttcaccattcccttcggaaactcgtccccaacggggatcacgggaaacatccaccatcggactgaatgtccagcaaataccatcagcactggcttcgatagcagcaatacgtgcagattcttcaatagctgtcatgttccaggtacaggataatcccagaggaataggaaataccgtttcgtatccatgaattacatccataccaaataaaagaggaatacccagacgactttcttctactgcctgtttctgaacgtcacgaatacgctccacgcctttcaagttaaagagtccgcccacttcaccggcacggatacgcttagccacattactactcttggcttgtccggtggttatttcacccgtaacaggcaagttcaactggccgattttctcttccagagtcatcttcttcatcagatcatcaataaagcgatccatatcgacaggagatttcatctttcctgtgtgattttcaataaatactatttacataccttaaattaatgtaataataaaaaccaacaccaatttggtatttttgcatactaaaaaggtatataaaaccaattagggcgtataaatggaactaagacacctcagatattttgtgaccgtggttgaagagcaaagcatttccaaagctgctgaaaagttgtgtattgcccagccgcccctcagccgacaaattcaaaaactcgaagaagaattgggaattcagctatttgaacgcggcttcagaccggctaaagtgactgaagcaggcatgtttttttatcagcatgctgtgcagattttgactcatactgcacaagcgtcctcaatggcaaaacggattgcaacggtcagtcaaaccttgagaattggttacgtcagctccttactgtatggtttgttacctgaaattatttatctgtttcgtcaacaaaatcctgaaattcacatcgaactcatcgaatgcggcaccaaagatcaaattaatgcccttaagcagggaaaaatcgatctgggttttggtcggctcaaaattaccgatcctgcaattcgacgtatcgtgttgcataaagaacagctcaaacttgcaatccataagcatcatcacctcaatcagtttgcagcaacaggggttcatctctctcaaattattgatgaaccgatgctgctgtacccagtctctcaaaagcccaattttgcgacctttattcagtcactctttaccgaactaggcctagtaccatccaaactcaccgaaattcgagaaattcaactggcactcggcttggtggcagcaggtgaaggcgtctgcatcgtaccggcgtctgccatggatattggggtgaagaatctactttatattccaattttagatgatgatgcctatagcccaatttcactcgcggtgcgaaatatggaccacagtaattacattcctaaaattctcgcctgtgtacaggaggtgtttgcaacgcaccatatcaggccactcatcgaataa

SEQ ID NO: 24 - DNA, 4396 bp, synthetic construct
  source 1..4396: mol_type = other DNA
ttattgaagcgaaaataaggccttattcacatcacggctatcaccacctatcattacttcaaagtcacccggttcgcacacaaaatccagattgtaattgtagaacttcaacatatcagctgttaacttgaaagacacttgtttggattcaccggctttcaagaagattttttcaaagcctttcagttctttcaccggacgtgttacactgcctataagatcgcggatatatagctgcactacttccgaaccgtcatacttacctgtattggttaccgttacagttgccataatctctccattgatattcatggacgatttatccaatgtaatatcactataagagaaagtagtatagctcaagccatatccaaacggataaagcggttcgttatctacatccagataattgctacggaacttctggaaccaggccccttgaggcaaaggacgaccagtattcttatggttatagaacaaaggaatctgtcctacactcttcggaaaagtagtagtaagtttgccactcggatttacatttccaaacagtacatcaccaatggcaagagcagcttcactaccaccaaaccacacattcagaatagcaggtacattttcctgctcccaattcaatactaacggacgaccggtaaacaacaccaataccaccggtttgccggttttcaacaattcctgcaaaagtacacgttgcgtatccggcatttcgaggtctgtacggcaactactttcaccgctcatctcggaagactcacccaaagcagcaacaatcacgtcagacttggcagctacagcaagcgcctcatccagcagttccttatctgtacgattgtcacgatgcagagtacggccaaacatagtagcacgttcttcgtattcggcatcactcatcagattacttcctttagccgtaagaattttagccttgcctcccactacttctttcaagccttcaatcaaagaaggatatttgttcatcacagcggccacactccacgtgcccggcatattgctacggctatcggccaaaggacctactacagcaatggtacctttctttgccagagggagtacactattctcattcttcaagagaacaaagctttccgaagctgtcttacgggctatagcgcggtgttcttttgtaaagatttgttttttaggacgggttatatcacaatatttatagggattatcaaaaagccccagcttatacttagcttccagaatacggcgacaagcagcatcaacagcctttactgaaacttttccttcttccagagattttttaagtgtgcttgtaaaagcatcgctcaccatatccatatcgacacctgcattcagagccagggctgcaactgtttgtgtatcacccataccatgatcggtcatttcagtgataccggtatagtccgtcacaacgaacccatcaaaattccactgcttacgaagtacatcggtcatcagccacttatttccggtagccggtacaccatccacttcattgaatgaagccatcacactacccacaccttcttccacggcagcctgataaggtaacatatattcgttgaacatacgttgatgactcatatccactgtattatagtcgcgtccggcttctgatgccccatataacgcaaagtgcttcacgcaagccataatttcatcattactactcatatctttcccttgataaccacgtaccatagcacgcgcaatctccgctcccaagaagggatcttcaccattcccttcggaaactcgtccccaacggggatcacgggaaacatccaccatcggactgaatgtccagcaaataccatcagcactggcttcgatagcagcaatacgtgcagattcttcaatagctgtcatgttccaggtacaggataatcccagaggaataggaaataccgtttcgtatccatgaattacatccataccaaataaaagaggaatacccagacgactttcttctactgcctgtttctgaacgtcacgaatacgctccacgcctttcaagttaaagagtccgcccacttcaccggcacggatacgcttagccacattactactcttggcttgtccggtggttatttcacccgtaacaggcaagttcaactggccgattttctcttccagagtcatcttcttcatcagatcatcaataaagcgatccatatcgacaggagatttcatctttcctgtgtgattttcaataaatactatttacataccttaaattaatgtaataataaaaaccaacaccaatttggtatttttgcatactaaaaaggtatataaaaccaattagggcgtataaatggaactaagacacctcagatattttgtgaccgtggttgaagagcaaagcatttccaaagctgctgaaaagttgtgtattgcccagccgcccctcagccgacaaattcaaaaactcgaagaagaattgggaattcagctatttgaacgcggcttcagaccggctaaagtgactgaagcaggcatgtttttttatcagcatgctgtgcagattttgactcatactgcacaagcgtcctcaatggcaaaacggattgcaacggtcagtcaaaccttgagaattggttacgtcagctccttactgtatggtttgttacctgaaattatttatctgtttcgtcaacaaaatcctgaaattcacatcgaactcatcgaatgcggcaccaaagatcaaattaatgcccttaagcagggaaaaatcgatctgggttttggtcggctcaaaattaccgatcctgcaattcgacgtatcgtgttgcataaagaacagctcaaacttgcaatccataagcatcatcacctcaatcagtttgcagcaacaggggttcatctctctcaaattattgatgaaccgatgctgctgtacccagtctctcaaaagcccaattttgcgacctttattcagtcactctttaccgaactaggcctagtaccatccaaactcaccgaaattcgagaaattcaactggcactcggcttggtggcagcaggtgaaggcgtctgcatcgtaccggcgtctgccatggatattggggtgaagaatctactttatattccaattttagatgatgatgcctatagcccaatttcactcgcggtgcgaaatatggaccacagtaattacattcctaaaattctcgcctgtgtacaggaggtgtttgcaacgcaccatatcaggccactcatcgaataacgatctcgatcccgcgaaattaatacgactcactataggggaattgtgagcggataacaattcccctctagaaataattttgtttaactttaagaaggagatatacatatgcaccatcatcatcatcattcttctggtgtagatctgtggtctcatccgcagttcgaaaagggtaccgagaacctgtacttccaatccaatgccatgaccgtgaaaatttcccacactgccgacattcaagccttcttcaaccgggtagctggcctggaccatgccgaaggaaacccgcgcttcaagcagatcattctgcgcgtgctgcaagacaccgcccgcctgatcgaagacctggagattaccgaggacgagttctggcacgccgtcgactacctcaaccgcctgggcggccgtaacgaggcaggcctgctggctgctggcctgggtatcgagcacttcctcgacctgctgcaggatgccaaggatgccgaagccggccttggcggcggcaccccgcgcaccatcgaaggcccgttgtacgttgccggggcgccgctggcccagggcgaagcgcgcatggacgacggcactgacccaggcgtggtgatgttccttcagggccaggtgttcgatgccgacggcaagccgttggccggtgccaccgtcgacctgtggcacgccaatacccagggcacctattcgtacttcgattcgacccagtccgagttcaacctgcgtcggcgtatcatcaccgatgccgagggccgctaccgcgcgcgctcgatcgtgccgtccgggtatggctgcgacccgcagggcccaacccaggaatgcctggacctgctcggccgccacggccagcgcccggcgcacgtgcacttcttcatctcggcaccggggcaccgccacctgaccacgcagatcaactttgctggcgacaagtacctgtgggacgactttgcctatgccacccgcgacgggctgatcggcgaactgcgttttgtcgaggatgcggcggcggcgcgcgaccgcggtgtgcaaggcgagcgctttgccgagctgtcattcgacttccgcttgcagggtgccaagtcgcctgacgccgaggcgcgaagccatcggccgcgggcgttgcaggagggctga

(19) United States
(12) Patent Application Publication
(10) Pub. No.: US 2026/0049366 A1
(43) Pub. Date: Feb. 19, 2026

(54) ENZYME-LINKED BIOSENSORS

(71) Applicant: UCHICAGO ARGONNE, LLC, Chicago, IL (US)

(72) Inventors: Gyorgy Babnigg, Countryside, IL (US); Jessica L. Johnson, Plainfield, IL (US); Andrzej Joachimiak, Bolingbrook, IL (US); Rosemarie Wilton, Elmhurst, IL (US); Philip D. Laible, Downers Grove, IL (US); Chase Akins, Marengo, IL (US)

(21) Appl. No.: 19/205,314

(22) Filed: May 12, 2025

Related U.S. Application Data
(60) Provisional application No. 63/670,559, filed on Jul. 12, 2024.

(51) Int. Cl. C12Q 1/6897; C12N 9/42; C12N 15/70

(52) U.S. Cl. CPC C12Q 1/6897; C12N 9/2445; C12N 15/70; C12Y 302/01021

(57) ABSTRACT

The disclosure provides a thermostable enzyme-linked biosensor expression cassette comprising a nucleic acid comprising a nucleotide sequence encoding a β-glucosidase reporter. The enzyme-linked biosensor expression cassette of the disclosure comprises a nucleic acid comprising a nucleotide sequence encoding (i) a transcription factor, (ii) a promoter, (iii) a terminator, and (iv) a cloning site for a gene of interest. The disclosure further provides novel variants of β-glucosidase that function as the reporter enzyme and exhibit superior properties (e.g., without limitation, pH stability and thermal stability) compared to existing β-glucosidase, providing improved biosensor expression cassettes.

### CROSS-REFERENCE TO RELATED APPLICATION

This application claims the benefit of priority to U.S. Provisional Application No. 63/670,559, filed Jul. 12, 2024, the disclosure of which is incorporated herein by reference in its entirety.

### GOVERNMENT SUPPORT CLAUSE

This invention was made with government support under GM115586, awarded by the National Institutes of Health (NIH), and DE-AC02-06CH1137, awarded by the Department of Energy. The government has certain rights in this invention.

### INCORPORATION BY REFERENCE OF ELECTRONICALLY SUBMITTED MATERIAL

This application contains, as a separate part of the disclosure, a Sequence Listing in computer-readable form (Filename: 21-080A_SeqListing.xml; Size: 70,593 bytes; Created: May 5, 2025) which is incorporated by reference herein in its entirety.

### FIELD

The disclosure relates to enzyme-linked biosensors, including biosensors comprising novel variants of β-glucosidase, and methods of their use.

### BACKGROUND

Up until now, transcription factor (TF)-based biosensors have often been used for metabolite detection, adaptive evolution, and metabolic flux control. For a TF-based biosensor, the transcriptional factor binds to its cognate ligand (effector molecule) and the TF-ligand complex binds to the operator region to enhance transcription (activation) or the binding of the cognate ligand (effector molecule) to the TF releases the TF from the operator (de-repression), making it accessible to the transcription machinery. Generally, a TF activates the expression of a reporter protein (e.g., GFP) in response to a target metabolite. The protein reporter for the TF-based biosensor is generally a fluorescent protein. Examples of fluorescent proteins used in TF-based biosensors include cyan fluorescent protein (CFP), yellow fluorescent protein (YFP), enhanced cyan fluorescent protein (eCFP), enhanced yellow fluorescent protein (eYFP), enhanced green fluorescent protein (eGFP), green fluorescent protein (GFP), tdTomato, Venus, and the like. In some aspects, the most commonly used fluorescent protein reporter for a TF-based biosensor is a green fluorescent protein (GFP). Such an FP-based biosensor is also commonly referred to as a “GFP-based biosensor.”

Although TF-based biosensors utilizing fluorescent protein (FP) reporters are capable of detecting a number of intracellular ligands in droplet-based microfluidic settings, alternative methods with enhanced signal-to-noise ratios are preferred for signal monitoring. Microfluidic screening methods are used in a wide variety of applications, such as for biomanufacturing new and/or improved enzymes to enable efficient and economical production of biofuels and bioproducts. Microfluidic screening methods, which contain very small reaction volumes and only a limited protein or a single cell is needed as input, are the widely used tool for biomanufacturing, but they are still limited by their ability to read out whether a candidate has improved or not.

Further, as reaction volumes decrease and the need to understand subtle differences in the analyte concentrations increases, a more robust signaling system is needed.

Thus, there remains a need for new and improved biosensors that are stable, sensitive, and applicable for detecting analytes in biomanufacturing as well as for monitoring analytes in microfluidic settings.

### SUMMARY

The disclosure provides enzyme-linked biosensors, including biosensors comprising novel variants of β-glucosidase, and methods of their use.

In one embodiment, the disclosure provides a thermostable enzyme-linked biosensor expression cassette comprising a nucleic acid comprising a nucleotide sequence encoding a p glucosidase reporter, wherein the nucleotide sequence comprises at least 80%, at least 85%, at least 90%, or at least 95% sequence identity to the nucleotide sequence of any one of SEQ ID NOs: 1-18. In some aspects, the nucleic acid further comprises a nucleotide sequence encoding (i) a transcription factor, (ii) a promoter, (iii) a terminator, and (iv) a cloning site for a gene of interest. In some aspects, the cloning site for the gene of interest is between the promoter and the terminator. In some aspects, the gene of interest is a nucleotide sequence encoding a product, an analyte, or an enzyme. In some aspects, the enzyme produces a product or analyte that binds the transcription factor and activates transcription of the reporter, producing a signal proportional to the product. In some aspects, the transcription factor is a CatM transcription factor. In some aspects, the nucleotide sequence encoding the CatM transcription factor comprises at least 80%, at least 85%, at least 90%, or at least 95% sequence identity to the nucleotide sequence of SEQ ID NO: 19. In some aspects, the nucleotide sequence encoding the CatM transcription factor comprises the nucleotide sequence of SEQ ID NO: 19. In some aspects, the promoter is a T7 promoter. In some aspects, the nucleotide sequence encoding the T7 promoter comprises at least 80%, at least 85%, at least 90%, or at least 95% sequence identity to the nucleotide sequence of SEQ ID NO: 20. In some aspects, the nucleotide sequence encoding the T7 promoter comprises the nucleotide sequence of SEQ ID NO: 20. In some aspects, the promoter is a CatM promoter. In some aspects, the nucleotide sequence encoding the CatM promoter comprises at least 80%, at least 85%, at least 90%, or at least 95% sequence identity to the nucleotide sequence of SEQ ID NO: 21. In some aspects, the nucleotide sequence encoding the CatM promoter comprises the nucleotide sequence of SEQ ID NO: 21. In some aspects, the terminator is a T7 terminator. In some aspects, the nucleotide sequence encoding the T7 terminator comprises at least 80%, at least 85%, at least 90%, or at least 95% sequence identity to the nucleotide sequence of SEQ ID NO: 22. In some aspects, the nucleotide sequence encoding the T7 terminator comprises the nucleotide sequence of SEQ ID NO: 22. In some aspects, the nucleotide sequence comprises at least 80%, at least 85%, at least 90%, or at least 95% sequence identity to the nucleotide sequence of SEQ ID NO: 23. In some aspects, the nucleotide sequence comprises at least 80%, at least 85%, at least 90%, or at least 95% sequence identity to the nucleotide sequence of SEQ ID NO: 24.

The disclosure also provides a vector comprising a thermostable enzyme-linked biosensor expression cassette comprising a nucleic acid comprising a nucleotide sequence encoding a β glucosidase reporter, wherein the nucleotide sequence comprises at least 80%, at least 85%, at least 90%, or at least 95% sequence identity to the nucleotide sequence of any one of SEQ ID NOs: 1-18. In some aspects, the nucleic acid further comprises a nucleotide sequence encoding (i) a transcription factor, (ii) a promoter, (iii) a terminator, and (iv) a cloning site for a gene of interest. In some aspects, the cloning site for the gene of interest is between the promoter and the terminator. In some aspects, the gene of interest is a nucleotide sequence encoding a product, an analyte, or an enzyme. In some aspects, the enzyme produces a product or analyte that binds the transcription factor and activates transcription of the reporter, producing a signal proportional to the product. In some aspects, the transcription factor is a CatM transcription factor. In some aspects, the nucleotide sequence encoding the CatM transcription factor comprises at least 80%, at least 85%, at least 90%, or at least 95% sequence identity to the nucleotide sequence of SEQ ID NO: 19. In some aspects, the nucleotide sequence encoding the CatM transcription factor comprises the nucleotide sequence of SEQ ID NO: 19. In some aspects, the promoter is a T7 promoter. In some aspects, the nucleotide sequence encoding the T7 promoter comprises at least 80%, at least 85%, at least 90%, or at least 95% sequence identity to the nucleotide sequence of SEQ ID NO: 20. In some aspects, the nucleotide sequence encoding the T7 promoter comprises the nucleotide sequence of SEQ ID NO: 20. In some aspects, the promoter is a CatM promoter. In some aspects, the nucleotide sequence encoding the CatM promoter comprises at least 80%, at least 85%, at least 90%, or at least 95% sequence identity to the nucleotide sequence of SEQ ID NO: 21. In some aspects, the nucleotide sequence encoding the CatM promoter comprises the nucleotide sequence of SEQ ID NO: 21. In some aspects, the terminator is a T7 terminator. In some aspects, the nucleotide sequence encoding the T7 terminator comprises at least 80%, at least 85%, at least 90%, or at least 95% sequence identity to the nucleotide sequence of SEQ ID NO: 22. In some aspects, the nucleotide sequence encoding the T7 terminator comprises the nucleotide sequence of SEQ ID NO: 22. In some aspects, the nucleotide sequence comprises at least 80%, at least 85%, at least 90%, or at least 95% sequence identity to the nucleotide sequence of SEQ ID NO: 23. In some aspects, the nucleotide sequence comprises at least 80%, at least 85%, at least 90%, or at least 95% sequence identity to the nucleotide sequence of SEQ ID NO: 24. In some aspects, the disclosure provides a composition comprising the vector and a diluent.

The disclosure further provides a host cell comprising a vector as disclosed herein. In some aspects, the host cell is any cell which, in various aspects, is cultured to express a product or analyte. In some aspects, the cell is a bacterial cell or a yeast cell. In some aspects, the cell is an Escherichia coli cell. In some aspects, the cell is an Escherichia coli BL21(DE3) cell. In some aspects, the disclosure provides a composition comprising such host cells and a diluent.

The disclosure provides a method of detecting the presence of a product or analyte in a sample comprising: (a) contacting the sample with the expression cassette of any one of claims 1-19 and a substrate of the reporter enzyme, and (b) detecting the expression of the reporter enzyme.

The disclosure provides a method of determining a concentration of a product or analyte present in a sample comprising: (a) contacting the sample with any of the expression cassettes disclosed herein; (b) detecting the expression of the reporter enzyme; (c) measuring the concentration of the reporter enzyme; and (d) comparing the concentration of the reporter enzyme to a control or standard to determine the concentration of the product or analyte present in the sample. In some aspects, the product or analyte is muconate. In some aspects, the expression cassette is in a vector. In some aspects, the vector is in a host cell.

Additional aspects and aspects of the presently disclosed expression cassette, vectors, host cells, compositions, and methods are provided below. All headings are simply for organization and are not intended to limit the disclosure in any manner. The content of any individual section may be equally applicable to all sections.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1C show a schematic representation of the transcription factor (TF)-based biosensors. The use of TF-based sensors in droplet-based microfluidic systems has been limited to fluorescent protein detection with limited sensitivities (signal-to-noise ˜1000). Increased dynamic and operational ranges have been obtained by the optimization or selection of transcription factor and promoter region pairs. TF-based biosensors have been used in cell sorting/cytometry screening methodologies when coupled to FP reporters; however, they are inefficient in a microfluidic setting/application, i.e., in monitoring signals in droplet-based microfluidic settings. FIG. 1A shows a TF-based biosensor in which the activation by a ligand causes activation of transcription. FIG. 1B shows a TF-based biosensor in which the ligand causes derepression of the transcription. FIG. 1C shows a TF-based biosensor in which a NOT gate is used when targeting a TF that is active in the apo form, which converts the cassette into a positive fluorescent protein response when the ligand is detected.

FIG. 2A depicts a schematic representation for constructing the enzyme-linked biosensor (or “enzyme-based biosensor”) of the disclosure (FIG. 2A) and an example of a mechanism of the biosensor of the disclosure (FIG. 2B). The native catM and promoter region were previously inserted into the pBTL-2 vector and optimized for Pseudomonas putida response. The transcription factor (TF) and promoter region were transferred along with a β-glucosidase reporter and cloned into the pMCSG68 vector. In order to increase the sensitivity of product detection and operational range in a picoliter setting, a β-glucosidase enzyme has been inserted into the biosensor. Thus, this enzyme-linked reporter provides a much greatly enhanced signal and a much more stable biosensor. Moreover, the construct and/or the biosensor, as described herein, allow for a significantly enhanced signal and a significantly more stable biosensor compared to a counterpart without the addition of the enzyme-linked reporter. Additionally, in some aspects, the new biosensor can accept genes for protein expression.

FIG. 2B depicts an example of a mechanism of the biosensor of the disclosure shown in FIG. 2A. In the example, a library of catA genes is tested for activities. The expression of the CatA enzymes is induced with a separate inducible promoter (T7) in the biosensor cell (e.g., E. coli BL21(DE3)), the enzyme is produced within hours, the substrate (catechol in this example) is added externally, taken up by the cells, converted into the product (for example, cis-cis-muconate in this example), which in turn detected by the product sensor circuit (TF and associated promoter region; catM and promoter region in this example). The expressed enzyme activity is proportional to the production of the reporter β-glucosidase enzyme and the observed fluorescent signal. The design can be easily adapted for the study of other systems by simply replacing the product sensor portion (labeled with a rectangle) sensing the given product produced by the cloned enzyme (Enzyme E1).

FIGS. 3A-3H depict experimental results comparing the sensitivity of product detection and operational range between the enzyme-linked biosensor of the disclosure and the traditional biosensor that uses a fluorescent protein (e.g., GFP) reporter. FIG. 3A shows the results from an experiment that compares the biosensor having the enzyme-linked reporter as provided in the disclosure and the traditional biosensor using GFP. The results show a significantly better detection by the enzyme-linked reporter (labeled enzyme-linked reporter) than by the GFP reporter (labeled as sfGFP reporter). FIG. 3B depicts a schematic representation of the major differences between the two biosensors. The enzyme-linked reporter construct of the disclosure has a gene for expressing β-glucosidase (top), whereas the traditional biosensor has a gene for expressing GFP (bottom). FIGS. 3C-3D compare the Relative Fluorescence Units (RFU) vs. Time between the biosensor with GFP (FIG. 3C) and biosensor with β-glucosidase (APC115086, the β-glucosidase variant with SEQ ID NO: 9.) (FIG. 3D). The results show that the biosensor with the β-glucosidase reporter has a slightly narrower dynamic range (e.g., without limitation, a linear detection between 4-14 mM for cis, cis-muconate compared to 2.5-20 mM for the sfGFP-based reporter), but the signal-to-noise measured is substantially higher favoring the β-glucosidase reporter (FIG. 3A). The signal amplification is due to the fact that even when similar number of sfGFP or β-glucosidase are produced in the presence of cis, cis-muconate, one enzyme (β-glucosidase) is capable of turning hundreds of substrate molecules into fluorescent products (FIG. 3E) versus measuring the production of the fluorescent sfGFP molecule via fluorescence (FIG. 3F). Since the enzyme-linked biosensor circuit is inserted into a vector (e.g., pMCSG68) that is normally used for protein expression in E. coli, the overall design enables for the screening of the activities of a given target enzyme with the following criteria: a) the substrate of the target enzyme can be introduced into E. coli cells, and b) the corresponding TF and promoter region is identified and cloned into the biosensor region (FIG. 3G and FIG. 3H). In the specific example in FIG. 3H, a library of catA genes is evaluated using this approach. The expression of the CatA enzymes is induced with a separate inducible promoter (e.g., a T7 promoter) in the biosensor cell (e.g., E. coli BL21(DE3)), the enzyme is produced within hours, the substrate (e.g., catechol) is added externally, taken up by the cells, converted into the product (e.g., cis, cis-muconate), which in turn detected by the product sensor circuit (e.g., TF and associated promoter region, e.g., catM and promoter region). The expressed enzyme activity is proportional to the production of the reporter β-glucosidase enzyme and the observed fluorescent signal. The design can be easily adapted for the study of other systems by simply replacing the product sensor portion (labeled with a rectangle) sensing the given product produced by the target enzyme (Enzyme E1). Almost all biomanufacturing applications rely on enzymes that have been optimized for increased performance (e.g., without limitation, activity, thermotolerance, chemical tolerance, etc.).

FIGS. 4A-4B show a schematic representation of the chemical reaction of the biosensor having the enzyme-linked reporter as provided in the disclosure (FIG. 4A) and the intensity of the fluorescence being detected when using the enzyme-linked reporter of the disclosure (FIG. 4B). FIG. 4A shows the detection of the expressed β-glucosidase by the addition of a lysis buffer to permeabilize the sensor cell such that the clear non-fluorescent substrate (fluorescein quenched by two glucose moieties), fluorescein di-β-D-glucopyranoside, can enter the cell and the glucose molecules cleaved off by the β-glucosidase reporter enzyme to release the highly fluorescent fluorescein product. FIG. 4B shows the reporter activity when the sensor cells are incubated with cis, cis-muconate for more than 4 hours, followed by the addition of a lysis buffer and the di-β-D-glucopyranoside substrate. The release of fluorescein is monitored via a fluorescent plate reader. While the signal developed after one hour is sufficient to calculate the cis, cis-muconate concentration in the solution, the figure shows that the signal develops over time linearly, producing even more intense fluorescence.

FIGS. 5A-5B illustrate the difference between the signals that are recovered from the fluorescent protein (FP)-based biosensor (FIG. 5A) and the enzyme-linked biosensor of the disclosure (FIG. 5B). Equivalent microfluidic test volumes were used between the FP-based biosensor that is linked to GFP fluorescent protein (FIG. 5A) and the enzyme-linked biosensor of the disclosure that uses β-glucosidase as a reporter for gene expression (FIG. 5B). FIG. 5 shows that the signals recovered from equivalent microfluidic test volumes for the enzyme-linked biosensor of the disclosure that uses β-glucosidase as a reporter for gene expression (FIG. 5B) are about 10-1000-fold higher in fluorescence intensity than the signals recovered from the traditional FP-based biosensor that is linked to GFP fluorescent protein (FIG. 5A). Fluorescent signal is observed where the sensor cells are located in the microfluidic droplet (FIG. 5A), while the fluorescent product generated by the enzyme-linked biosensor after the addition of a lysis buffer and the clear substrate occupies the entire droplet with more intense fluorescent signal, providing a signal with higher signal-to-noise ratio required for downstream droplet manipulations (e.g., droplet sorting to identify droplets with the highest cis, cis-muconate concentration). Bar scale=10 μm.

FIG. 6 shows the application of cell-based enzyme-linked biosensors of the disclosure. FIG. 6 shows the application of the FP-based biosensor (cis, cis-muconate sensor) when inserted into the engineered Pseudomonas putida cells producing the product of interest (cis, cis-muconate, CCM). The cells are secreting CCM into the media. The sensor is activated only by the CCM produced inside the cell since P. putida cannot take up external CCM. This is in contrast to the E. coli-based sensor shown in FIGS. 7A-7C, where the E. coli enzyme-based sensor is deployed to measure CCM produced by the engineered P. putida cells. E. coli can take up the CCM present in the media and activate the enzyme-based biosensor. After a 4-hour incubation, the produced reporter is measured by the addition of lysis buffer and clear substrate to produce the fluorescent product occupying the entire well content.

FIGS. 7A-7C depict an application of the cell-based enzyme-linked biosensor in which the muconate (product or analyte) is sensed by a cell-based sensor with an enzyme (R-glucosidase) reporter. The biosensor cells are added to the medium and incubated, followed by the addition of a cocktail of lysis buffer and clear substrate. The addition of the low concentration of lysis buffer does not completely destroy the cells but permeabilizes them to allow the enzyme reporter substrate and the product to diffuse freely into and out of the cell. The amount of fluorescent product (fluorescein), as measured by the fluorescence, is proportional to the CCM concentration in the medium. FIG. 7A depicts Pseudomonas putida isolates in a 96-well plate (e.g., 96 different isolates or 30 isolates in triplicates, etc.), and the E. coli depicted are sensor cells in regular LB medium, in which the cells reach late exponential phase growth. The cells do not express the β-glucosidase under these conditions (no CCM added). FIG. 7B depicts two constructs in which the upper construct is displaying the minimal biosensor design with the transcription factor (TF, catM for CCM sensing), a promoter region, and the β-glucosidase reporter gene in the arrangement normally found in transcription regulation circuits in bacteria. The bottom construct in FIG. 7B is a configuration where the biosensor can be coupled to the evaluation of a library of enzyme variants that produce the said product sensed by the sensor using the pMCSG68 plasmid. FIG. 7C depicts the mechanism of the E. coli cell-based CCM sensor. E. coli sensor cells are added to the media containing CCM (bioproduct). The cell can take up the muconate (panel 1), which can bind to the TF (catM) and activate the transcription of the reporter enzyme (β-glucosidase). The cells are incubated for at least 4 hours (panel 2, panel 3). The sensor cells will be dividing during this time, leading to possible differing levels of enzyme reporter on the cell-to-cell basis (panel 4). The differences are averaged out when the lysis buffer and substrate are added, resulting in the conversion of the clear substrate into a fluorescent product.

FIG. 8A depicts the characterization of a cell-based biosensor where the pBTL2_catM_GFP sensor was transformed into E. coli cells and the response to cis, cis-muconate (CCM) in the extracellular medium was measured to evaluate the effects of the increase in CCM concentration on the ability of the bacterial cell to produce a green fluorescent protein (GFP). An E. coli cell that had been engineered into a cell-based biosensor takes up extracellular cis, cis-muconate (CCM) from the broth, which leads the cell-based biosensor to express either a fluorescent protein (if engineered as a TF-based biosensor that is linked to a fluorescent protein) or a reporter enzyme (if engineered as an enzyme-linked reporter) that is proportional with the extracellular CCM concentration. The performance of pBTL2_CatM_C21 (green fluorescent protein reporter) and pBATS_0004 (enzyme-linked reporter) sensors were compared after transforming the respective constructs into E. coli cells. The results in FIG. 8A shows that a linear response was observed when CCM concentration and the amount of GFP produced were compared. The results for the pBATS_0004 (enzyme-linked reporter) sensor were not shown in FIG. 8A.

FIG. 8B shows a comparison of the traditional fluorescent protein-based (e.g., GFP-based) biosensor and the enzyme-linked biosensor of the disclosure. The experiment is set up as follows: sensor cells (a: E. coli with GFP reporter or b: E. coli with β-glucosidase reporter) are incubated with different concentrations of CCM for at least 4 hours (overnight is more convenient for the sfGFP sensor to get a good signal). The evolution of sfGFP production is measured for the sfGFP reporter by correlating the slope observed in FIG. 8A versus CCM concentration. For the β-glucosidase reporter, the P. putida culture (with CCM in the medium) is mixed with the E. coli enzyme reporter, incubated for at least 4 hours, and a cocktail of lysis buffer and clear substrate is added. As the clear substrate is converted into the fluorescent product (fluorescein), the fluorescence increases over time. The slope of this fluorescence evolution is correlated with CCM concentration to get the slope vs. CCM graph. The pBTL2_catM_GFP and pBATS_0004 sensors were transformed into E. coli cells and their responses to the extracellular CCM were quantified for either GFP or β-glucosidase production, respectively. The results show that the cell-based enzyme-linked biosensor (i.e., cells transformed with pBATS_0004) produced significantly stronger signals for detection compared to those the weaker signals produced by the traditional TF-cell-based biosensor that is linked to a GFP protein (i.e., cells transformed with pBTL2_catM_GFP). The figure also shows the sensor response in the presence of different glucose concentrations in the media, mimicking bioreactor conditions where a mixture of glucose and CCM might be present. The sensor cells were not affected by varying glucose concentrations in the medium.

FIGS. 9A-9B depict a workflow of conducting a screening experiment of P. putida isolates for CCM production using the whole cell-based enzyme-linked biosensor of the disclosure. FIG. 9A depicts a workflow of using a 96-deep-well plate to grow P. putida cells to produce CCM. At the end of the production phase, the OD₆₀₀ is measured to gauge cell densities. The muconate concentration is measured next with a whole-cell-based biosensor (E. coli-based). FIG. 9B shows a culture of E. coli cells carrying the pBATS_0004 sensors incubated with engineered P. putida broth overnight to induce the reporter enzyme production. The enzyme level is measured in a functional assay in which the lysis buffer and substrates are added, and the fluorescence is monitored.

FIG. 10 shows the application of the enzyme-based reporter for the detection of products in a microfluidic setting. The level of CCM in the droplet produced by the engineered P. putida is detected by picoinjection of the E. coli enzyme-based reporter biosensor. The CCM is taken up by the E. coli cells, and β-glucosidase is produced proportional to the CCM level. After at least 4 hours of incubation, there are two ways to initiate the β-glucosidase detection (reporter readout): a) by picoinjection of the substrate and lysis buffer, or b) the substrate is already present, and the E. coli cells are permeabilized by external stimuli to make the substrate accessible to the enzyme.

FIG. 11 depicts a list of gut microbiota that encode a large repertoire of Carbohydrate-Active enZymes (CAZymes). Several gut microorganisms were cultivated to extract genomic DNA for the cloning, expression, and characterization of exo-acting CAZymes (mostly β-glucosidases and p-galactosidases). The table lists the gut microbes used for the selection of CAZymes. Cellulose is the most abundant biopolymer on Earth, produced mostly by plants via photosynthesis and by microorganisms via alternative pathways. The long chains of cellulose are broken down via enzymes acting on non-reducing and reducing ends of the polymer or endo-acting enzymes, cleaving the long chain into smaller products. The gut microbes use cellulose and other sugar polymers as carbon sources to convert them into energy while secreting short-chain fatty acids that are important to gut health. Since these gut microbes solely rely on the degradation of complex polysaccharides, their genome encodes for a plethora of CAZymes, providing an opportunity to identify enzymes with superior activities.

FIG. 12 depicts the structure of one of the β-glucosidases. The functional protein is a homodimer where residues from both subunits contribute to the enzyme activity. This figure shows how the terminal glucosyl moiety of a maltose molecule fits into the active site of the β-glucosidase protein.

FIG. 13 depicts a list of β-glucosidase orthologs and a survey of β-glucosidase activities. Some gut microbes have many enzymes in their genomes that are annotated as β-glucosidases. The orthologs were cloned, expressed, purified, and characterized. A wide range of activities were identified, suggesting that the different orthologs have preferences for slightly different terminal sugars.

FIGS. 14A-14D depict various characteristics of two β-glucosidase variants. Two candidate β-glucosidases, APC115045 and APC115086, from a collection of more than 40 enzymes were selected for further analysis. The enzymes were tested for melting temperature (FIG. 14A), activity profile over various temperatures, e.g., a temperature optimum (FIG. 14B), activity profile over various pH conditions, e.g., a pH optimum (FIG. 14C), and for compatibility in a microfluidic setting (FIG. 14D). FIG. 14A shows the two β-glucosidases tested for melting temperature, indicating the relative stability of the proteins. FIG. 14B shows the two β-glucosidases tested for temperature optimum. FIG. 14B shows the activity profile of the leading candidate of β-glucosidase variants, APC115045. Aliquots of APC115045 were incubated in various temperatures ranging from 23° C. to 50° C. for 5 min, followed by cooling down and an analysis of their relative activities. The results show that the enzyme retains most of its activity up to 46° C. The enzyme is not particularly thermotolerant since gut microbes do not experience extreme temperatures, and enzymes are evolved to display maximum activities around the normal body temperature (37° C.). APC115045 is the β-glucosidase variant/ortholog with SEQ ID NO: 5. FIG. 14C shows the two β-glucosidases tested for pH optimum. FIG. 14C shows the activity profile of the leading candidate of β-glucosidase variants, APC115086. Aliquots of APC115086 were incubated at pH 5.5, pH 6.0, pH 6.5, pH 7.0, pH 7.5, pH 8.0, and pH 8.5 for 10 min, followed by an analysis of their relative activities under those pH conditions. The results show that the enzyme activity is maximal around a neutral pH, retaining at least 40% activity between pH 6 and pH 8, suggesting an adaptation to the human gut environment. APC115086 is the β-glucosidase variant/ortholog with SEQ ID NO: 9. FIG. 14D shows the two β-glucosidases tested for compatibility in a microfluidic setting. The β-glucosidases were tested for enzyme activities in a droplet microfluidic setting. This is an important aspect for microfluidic applications where the surfactants that are used to stabilize droplets might interfere with some enzyme activities. The β-glucosidase is active in droplets using cell-based and cell-free systems.

FIG. 15 depicts enzyme stability measurement of four variants of β-glucosidases with each being stored at −80° C., 4° C., and at room temperature for about 5 years. The results from the SDS-PAGE analysis show that the APC115045 and APC115086 enzymes remained stable for a significant amount of time at room temperature, confirming that these enzymes in the biosensor will be stable in a kit.

FIGS. 16A-16B depict the characterization of a Bacteroides intestinalis β-glucosidase. The structure of the APC115045 enzyme (FIG. 16A) was determined and used to design mutant libraries around the active site. The mutants were tested in microfluidic droplet assays (FIG. 16B). FIG. 16A shows a structure of the APC115045 variant of β-glucosidase. The active form is a homodimer with both subunits contributing to the activity (residues part of the active site). Residues in the vicinity of the active site were selected and mutated to alanine. FIG. 16B shows activity profiles of the wild-type and mutant enzymes. Most mutations destroyed activities as expected. The relative activities observed in the microfluidic droplets followed those observed in traditional plate-based assays.

FIG. 17 depicts a table of sugar hydrolase activities of selected β-glucosidase candidate enzymes. FIG. 17 shows comparisons of 19 candidates/variants/orthologs of β-glucosidase, each undergoing enzymatic assays using a panel of ten substrates. The numbers depict the relative activities. The results show that APC115045.102 and APC115086.102 performed well under p-Nitrophenyl-β-D-glucopyranoside. These enzymes did not show activities against fucopyranoside, glucuronide, galactopyranoside, and xylopyranoside substrates, while others showed preferences for some of these substrates. APC115045.102 and APC115086 did not cleave substrates efficiently when a hydroxyl group was in close proximity to the cleavage site. These results suggest that APC115045.102 and APC115086 are specific β-glucosidases and may be good candidates for reporter development. APC115045.102 is the β-glucosidase variant/ortholog with SEQ ID NO: 5. APC115086.102 is the β-glucosidase variant/ortholog with SEQ ID NO: 9.

FIG. 18 provides a table of enzyme kinetics results from two β-glucosidase variants, i.e., APC115045.102 and APC115086.102. The APC115086.102 enzyme has a lower Km value, reaching half-max reaction velocity at lower substrate concentration than the APC115045.102 enzyme, albeit with lower catalytic efficiency (kcat). Overall, the two enzymes are very similar. For reporter development, the APC115086.102 enzyme was selected.

FIG. 19 shows a map of the β-glucosidase-based muconate sensor. The pMCSG68 protein expression vector, as described in Eschenfeldt et al., “New LIC vectors for production of proteins from genes containing rare codons.” Journal of Structural and Functional Genomics 14 (2013):135-144, was further modified to incorporate a sensor circuit. The plasmid carries an ampicillin resistance gene for selection and maintenance in E. coli. The vector was used for protein expression, where the DNA sequence encoding the protein of interest was inserted downstream of the ‘TEV-site’. Protein expression was initiated by high-level transcription of the T7-inducible system. RNA copies were generated from the T7-promoter to the T7-terminator region. A strong ribosomal binding site (RBS) ensured that the mRNA copies were efficiently used by the ribosomes to produce large quantities of the target protein. The disclosure introduced the sensor circuit into a ‘silent’ region of the vector upstream of the T7 promoter. The transcription of the reporter enzyme gene, therefore, was solely induced by the transcription factor binding to the upstream promoter region. Basal transcription of the reporter circuit was very low. In order to produce the enzyme efficiently, a relatively strong ribosome binding site was introduced upstream of the reporter enzyme gene. Due to the high copy number of this plasmid in E. coli, efficient expression of the reporter enzyme was possible. The nucleotide sequence of the β-glucosidase-based muconate biosensor construct is provided as SEQ ID NO: 24.

FIG. 20 shows a schematic map of the β-glucosidase-based muconate sensor along with the coding sequence of P. putida KT2440 CatA enzyme. The catA gene is in the protein expression region, i.e., a T7 promoter-driven, of the vector. It is hypothesized that the vector depicted herein, which has not yet been constructed, could be used for the engineering of CatA enzyme variants with higher efficiencies (higher catalytic rate (kcat), more optimal Km values, or a combination of the two) in the following manner. The vector would be introduced into E. coli BL21(DE3) host tailored for high-level protein expression. The protein expression (CatA expression) can be induced by the addition of IPTG to the media when cells are grown in the exponential phase. The IPTG induces the expression of T7 polymerase encoded on the E. coli BL21(DE3) genome. The elevated T7 polymerase expression, in turn, induces the transcription of the T7p-T7t portion of the plasmid, generating large copies of mRNA. The E. coli BL21(DE3) cells are ‘tricked’ and will allocate up to 40-50% of resources to producing the foreign protein (CatA in this case). This, in turn, would allow for a simple screening of enzyme activities of introduced variants. The reaction converting catechol to cis, cis-muconate can be followed by the addition of catechol. The catechol would enter the cells and get converted to muconate by the CatA enzyme. The novelty is in the notion that the plasmid encodes for an enzyme-linked muconate sensor. The muconate turns on the production of the reporter enzyme, i.e., β-glucosidase. A researcher could simply monitor the level of β-glucosidase via a fluorescent signal. It is envisaged that the cloning of 96 variants of CatA and their screening is performed in a 96-cell well plate format. The cells would be grown in the plate, the expression of CatA enzyme would be induced with IPTG when the cells reach a certain cell density (e.g., OD₆₀₀=0.4-0.6), the enzyme variants would be expressed within 4-16 h (based on induction temperature, i.e., 37-18° C., respectively). The enzyme substrate could be added to the cultures (e.g., enzyme substrate of catechol in the present example), and the level of produced enzyme reporter (after 4-16 h incubation) could be measured after the addition of fluorogenic substrate by monitoring the evolution of the fluorescent product.

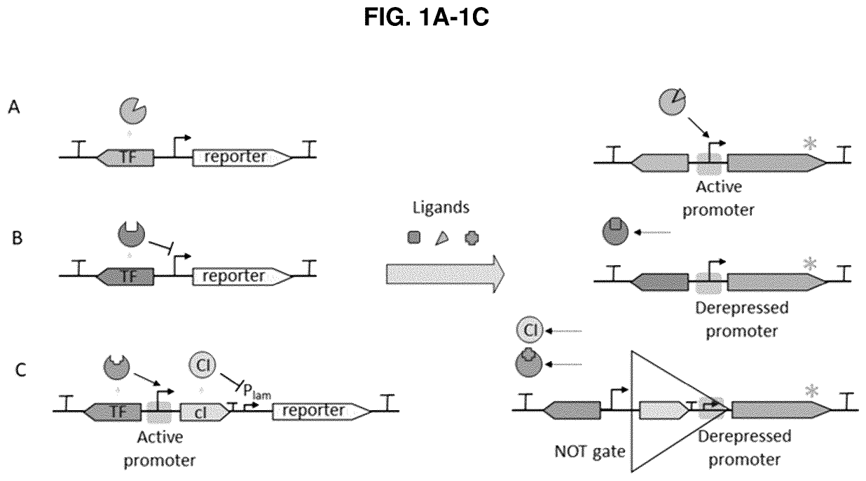

### DETAILED DESCRIPTION

The disclosure provides a thermostable enzyme-linked (alternatively known as an enzyme-based, cell-based enzyme-linked, or cell-based) biosensor or biosensor expression cassette comprising a nucleic acid comprising a nucleotide sequence encoding a β-glucosidase reporter, wherein the nucleotide sequence comprises at least 80%, at least 85%, at least 90%, or at least 95% sequence identity to the nucleotide sequence of any one of SEQ ID NOs: 1-18. The thermostable enzyme-linked biosensor or biosensor expression cassette of the disclosure further comprises a nucleic acid comprising a nucleotide sequence encoding (i) a transcription factor, (ii) a promoter, (iii) a terminator, and (iv) a cloning site for a gene of interest. The disclosure further provides novel components of the reporter enzyme, i.e., novel variants of β-glucosidase that exhibit superior properties (e.g., without limitation, more stable, more active, and/or exhibiting thermostability and pH tolerance relevant for sensor development) compared to commercially available β-glucosidase.

The disclosure is based, at least in part, on the discovery that the substitution of the fluorescent protein (e.g., without limitation, a green fluorescent protein (GFP)) in a transcription factor (TF)-based biosensor with an enzyme that catabolizes a fluorescent substrate (e.g., without limitation, a β-glucosidase), converts the fluorescent protein (FP)-based biosensor into an enzyme-based biosensor, which produces signals that, when (a) recovered from an equivalent microfluidic test volume as that of the TF-based biosensor, are (b) up to 1000-fold higher in fluorescence intensity than those of the TF-based biosensor that is linked to GFP fluorescent protein. Thus, the disclosure provides a new enzyme-linked biosensor comprising β-glucosidase as the reporter enzyme.

Enzyme-linked reporters are commonly used in large-scale reactions for the detection of analytes (i.e., products or bioproducts) at low concentrations, such as those encountered in clinical tests (e.g., hormones in the blood). The most commonly used enzymes have been horse radish peroxidase, β-galactosidase, and alkaline phosphatase. Up until the instant disclosure, the use of β-glucosidase as a reporter for gene expression has not been used for the detection of analytes in biomanufacturing.

The disclosure aims to overcome the significant drawbacks of FP-based biosensors by providing an enzyme-linked biosensor in the form of an enzyme-linked biosensor expression cassette. The terms “enzyme-linked biosensor” and “cell-based enzyme-linked biosensor” are used interchangeably herein. Such biosensor produces signals that are, upon being recovered and quantified, significantly higher in fluorescence intensity compared to signals of an FP-based biosensor.

In some aspects, the signal from a sensor cell expressing an FP-based biosensor can be enhanced when the FP is replaced with an enzyme reporter. While the FP-signal is detected from cells occupying a small proportion of the droplet, enzyme reporting converts the entire droplet fluorescent. In addition, and in aspects, when an enzyme reporter is used, the signal is amplified since an enzyme molecule may turn over hundreds or more of substrate molecules into fluorescent product molecules, thereby, increasing the fluorescent signal by more than 100-fold.

### Expression Cassette

In some aspects, the disclosure provides a biosensor expression cassette. In some aspects, the biosensor expression cassette of the disclosure is an “enzyme-linked,” also referred to as an “enzyme-based,” biosensor expression cassette. In some aspects, the biosensor expression cassette of the disclosure is a “cell-based enzyme-linked,” also referred to as “cell-based” or “whole-cell,” biosensor expression cassette.

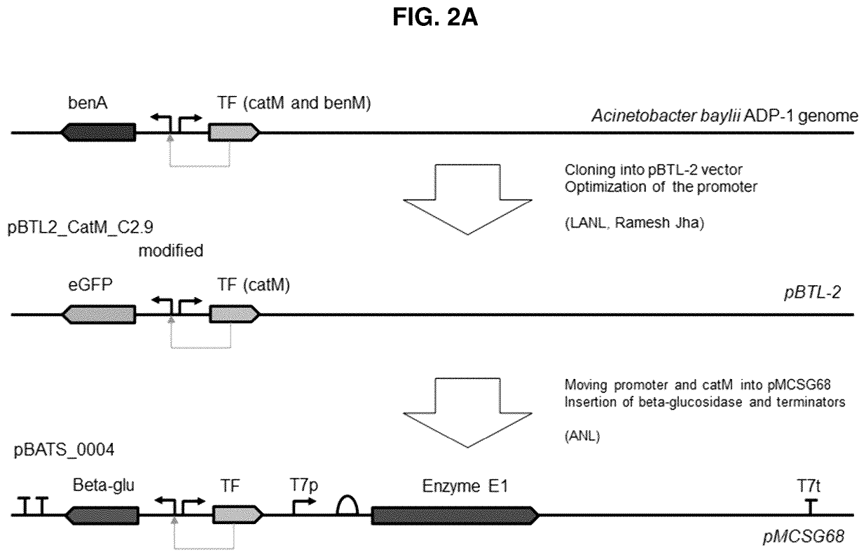

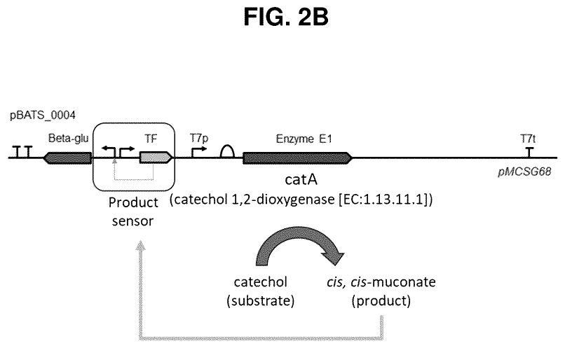

In other aspects, the disclosure provides a method of constructing a biosensor expression cassette of the disclosure. In some aspects, the disclosure provides a method of constructing an enzyme-linked biosensor expression cassette of the disclosure. In some aspects, the disclosure provides a method of constructing a cell-based enzyme-linked biosensor expression cassette of the disclosure. A schematic representation of a design and the steps utilized to construct a biosensor expression cassette of the disclosure is shown in FIG. 2A.

“Expression cassette” as used herein refers to a DNA sequence capable of directing expression of a particular nucleotide sequence in an appropriate host cell, comprising a promoter operably linked to the nucleotide sequence of interest, which is operably linked to termination signals (Papadakis et al. (2004). “Promoters and Control Elements: Designing Expression Cassettes for Gene Therapy”. Current Gene Therapy. 4(4): 89-113). The expression cassette can be incorporated into a plasmid, chromosome, mitochondrial DNA, plastid DNA, virus, or nucleic acid fragment. Typically, the expression cassette portion of an expression vector includes, among other sequences, a nucleic acid sequence in the coding region for encoding one or more genes to be transcribed (i.e., a target gene, i.e., encoding for a target protein) and the sequences controlling its expression (Vickers et al., “Dual gene expression cassette vectors with antibiotic selection markers for engineering in Saccharomyces cerevisiae.” Microbial Cell Factories 12 (2013):1-11). The phrases “target gene” or “gene of interest” and “target protein” or “protein of interest” may be used interchangeably herein throughout the disclosure when referring to the gene or nucleic acid sequence encoding the gene that would later be translated into the protein of interest.

In some aspects, the expression cassette comprises polynucleotide sequences that encode a purification tag, a protein cleavage site by Tobacco Etch Virus (TEV), and the gene of interest as disclosed herein.

In some aspects, the expression cassette of the disclosure comprises a nucleic acid comprising a nucleotide sequence encoding (i) a transcription factor, (ii) a promoter, (iii) a terminator, and (iv) a cloning site for a gene of interest.

In some aspects, the nucleotide sequence of the expression cassette of the disclosure further comprises at least one site for inserting one or more genes of interest. The site configured for inserting the target gene is any site allowing insertion of the target gene therein. For example, a target gene cloning site configured for inserting a target gene may comprise but is not necessarily limited to a multiple cloning site (MCS). In other words, in some aspects, the cassette of the biosensor of the disclosure comprises a target gene cloning site configured for inserting the target gene. This may be a multiple cloning site or any recognition site allowing integration of the target gene therein. Examples of target gene cloning sites are multiple cloning sites allowing integration of a gene after enzymatic digestion of the cloning site and recognition sites for an endonuclease such as a Zinc-finger nuclease or a TALEN or a CRISPR/Cas-derived system. The design of a target gene cloning site for integration of the target gene is known to the skilled person.

The addition of a site for inserting one or more genes of interest gives the reporter gene, which encodes the reporter enzyme provided in the expression cassette, great flexibility of use and can be adapted to various target genes and reporter genes. Cloning sites configured for inserting a gene can be used with many different genes (e.g., by adapting the sequence of these genes).

A multiple cloning site (MCS), also called a polylinker, is a short segment of DNA which contains many (up to ˜20) restriction endonuclease sites (also referred to as restriction sites) and is a standard feature of engineered plasmids. Thus, in some aspects, the target gene cloning site and/or the reporter gene cloning site includes a multiple cloning site. In some aspects, any of the cloning sites of the disclosure may be but are not necessarily limited to, a multiple cloning site.

Another cloning strategy is ligation-independent cloning, also referred to as “LIC” or “LIC cloning” in the disclosure. LIC cloning is a form of molecular cloning that is able to be performed without the use of restriction endonucleases or DNA ligases. This allows genes that have restriction sites to be cloned without being limited by the presence/absence of specific restriction sites. Many strategies for ligation-independent cloning exist and are known in the art. In some aspects, the site for inserting a target gene includes but is not necessarily limited to, a ligation-independent cloning site. The bacterial vector pMCSG68, as described in the Examples section and utilized in the disclosure, comprises a ligation-independent cloning site. Nevertheless, the site can be designed using any strategy so long as the gene of interest can be inserted into the vector for expression.

The cloning sites of the disclosure may be a combination of cloning sites. Thus, in some aspects, the cloning sites of the disclosure are different from each other. In aspects, the cloning sites of the disclosure are a combination of multiple cloning sites and ligation-independent cloning sites.

In aspects, the expression cassette of the disclosure comprises a TEV. In aspects, TEV is a 7 amino acid long peptide that is recognized and cleaved by TEV enzyme. In some aspects, the TEV enzyme cleaves off the purification tag from the target protein when destined for biochemical, biophysical, and structural biology studies.

In some aspects, the disclosure provides a complex cassette from the generic protein expression plasmid (pMCSG68—from T7p to T7t) by inserting the sensor cassette upstream of the T7 promoter (T7p). In some aspects, the expression cassette of the disclosure is set up to enable the expression of one or more enzymes (e.g., without limitation, an enzyme or a library of enzymes, e.g., without limitation, ‘Enzyme 1’ as described and depicted herein) as well as sense (detect) the product of a reaction, coupling enzyme discovery and detection in a single plasmid. In some aspects, the disclosure provides an option to monitor the activity of both a single enzyme (e.g., without limitation, Enzyme 1) as well as a small pathway (e.g., without limitation, Enzyme 1, 2, 3 . . . n).

In some aspects, the expression cassette of the disclosure comprises a cloning site for the gene of interest that is between the promoter and the terminator. In some aspects, the expression cassette of the disclosure comprises a cloning site for the gene of interest that is between the T7 promoter and the T7 terminator. In some aspects, the expression cassette of the disclosure utilizes the canonical T7 terminator. In some aspects, said T7 terminator may be commonly used for protein expression vectors where protein expression may be regulated at the mRNA (message) level by an inducible T7 RNA polymerase.

In some aspects, the expression cassette of the disclosure comprises a gene of interest. In some aspects, the expression cassette of the disclosure comprises a gene of interest, which is an enzyme.

In some aspects, the expression cassette of the disclosure may be capable of screening candidate enzymes (e.g., without limitation, CatA, catechol 1,2-dioxygenase [EC:1.13.11.1]). In some aspects, such screening of the candidate enzymes may be the last step in CCM production.

In some aspects, employing the expression cassette of the disclosure and such cassette in E. coli in such capability may be possible for screening CatA variants with a direct readout of enzyme activity. For clarity, variants of the catA genes may be inserted into the expression cassette; cells may be fed with catechol; the expressed CatA enzymes may convert catechol (1,2-Benzenediol) to muconate; muconate may be sensed by its cognate transcription factor (CatM) and drive the expression of glucosidase via binding to an upstream promoter. In some aspects, the glucosidase activity may be measured as a proxy of CatA activity without the need for analytics. In some aspects, such a method as described herein may be scaled to HTP since single-cell measurements are possible using microfluidics.

### Vector

In aspects, the disclosure provides a vector. In some aspects, the vector comprises the biosensor expression cassette of the disclosure. The expression cassette of the disclosure was assembled into the vector pMCSG68. pMCSG68 is a known compact bacterial vector encoding tRNA genes for rare Arg and Ile codons, with a 6×His-Strep-Tag II-TEV commonly used for high-throughput purification of recombinant proteins. Any number of other vectors may be used as is known to those persons skilled in the art. However, for instance, if the selected host cell is a bacterial cell, then a suitable bacterial vector may be optimal for expression and toxicity factors.

In some aspects, the vector of the disclosure can be used to construct the biosensor of the present disclosure. In aspects, the vector of the disclosure can be used to construct the enzyme-linked biosensor of the present disclosure. In aspects, the vector of the disclosure can be used to construct the cell-based enzyme-linked biosensor of the present disclosure.

It should be noted that the various aspects described in the disclosure can be applicable to another aspect. For instance, the aspects described for an enzyme-linked biosensor are also applicable to a cell-based enzyme-linked biosensor. Similarly, aspects described for an expression cassette are also applicable to the biosensor (i.e., both the enzyme-linked biosensor and the cell-based enzyme-linked biosensor). Further, the aspects described in relation to a β-glucosidase and, for instance, an enzyme-linked biosensor are also applicable to a cell-based enzyme-linked biosensor.

### Enzyme-Linked Biosensor

As mentioned above, the disclosure provides an enzyme-linked biosensor. Moreover, as mentioned above, the disclosure provides a method of constructing an enzyme-linked biosensor of the disclosure.

Biosensors are biological devices combining two essential components: a sensing component that detects a particular input, typically the presence of a chemical, a promoter region where the transcription factor binds, and a reporter that produces a measurable output after receiving the signal transduced by the sensing component (Fernandez-López et al., “Transcription factor-based biosensors enlightened by the analyte.” Frontiers in Microbiology 6 (2015):135038).

Biosensors have been used extensively in synthetic biology and metabolic engineering to easily measure bioproduct formation or changes in the environment. Many different types of biosensors have been developed and characterized, including aptamers, riboswitches, fluorescence resonance energy transfer (FRET)-based sensors, and transcription factor (TF)-based biosensors. TF-based biosensors are the most widely used due to their ease of use and their wide range of applications (Zhou et al., “Applications and tuning strategies for transcription factor-based metabolite biosensors.” Biosensors 13.4 (2023):428). A genetically encoded transcription factor driving the expression of a fluorescent protein reporter is especially useful in biomanufacturing and clinical applications, for instance, to get rapid readout of bioproduct formation. They can readily detect product formation in an in vitro assay or inside the cell. Thus TF-based biosensors are widely used for the detection of metabolites and the regulation of cellular pathways in response to metabolites. An intracellular biosensor enables the high-throughput screening of variants of producers and the signal used for isolating the best candidates (Tellechea-Luzardo et al., “Transcription factor-based biosensors for screening and dynamic regulation.” Frontiers in Bioengineering and Biotechnology 11 (2023):1118702; “Tellechea-Luzardo”).

Enzymes are common biocatalysts that are efficient at increasing the biological reaction rate. The working principle of an enzyme-based biosensor depends on the catalytic reaction and binding capabilities for the target analyte detection. Morrison et al., “Clinical applications of micro- and nanoscale biosensors.” Biomedical Nanostructures 1 (2008):433-458. Various possible mechanisms are involved in the analyte recognition process: (i) the analyte is metabolized by the enzyme, so the enzyme concentration is estimated by measuring the catalytic transformation of the analyte by the enzyme, (ii) an enzyme inhibited or activated by analyte, so the analyte concentration is related to decreased enzymatic product formation, and (iii) tracking of the alteration of enzyme characteristics. Justino et al., “Recent developments in recognition elements for chemical sensors and biosensors.” TrAC Trends in Analytical Chemistry 68 (2015):2-17. Owing to the long history of enzyme-based biosensors, various biosensors can be produced on the basis of enzyme specificity. However, the enzyme structure is extremely sensitive, which makes it expensive and complicated to improve its sensitivity, stability, and adaptability. Liu et al., “Advanced biomaterials for biosensor and theranostics.” Biomaterials in Translational Medicine. Academic Press, 2019 (pp. 213-255). Electrochemical transducers are most commonly used for enzyme-based biosensors. The most common enzyme-based biosensors are glucose and urea biosensors. In some aspects, enzyme-based biosensor is the most common class of biosensor.

In an enzyme-based biosensor, the enzyme is utilized as the recognition element and is immobilized on/within the support matrix on the transducer surface in order to maintain enzyme activity. The advantages of using enzymes, such as the high specificity of enzyme-substrate interactions and the high turnover rates of biocatalysts (i.e., the product of catalyst activity and lifetime), have made enzyme-based biosensors one of the most extensively studied areas. The sensing principle of the enzyme-based biosensor is to detect the presence of certain analytes by measuring changes such as proton concentration (H⁺), the release or uptake of gases (i.e., CO₂, NH₃, O₂, etc.), light emission, absorption, or reflectance, heat emission, and so forth, which occurs during substrate consumption or product formation of an enzymatic reaction. The transducer then converts those changes into measurable signals (electrical, optical, or thermal signals) that are used to identify analytes of interest, such as products and bioproducts (e.g., muconate or cis, cis-muconate (CCM)).

In various aspects, the disclosure provides an enzyme-linked biosensor and/or an enzyme-linked biosensor expression cassette. In some aspects, the enzyme-linked biosensor and/or the expression cassette comprises a nucleic acid comprising a nucleotide sequence encoding (i) a transcription factor, (ii) a promoter, (iii) a terminator, and (iv) a cloning site for a gene of interest.

In some aspects, the phrase “enzyme-linked biosensor” is also known as “enzyme-based biosensor,” “enzyme-based linked biosensor,” “enzymatic biosensor,” “biosensor having the enzyme-linked reporter,” and the like and phrases may be used interchangeably herein throughout the disclosure.

In some aspects, the enzyme-linked biosensor of the disclosure comprises a β-glucosidase enzyme reporter. In some aspects, the enzyme-linked biosensor of the disclosure utilizes β-glucosidase as a reporter for gene expression. In some aspects, the enzyme-linked reporter construct of the disclosure has a gene for expressing β-glucosidase.

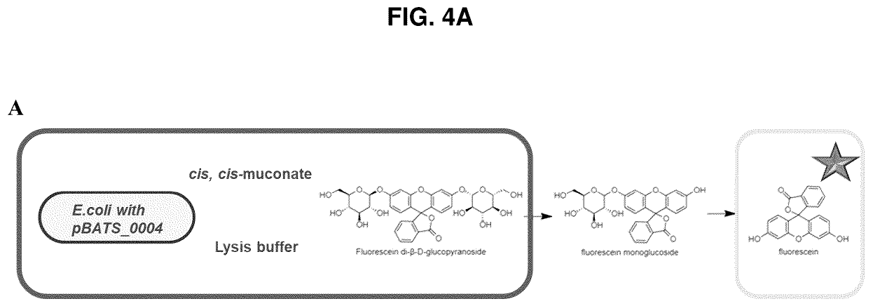

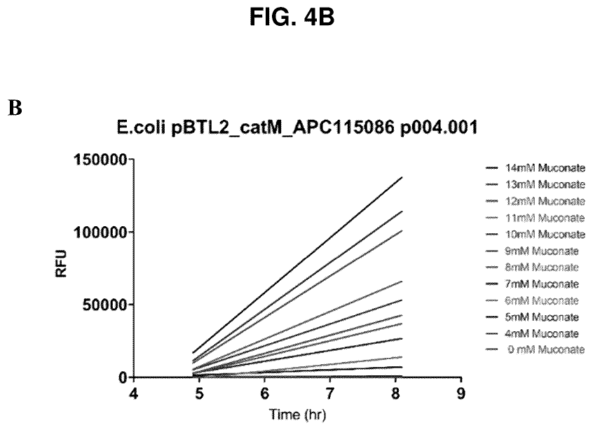

In some aspects, the enzyme-linked biosensor of the disclosure is an enzyme-linked muconate biosensor. In some aspects, an enzyme-linked muconate biosensor shows the conversion of clear substrate into fluorescent product (fluorescein). The results are depicted in FIG. 4B.

In some aspects, the disclosure provides a method for constructing an enzyme-linked biosensor of the disclosure. A schematic representation for constructing the enzyme-linked biosensor of the disclosure is shown in FIG. 2A.

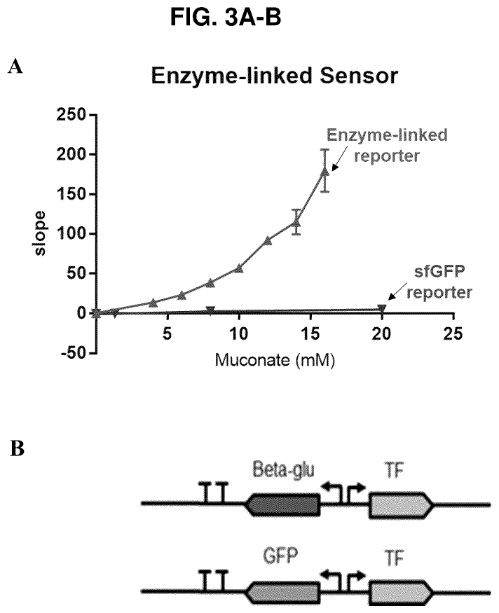

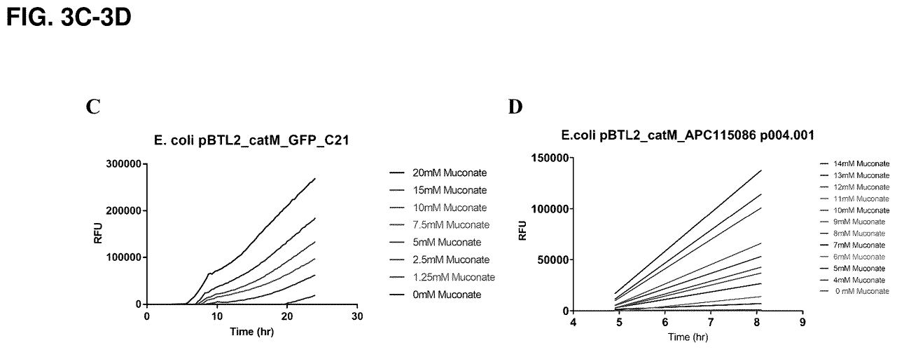

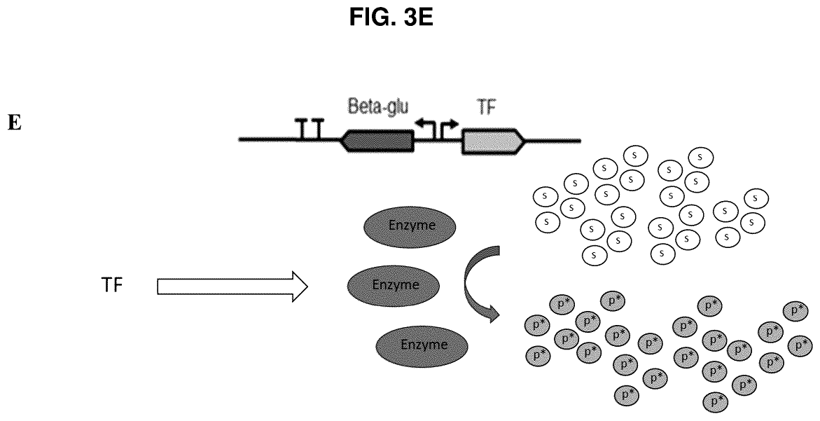

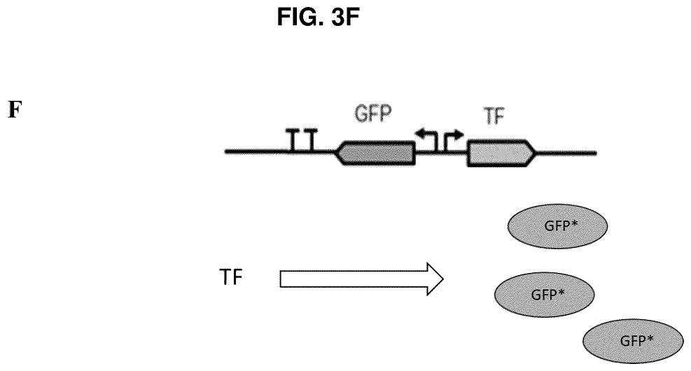

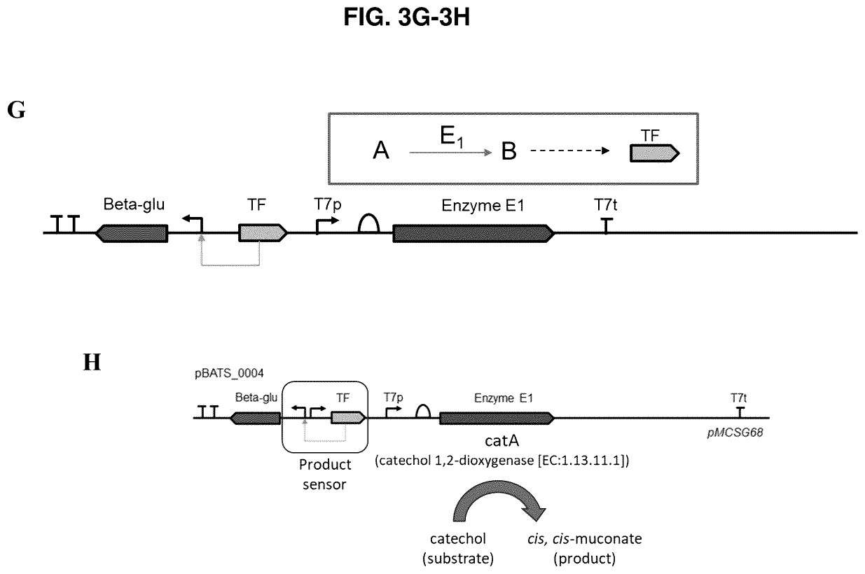

In some aspects, the disclosure provides an enzyme-linked reporter. In some aspects, the enzyme-linked reporter of the disclosure exhibits a significantly stronger signal for detection than a reporter enzyme GFP reporter. In some aspects, the enzyme-linked reporter of the disclosure exhibits a significantly stronger signal for detection than a GFP. See FIGS. 3A-3H.

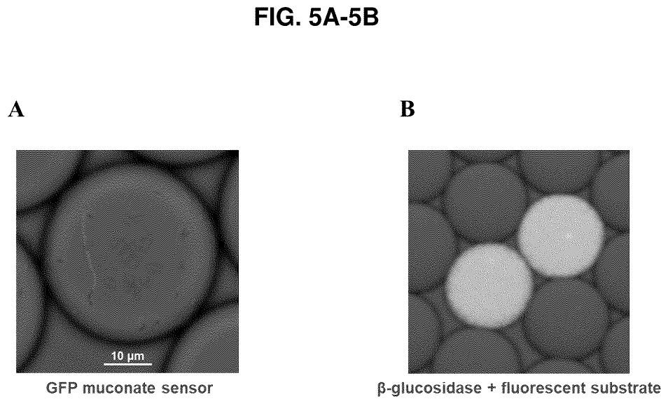

In some aspects, the signals that are recovered from equivalent microfluidic test volumes for the enzyme-linked biosensor of the disclosure that uses β-glucosidase as a reporter for gene expression are about 10-1000-fold higher in fluorescence intensity than those found with the traditional FP-based biosensor that is linked to GFP fluorescent protein. See FIGS. 5A-5B.

In various aspects, the disclosure provides an enzyme-linked biosensor. In some aspects, the disclosure provides an enzyme-linked biosensor that is more sensitive than fluorescent protein-based variants.

In various aspects, the enzyme-linked biosensor of the disclosure is stable, sensitive, and applicable to many assay types. In various aspects, the enzyme-linked biosensor of the disclosure works in a microfluidic setting.

In various aspects, the enzyme-linked biosensor of the disclosure replaces the fluorescent protein in the TF-based biosensor with an enzyme that catabolizes commercially available fluorescent substrates. In various aspects, the signals recovered from equivalent microfluidic test volumes are about 10-1000-fold higher in fluorescence intensity for the enzyme-linked biosensor of the disclosure than those found with fluorescent protein-based counterparts. The difference between the signals that are recovered from the traditional FP-based biosensor and the enzyme-linked biosensor of the disclosure is shown in FIGS. 5A-5B. In some aspects, β-glucosidase is capable of catabolizing commercially available fluorescent substrates.

In some aspects, the biosensor provided herein can be used in industry. In some aspects, the biosensor provided herein can be used in academia. In some aspects, the biosensor provided herein is broadly applicable in biomanufacturing. In some aspects, the biosensor provided herein is broadly applicable in new research driving the decarbonization of the economy. In some aspects, the biosensor provided herein has applications in propelling droplet-based microfluidic workflows.

In various aspects, the enzyme-linked biosensor of the disclosure can allow for the rapid screening of larger numbers of libraries of biocatalysts and/or more diverse sets of catabolic and metabolic processes. In some aspects, the enzyme-linked biosensor of the disclosure can increase opportunities to optimize metabolic throughput in strains designed to produce new, functionally superior biofuels and/or bioproducts. In some aspects, the design of the enzyme-linked biosensor presented herein will propel droplet-based microfluidic workflows.

In some aspects, the enzyme-linked biosensor of the disclosure is more sensitive than fluorescent protein-based biosensors. In some aspects, an enzyme-linked biosensor of the disclosure is about 10-1000-fold more sensitive than fluorescent protein-based biosensors.

In some aspects, the enzyme-linked biosensor of the disclosure is suited for high-throughput (HTP) applications. In some aspects, the assay provided in the disclosure can be easily automated and provides rapid monitoring of analytes with the corresponding transcription factor, operator, and promoter sequences driving the transcription of the enzyme reporter.

### Cell-Based Enzyme-Linked Biosensor

In some aspects, the disclosure further provides a host cell. In some aspects, the host cell comprises a biosensor (or “biosensor expression cassette”) of the disclosure. In various aspects, the terms “biosensor” and “biosensor expression cassette” are used interchangeably. In some aspects, the biosensor expression cassette is in a vector of the disclosure.

In some aspects, the host cell of the disclosure is derived from microbial cells such as bacteria, yeast, fungi, and algae. In some aspects, the host cell of the disclosure is derived from other higher eukaryotes, including fish, rat, and human cells. In the context of cell type selection for biosensing, microbial cells have largely been utilized in biosensors for water quality monitoring and toxicity assessment (Gao et al., “A double-mediator based whole-cell electrochemical biosensor for acute biotoxicity assessment of wastewater.” Talanta 167 (2017):208-216; Vopdlenskd et al., “New biosensor for detection of copper ions in water based on immobilized genetically modified yeast cells.” Biosensors and Bioelectronics 72 (2015):160-167; and Yang et al., “Fast and sensitive water quality assessment: a μL-scale microbial fuel cell-based biosensor integrated with an air-bubble trap and electrochemical sensing functionality.” Sensors and Actuators B: Chemical 226 (2016):191-195). On the other hand, higher eukaryotic cells have prominent applications in the study of basic cellular functions and disease pathogenesis (Gupta et al., “Cell-based biosensors: Recent trends, challenges, and future perspectives.” Biosensors and Bioelectronics 141 (2019):111435). Other cell types, including, but not limited to, those derived or isolated from specific diseases (e.g., small-cell lung cancer cells) or various stages of the cell lineages (e.g., cardiomyocytes), may also be utilized for biosensing applications (Hu et al., “High-performance beating pattern function of human induced pluripotent stem cell-derived cardiomyocyte-based biosensors for hERG inhibition recognition.” Biosensors and Bioelectronics 67 (2015):146-153).

Any cell type for expression of a product or analyte is contemplated, including mammalian cells (e.g., embryonic stem cells), bacterial cells (e.g., E. coli cells), yeast cells, and the like. In some aspects, the cell is a microbial cell or a bacterium. In some aspects, the bacterium is an Escherichia coli. In some aspects, the disclosure provides a host cell comprising the vector of the disclosure. In some aspects, the vector comprises the expression cassette of the disclosure. In some aspects, the host cell of the disclosure is an Escherichia coli. In some aspects, the host cell of the disclosure is an Escherichia coli BL21(DE3). E. coli BL21(DE3), a derivative of BL21, is probably the most widely used in high-level expression of recombinant proteins, and it harbors a prophage DE3 derived from a bacteriophage A, which carries the T7 RNA polymerase gene under the control of the lacUV5 promoter (Jeong et al., “Complete genome sequence of Escherichia coli strain BL21.” Genome Announcements 3.2 (2015):10-1128).

In some aspects, the cell-based enzyme-linked biosensors utilize microbes since they possess potential biorecognition elements in the construction of cell-based enzyme-linked biosensors. Examples of microbes for constructing cell-based enzyme-linked biosensors provided in the disclosure include, but are not limited to, bacteria, fungi (yeasts and molds), algae, protozoa, and viruses. They are self-replicating and can produce recognition elements, such as antibodies, without the need for extraction and purification. Gui et al., “The application of whole cell-based biosensors for use in environmental analysis and in medical diagnostics.” Sensors 17.7 (2017):1623. Compared with animal or plant cells, whole-cell-based biosensors are easy to handle and rapidly proliferating. The cells can interact with a wide variety of analytes, display the electrochemical response that a transducer can register, and transmit (whole-cell-based biosensor principle). Ron and Rishpon. “Electrochemical cell-based sensors.” Whole Cell Sensing Systems I: Reporter Cells and Devices (2010):77-84. Without wishing to be bound by any particular theory, it is believed that the good sensitivity, high selectivity, and capability of detection of these biosensors allow them to be successfully employed in environmental monitoring, food analysis, pharmacology, heavy metals, pesticides, detection of organic contaminants, and drug screening. Berepiki et al., “Development of high-performance whole cell biosensors aided by statistical modeling.” ACS Synthetic Biology 9.3 (2020):576-589. Cell-based enzyme-linked biosensors are further described in, e.g., Naresh and Lee. “A review on biosensors and recent development of nanostructured materials-enabled biosensors.” Sensors 21.4 (2021):1109 which is cell-based enzyme-linked-based biosensors.

In various aspects, the disclosure provides a cell-based enzyme-linked biosensor. In some aspects, the disclosure provides a whole-cell biosensor. As mentioned above and in other aspects, the disclosure provides a cell-based enzyme-linked biosensor. Also, as mentioned above and in other aspects, the disclosure provides a method of constructing a cell-based enzyme-linked biosensor of the disclosure.

In aspects, the phrase “cell-based enzyme-linked biosensor” is also known as “cell-based biosensor,” “whole-cell-based biosensor,” “whole-cell biosensor,” “cell-based biosensor (enzyme-linked)”, “whole cell-based enzyme-linked biosensor,” “microbial biosensor,” and the like and phrases may be used interchangeably herein throughout the disclosure.

In some aspects, the phrase “cell-based muconate biosensor (enzyme-linked)” is also known as “cell-based muconate enzyme-linked biosensor,” “cell-based enzyme-linked muconate biosensor,” and the like and phrases may be used interchangeably herein throughout the disclosure.

In some aspects, the disclosure provides a method for constructing a cell-based enzyme-linked biosensor of the disclosure.

In some aspects, the enzyme-linked biosensor and/or the expression cassette further comprises an antibiotic-resistance gene. In some aspects, the antibiotic resistance gene is necessary for maintaining the expression cassette in a plasmid in a microbial host cell.

In some aspects, the cell-based enzyme-linked biosensor of the disclosure is capable of the extracellular cis, cis-muconate (CCM) uptake from the broth, which leads the cell-based biosensor to express either a fluorescent protein (if engineered as a TF-based biosensor that is linked to a fluorescent protein) or a reporter enzyme (if engineered as an enzyme-linked reporter) that is proportional to the extracellular CCM concentration.

In some aspects, the high concentration of analyte in the medium does not affect the growth of the bacterial host cells. In some aspects, a high concentration of cis, cis-muconate (CCM) in the medium does not affect the growth of the bacterial host cells.

In some aspects, the CCM concentration and the amount of GFP produced are proportional. In some aspects, a linear response is observed when comparing the CCM concentration and the amount of GFP produced. In some aspects, the response to cis, cis-muconate (CCM) in the extracellular medium was measured to evaluate the effects of the increase in CCM concentration on bacterial cell growth. In some aspects, the response to cis, cis-muconate (CCM) in the extracellular medium was measured to evaluate the effects of the increase in CCM concentration on the ability of the bacterial cell to produce a green fluorescent protein (GFP).

In some aspects, the cell-based enzyme-linked biosensor of the disclosure (i.e., cells transformed with pBATS_0004) produces significantly stronger signals for detection compared to weaker signals produced by the traditional TF-cell-based biosensor that is linked to a GFP protein.

In some aspects, the cell-based enzyme-linked muconate biosensor of the disclosure is capable of screening evolved isolates for muconate.

In some aspects, the disclosure provides the construction of a cell-based muconate enzyme-linked biosensor.

In some aspects, the cell-based enzyme-linked biosensor provided in the disclosure is stable. In some aspects, the cell-based enzyme-linked biosensor provided in the disclosure is sensitive. In some aspects, the cell-based enzyme-linked biosensor provided in the disclosure is applicable to many assay types.

In some aspects, the cell-based enzyme-linked biosensor provided in the disclosure is capable of measuring a wide concentration range of analytes in production broth.

In some aspects, the cell-based enzyme-linked biosensor provided in the disclosure works well in a microfluidic setting. In some aspects, the cell-based enzyme-linked biosensor provided in the disclosure is highly sensitive. In some aspects, the cell-based enzyme-linked biosensor provided in the disclosure is a flexible cell-based biosensor.

In some aspects, the disclosure provides a cell-based enzyme-linked biosensor. In some aspects, the cell-based enzyme-linked biosensor of the disclosure is stable. In some aspects, the cell-based enzyme-linked biosensor of the disclosure is sensitive. In some aspects, the cell-based enzyme-linked biosensor of the disclosure is applicable for many assay types. In some aspects, the cell-based enzyme-linked biosensor of the disclosure is capable of measuring a wide concentration range of analyte. In some aspects, the cell-based enzyme-linked biosensor of the disclosure is capable of measuring a wide concentration range of analyte in production broth.

In some aspects, the cell-based enzyme-linked biosensor of the disclosure is capable of working well in a microfluidic setting.

In some aspects, the cell-based enzyme-linked biosensor of the disclosure is capable of working in high-throughput applications. In some aspects, the cell-based enzyme-linked biosensor of the disclosure is capable of working in high-throughput assays.

### Reporter Enzyme and B-Glucosidase

In some aspects, the expression cassette and/or biosensor of the disclosure comprises a reporter enzyme. In additional aspects, the expression cassette and/or biosensor of the disclosure comprises a reporter enzyme, wherein the reporter enzyme is capable of hydrolyzing a fluorescent substrate. Moreover, in some aspects, the reporter enzyme of the disclosure is a β-glucosidase.

Enzyme reporters with a range of colorimetric and fluorometric substrates. Colorimetric reporter enzymes are useful for generating eye-readable biosensor readouts that do not require a device to interpret, which is an attractive property for applications in remote or developing parts of the world.

In some aspects, the reporter enzyme of the disclosure can be used with fluorometric substrates. In some aspects, the reporter enzyme of the disclosure can be used with colorimetric substrates. In some aspects, the reporter enzyme of the disclosure is a β-glucosidase. In some aspects, the β-glucosidase of the disclosure, when used as a reporter enzyme, for instance, can be used with fluorometric substrates, e.g., without limitation, a fluorescein di-β-D-glucopyranoside. In some aspects, the β-glucosidase of the disclosure, when used as a reporter enzyme, for instance, can be used with colorimetric substrates, e.g., without limitation, a p-nitrophenyl-β-D-glucopyranoside.

In some aspects, the reporter enzyme of the disclosure is a β-galactosidase. In some aspects, the β-galactosidase of the disclosure, when used as a reporter enzyme, for instance, can be used with fluorometric substrates, e.g., without limitation, a fluorescein di-β-D-galactopyranoside. In some aspects, the β-galactosidase of the disclosure, when used as a reporter enzyme, for instance, can be used with colorimetric substrates, e.g., without limitation, a p-nitrophenyl-β-D-galactopyranoside.

In some aspects, the reporter enzyme of the disclosure is an alkaline phosphatase. In some aspects, the alkaline phosphatase of the disclosure, when used as a reporter enzyme, for instance, can be used with fluorometric substrates, e.g., without limitation, a 4-methylumbelliferyl phosphate. In some aspects, the alkaline phosphatase of the disclosure, when used as a reporter enzyme, for instance, can be used with colorimetric substrates, e.g., without limitation, a p-nitrophenyl phosphate.

Reporter enzymes are commonly used in cell biology to study the transcriptional activity of genes. Reporter enzymes are commonly used in a variety of assays. The most frequently used reporters are the Escherichia coli lacZ gene encoding for β-galactosidase (EC 3.2.1.23) (βgal), the green fluorescent protein (GFP) of Aequorea victoria, and, to a lesser degree, the human placental alkaline phosphatase. The most commonly conjugated reporter enzymes are horseradish peroxidase (HRP) from the horseradish plant Armoracia rusticana and alkaline phosphatase (AP) from calf intestines. Additional commonly conjugated reporter enzymes include glucose oxidase (GOD) and β-galactosidase from Escherichia coli.

Horseradish peroxidase, alkaline phosphatase, glucose oxidase (GOD), and β-galactosidase offer different benefits depending on the application requirements.

In some aspects, the reporter enzyme utilized in the disclosure is β-galactosidase. β-galactosidase is well known to signal its presence by hydrolyzing X-gal to produce a blue product. Juers et al., “LacZ β-galactosidase: structure and function of an enzyme of historical and molecular biological importance.” Protein Science 21.12 (2012):1792-1807. The promoter (e.g., without limitation, an SV40 early promoter) and an enhancer drive the transcription of the lacZ gene, which encodes the β-galactosidase enzyme (βgal). β-galactosidase is generally an excellent reporter enzyme that can be assayed quickly and directly in cell extracts. In some aspects, the β-galactosidase, as described in the disclosure, is assayed quickly and directly from the cell extracts using, for instance, and without limitation, a spectrophotometric assay, a fluorescent, and/or a chemiluminescent assay. In some aspects, the β-galactosidase is for use in in situ histochemical analysis. In some aspects, the β-galactosidase utilizes the substrate X-Gal (5-bromo-4-chloro-3-indolyl-β-d-galactopyranoside).

In various aspects, the biosensor of the disclosure (e.g., an enzyme-linked and/or a cell-based enzyme-linked biosensor) differentiates from those known in the art by utilizing β-glucosidase as its reporter enzyme. In various aspects, the method of disclosure differentiates from the contemporary methods by utilizing β-glucosidase as its reporter enzyme.

Yet, in other various aspects, the β-glucosidase of the disclosure is very stable compared to another β-glucosidase in the art. In some aspects, the β-glucosidase of the disclosure is capable of increasing the shelf-life of test kits (e.g., assays described herein) that utilize the β-glucosidase of the disclosure compared to another β-glucosidase in the art. The use of this specific enzyme is unique and novel. While there may be some other methodologies that have attempted to use β-glucosidases as reporter enzymes, the β-glucosidases utilized are distinguishable from the β-glucosidases of the disclosure.

In various aspects, the disclosure provides novel variants of β-glucosidase. In some aspects, the disclosure provides novel orthologs of β-glucosidase.

In various aspects, the disclosure provides methods for the identification of β-glucosidases. In some aspects, the disclosure provides methods for the identification of stable β-glucosidases. In some aspects, the disclosure provides methods for the identification of highly active β-glucosidases. In some aspects, the disclosure provides methods for the identification of stable and highly active β-glucosidases.

In some aspects, the terms “beta-glucosidase” and “R-glucosidase” are used interchangeably herein and refer to the reporter enzyme disclosed in the disclosure. β-glucosidases are a class of enzymes (EC: 3.2.1.21) that can hydrolyze the terminal, nonreducing β-D-glucosyl residues by hydrolyzing the β-1,4 glycosidic bond of various glycoconjugates including glucosides, oligosaccharides, and 1-O-glucosyl esters, to form glucose. The β-glucosidases substrates are widely distributed in nature, and the enzyme is present across archaea, bacteria, and eukaryotes. β-glucosidases are classified into different glycoside hydrolase (GH) families based on structural and sequence differences.

β-glucosidase has not been used as an enzyme reporter to date. While there are examples of the use of β-glucosidase enzymes in the literature, such as thermotolerant versions, there has not been a comprehensive study to identify β-glucosidases that can perform well in traditional high-throughput assays as well as in microfluidic droplets. In some aspects, the reporter enzyme utilized in the disclosure is β-glucosidase. In various aspects of the disclosure, the present discourse provides β-glucosidases that can perform well in traditional high-throughput assays. In various aspects of the disclosure, the present discourse provides β-glucosidases that can perform well in microfluidic droplets.

In various aspects, the disclosure further provides stable and highly active variants of β-glucosidase that exhibit superior properties (e.g., without limitation, optimal pH range, and adequate thermal stability).

In various aspects, the disclosure further provides an enzyme-linked biosensor that expresses a reporter enzyme. In some aspects, the reporter enzyme is derived from a β-glucosidase gene (pBATS_0004). In some aspects, pBATS_0004 performs well in microfluidic and plate-reader experiments, providing an ideal readout in ultra-high-throughput screens.

In various aspects, the disclosure further provides 13 novel β-glucosidase orthologs. Of the 13 β-glucosidase orthologs, APC115045 and APC115086 have the highest relative activities. In some aspects, APC115045.102 is the β-glucosidase ortholog with SEQ ID NO: 5. In some aspects, APC115086.102 is the β-glucosidase ortholog with SEQ ID NO: 9. of the order Bacteroidales from the human gut symbiont Bacteroides intestinalis. isolation from human fecal material. an anaerobe, gram-negative, rod-shaped human pathogen that was isolated from human feces.

In some aspects, variants APC115045.102 and APC115086.102 of β-glucosidase performed the best when p-Nitrophenyl-β-D-glucopyranoside was used as a substrate.

In some aspects, the reporter enzyme is a β-glucosidase. In some aspects, the β-glucosidase is APC115086.

In some aspects, the enzyme-linked biosensor expression cassette of the disclosure comprises a nucleic acid. In some aspects, the nucleic acid comprises a nucleotide sequence encoding a reporter enzyme. In some aspects, the reporter enzyme used in the construct is capable of hydrolyzing a fluorescent substrate. In some aspects, the reporter enzyme is a β-glucosidase.

In some aspects, the nucleotide sequence encoding β-glucosidase comprises at least 80% sequence identity (e.g., at least 85% sequence identity, at least 90% sequence identity, at least 95% sequence identity, at least 98% sequence identity, at least 99% sequence identity, or 100% sequence identity) to the nucleotide sequence of SEQ ID NO: 1.

In some aspects, the nucleotide sequence encoding β-glucosidase comprises at least 80% sequence identity to the nucleotide sequence of SEQ ID NO: 1.

In some aspects, the nucleotide sequence encoding β-glucosidase comprises the nucleotide sequence of SEQ ID NO: 1.

| SEQ ID NO: 1: Nucleotide sequence of the pBATS_0004 β-glucosidase; Glucosidase | |
|---|---|
| (APC115086_29_766) (2220 nt) | |
| 1 | ATGAAATCTC CTGTCGATAT GGATCGCTTT ATTGATGATC TGATGAAGAA |
| 51 | GATGACTCTG GAAGAGAAAA TCGGCCAGTT GAACTTGCCT GTTACGGGTG |
| 101 | AAATAACCAC CGGACAAGCC AAGAGTAGTA ATGTGGCTAA GCGTATCCGT |
| 151 | GCCGGTGAAG TGGGCGGACT CTTTAACTTG AAAGGCGTGG AGCGTATTCG |
| 201 | TGACGTTCAG AAACAGGCAG TAGAAGAAAG TCGTCTGGGT ATTCCTCTTT |
| 251 | TATTTGGTAT GGATGTAATT CATGGATACG AAACGGTATT TCCTATTCCT |
| 301 | CTGGGATTAT CCTGTACCTG GAACATGACA GCTATTGAAG AATCTGCACG |
| 351 | TATTGCTGCT ATCGAAGCCA GTGCTGATGG TATTTGCTGG ACATTCAGTC |
| 401 | CGATGGTGGA TGTTTCCCGT GATCCCCGTT GGGGACGAGT TTCCGAAGGG |
| 451 | AATGGTGAAG ATCCCTTCTT GGGAGCGGAG ATTGCGCGTG CTATGGTACG |
| 501 | TGGTTATCAA GGGAAAGATA TGAGTAGTAA TGATGAAATT ATGGCTTGCG |
| 551 | TGAAGCACTT TGCGTTATAT GGGGCATCAG AAGCCGGACG CGACTATAAT |
| 601 | ACAGTGGATA TGAGTCATCA ACGTATGTTC AACGAATATA TGTTACCTTA |
| 651 | TCAGGCTGCC GTGGAAGAAG GTGTGGGTAG TGTGATGGCT TCATTCAATG |
| 701 | AAGTGGATGG TGTACCGGCT ACCGGAAATA AGTGGCTGAT GACCGATGTA |
| 751 | CTTCGTAAGC AGTGGAATTT TGATGGGTTC GTTGTGACGG ACTATACCGG |
| 801 | TATCACTGAA ATGACCGATC ATGGTATGGG TGATACACAA ACAGTTGCAG |
| 851 | CCCTGGCTCT GAATGCAGGT GTCGATATGG ATATGGTGAG CGATGCTTTT |
| 901 | ACAAGCACAC TTAAAAAATC TCTGGAAGAA GGAAAAGTTT CAGTAAAGGC |
| 951 | TGTTGATGCT GCTTGTCGCC GTATTCTGGA AGCTAAGTAT AAGCTGGGGC |
| 1001 | TTTTTGATAA TCCCTATAAA TATTGTGATA TAACCCGTCC TAAAAAACAA |
| 1051 | ATCTTTACAA AAGAACACCG CGCTATAGCC CGTAAGACAG CTTCGGAAAG |
| 1101 | CTTTGTTCTC TTGAAGAATG AGAATAGTGT ACTCCCTCTG GCAAAGAAAG |
| 1151 | GTACCATTGC TGTAGTAGGT CCTTTGGCCG ATAGCCGTAG CAATATGCCG |
| 1201 | GGCACGTGGA GTGTGGCCGC TGTGATGAAC AAATATCCTT CTTTGATTGA |
| 1251 | AGGCTTGAAA GAAGTAGTGG GAGGCAAGGC TAAAATTCTT ACGGCTAAAG |
| 1301 | GAAGTAATCT GATGAGTGAT GCCGAATACG AAGAACGTGC TACTATGTTT |
| 1351 | GGCCGTACTC TGCATCGTGA CAATCGTACA GATAAGGAAC TGCTGGATGA |
| 1401 | GGCGCTTGCT GTAGCTGCCA AGTCTGACGT GATTGTTGCT GCTTTGGGTG |
| 1451 | AGTCTTCCGA GATGAGCGGT GAAAGTAGTT GCCGTACAGA CCTCGAAATG |
| 1501 | CCGGATACGC AACGTGTACT TTTGCAGGAA TTGTTGAAAA CCGGCAAACC |
| 1551 | GGTGGTATTG GTGTTGTTTA CCGGTCGTCC GTTAGTATTG AATTGGGAGC |
| 1601 | AGGAAAATGT ACCTGCTATT CTGAATGTGT GGTTTGGTGG TAGTGAAGCT |
| 1651 | GCTCTTGCCA TTGGTGATGT ACTGTTTGGA AATGTAAATC CGAGTGGCAA |
| 1701 | ACTTACTACT ACTTTTCCGA AGAGTGTAGG ACAGATTCCT TTGTTCTATA |
| 1751 | ACCATAAGAA TACTGGTCGT CCTTTGCCTC AAGGGGCCTG GTTCCAGAAG |
| 1801 | TTCCGTAGCA ATTATCTGGA TGTAGATAAC GAACCGCTTT ATCCGTTTGG |
| 1851 | ATATGGCTTG AGCTATACTA CTTTCTCTTA TAGTGATATT ACATTGGATA |
| 1901 | AATCGTCCAT GAATATCAAT GGAGAGATTA TGGCAACTGT AACGGTAACC |
| 1951 | AATACAGGTA AGTATGACGG TTCGGAAGTA GTGCAGCTAT ATATCCGCGA |
| 2001 | TCTTATAGGC AGTGTAACAC GTCCGGTGAA AGAACTGAAA GGCTTTGAAA |
| 2051 | AAATCTTCTT GAAAGCCGGT GAATCCAAAC AAGTGTCTTT CAAGTTAACA |
| 2101 | GCTGATATGT TGAAGTTCTA CAATTACAAT CTGGATTTTG TGTGCGAACC |
| 2151 | GGGTGACTTT GAAGTAATGA TAGGTGGTGA TAGCCGTGAT GTGAATAAGG |
| 2201 | CCTTATTTTC GCTTCAATAA |

In various aspects, the β-glucosidase enzyme of the disclosure is a variant of β-glucosidase. In various aspects, the β-glucosidase enzyme of the disclosure is a natural variant of β-glucosidase. In various aspects, the β-glucosidase enzyme of the disclosure is an ortholog of β-glucosidase. In various aspects, the β-glucosidase enzyme of the disclosure is a naturally occurring ortholog of β-glucosidase. In various aspects, the β-glucosidase enzyme of the disclosure is a mutant of a naturally occurring ortholog of β-glucosidase.

In some aspects, the nucleotide sequence encoding β-glucosidase comprises at least 80% sequence identity (e.g., at least 85% sequence identity, at least 90% sequence identity, at least 95% sequence identity, at least 98% sequence identity, at least 99% sequence identity, or 100% sequence identity) to the nucleotide sequence of any one of SEQ ID NOs: 2-18.

In some aspects, the nucleotide sequence encoding β-glucosidase comprises at least 80% sequence identity to the nucleotide sequence of any one of SEQ ID NOs: 2-18.

In some aspects, the nucleotide sequence encoding β-glucosidase comprises the nucleotide sequence of any one of SEQ ID NOs: 2-18.

Thus, in some aspects, the disclosure provides a novel variant of β-glucosidase. In aspects, the novel variant of β-glucosidase of the disclosure comprises any one of the nucleotide sequences of SEQ ID NOs: 2-18 as provided in Table 1 below.

TABLE 1
Nucleotide Sequences of Beta(B)-Glucosidase Variants of the Disclosure.

| | | SEQ | |
|---|---|---|---|
| | CloneID. | ID | |
| CloneID | F | NO: | Nucleotide Sequence |
| APC1150 | APC1150 | 2 | AAGTCACCGCAAGACATGGATCGCTTCATCGACGCACTGATGAAGAAGATGACCGTGGAAGAGAAAATC |
| 38.102 | 38.26- | | GGACAATTGAACCTACCCGTCACGGGAGACATCACCACGGGACAGGCCAAAAGTAGCGACGTGGCACAA |
| | 783.P | | AAGATTGAAAAAGGATTGGTGGGCGGACTCTTCAACCTAAAAGGTGTAGACCGTATTCTTGAAGTGCAA |
| | (785).. | | AAGCTGGCAGTAGAGAAATCACGCCTCGGTATTCCCCTGCTGTTCGGCATGGATGTGATACATGGCTAC |
| | pMCSG68 | | GAAACCATCTTCCCCATTCCATTGGGATTGTCCTGCACCTGGGATATGGCGGCTATCGAGAAATCCGCC |
| | | | CGTATTGCAGCCATCGAAGCAAGTGCCGATGGCATTTCCTGGACATTCAGTCCGATGGTAGACATCAGT |
| | | | CGCGACCCACGTTGGGGACGTGTCAGCGAGGGCTCGGGAGAAGATCCGTTTCTGGGTGGAGCTATCGCA |
| | | | CAGGCAATGGTATACGGATACCAGGGTGCCAATCTGCAAGACCAATTGCACCGCAACGATGAAATCATG |
| | | | GCTTGCGTAAAACACTTTGCATTGTATGGAGCCGGAGAAGCCGGACGCGACTATAATACAGTAGATATG |
| | | | AGCCGCAACCGGATGTTCAATGAATTCATGTACCCGTATGAGGCTGCCGTAGAGGCCGGAGTGGGTAGT |
| | | | GTGATGGCGTCATTCAATGAAATAGACGGTATTCCCGCCACCGGAAACAAATGGCTGCTGAGCGATTTG |
| | | | CTGCGTGGCCAGTGGGGCTTCGAAGGGTTTGTGGTAACGGACTTCACAGGCATTTCAGAGATGATAGAG |
| | | | CATGGTGTCGGCGACTTGCAAACCGTCAGTGCACTCGCTCTTAATGCAGGGGTGGACATGGATATGGTA |
| | | | AGTGAGGGCTTCGTCGGTACACTGATGAAATCAATTAAAGAAGGAAAAGTAAGAATGGGCACGTTGAAT |
| | | | ACAGCCTGCCGCCGGATATTGGAAGCGAAATACAAGCTGGGACTGTTTGACAATCCTTATAAATACTGC |
| | | | GACGTGAACCGTCCGAAGCGGGATATCTTCACAAAAGAGCATCGTGACGCCGCCCGCAAGATTGCCGGC |
| | | | GAAAGTTTTGTTCTTCTGAAGAATGCCCCCGCCACCGCACAGCCACTCGCAGCTCATAGCTCGTCACCC |
| | | | GTAACTGCTTCCCCCGTGCTTCCGTTGAAGAAACAAGGTACAGTTGCCGTCATCGGCCCTCTCGGAAAT |
| | | | ACCCGCAGCAACATGCCGGGCACCTGGAGCGTAGCCGCACGCCTCAACGATTATCCTTCTTTATACGAA |
| | | | GGCTTGAAAGAAATGATGGCAGGCAAGGTGAACATCACCTATGCCAAAGGTAGTAACCTCATCGGCGAT |
| | | | GCAGCTTACGAAGAACGTGCCACCATGTTCGGCCGTTCATTGAACCGCGATAATCGCACGGACCAGGAG |
| | | | TTACTGGACGAAGCACTGAAAATTGCAGCCGGCGCCGATGTTATCGTAGCTGCCCTGGGAGAATCTTCT |
| | | | GAAATGAGCGGTGAAAGTTCAAGCCGCACCGAACTCGGCTTGCCCGATGTACAACATACCCTGTTGGAA |
| | | | GCCTTACTGAAAACGGGTAAACCCGTAGTACTAACCCTCTTTACCGGTCGGCCGTTGACGCTGAACTGG |
| | | | GAACAGGAGCATGTACCTGCCATCTTGAATGTATGGTTCGGAGGCAGTGAAGCGGCTTATGCCATTGGC |
| | | | GATGTTCTGTTCGGTGACGTCAATCCGAGTGGAAAACTAACCATGACTTTCCCGAAGAATGTAGGCCAG |
| | | | ATACCTTTGTTCTACAATCATAAGAATACCGGTCGGCCACTGGCGGCAGGCAAATGGTTCGAAAAGTTC |
| | | | CGTTCAAACTATCTGGATGTGGATAACGAACCGCTGTATCCCTTCGGTTATGGATTGTCGTATACCACT |
| | | | TTCCAGTACAGTGACATTGCATTGAGCACACCGACATTGGGAAAAGATGGTTCCGTTACAGCCGTAGTC |
| | | | ACCGTCACCAATACTGGTAAACATGACGGTGCGGAAGTAGTTCAACTCTATATCCGCGACCTCGTAGGA |
| | | | AGTATCACCCGCCCTGTACGCGAGTTGAAAGGTTTCAATAAAATCTTCCTTCGCGCCGGAGAAAGCAAA |
| | | | ACGGTATCATTCACTATCACGCGTGATCTGCTTCGCTTCTATGATTACGACCTGAACTACGTAGCCGAA |
| | | | CCGGGTGACTTTGACATCATGATCGGTGGAAACAGCCAGGCTGTGAAGACGGCGAAGTTGACACTT |
| APC1150 | APC1150 | 3 | AAGAGTGGGGATGCGTCGATGAACAAATTTATTGATAAACTGATGGACAGGATGACCTTGGAAGAGAAG |
| 43.102 | 43.26- | | ATTGGTCAGCTTAATCTTCCCAGCTCGGGAGATATAACCACCGGACAGGCACGCAGCAACAATATTGCA |
| | 768.P | | GACAAAATCAGAGCAGGTGCAGTGGGTGGCTTATTCAATATAAAAGGAGTTGAGAAGATACAGGAAGTA |
| | (769).. | | CAACGTATTGCTGTAGAGGAGAGTCGCCTGAAAATTCCTTTACTCTTTGGCATGGATGTTATTCATGGG |
| | pMCSG68 | | TATGAAACTGTTTTCCCTATTCCTTTGGGTATGGCTGCCACATGGGATATGAAGGCTATAGAACAATCT |
| | | | GCTCGTATAGCGGCGATAGAAGCCAGTGCCGATGGCATCTGCTGGACATTTAGTCCGATGGTTGATATC |
| | | | AGCCGTGATCCACGTTGGGGACGTGTATCCGAAGGTAGCGGAGAAGATCCTTTTTTAGGTGGTGAAATT |
| | | | GCTAAGGCGATGGTATATGGCTATCAGGGTAAAGGTGATAGCGCATATCGTGAAAAGACTAATATTATG |
| | | | GCTTGTGTGAAGCACTATGCCTTGTATGGGGCAGCAGAAGCCGGTTTGGACTATAATACAACTGACATG |
| | | | AGCCGTATTCGTATGTTTAATGAATATATGTATCCTTATCAGGCGGCTGTGGATGCGGGTGCCGGCAGT |
| | | | GTCATGTCTTCTTTCAACGAGGTCGATGGAATTCCTGCAACAGCCAACAAATGGTTGATAACTGATGTC |
| | | | CTGCGTAAACAGTGGGGATTCGGTGGTTTTGTCGTTACGGACTATACCGGTATCATGGAAATGGTAAAT |
| | | | CATGGTATTGGAGATATGCGAGAAGTCTCTGCCCGTGCTTTGAGTGCAGGAGTGGATATGGATATGGTG |
| | | | AGCGAAGGTTATCTTTCTACACTTCAACAATCATTGAAGGAGGGTAAGATAACAGAGAAAGAGATAGAT |
| | | | CAAGCTTGCCGTCGTATTTTGGAGGCAAAATATAAGCTGGGATTATTTGATAATCCTTATAAGTATTGT |
| | | | GATACTGAACGTGCCAAAACGGATATCTACACTGATGAACATCGGAGTATTGCACGCCGGATCTCTGCT |
| | | | GAAAGCTTTGTTCTTTTAAAGAATGATAAACAGACACTGCCTATAAAGAAAAAAGGTAAGATTGCTGTA |
| | | | GTTGGGCCGTTGGCGAATACGAGTTCTAATATGCCCGGAACGTGGAGTGTAGCGGTCAATATGGAAGCT |
| | | | CCAGCTACGCTTGTGGAGGGTTTGAAAGAAGTGGCAGGTGATAAAGTTGAAATTGTGTATGCTAAGGGT |
| | | | AGCCATCTGATGAGTGATGCGGCTTATGAGGAACGTGCAACACTCTTTGGACGTACATTATACCGGGAT |
| | | | AAGGAAAAACGTTCCGATATCCAGATGCTGAATGAAGCATTAAATGTTGCTCATGGTGCCGATGTTGTT |
| | | | GTTGCGGCATTAGGTGAATCTTCTGAAATGAGTGGTGAATCGAGTAGTCGAACAGATTTGAATATTCCT |
| | | | GATGTTCAAAAAACATTATTGGAAGAATTAGTGAAAACAGGTAAACCTGTCGTTCTGGTATTATTCACT |
| | | | GGGCGTCCGTTGACCCTGACATGGGAAGACAAAAATGTATCTGCTATTCTGAATGTTTGGTTTGGAGGT |
| | | | ACCGAAGCCGCTTATGCTATAGGAGATGTCCTATTCGGAAATGTAAATCCTGGAGGTAAGCTGCCTGTA |
| | | | ACATTTCCTCAGAATGTAGGGCAGATTCCTTTATTCTATAACCATAAAAATACTGGACGTCCGCTGGCT |
| | | | GAGGGCGGTTGGTTTGAGAAGTTCCGGGCAAATTATCTGGATGTAACGAATGAACCTCTTTATCCATTT |
| | | | GGCTATGGACTAAGTTATGCACAATTTGATTATAGCGATGTGAGATTAAGTACGGATCAAATAGACCGG |
| | | | AATGGCATGTTAACCGCAAGTGTGACTGTAACCAATAACAGTGAGTGTGATGGAGATGAAATTGTTCAG |
| | | | TTGTATATTCGCGATTTGGTCGGTAGTGTTACTCGTCCGGTGAAAGAATTGAAAGGATTTGAAAAAGTA |
| | | | ACAATTAGAGCAGGGGAGTCAAAAGATATTTCTTTTAAGATCACTCCGGAAATGCTTAAGTTCTACAAT |
| | | | TCGGATATCCAGTTTGTGAATGAAGTTGGTGAATTCGAAGTAATGATCGGAACGAACAGCAGGGATGTG |
| | | | AAAAAAGCAACGTTTAGCTTG |
| APC1150 | APC1150 | 4 | GTAGAATCTCTCCTGTCTAAGATGACCCTTGAGGAGAAAATCGGTCAGATGAACCAGATTTCCTCTTAC |
| 44.102 | 44.33- | | GGTAATATCGAGGATATGAGTGCTTTGATTAAGAAAGGTGAAATCGGTTCCATCTTGAATGAGGTGGAT |
| | 744.P | | CCGGTGCGTATTAATGCGCTACAGCGCGTGGCAATGGAAGAATCCCGTTTGGGTATTCCTTTATTGATA |
| | (748).. | | GCGCGTGATGTCATTCACGGGTTTAAAACAATTTTCCCTATTCCCTTGGGACAAGCGGCTTCGTTCAAT |
| | pMCSG68 | | CCGCAGGTAGCGAAAGACGGTGCACGGATAGCAGCTATTGAAGCTTCGTCTGTAGGTATCCGGTGGACT |
| | | | TTTGCGCCAATGATTGATATTGCCCGCGATCCTCGCTGGGGACGTATTGCCGAAGGGTGTGGTGAAGAT |
| | | | ACGTACCTTACTTCCGTAATGGGAGCAGCTATGGTAGAAGGTTTTCAGGGAGATTCGCTGAATAGTCCT |
| | | | ACTTCAATTGCAGCTTGCCCTAAACATTTTGTAGGTTACGGTGCAGCCGAAGGAGGACGTGATTATAAT |
| | | | TCCACGTTCATTCCCGAACGTCGTCTGCGCAATGTTTATTTGCCACCTTTTGAAGCTGCCACCAAAGCG |
| | | | GGTGCAGCCACGTTTATGACTTCATTTAATGATAATGATGGAATCCCTTCTACCGGGAATGCTTTTATT |
| | | | TTGAAGAATGTACTCCGTGACGAGTGGGGATTCGATGGTTTTGTTGTGACGGACTGGGCTTCTGCCAGC |
| | | | GAAATGATAAGCCATGGTTTTGCCGCCGGTTCAAAAGAAGTGGCAATGAAATCTGTGAATGCAGGAGTA |
| | | | GATATGGAAATGGTGAGTTACACTTTTGTGAAGGAACTGCCGGAATTAGTGAAAGAGGGAAAGGTGAAG |
| | | | GAAAGCACTATCGATGAGGCTGTTCGTAATATTTTGCGTATAAAGTATCGTTTAGGATTGTTTGATACA |
| | | | CCTTATGTAGATGAACAACAAACATCTGTCATGTATGCTCCTTCTCATTTGGAAGCAGCTAAGCAAGCC |
| | | | GCTGTTGAATCGGCTATTCTGTTGAAGAATGATAAGGAAGTGTTGCCGTTACAGCCATCTGTGAAAACT |
| | | | GTTGCAGTGGTAGGACCTATGGCTAATGCACCTTATGAACAGTTAGGTACTTGGATATTTGATGGTGAG |
| | | | AAAGCTCGTACTCAGACTCCGTTGAACGCTATTAAAGAAATGGTTGGCGATAAAGTACAGGTGATTTAT |
| | | | GAACCGGGACTAGCATATAGTCGTGAGAAAAATCCGGCAAGTGTGGCTAAAGCAGCTGCCGCCGCTGCA |
| | | | CGTGCAGATGTCATTCTTGCTTTTGTGGGTGAAGAATCTATTCTTTCGGGTGAAGCTCACTGTTTGGCT |
| | | | GATCTGGATTTGCAGGGTGATCAGGGAGCTTTGATTACAGCTTTGGCTAAGACGGGTAAACCTGTAGTG |
| | | | ACTATTGTGATGGCGGGTCGTCCGTTGACTATCGGTAAAGAAGTCGAAGAGTCGACTGCTGTTCTCTAT |
| | | | TCATTCCATCCGGGCACAATGGGCGGTCCTGCATTGGCTGATTTGCTTTGGGGGAAGGCTGTGCCGAGT |
| | | | GGAAAGGCGCCGGTCACTTTCCCGAGGATGGTGGGACAAATTCCTGTGTACTACGCTCATAATAATACC |
| | | | GGACGTCCGGCTACACGGAATGAAGTGTTGCTGAATGATATTGCTGTTGAGGCAGGACAGACTTCACTG |
| | | | GGCTGTACTTCCTTCTATATGGATGCGGGTTTTGATCCCTTGTTTCCGTTTGGTTATGGCTTGTCGTAC |
| | | | ACCACATTTAAGTATAGCAACATCAAACTGGCGTCTGATGTACTGAAAAAAGATGATGTGCTGACAGTG |
| | | | ACATTCGATCTGGAAAATACCGGGAAATATGAAGGAACGGAAGTAGCTCAATTGTATATACAAGATAAG |
| | | | ATTGGTTCCGTGACTCGTCCGGTGAAAGAACTGAAACGCTTCACTCGTGTGACATTGAAGCCGGGTGAG |
| | | | AAAAAAAGCGTTTCGTTTGAACTCCCTGTTAGTGAACTTGCATTTTGGAACATAGATATGGCTAAAGTT |
| | | | GTGGAACCCGGAGACTTTGGGCTTTGGGTGGCAACGGATAGTCAGTCCGGAGAAGAAGTTTTCTTC |
| APC1150 | APC1150 | 5 | AAGTCTCCGCAGGACATGGATCGCTTCATCGATGCATTGATGAAGAAGATGACTGTAGAGGAAAAGATC |
| 45.102 | 45.26- | | GGTCAGCTGAACCTACCCGTTTCCGGCGAGATCGTCACCGGGCAGGCACAAAACAGCGATGTGGCAAAA |
| | 772.P | | AAGATTGAACAAGGGCTCGTGGGCGGACTCCTCAACCTGAAAGGGGTGGAGAAGATACGCGATGTACAA |
| | (773).. | | AAACTGGCCATAGAGAAGTCACGCCTGGGCATCCCCCTGATATTCGGCATGGACGTAGTGCATGGTTAC |
| | pMCSG68 | | GAAACCATTTTCCCTATTCCATTAGGCCTCTCCTGTTCCTGGGATATGGAAGCCATCAGGAAATCTGCC |
| | | | CGCGTTGCAGCCATCGAGGCCAGTGCTGATGGTATTTCCTGGACATTCAGCCCGATGGTAGACATCAGC |
| | | | CGTGATCCGCGCTGGGGACGCGTCAGCGAGGGTAACGGCGAAGACCCATTCTTGGGTGGAGCCATCGCT |
| | | | AAAGCAATGGTATCGGGTTATCAGGGTATCGACCTCAACAACCAACTGAAGCGCAACGATGAAATTATG |
| | | | GCATGTGTAAAGCACTTCGCACTGTATGGTGCCGGAGAAGCCGGACGTGATTACAATACCGTAGATATG |
| | | | AGTCGTAACCGTATGTTCAACGAATACATGTATCCCTACCAAGCTGCCGTAGATGCAGGTGTAGGCAGC |
| | | | GTAATGGCGTCTTTCAACGAAATAGACGGCATACCAGCCACGGCCAATAAATGGCTGATGACCGACGTA |
| | | | CTGCGCAAGCAATGGGGCTTCGACGGCTTTGTGGTGACAGACTTTACCGGTATCTCCGAAATGATAGCG |
| | | | CACGGCATCGGTGACTTGCAGACTGTTTCCGCACGTGCACTCAATGCAGGCGTGGATATGGACATGGTA |
| | | | AGTGAAGGCTTCACGGGTACAATCAAGAAATCCATAGACGAAGGCAAGATCAGTATGGAAACCCTGGAC |
| | | | AAAGCCTGTCGCCGCATCCTTGAAGCCAAATACAAACTGGGATTATTCGACAATCCTTATAAGTACTGC |
| | | | GACCTGAAACGCCCGAAGCGTGACATCTTCACCAAGGAACATCGCGACGCTGCTCGTAAGATTGCGGGA |
| | | | GAGAGCTTTGTACTCCTGAAAAACGACAAGTCAGGTTCCTCTGCAAACCCAACACTTCCTTTGAAAAAA |
| | | | GAAGGTACGGTGGCTGTCATCGGCCCACTGGCAAATACCCGCAGTAACATGCCGGGTACCTGGAGTGTA |
| | | | GCCGCACGCCTCAACGACTATCCTTCTGTGTACGAAGGATTGAAAGAGATGATGAAAGGCAAGGTAAAC |
| | | | ATCACTTATGCCAAAGGTAGTAACCTCATCAGTGATGCAGCCTACGAAGAACGTGCCACAATGTTCGGC |
| | | | CGTTCATTAAATCGTGATAATCGTACAGACAAAGAGATGCTGGATGAGGCGCTGAAAGTGGCCGCTAAT |
| | | | GCAGATGTAATAATAGCCGCATTGGGAGAATCATCTGAAATGAGTGGTGAAAGTTCAAGCCGCACTAAC |
| | | | CTGGCTCTTCCCGATGTACAGCGCACTCTATTGGAAGCTTTGCTGAAAACTGGAAAGCCTGTTGTACTG |
| | | | ACGCTCTTTACAGGTCGCCCACTAACGTTGACTTGGGAACAGGAGCATGTGCCCGCCATCCTGAATGTA |
| | | | TGGTTCGGTGGAAGTGAGGCAGCATACGCCATTGGCGATGTATTGTTCGGCGATGTAAATCCCAGCGGC |
| | | | AAACTAACGATGACATTCCCCAAAAACGTAGGCCAAATACCTTTGTTTTACAATCATAAAAATACCGGT |
| | | | CGTCCTTTACTTGAAGGCAAATGGTTCGAAAAATTCCGTAGTAATTACCTGGATGTAGACAACGATCCA |
| | | | TTGTATCCATTCGGCTATGGTTTGTCGTATACCAACTTTCAATACAGCGACATAACTCTGAGCGCCCCG |
| | | | ACTATGGGACAGGATGGTTCTGTTACTGCTATGGTCACGGTAACCAATACCGGTAAGTACGATGGTGCA |
| | | | GAAGTAGTGCAACTTTATATCCGTGACCTTGTAGGAAGCATCACCCGTCCGGTAAAAGAACTGAAAGGG |
| | | | TTTGATAAAATTTTCCTCAAAGCGGGTGAAAGTAAGACTGTATCTTTCAAAATCACTCCGGAATTACTG |
| | | | CGCTTCTACGACTATGAACTCAACTACGTAGCCGAACCGGGAGACTTCGACATAATGATCGGGGGGAAC |
| | | | AGCCAAAGTGTAAAAACGACTCATCTGAGTTTG |
| APC1150 | APC1150 | 6 | AAGTCCCCCCAAGACATGGACCGCTTCATCGATGCGCTGATGAAGAAAATGACTGTGGAAGAGAAAATC |
| 68.102 | 68.26- | | GGACAGTTGAACCTACCCGTCACGGGAGACATCACCACAGGACAGGCCAAGAGCAGCGACGTAGCCGCA |
| | 774.P | | AAGATTGAAAAAGGATTGGTAGGCGGACTCTTCAACCTGAAAGGGGTAGACCGCATTCTTGAAGTGCAA |
| | (775).. | | AAGCTGGCAGTAGAGAAATCACGTCTCGGTATTCCCCTGTTATTCGGCATGGACGTGATACATGGATAC |
| | pMCSG68 | | GAAACCATCTTCCCCATCCCATTGGGGCTGTCCTGCACTTGGGATATGGCCGCCATCGAGAAGTCTGCC |
| | | | CGTATCGCAGCCATCGAAGCAAGTGCCGATGGCATCTCCTGGACATTCAGTCCGATGGTAGACATCAGC |
| | | | CGTGATCCACGTTGGGGACGTGTCAGCGAAGGTTCGGGAGAAGACCCTTTCCTGGGTGGAGCTATCGCA |
| | | | CAGGCAATGGTATACGGATACCAGGGTGCCAATCTGCAAGACCAGTTGCGCCGTAATGATGAAATCATG |
| | | | GCCTGCGTTAAACATTTCGCCCTGTATGGAGCCGGAGAGGCCGGACGCGATTATAACACAGTGGACATG |
| | | | AGCCGCAACCGGATGTTCAATGAATTTATGTATCCGTACGAAGCTGCCGTAGAGGCAGGTGTAGGTAGC |
| | | | GTAATGGCTTCATTCAATGAAATAGACGGGATACCGGCTACCGGGAACAAATGGCTATTGAGCGACTTG |
| | | | CTGCGTGGCCAATGGGGGTTTGAAGGGTTTGTGGTAACAGACTTTACAGGTATTGCGGAGATGATAGAA |
| | | | CATGGTGTCGGCGACTTACAAACCGTCAGTGCACTTGCCCTGAATGCAGGTGTGGATATGGATATGGTA |
| | | | AGTGAAGGTTTTGTCGGCACGCTGATGAAATCCATTAAAGAAGGAAAAGTGAGAATGGGTACGCTAAAT |
| | | | ACGGCTTGCCGCCGGATATTGGAAGCAAAATATAAATTGGGCCTGTTCGACAATCCTTATAAATATTGT |
| | | | GATGTGAACCGTCCGAAGCGGGACATCTTTACAAAAGAACATCGGGATGCCGCCCGTAAGATTGCCAGT |
| | | | GAAAGTTTTGTACTTTTAAAGAACGCTCCCTTAGCAGCACAGAAAAATGCCGCCCCCGTGCTTCCATTA |
| | | | AAGAAGCAAGGCACCGTTGCAGTAATCGGTCCTCTCGGCAATACGCGTAGCAATATGCCGGGCACTTGG |
| | | | AGTGTAGCTGCACGCCTCAACGATTATCCTTCTTTGTACGAAGGACTGAAAGAGATGATGGCAGGCAAA |
| | | | GTCAACATCACCTACGCCAAGGGCAGCAACCTTATCGGTGATGCTGCTTACGAAGAACGTGCCACCATG |
| | | | TTCGGTCGCTCACTGAACCGCGACAACCGTACGGATCAGGAATTATTGGACGAAGCGCTGAAAGTGGCA |
| | | | GCCGGAGCCGATGTCATCGTAGCCGCACTGGGGGAATCTTCTGAAATGAGTGGTGAAAGTTCAAGCCGC |
| | | | ACAGAACTCGGCTTACCCGATGTGCAGCATACTTTACTGGAAGCCTTACTAAAAACAGGCAAGCCTGTA |
| | | | GTACTTACTCTGTTTACCGGTCGCCCGTTGACACTGAACTGGGAACAGGAACATGTACCTGCTATCCTC |
| | | | AATGTATGGTTCGGAGGTAGCGAGGCAGCTTATGCCATTGGCGATGTATTGTTCGGCGACGTAAATCCA |
| | | | AGTGGAAAGCTGACGATGACGTTCCCGAAGAATGTAGGCCAGATACCTTTGTTCTACAATCATAAGAAT |
| | | | ACCGGTCGCCCGTTGGCAGAAGGTAAATGGTTCGAAAAGTTCCGTTCAAATTATCTGGATGTGGATAAT |
| | | | GAACCATTGTACCCCTTCGGTTATGGATTATCATATACCAACTTCCAGTATAGTGACATTGCACTGAGC |
| | | | ACGCCTACACTGGGAAAAGACGGTTCTGTTACCGCCGTAGTTACTGTAACCAATACGGGTAAATACGAT |
| | | | GGTGCGGAAGTAGTACAACTCTATATCCGTGATCTTGTAGGAAGCATCACCCGTCCGGTGCGCGAGCTG |
| | | | AAGGGGTTCAATAAGATCTTCCTTCGTGCCGGAGAAAGTAAAACAGTATCATTCACCATCACGCGCGAC |
| | | | CTGCTCCGGTTCTATGATTATGATATGAATTACGTAGCCGAACCCGGTGATTTCAATATTATGATCGGT |
| | | | GGAAACAGCCAGACGGTGAAGACGGCAAAATTAACACTT |
| APC1150 | APC1150 | 7 | CGGGAACAATCTTTCGATGAGGCATGGTTATTTCATCGTGGAGATATTGCCGAAGGAGAAAAGCAATCT |
| 77.102 | 77.27- | | TTAGATGACTCACAATGGCGTCAGATAAATCTTCCTCATGATTGGAGTATTGAAGATATTCCTGGAACC |
| | 740.P | | AATTCTCCTTTTACAGCGGATGCTGCAACGGAAGTTGCAGGTGGTTTTACTGTAGGTGGTACGGGATGG |
| | (800).. | | TATAGAAAGCACTTCTACATAGATGCGGCTGAAAAAGGTAAATGTATTGCTGTCTCTTTCGATGGAATT |
| | pMCSG68 | | TATATGAATGCAGATATCTGGGTGAATGATCGCCATGTAGCCAATCATGTTTATGGATATACTGCATTT |
| | | | GAACTGGATATAACCGATTATGTACGTTTCGGAGCTGAAAATCTGATAGCTGTCCGTGTGAAGAATGAA |
| | | | GGTATGAATTGCCGTTGGTATACAGGTTCGGGTATTTACAGGCATACTTTCTTGAAGATAACCAATCCG |
| | | | CTTCATTTTGAAACTTGGGGAACGTTTGTCACGACTCCCGTTGCAACGGCGGATAAAGCAGAGGTACAT |
| | | | GTACAGAGTGTTCTGGCAAATACTGAAAAAGTAACCGGAAAAGTGATTCTGGAAACGCGGATTGTAGAT |
| | | | AAGAATAACCATACTGTAGCTCGGAAAGAGCAACTGGTAACATTGGATAACAAAGAAAAAACAGAGGTT |
| | | | GGCCATGCGTTGGAAGTGCTTGCTCCGCAATTATGGTCTATAGACAATCCTTACTTATATCAGGTTGTA |
| | | | AACCGTCTTCTGCAAGATGATAAAGTTATAGATGAGGAATATATTTCAATAGGTATACGCAATATTGCA |
| | | | TTTAGTGCGGAGAATGGTTTCCAGCTGAATGGTAAATCCATGAAACTAAAAGGCGGATGTATCCATCAT |
| | | | GACAATGGTCTTTTGGGTGCAAAGGCTTTTGACCGGGCAGAGGAAAGGAAAATAGAACTACTGAAAGCG |
| | | | GCTGGTTTCAATGCGCTGCGCTTGTCTCATAATCCTCCCTCAATCGCTTTACTCAATGCCTGCGACCGC |
| | | | TTAGGTATGCTGGTCATAGATGAGGCTTTTGATATGTGGCGCTATGGTCATTATCAGTATGATTATGCA |
| | | | CAATACTTTGATAAATTGTGGAAAGAAGATTTGCATAGTATGGTTGCACGGGATAGGAATCATCCTAGT |
| | | | GTTATCATGTGGAGTATTGGTAATGAAATCAAGAACAAAGAAACTGCTGAAATTGTGGATATATGCAGG |
| | | | GAGTTGACAGGTTTTGTGAAGACGCTTGATACAACGCGGCCTGTTACGGCGGGAGTTAATTCTATTGTT |
| | | | GATGCAACGGATGATTTTCTGGCTCCTCTGGATGTTTGTGGTTATAATTACTGTTTAAACCGTTATGAA |
| | | | TCGGATGCCAAACGTCATCCGGACCGTATTATCTATGCTTCGGAGTCCTACGCATCCCAGGCTTATGAT |
| | | | TATTGGAAAGGAGTAGAAGATCATTCATGGGTGATCGGTGATTTTATCTGGACTGCTTTTGACTATATT |
| | | | GGTGAGGCAAGTATCGGCTGGTGTGGGTATCCGCTTGATAAACGTATTTTCCCTTGGAATCATGCCAAT |
| | | | TGTGGTGATTTGAATCTTTCGGGCGAACGTCGTCCCCAGTCCTATTTGCGTGAAACGTTATGGAGTGAT |
| | | | GCACCGGTATCCCATATTGTTGTGACGCCTCCTGTTCCTTCTTTTCCTCTGAATCCGGATAAGGCGGAT |
| | | | TGGAGTGTATGGGATTTTCCGGATGTTGTGGATCATTGGAATTTCCCGGGATATGAGGGGAAAAAGATG |
| | | | ACAGTATCTGTATACTCCAATTGTGAACAGGTTGAACTGTTCTTGAATGGGGAATCTTTAGGAAAACAA |
| | | | GAAAATACTGCCGATAAGAAAAATACGCTTGTCTGGGAAGTACCTTATGCTCATGGAATATTGAAAGCC |
| | | | GTAAGTTATAATAAAGGCGGTGAAGTGGGCACTGCAACGTTGGAAAGTGCTGGTAAGGTTGAAAAGATC |
| | | | AGATTATCTGCGGACAGAACGGAAATCGTAGCTGATGGTAATGATCTAAGCTATATCACATTAGAATTG |
| | | | GTAGATAGTAAAGGCATTAGAAATCAGTTGGCTGAAGAATTGGTAGCATTTTCTATAGAAGGAGATGCT |
| | | | ACG |
| APC1150 | APC1150 | 8 | GGGGAAAAAGATTCCACACTTCGGGAACAATCTTTCGATGAGGCATGGTTATTTCATCGTGGAGATATT |
| 77.103 | 77.20- | | GCCGAAGGAGAAAAGCAATCTTTAGATGACTCACAATGGCGTCAGATAAATCTTCCTCATGATTGGAGT |
| | 800.P | | ATTGAAGATATTCCTGGAACCAATTCTCCTTTTACAGCGGATGCTGCAACGGAAGTTGCAGGTGGTTTT |
| | (800).. | | ACTGTAGGTGGTACGGGATGGTATAGAAAGCACTTCTACATAGATGCGGCTGAAAAAGGTAAATGTATT |
| | pMCSG68 | | GCTGTCTCTTTCGATGGAATTTATATGAATGCAGATATCTGGGTGAATGATCGCCATGTAGCCAATCAT |
| | | | GTTTATGGATATACTGCATTTGAACTGGATATAACCGATTATGTACGTTTCGGAGCTGAAAATCTGATA |
| | | | GCTGTCCGTGTGAAGAATGAAGGTATGAATTGCCGTTGGTATACAGGTTCGGGTATTTACAGGCATACT |
| | | | TTCTTGAAGATAACCAATCCGCTTCATTTTGAAACTTGGGGAACGTTTGTCACGACTCCCGTTGCAACG |
| | | | GCGGATAAAGCAGAGGTACATGTACAGAGTGTTCTGGCAAATACTGAAAAAGTAACCGGAAAAGTGATT |
| | | | CTGGAAACGCGGATTGTAGATAAGAATAACCATACTGTAGCTCGGAAAGAGCAACTGGTAACATTGGAT |
| | | | AACAAAGAAAAAACAGAGGTTGGCCATGCGTTGGAAGTGCTTGCTCCGCAATTATGGTCTATAGACAAT |
| | | | CCTTACTTATATCAGGTTGTAAACCGTCTTCTGCAAGATGATAAAGTTATAGATGAGGAATATATTTCA |
| | | | ATAGGTATACGCAATATTGCATTTAGTGCGGAGAATGGTTTCCAGCTGAATGGTAAATCCATGAAACTA |
| | | | AAAGGCGGATGTATCCATCATGACAATGGTCTTTTGGGTGCAAAGGCTTTTGACCGGGCAGAGGAAAGG |
| | | | AAAATAGAACTACTGAAAGCGGCTGGTTTCAATGCGCTGCGCTTGTCTCATAATCCTCCCTCAATCGCT |
| | | | TTACTCAATGCCTGCGACCGCTTAGGTATGCTGGTCATAGATGAGGCTTTTGATATGTGGCGCTATGGT |
| | | | CATTATCAGTATGATTATGCACAATACTTTGATAAATTGTGGAAAGAAGATTTGCATAGTATGGTTGCA |
| | | | CGGGATAGGAATCATCCTAGTGTTATCATGTGGAGTATTGGTAATGAAATCAAGAACAAAGAAACTGCT |
| | | | GAAATTGTGGATATATGCAGGGAGTTGACAGGTTTTGTGAAGACGCTTGATACAACGCGGCCTGTTACG |
| | | | GCGGGAGTTAATTCTATTGTTGATGCAACGGATGATTTTCTGGCTCCTCTGGATGTTTGTGGTTATAAT |
| | | | TACTGTTTAAACCGTTATGAATCGGATGCCAAACGTCATCCGGACCGTATTATCTATGCTTCGGAGTCC |
| | | | TACGCATCCCAGGCTTATGATTATTGGAAAGGAGTAGAAGATCATTCATGGGTGATCGGTGATTTTATC |
| | | | TGGACTGCTTTTGACTATATTGGTGAGGCAAGTATCGGCTGGTGTGGGTATCCGCTTGATAAACGTATT |
| | | | TTCCCTTGGAATCATGCCAATTGTGGTGATTTGAATCTTTCGGGCGAACGTCGTCCCCAGTCCTATTTG |
| | | | CGTGAAACGTTATGGAGTGATGCACCGGTATCCCATATTGTTGTGACGCCTCCTGTTCCTTCTTTTCCT |
| | | | CTGAATCCGGATAAGGCGGATTGGAGTGTATGGGATTTTCCGGATGTTGTGGATCATTGGAATTTCCCG |
| | | | GGATATGAGGGGAAAAAGATGACAGTATCTGTATACTCCAATTGTGAACAGGTTGAACTGTTCTTGAAT |
| | | | GGGGAATCTTTAGGAAAACAAGAAAATACTGCCGATAAGAAAAATACGCTTGTCTGGGAAGTACCTTAT |
| | | | GCTCATGGAATATTGAAAGCCGTAAGTTATAATAAAGGCGGTGAAGTGGGCACTGCAACGTTGGAAAGT |
| | | | GCTGGTAAGGTTGAAAAGATCAGATTATCTGCGGACAGAACGGAAATCGTAGCTGATGGTAATGATCTA |
| | | | AGCTATATCACATTAGAATTGGTAGATAGTAAAGGCATTAGAAATCAGTTGGCTGAAGAATTGGTAGCA |
| | | | TTTTCTATAGAAGGAGATGCTACGATTGAAGGAGTAGGTAATGCCAACCCTATGAGCATAGAAAGTTTC |
| | | | GTTGCTAATAGTCGGAAGACGTGGCGCGGAAGTAACTTATTGGTTGTTCGTTCCGGGAAATCTTCAGGA |
| | | | CGGATTATTGTAACAGCAAAGGTAAAGGCACTTCCGGTTGCGAGTATTACTATAACTCAGAAAAAA |
| APC1150 | APC1150 | 9 | AAATCTCCTGTCGATATGGATCGCTTTATTGATGATCTGATGAAGAAGATGACTCTGGAAGAGAAAATC |
| 86.102 | 86.29- | | GGCCAGTTGAACTTGCCTGTTACGGGTGAAATAACCACCGGACAAGCCAAGAGTAGTAATGTGGCTAAG |
| | 766.P | | CGTATCCGTGCCGGTGAAGTGGGCGGACTCTTTAACTTGAAAGGCGTGGAGCGTATTCGTGACGTTCAG |
| | (766).. | | AAACAGGCAGTAGAAGAAAGTCGTCTGGGTATTCCTCTTTTATTTGGTATGGATGTAATTCATGGATAC |
| | pMCSG68 | | GAAACGGTATTTCCTATTCCTCTGGGATTATCCTGTACCTGGAACATGACAGCTATTGAAGAATCTGCA |
| | | | CGTATTGCTGCTATCGAAGCCAGTGCTGATGGTATTTGCTGGACATTCAGTCCGATGGTGGATGTTTCC |
| | | | CGTGATCCCCGTTGGGGACGAGTTTCCGAAGGGAATGGTGAAGATCCCTTCTTGGGAGCGGAGATTGCG |
| | | | CGTGCTATGGTACGTGGTTATCAAGGGAAAGATATGAGTAGTAATGATGAAATTATGGCTTGCGTGAAG |
| | | | CACTTTGCGTTATATGGGGCATCAGAAGCCGGACGCGACTATAATACAGTGGATATGAGTCATCAACGT |
| | | | ATGTTCAACGAATATATGTTACCTTATCAGGCTGCCGTGGAAGAAGGTGTGGGTAGTGTGATGGCTTCA |
| | | | TTCAATGAAGTGGATGGTGTACCGGCTACCGGAAATAAGTGGCTGATGACCGATGTACTTCGTAAGCAG |
| | | | TGGAATTTTGATGGGTTCGTTGTGACGGACTATACCGGTATCACTGAAATGACCGATCATGGTATGGGT |
| | | | GATACACAAACAGTTGCAGCCCTGGCTCTGAATGCAGGTGTCGATATGGATATGGTGAGCGATGCTTTT |
| | | | ACAAGCACACTTAAAAAATCTCTGGAAGAAGGAAAAGTTTCAGTAAAGGCTGTTGATGCTGCTTGTCGC |
| | | | CGTATTCTGGAAGCTAAGTATAAGCTGGGGCTTTTTGATAATCCCTATAAATATTGTGATATAACCCGT |
| | | | CCTAAAAAACAAATCTTTACAAAAGAACACCGCGCTATAGCCCGTAAGACAGCTTCGGAAAGCTTTGTT |
| | | | CTCTTGAAGAATGAGAATAGTGTACTCCCTCTGGCAAAGAAAGGTACCATTGCTGTAGTAGGTCCTTTG |
| | | | GCCGATAGCCGTAGCAATATGCCGGGCACGTGGAGTGTGGCCGCTGTGATGAACAAATATCCTTCTTTG |
| | | | ATTGAAGGCTTGAAAGAAGTAGTGGGAGGCAAGGCTAAAATTCTTACGGCTAAAGGAAGTAATCTGATG |
| | | | AGTGATGCCGAATACGAAGAACGTGCTACTATGTTTGGCCGTACTCTGCATCGTGACAATCGTACAGAT |
| | | | AAGGAACTGCTGGATGAGGCGCTTGCTGTAGCTGCCAAGTCTGACGTGATTGTTGCTGCTTTGGGTGAG |
| | | | TCTTCCGAGATGAGCGGTGAAAGTAGTTGCCGTACAGACCTCGAAATGCCGGATACGCAACGTGTACTT |
| | | | TTGCAGGAATTGTTGAAAACCGGCAAACCGGTGGTATTGGTGTTGTTTACCGGTCGTCCGTTAGTATTG |
| | | | AATTGGGAGCAGGAAAATGTACCTGCTATTCTGAATGTGTGGTTTGGTGGTAGTGAAGCTGCTCTTGCC |
| | | | ATTGGTGATGTACTGTTTGGAAATGTAAATCCGAGTGGCAAACTTACTACTACTTTTCCGAAGAGTGTA |
| | | | GGACAGATTCCTTTGTTCTATAACCATAAGAATACTGGTCGTCCTTTGCCTCAAGGGGCCTGGTTCCAG |
| | | | AAGTTCCGTAGCAATTATCTGGATGTAGATAACGAACCGCTTTATCCGTTTGGATATGGCTTGAGCTAT |
| | | | ACTACTTTCTCTTATAGTGATATTACATTGGATAAATCGTCCATGAATATCAATGGAGAGATTATGGCA |
| | | | ACTGTAACGGTAACCAATACAGGTAAGTATGACGGTTCGGAAGTAGTGCAGCTATATATCCGCGATCTT |
| | | | ATAGGCAGTGTAACACGTCCGGTGAAAGAACTGAAAGGCTTTGAAAAAATCTTCTTGAAAGCCGGTGAA |
| | | | TCCAAACAAGTGTCTTTCAAGTTAACAGCTGATATGTTGAAGTTCTACAATTACAATCTGGATTTTGTG |
| | | | TGCGAACCGGGTGACTTTGAAGTAATGATAGGTGGTGATAGCCGTGATGTGAATAAGGCCTTATTTTCG |
| | | | CTTCAA |
| CMR200 | CMR200 | 10 | CAATGGAAACCGGCCGGAGATAGAATAAAGACAAAGTGGGCAGAACAGATCAATCCTTCCGATGTATTG |
| 017.102 | 017.21- | | CCCGAGTATCCAAGGCCCATCATGCAGCGTAATGACTGGAAAAACCTGAATGGTTTGTGGGATTATGCT |
| | 605.P | | ATTATTGATAAAGGTGGACGCATTCCAACGGATTTTGAAGGCCAAATTCTCGTACCTTTTGCTGTAGAA |
| | (605).. | | TCGTCTTTGTCCGGAGTAGGAAAAAGAGTGAACGAAAATCAGGAAGTAATCTATCAGCGGAGCTTTGAG |
| | pMCSG68 | | ATACCTTCAGCCTGGAGAGGAAAACAGGTTTTGCTACATTTTGGTGCCGTTGACTGGAAAACCGATGTA |
| | | | TGGGTGAACGATATTAAGGTTGGAAGTCATACCGGAGGATTTACTCCATTCTCCTTTGATATAACTCCT |
| | | | GCCTTGTCGGCTAAAGGTAACAACCGTCTGGTTGTAAAGGTTTGGGACCCTACGGACAGAGGCCCTCAA |
| | | | CCACGTGGTAAGCAAGTCAGCAGACCGGAAGGTATCTGGTACACTCCTGTAACAGGTATCTGGCAAACT |
| | | | GTATGGCTGGAACCTGTTGCTGGTAAACATATTGAGAATCTTCGTATTACTCCTGATATTGACCGTCAT |
| | | | CTGTTAACGGTAAAAGCTGAACTGAACACCAACAGCACATCAGACTTCGTGGAGGTGAATGTGTATGAT |
| | | | GGTAATCAATTAATTGCTGCCGGTAAGAGTATTAATGGGGAACCTGTAGAAGTGGCAATGCCTGAAAAT |
| | | | GCAAAACTGTGGAGCCCTGATTCTCCTTTTCTCTATACTTTGAAAGTTACTTTAAAAGAGGGGAATAAG |
| | | | ATTGTGGATAAGGTGGATAGCTATGCGGCCATGCGTAAATATTCCACTCGCAGGGATGCCAATGGTATC |
| | | | GTACGTTTGGAACTGAATAATGAAGCGCTGTTCCAGTTTGGCCCGCTTGATCAAGGTTGGTGGCCTGAC |
| | | | GGTCTGTATACGGCTCCTACGGATGAAGCTTTGCTGTACGACATTCAGAAGACAAAAGATTTTGGTTAT |
| | | | AATATGATCCGTAAACATATTAAAGTAGAGCCTGCCCGTTGGTATACATATTGCGACCAGCTTGGAATT |
| | | | ATTGTGTGGCAAGACATGCCGAGTGGTGACCGCAACCCGCAATGGCAGAACCGGAAGTACTTTGATGGT |
| | | | ACGGAAATGAAGCGTTCAGCCGAATCAGAAGCTTATTATCGCAAAGAATGGAAAGAAATAATGGACTGT |
| | | | CTGTATTCTTATCCTTGCATTGGTACCTGGGTGCCATTTAATGAGGCTTGGGGACAGTTTAAGACCGTT |
| | | | GAAATTGCTGAATGGACGAAACAATATGATCCGACCCGTTTGGTGAATCCAGCAAGTGGCGGTAATCAT |
| | | | TATACTTGTGGTGATATGCTTGACCTGCATAATTATCCGGCACCTGAGATGTACTTGTATGATGCTCAG |
| | | | CGTGCAACTGTTTTGGGTGAATACGGTGGTATCGGTCTTGTTCTGAAGGATCATATCTGGGAGCCGAAC |
| | | | CGTAACTGGGGTTATGTTCAATTTAATTCTTCCAAAGAAGCTACGGATGAATATGTGAAGTATGCCGAT |
| | | | ATGCTGTATAAGATGGTAGACAGAGGATTCTCCGCAGCTGTCTATACACAGACTACTGACGTGGAAGTG |
| | | | GAAGTGAATGGCCTGATGACCTATGACCGTAAGGTTATTAAACTGGATGAAAAGCGTGCTAAAGAAATA |
| | | | AATACACGTATCTGTAATTCGTTGAAAAAG |
| CMR200 | CMR200 | 11 | CAGACACTTCCGCAGACAGAGCGGCAATACCTCTCCGGCCACGGATGCGACGACACAGTAGAATGGGAC |
| 018.102 | 018.20- | | TTTTTCTGTACCGACGGACGTAACTCCGGTCGATGGACGAAAATAGGCGTCCCCTCTTGCTGGGAGTTG |
| | 949.P | | CAGGGTTTTGGTACCTATCAGTATGGAATTAGTTTTTATGGTAAAGCCTTTCCCGAAGGCATTGCCGGT |
| | (949).. | | GAGAAAGGAATGTATAAATATGAGTTTGAAGTTCCCGAGGAATTTCGTGGCAAGCAGGTCAGCCTTGTG |
| | pMCSG68 | | TTCGAAGCATCCATGACCGATACGGAAGTTAAGGTTAACGGACGTAAGGCAGGATCGAAACACCAGGGA |
| | | | GCCTTCTATTGCTTTTCATATAATGTCACGGATTTACTGAAATATGGCAAGAAGAATCAGCTGGAAGTA |
| | | | ACAGTTTCCAAGGAGAGTGAGAATGCCAGTGTGAATCTTGCCGAACGGCGCGCCGATTATTGGAACTTT |
| | | | GGCGGTATCTTCCGCCCGGTATTTCTGGAAGTAAAACCTGCCGTCAATCTCCGTCATATTGCTATTGAT |
| | | | GCACAAATGGACGGATCATTCCGTGCCAATTGCTACACGAATATCTCCGGTGACGGAATGAGTATCCGT |
| | | | GCACAGATTTTGGACGGTAAAGGGAAGAAACTGGCAGATACCACCGTACCCCTAAAAGCCGGAAGCGAC |
| | | | TGGACTACTTTACAATTGAACGTTTCTGCCCCTGCCTTATGGACGGCAGAAACTCCGAATCTTTATAAA |
| | | | GCTCAATTTTCACTGTTGGATAAAGGAGGTAAAGTCCTGCATCATGAGACCGAGACATTCGGTTTCCGT |
| | | | ACTATCGAAGTTCGTGAAAGTGACGGATTGTACGTGAACGGGGTGCGTATCAACGTGCGTGGTGTCAAC |
| | | | CGTCATAGTTTCCGTCCCGAAAGCGGTCGTACCCTAAGTAAAGCGAAGAATATTGAAGATGTACTTCTG |
| | | | ATGAAGGGCATGAATATGAATTCTGTCCGTCTGAGCCACTATCCGGCGGACCCGGAATTTCTGGAAGCA |
| | | | TGCGACTCTCTTGGACTCTATGTTATGGATGAACTGGGTGGCTGGCATGGCAAGTACGACACCCCTACG |
| | | | GGAGTACGTCTGATTGAAGGCATGATAGAACGTGATGTGAACCATCCGTCCATTATCTGGTGGAGCAAT |
| | | | GGTAATGAAAAAGGCTGGAACATTGAACTGGACGGAGAATTCCATAAATACGATCTGCAGAAACGCCCG |
| | | | GTCATCCATCCGCAAGGTAACTTCTCCGGTTTCGAAACCATGCACTATCGTTCGTATGGAGAAAGCCAG |
| | | | AACTACATGCGCCTGCCGGAAATCTTTATGCCTACTGAATTCCTGCATGGTTTGTACGACGGAGGTCAT |
| | | | GGTGCCGGCCTGTATGATTACTGGGAAATGATGCGTAAACATCCGCGTTGTATCGGTGGTTTCCTGTGG |
| | | | GTATTGGCGGATGAAGGCGTGAAGCGCGTGGATATGGACGGGTTCATAGACAATCAGGGAAATTTCGGA |
| | | | GCTGACGGAATTGTAGGCCCTCACCATGAAAAGGAAGGCAGCTATTACACTATCAAGCAGCTATGGAGC |
| | | | CCGGTGCAGGTTATGAATACCGCTATCGACCGGAATTTCGACGGTAAACTCTCTGTGGAGAACCGTTAT |
| | | | GATTATCTGAACCTGAACACCTGTCGTTTTATCTGGCAGCAAGTGAAGTTCCCGTCGGTAACGGATGCT |
| | | | TCCAATACAACTACACGGATTCTGAAACAAGGTGAAGTGCAAGGAAGCGATGTAGCAGCCCATGGAGTG |
| | | | GGAGTGGTGGATATCAAGACTTCTATTCTTCCCGAAGCGGATGCTCTTTTCCTGACAGTTATAGATAAA |
| | | | TATGGGTATGAACTTTGGCGCTGGACTTTCCCCGTAGATAAACTGAATCGGGAAACAGAACAGTTTTCT |
| | | | GCATCATCCGGCCGTGTATCCTATACGGAAACAGAAAAAGGTATTACGGTAAAAGCAAACGGGCGTACT |
| | | | TTTGTCTTTTCAAAGAAAGACGGGCAGCTGAAAGATGTATCCGTCAATAACCGTAAGATTAGTTTTGCT |
| | | | AACGGTCCCCGTTTTATCGGTGCACGTCGTGCAGACCGTTCCCTAGATCAGTTCTATAATCATGATGAC |
| | | | GAAAAAGCCAAGGCAAAGGACCGTACTTACAGTGAATTTACCGATGCGGCAGTCTTCACGAAACTGGAT |
| | | | GTGAAAGAAGAGGGGGGGAATCTGATCCTCACCGCTAATTATAAACTGGGTAATTTAGATAAAGCTCAG |
| | | | TGGACAATTCATCCGGACGGCATGGCTACTCTTGATTATACCTACAACTTCTCCGGTGTGGTAGACCTG |
| | | | ATGGGTATTTGCTTTGATTACCCTGAAGAACAAGTGCTCAGCAAGCGTTGGTTGGGAGCAGGTCCGTAT |
| | | | CGTGTATGGCAGAATCGTATTCATGGCACGCAGTATGATATCTGGGAGAATGATTATAACGATCCTATT |
| | | | CCGGGTGAGACATTCACCTATCCTGAATTCAAGGGATATTTTGGCAGTGTCTCTTGGATGAGTATTCGC |
| | | | ACGAAAGAGGGAACCATCAGCCTGACGAATGAAACACCTGATTCCTATATCGGAGTATATCAACCCCGT |
| | | | GATGGTCGTGACCGGTTACTGTATACACTTCCCGAAAGCGGAATTTCTGTTTTGAATGTAATTCCTCCG |
| | | | GTGCGTAATAAAGTAAATTCCACGGACTTGTGCGGTCCTTCTTCACAACCAAAATGGGTGGATGGCTCG |
| | | | CAAACGGGACGCCTTGTTATCCGGTTTGAA |
| CMR200 | CMR200 | 12 | CAGCGCAGTGAGTATCTACTTGAAAAGAACTGGAAGTTCATGAAGGGGGAAGCTCCGGAAGCCATGAAG |
| 027.102 | 027.20- | | CCGGAATTTGACGACCGGAAGTGGGAAACCGTAACCGTGCCTCACGACTGGGCCATTTTTGGTCCCTTC |
| | 824.P | | GATCGCAGCAACGATTTGCAGGAAGTGGCGGTAACGCAGAACTTCGAGAAGAAAGCTTCCGTCAAGACC |
| | (824).. | | GGACGTACCGGTGGACTTCCTTATGTTGGCATCGGATGGTATCGTACTAGGTTCGATGCCCCCGTCAAT |
| | pMCSG68 | | CAACAGACGACACTTGTCTTTGATGGTGCCATGAGCGAAGCCCGTGTATATGTCAATGGACAAGAAGCA |
| | | | TGCTTCTGGCCATTTGGTTATAATTCTTTCCATTGTGATGTCACCGGACTTTTGAATAAAGACGGTAAA |
| | | | AACAATACGCTTGCCGTGCGTTTGGAAAATAAACCACAATCTTCCCGTTGGTATCCTGGCGCAGGACTT |
| | | | TATCGCAATGTGCGTGTAGTGAGTACCGATAAAGTACATGTTCCTGTATGGGGTACTCAGCTGACTACT |
| | | | CCTCATGTTTCTGATGAGTATGCTTCAGTACGTCTGTTGACCACTATTGCCAATGATGAAGAAAGAGAT |
| | | | ATCCGTATCGTGACAGAGATAATCTCTCCCGATGGGAAAGTCGTTGCAACGAAGGATAATACCCGTAAG |
| | | | ATTAATCATGGTCAGCCTTTTGAACAAAACTTCCTGGTGAATGCTCCTTGCTTGTGGTCGCCGGAGACA |
| | | | CCTTATTTATATAAAGCTGTTTCTAAAATCTATGCCGATGGCAAGCAAACGGATGAATACACTACTCGT |
| | | | TTCGGCATCCGCAGCATAGAAATCATTGCCGACAAAGGATTTTTCCTGAACGGTAAGCATCGCAAGTTC |
| | | | CAGGGGGTGTGCAATCACCACGATCTTGGTCCGTTAGGCGCTGCCATCAATGTTGCTGCATTGCGCCGT |
| | | | CAACTTACGATGCTGAAAGATATGGGTTGTGATGCCATCCGCACCGCTCACAATATGCCGGCACCGGAG |
| | | | TTAGTGCAACTTTGTGATGAAATGGGTTTTATGATGATGCTGGAACCTTTCGACGAATGGGACATTGCC |
| | | | AAATGTGAGAATGGCTATCACCGTTATTTCAACGAGTGGGCAGAACGTGATATGATAAATATGTTGCAT |
| | | | CAGTTCCGCAACAATCCTTGTGTCGTAATGTGGAGTATCGGTAATGAAGTTCCTACCCAATGTAGTCCC |
| | | | GTAGGCTATAAAGTCGCTTCTTTCTTGCAGGATATCTGTCATCGTGAAGATCCGACACGTCCTGTTACT |
| | | | TGCGGCATGGATCAGGTGACTTGTGTTCTTGCTAATGGTTTTGCCGCCATGATTGATGTGCCCGGTTTT |
| | | | AATTATCGCGCACACCGTTATCTGGAAGCTTATGAACTGTTGCCGCAGAATATAGTACTTGGTTCTGAA |
| | | | ACATCCTCTACCGTTAGTTCTCGTGGCGTATATAAATTTCCTGTAGAGAAACGCGGGGATGCGAAGTAC |
| | | | GATGATCACCAGTCTTCCGGATATGACTTGGAGCATTGTGCCTGGTCTAATGTTCCAGATGAAGATTTT |
| | | | GCTTTAGCGGATGATTATGACTGGACTATCGGTCAATTCGTTTGGACAGGATTCGATTATCTGGGTGAG |
| | | | CCTTCTCCTTATGATACGGATGCATGGCCAAGTCATAGCTCTTTGTTTGGTATCATTGACCTTGCCAGT |
| | | | TTGCCAAAAGACCGCTACTATCTGTACCGTAGTCTTTGGAATAAGAATGTGAATACACTCCATATACTT |
| | | | CCTCACTGGACATGGCCGGGTAGGGAAGGAGAGAATACTCCTGTCTTTGTTTACACAAACTATCCTGCT |
| | | | GCCGAACTTTTCGTTAATGGAAAAAGCTATGGTAAACAGCATAAACTGACAGCCGAAGAGAGTAAAGCT |
| | | | ATTCAGGACAAAGATACACTTGCCCTCCAGCGTCGTTACCGCCTGATGTGGATGGACGTTCCTTATGAG |
| | | | CCGGGTGAAGTGAAAGTGGTGGCTTACGATGCTTCCGGCAAACCTGCTGAAGAAAAAGTAGTTCGTACT |
| | | | TCCGGCAAACCTCATCATCTGGAAGTCATTGCTGACCGTGACCAACTCACTGCCGATGGTAAAGATTTG |
| | | | GCATACATCACTGTTCGTGTGGTTGATAAAGACGGAAACCTTTGTCCTGCTGATAATCGTCTTGTAAAC |
| | | | TTTACGGTGAAAGGCGCGGGGCGTTATCGTGCTGCCGCTAATGGAGATGCAACTTCACTTGATTTATTC |
| | | | CACTTGCCGAAGATGCCCGCTTTCAGTGGTCAGCTGACAGCCATTGTTCAAATGACCGAACAGCCCGGT |
| | | | GAAATTATTTTCGAGGCTAAGGCTAAAGGGGTGAAATCTGGTAAGCTTGTGCTGAGGTCTGTTAGAGAG |
| CMR200 | CMR200 | 13 | GGTGAAAAGGCAGAAAAAATACAGGATTTTGCTGAGTTTATAACCATTCAGGGGCAAGACCTGATAAAA |
| 113.102 | 113.22- | | CCTGATGGTACGAAACTCTTTATCATGGGTACCAATCTGGGCAATTGGCTGAATCCGGAAGGGTATATG |
| | 415.P | | TTTAAGTTTAACAAAACGAATTCTCCCCGGTTTATCAATGAAATGTTCTGCCAATTGGTAGGACCCGAC |
| | (415).. | | TTTACTGCTGAGTTTTGGAAAGCTTTCAAAGACAATTATATCATTCGTGAAGATATTCAGTTTATTAAG |
| | pMCSG68 | | AATACAGGTGCGAATACCATTCGTCTTCCATTCCATTATAAGCTTTTCACGGATGAGGACTTTATGGGG |
| | | | TTGACTGCCGGTCAGGATGGTTTTGCCCGTGTAGACAGTGTTGTGGAATGGTGCCGTGAAGCCGATCTT |
| | | | TATCTGATTCTTGATATGCATGATGCTCCGGGTGGACAAACGGGTGATAATATAGATGATAGCTACGGA |
| | | | TATCCTTGGTTGTTTGAAAGTGAAGCCAGCCAGCAATTGTATTGCGATATCTGGCGCAAGATTGCAGAC |
| | | | CGGTATAAGAATGAACCGGTGATTCTCGGTTATGAGCTTTTCAATGAACCTATCGCTCCGTATTTTCCG |
| | | | AATATGGAAGAATTGAACGGTAAACTGGAAGATATTTATAAGAAAGGGGTAGCTGCTATCCGCGAGGTG |
| | | | GACAATAACCATATTATTCTGTTGGGTGGCGCTCAGTGGAACGGTAACTTCAAGCCGTTCAAGGATTCT |
| | | | AAGTTTGATGATAAAATAATGTATACTTGCCATCGTTATGGAGGTGATCCTACTAAAGATGATATTCAA |
| | | | ACTATAATAGACTTCCGCGACAGTGTGAACTTACCAATGTATATGGGTGAGATAGGACATAACACGGAC |
| | | | GAATGGCAAGCTGCTTTTTGCCAGACGATGCGTGAGAATAATATCGGTTATACCTTCTGGCCGTATAAG |
| | | | AAGATGGATGGTTCCAGCTTTGTAGGTATTACTCCGCCGGAAAATTGGGCGAATATCCTTTATTTCTCC |
| | | | GAATCTCCACGCACATCTTATAAAGAAATCCGGGATGCCCGTCCCGACCAGATGATGGTACGCAAGGCA |
| | | | ATGATGGATTTCATTGAGGCTTGCAAACTGAAGAACTGTGTGGTGCAGGAAGGGTATATTCAGTCGTTA |
| | | | GGTATGAAA |
| CMR200 | CMR200 | 14 | ACACAAGTGGCAAATAAAGGTAGCGATGCGGCAACCGAGAAAAAAGTAGAGTCTCTTTTATCCAGAATG |
| 122.102 | 122.20- | | ACCCTTGAAGAGAAAATCGGTCAGATGAACCAGATTACCTCTTACGGGAATATTGAGGATATGAGTAGT |
| | 750.P | | TTAATTAAGAAAGGTGAAGTCGGGTCTATCCTGAATGAGGTGGATCCGGTACGTATTAATGCGTTGCAA |
| | (750).. | | CGCGTAGCGATGGAGGAGTCCCGGTTGGGAATCCCTTTGTTGATAGCTCGCGATGTTATTCACGGGTTT |
| | pMCSG68 | | AAAACCATTTTTCCCATCCCATTGGGACAAGCGGCTTCGTTCAATCCGCAGATTGCGAAAGACGGTGCA |
| | | | CGGGTAGCGGCTATTGAGGCTTCTTCCGTAGGTATCCGTTGGACTTTTGCACCGATGATCGACATTGCC |
| | | | CGTGATCCTCGCTGGGGGCGCATTGCCGAAGGATGTGGTGAAGACACTTACCTGACTTCTGTAATGGGA |
| | | | GCTGCCATGGTAGAAGGTTTTCAGGGAGATTCTTTGAATAGTCCCACTTCCATAGCTGCCTGTCCTAAA |
| | | | CATTTTGTGGGCTATGGTGCAGCTGAAGGCGGACGTGACTATAATTCGACATTTATTCCTGAACGTCGC |
| | | | CTGCGTAATGTTTACTTGCCACCGTTTGAAGCGGCAACGAAAGCGGGTGCAGCTACGTTTATGACTTCC |
| | | | TTTAATGATAATGATGGGATACCCTCTACCGGAAATGCTTTCATATTGAAAGATGTGCTTCGTGGCGAG |
| | | | TGGGGATTTGATGGTTTGGTAGTGACAGACTGGGCTTCTGCCAGCGAAATGATAAGTCATGGTTTTGCT |
| | | | GCCGATTCTAAAGAGGTAGCCATGAAATCAGTGAATGCTGGGGTGGATATGGAAATGGTAAGTTATACC |
| | | | TTTGTAAAAGAATTGCCTGCATTGATAAAAGAAGGAAAGGTGAAAGAAAGCACCATTGATGAAGCCGTT |
| | | | CGTAATATATTGCGCGTCAAGTATCGTCTGGGATTGTTTGATGTTCCTTATGTAGATGAAAAGCAACCC |
| | | | TCTGTCATGTATGATCCTTCTCATCTGAAAGTAGCTAAGCAGGCTGCTGTAGAATCGGCTATCCTGTTG |
| | | | AAGAATGATAAAGAAGTACTGCCGTTACAGGAGTCTCTGAAAACCATTGCTGTGGTAGGACCTATGGCC |
| | | | AATGCGCCTTATGAACAATTGGGTACCTGGATCTTTGATGGTGAGAAAGCTCATACTCAGACACCACTG |
| | | | AATGCTATTAAGGAAATAGTTGGCGACAAAGTACAGGTGATTTATGAACCCGGATTAGCTTATAGCCGT |
| | | | GAGAAAAATCCGGCAGGCGTAGCAAAAGCTGCTGCTGTTGCTGCACGTGCAGATGTCATTCTTGCTTTT |
| | | | GTGGGTGAAGAAGCCATTCTTTCGGGTGAAGCACACTGTCTGGCAGATTTGAATCTTCAGGGTGATCAA |
| | | | AGTGCTTTGATTACGGCTTTGGCTAAGACAGGTAAACCTGTAGTAACCATTGTGATGGCAGGTCGTCCG |
| | | | TTGACTATCGGTCAGGAAGTGGAAGAATCAACAGCTGTTCTTTATTCATTCCATCCGGGTACGATGGGT |
| | | | GGACCGGCATTGGCCGATCTGCTGTGGGGTAAGGCGGTTCCAAGTGGAAAAACACCGGTTACTTTCCCG |
| | | | AAGATGGTAGGACAAATTCCGGTATATTATGCTCATAACAATACCGGGCGGCCGGCTACACGTAATGAG |
| | | | GTGTTGCTGGATGATATTGCTGTTGAGGCTGGACAAACTTCATTGGGATGTACTTCTTTCTATATGGAT |
| | | | GCCGGTTTTGATCCTTTATTCCCATTTGGCTATGGCTTGTCGTATACAACGTTCAAGTATAGTAATGTC |
| | | | AAACTTTCATCAGCGTCATTGAAGAAAGATGATGTATTGACTGTGACATTTGATCTGGAAAATACAGGT |
| | | | AAATATAAGGGGACGGAAGTTGCTCAATTGTATATACAAGATAAGGTTGGTTCTGTAACTCGTCCGGTG |
| | | | AAAGAACTGAAACGTTTTACTCGGGTAACCTTGAAACCGGGCGAGAAAAAGAATGTTTCGTTTGAACTA |
| | | | CCCGTTAGTGAACTTGCATTTTGGAACATCGATATGGTGAAAGTTGTGGAACCCGGAGACTTTGGACTT |
| | | | TGGGTGGCAACAGACAGCCAATCGGGAGAAGAAGTTTTCTTTAAGGTGGTAGAT |
| CMR200 | CMR200 | 15 | TCTGATTCAAATGTTGATTTCAATAAAGATTGGAAATTCGTACTGAAAGATTCTGCTCATTATTCATAT |
| 130.102 | 130.32- | | ACTTCTTATGTCCCTGGTGATGAATGGAAGAAAGTGAACCTGCCACACGACTGGAGTGTTGGTCTGCCT |
| | 851.P | | TACGACTCCATCTCTGGCGAAGGGTGTGTAGCTTTCCTTCAGGGAGGAATAGGATGGTATAGCAAATCA |
| | (851).. | | TTTCCCACAACAATCAGCGCAAATCAGAAATGCTATATAGTGTTCGATGGAGTATATAATAATTCTGAG |
| | pMCSG68 | | TATTGGATAAATGGCAAAAAACTTGGATATCATCTTTCGGGATATGCTCCTTTTTATTTTGATGTCACA |
| | | | GACTATCTCAATCCCAATGAGGATAACCGCATGACTGTAAGGGTCGACCACAGCCATTATGCCGACAGC |
| | | | AGATGGTACACCGGTTCAGGTATATACAGGGATGTGAAAATGATTGTAACCGACAGACTGCATATTCCG |
| | | | GTTTGGGGAACATTTGTCACTACTCCCGTGGTTACTGATAAATATGCTAAAGTAAACAACCAAATTACC |
| | | | GTGCGCAACAGTTACTCTGAACCCAGAACAGCTGTTGTTGAGATAGTGTATAAAGATAATAAAGGCAAT |
| | | | ATCGCAGCCTTTGAGGTCTTCAGTATAAAACTGAATGCTGGTGAGGAGAAAATTATCGACATCGTATCG |
| | | | GAGATAAAACAGCCGGATTTGTGGAGCGTCGAGATACCAGTCCTCTATACAGCCGAGACCCGTATTAAG |
| | | | AATGGCGATGAAGTCATTTCTGAAAACACTGTCAGGTTCGGTATACGAACATTCCACTTTGATGCAGAC |
| | | | AAAGGTTTCTTCCTTAACGGAAAAAATATGAAGATAAAAGGAGTATGCCTGCATCATGATGCCGGTATA |
| | | | GTTGGCACAGCAATGATACGCGATGTGTGGTACCGACGTCTGAAAACCCTTAAGGAAGGAGGATGTAAC |
| | | | GCCATCCGCCTTTCGCACAATCCGGGAGCGGATGAGTTTCTGTCTTTGTGCGATGAGATAGGTCTTCTG |
| | | | GTCCAGGAAGAGTTCTTCGATGAGTGGGATTATCCCAAAGATAAAAGGCTCAATATGAAGGAAACGGTA |
| | | | GAAGACTATCCTACTCATGGTTATTGTGAGCATTTCCAGGAATGGGCTGAAAGGGATTTGAAAAACGTA |
| | | | ATGAGGAGAAGCCGTAATCATGCCTGTATCTTCCAGTGGAGTATAGGTAATGAAATAGAATGGACTTAT |
| | | | ACCGGATGCCGTGAGGCAACAGGTTTCTTTGGAGCCGATTCCAACGGTAATTACTTCTGGAACCAGCCT |
| | | | CCATACTCTAAAGAAAAAATCAGAGAAATGTGGAAAATCCAGCCTAAACAAGCATACGACATTGGTCGT |
| | | | ACAGCGCAAAAATTAGCAGCATGGACACGCCAGATGGATACTACACGAGTGGTTACCGCCAACTGCATC |
| | | | CTGCCTTCCATAAGTTTTGAGACAGGATATATCGATGCACTTGATGTGGCTGGTTTCAGCTACAGACGC |
| | | | GTGATGTATGATTATGCTAAGAAGAATTATCCTGACAAACCTATAATGGGTACAGAAAATCTTGGTCAG |
| | | | TGGCACGAATGGAAGGCGGTGATTGAAAGAGATTTCGTTCCGGGTATGTTTATATGGACAGGAGTCGAT |
| | | | TATCTGGGAGAAAGTGGAAGCCGCCTTTCAAGATGGCCTCAAAAGTCAATAGGATGTGGTCTCCTGGAT |
| | | | ATGTGCGGCTATGTGAAGCCTTCGTACGACATGATGAAATCATTGTGGACTGACAAGCCTTTTATTGCT |
| | | | ATATATTCACAGACTCCAGACAAATCTTCGTATCTCCAGGTAAAAGATGGCTTTACTGATAAGAAAGGA |
| | | | CATGAATGGGATAGAAGATTATGGGTTTGGGATGATGTAAACTCTCACTGGAATTATCAGAAAGGTGAC |
| | | | TCGGTAATAGTAGAAATATATTCCAATTGTGATGAAGTGGAACTTTTCGTTAACGGCAAGTCGATGGGA |
| | | | AAGAAGTATATAGACGATTTTGAGGATCATATCTATAAATGGGCAGTTCAGTACAAGCCTGGCACTATT |
| | | | ACCGCAAAAGGAAAAAATAAGTTAGGTAATACCACTACAGCTATAAGGACTTCAGGCAAAGAACATTCG |
| | | | ATATTGCTAGCGGTTGACAAACAAAGTATCGCAGCAAATGGAAAGGATGTTCTGCATGTCACAGCCCAG |
| | | | CTTACAGACAAAAAAGGTAATCCTGTAAAGACAACAGAACAGATGCTTAAGTTCAACATCGATGGAGAG |
| | | | TACCGTCTGTTGGGTATAGACAATGGAAATGTAAAGAACGTATCTCCATATCAAAGCAAGGAGATTATG |
| | | | ACATATCAGGGAAGATGTATGCTGATGCTTCAGTCAACAGAAAAAACATCGGTACTGAATATCAGTGCA |
| | | | GAAACAAGTGAATTACAGTCGAATAAACTAACAATTAATATAAAA |
| CMR200 | CMR200 | 16 | CAGCGACATGAACAACTCTTGGAAACCGGCTGGAAATTCCACAAAGGAGAAACCAATGGAGCTGAAACT |
| 135.102 | 135.22- | | GTTTCATTTAATGATTCTCAATGGGAATCTGTCTGTATTCCACACGACTGGGCCATTTATGGACCGTTT |
| | 812.P | | GACCGTAATAATGATTTACAAAATGTAGCCATTACTCAGAACTTGGAGAAACAGGCATCTGTCAAGACC |
| | (812).. | | GGACGTACCGGAGGACTTCCTTATGTGGGAGTAGGATGGTATCGCACCCGTTTCGATGCAGACCCTGAC |
| | pMCSG68 | | AAAAAGACAACACTGGTTTTTGATGGAGCCATGAGTGAAGCCCGCGTGTATGTCAATGGAAAAGAAGCC |
| | | | TGCTTCTGGCCTTTCGGTTACAATTCCTTCCATTGTGACATTACTGAGCTTCTGCACAAAGAAGGAAAA |
| | | | GACAATGTATTGGCTGTACGTCTGGAAAACCGTCCTCAATCTTCCCGCTGGTATCCGGGAGCCGGACTT |
| | | | TACCGGAATGTCCATCTGATTACTGCAGAAAAAATACATGTACCTGTATGGGGAACACAGGTTACCACC |
| | | | CCACACGTAGCTAATGACTATGCTTCTGTTTGCCTTCGTACCTCTTTACAGAATGTGGGAAAAGAAGAA |
| | | | ATTACCATAGAAACAGAAATACTGGACCCGAACGGGAAAAAAGTTTCTTTCAAGAAGAACAGCGGACGC |
| | | | ATCAATCACGGGCAACCGTTTACACAAAATTTCATTGTGGAAAACCCGCAATTGTGGTCACCTGAAACA |
| | | | CCGTTCTTATATCAGGCCGTATCTAAAATCTATGCCAACGGAAAACTTACAGATACTTATACCACCCGC |
| | | | TTTGGTATCCGTTCCATCGAATTTGTAGCCGACAAGGGCTTTTTCCTGAACGGCCAGCACCGTAAATTC |
| | | | CAGGGGGTATGCAACCACCACGACTTAGGTCCTTTAGGAGCTGCCATCAACGTATCGGCTCTACGCCAC |
| | | | CAGCTTACATTATTAAAAGACATGGGCTGCGATGCCATTCGTACCGCACACAACATGCCGGCACCCGAG |
| | | | CTTGTCAGACTCTGCGATGAAATGGGATTCATGATGATGATTGAGCCTTTCGATGAATGGGACATTGCC |
| | | | AAGTGTGAAAACGGATACCACCGCTATTTCAACGAATGGGCCGAAAAAGACATGGTAAACATGCTACGG |
| | | | CAATACCGGAATAATCCCTGTGTGGTGATGTGGAGTATCGGTAATGAAGTACCCACCCAATGCAGCAGT |
| | | | GAAGGATACAAAGTAGCCAAGTTCCTGCAAGACATTTGCCATCGGGAAGACCCTACCCGTCCGGTTACC |
| | | | TGCGGCATGGACCAGGTTAGTTGTGTACTCGACAACGGATTTGCGGCCATGCTCGACATTCCGGGATTC |
| | | | AATTATCGCGCACACCGCTATGAAGAAGCTTACCAACGCCTGCCTCAAAATCTTGTATTAGGCTCAGAA |
| | | | ACCTCTTCTACCGTCAGTTCACGCGGTGTATACAAATTCCCGGCAGAGCGTAAAGCCGATGCAAAATAC |
| | | | GAAGACCATCAGTCTTCTTCTTACGACTTGGAATACTGCTCCTGGTCTAACATTCCCGATATAGACTTT |
| | | | GCTCTGGCTGATGACCACCAATGGACTTTGGGGCAGTTTGTCTGGACAGGTTTTGATTATCTGGGTGAA |
| | | | CCCAGTCCATACGATACGGATGCATGGCCCAACCACAGCTCTATGTTCGGTATTATCGACCTGGCTTCC |
| | | | TTACCCAAAGACCGGTACTATTTATACCGCAGCATATGGAACAAGCAAGCTGAAACACTTCATATTCTT |
| | | | CCTCATTGGAACTGGGAGGGCAGAGAAGGAAAAGAAGTACCTGTATTCGTCTATACCAACTATCCGACA |
| | | | GCCGAACTTTTCATCAACGGAAAAAGTTATGGGAAACAGACGAAGAACAACCAAAGCGTAGAGAACCGT |
| | | | TACCGCCTGATGTGGCACAACGCCATTTACGAACCGGGAGAAGTAAAAGTCGTGGCATACGATGAACAC |
| | | | GGTACGGCTAAAGCAGAAAAGATAATCCGCACGGCAGGCAAACCTCACCATATTGAATTGGTTTCTTCA |
| | | | CGCCAGTCGCTCACAGCCGATGGAAAAGATTTGGCTTACGTAACCGTACGTGTTGTGGACAAAGACGGA |
| | | | AATCTCTGCCCCACAGATATGCGCTTGGTGAAATTTAAAGTAAAAGGAGCTGGAAGCTACAAAGCCTCA |
| | | | GCCAATGGAGATCCAACTTGTCTGGATTTGTTCCACCTGCCTCAGATGCACGCCTTCAACGGCATGCTG |
| | | | ACTGCAATTGTGCAATCAGGAAAAGAAGCAGGTACCCTTGAGTTACAAGTCACCGCAAAAGGGCTGAAA |
| | | | TCAGGAAAGATACAAATCGAAGTAAAA |
| CMR200 | CMR200 | 17 | CAGACTGATAAGATTGACCTGGCCGGCTCGTGGACATTTTCTACGGACAGCATGGACTGGAGCCGGGTG |
| 137.102 | 137.19- | | ATTGAACTGCCGGGTTCAATGGCTTCCAATGGTTTTGGGGAAGATATTGCCGTGGGTACTGATTGGACG |
| | 931.P | | GGCGGTATTGTGGATTCTTCTTATTTCTTTAAACCTTCGTATGCCAAATACCGTGAGGCAGGAAATATC |
| | (931).. | | AAGGTACCTTTCTGGCTTCAGCCGGTAAAATATTACAAGGGTAAGGCGTGGTATCAGAAAGAGGTGGTG |
| | pMCSG68 | | ATTCCGGACAGTTGGGAAGGAAAGGACATTTCTCTCTTTTTGGAACGATGCCATTGGGAGAGCCGTTTG |
| | | | TATATAGACGGAAAGGAAATCGGCATGCAAAATGCTTTGGGGGCGCCCCATCGTTATGACCTGACAGGC |
| | | | AAGCTTTCAGCAGGGAAACATGTGTTGATGCTGTGTGTAGACAATCGGGTGAAAAACATTGATCCGGGG |
| | | | GAGAACTCACATAGTATTTCCGACCATACACAAGGAAACTGGAACGGGGGGTAGGCGATATGTTCCTG |
| | | | GAAGTAAAGCCGGAAGTGAATGTGTCTTCCGTCAAGATTATGCCGGAGCGTCTGGCTAAGAAAGTCAGT |
| | | | GTGTCGGCTTCCTTGATGAACCGTTATGAAAAAGATGCCAATGTGGTACTGGAGATGACGGTAGGTAAT |
| | | | GAAAAAGTACAGCAACAATGTACGTTGAAGCCGGGCGAAAATCAAGTGATGATGTCGCTGGCCATGAAG |
| | | | GGAGACATTAAGTGCTGGGATGAGTTTTCTCCATCCTTATATGATTTGAAGCTGAGTGTGAAGGATGCG |
| | | | GATAGCGGTGAAACGGATGTCTATGCGGAACGTTTTGGTTTCCGTGATGTGAAGGTGAAAGACGGCAAA |
| | | | CTCACCATCAACGACCGCCGTTTGTTCCTGCGTGGTACGCTGGATTGTGCCGTATTTCCGAAGACCGGT |
| | | | TTCCCGCCCACGGATGTAGAATCCTGGAAAAAGATTTATACCACCTGTCGGCAGCACGGACTGAACCAT |
| | | | GTGCGCTTCCATTCCTGGTGTCCGCCCGAAGCTGCTTTTGCAGCTGCCGATGGGATGGGTATGTACCTG |
| | | | GAGATAGAATGTTCTTCCTGGGCTAACCAGTCGACTACCATTGGCGATGGAGGCGATCTGGACCGCTTT |
| | | | ATCTGGGAGGAAAGTGAACGCATCGTCCGTGAGTTTGGTAACCATCCTTCTTTCTGCATGATGATGTAC |
| | | | GGTAACGAACCGGCTGGTGAGGGAAGTAATGCCTATCTGACTAATTTTGTTACTACCTGGAAAGAGCGC |
| | | | GATGCCCGCCGTTTATATTGTTCGGGTGCCGGATGGCCCAATTTGCCGGTTAACGACTTCTTGAGCGAT |
| | | | TCCAATCCTCGTATTCAGGCGTGGGGACAAGGTGTGAAGAGTATTATCAACGCACAGGCTCCGCGTACC |
| | | | GACTATGACTGGTCAGAATACATCGGACGTTTCCAGCAGCCGATGGTGAGCCACGAAATCGGGCAGTGG |
| | | | TGTGTATATCCCAACTTCAAGGAAATGGCCAAATACGACGGGGTGATGCGCCCGCGTAATTTTGAGATA |
| | | | TTCCAGGAAACACTGGCTGAAAACGGTATGGCACATTTGGCTGACAGCTTCCTGCTGGCTTCCGGAAAA |
| | | | TTGCAGGCGTTGTGTTATAAGGCCGATATCGAAGCTGCTTTGCGTACAAAAGACTTCGGTGGATTCCAG |
| | | | TTACTGGGCTTGTCTGATTTCCCGGGGCAGGGTACGGCTTTGGTAGGAGTGCTCGATGCGTTCTGGGAA |
| | | | GAAAAAGGCTACATCCGTCCGGAAGAATACCGTCGTTTCTGTAATAGTACGGTACCATTACTGCGCTTG |
| | | | CCGAAGTTGATTTATACCAACCAGGAAACGGTGAAAGGAAGTCTGGAAGTGGCACATTTCGGAGCTGCT |
| | | | CCGCTGGAGGTGACTTCTACTGTCTGGACCCTGAAAACAAAAGAAGGAAAGACAATTGCTTCGGGCACG |
| | | | CTGGCACACCAGCCGGTAGGTATCGGCAATTGTATTCCGTTGGGGCAGCTGGAGATTCCATTGGATAAG |
| | | | GTGGACGTCCCTTCATGTCTGACACTGGAAGCTACATTGGGAGATTACGCCAACAGCTGGCACATCTGG |
| | | | GTATATCCTGCTGCGGTACAGAAAGTAGCTGATGAAGCACAATTGCTGATGACCGACCGTCTGGATGCA |
| | | | AAAGCTTTGCAACGTCTTCAGGAAGGTGGCAACGTACTGCTTTCTTTACGGAAAGGCTCCTTGCCTGCC |
| | | | GAAGCGGGAGGCGAAGTAGTGATAGGTTTCTCTAGCATCTTCTGGAACACGGCCTGGACGCTGGGACAA |
| | | | GCACCGCACACACTGGGTATCCTGTGTAACCCCGCTCATCCGGCACTTTCAGAGTTCCCTACAGAGTAT |
| | | | TACAGTGATTATCAGTGGTGGGATGCCATGAGCCATTCCGGTGCCATCGAAGTGGTCAAGATTGATAAA |
| | | | AACTTGCAGCCGATTGTACGAGTTATCGACGACTGGTTTACGAACCGTCCGCTGGCTTTGTTGTTCGAA |
| | | | GTGAAGGTGGGTAAGGGTAAATTGCTTGTGTCAGGAATTGATTTCTGGCAGGATATGGACAAGCGTACG |
| | | | GAAGCCCGTCAGTTACTCTACAGCTTGAAGAAATATATGTGCGGTAATCGCTTCAATCCCTCTTCTGAA |
| | | | GTCGATGCGAAAGATTTAAGTATTTTGTTTTCCATTAAAAATCAAAAA |
| CMR200 | CMR200 | 18 | AAGGATGCGGAGATGGACCGCTTTATCAGTGACCTGATGGGAAGGATGACCTTGCAGGAAAAGTTAGGA |
| 148.102 | 148.28- | | CAGTTGAATCTGCCGGCTGGGAATGACCTGGTGTCGGGAGCAGTGAAGAACAGCAAGATGGCAGAAGCT |
| | 761.P | | ATCCGAGCTGGTGAGGTCGGCGGCTTTTTCAATGTGAAGGGAGTGGATAAGATTTACCAGATGCAGCGT |
| | (761).. | | ATGGCGGTGGAGGAAACTCGTCTGGGAATTCCTTTGATAGTGGGTGCCGATGTGATTCACGGGTACGAA |
| | pMCSG68 | | ACAATCTTCCCGATTCCGTTGGCCCTGTCTTGTAGCTGGGATACGGCGGCGGTGACACGTATGGCACGT |
| | | | ATTTCTGCCACGGAAGCCAGTGCCGATGGAATCAGCTGGACCTTCAGTCCGATGGTAGACATCTGTCGG |
| | | | GATGCCCGCTGGGGACGTATTGCAGAAGGAAGTGGAGAGGACCCGTACCTCGGGGCGTTGATGGCTGGA |
| | | | GCCTATGTGCGCGGTTATCAGGGTGACGGCATGAAGCAGAACAATGAAATCATGGCCTGTGTGAAGCAC |
| | | | TTTGCGCTGTATGGAGCTTCGGAATCGGGACGTGACTACAATTCGGTGGATATGAGTCGAAACCTGATG |
| | | | TATAATGTGTACCTGGCTCCTTATAAAGGGGCGGTGGAAGCCGGAGTGGGTTCGGTGATGAGCTCGTTC |
| | | | AATACCATCAACGGGGTACCTGCTACAGCTGACAAATGGCTGCTGACGGATTTGCTCCGCAATGAGTGG |
| | | | GGGTTCACGGGGTTTGTGGTGACCGACTACAATTCGATTGGTGAGATGAAGACTCATGGGGTGGCCGAC |
| | | | TTGAAGGAGGCTTCTGCACGGGCGTTGAATGCAGGAACGGACATGGATATGGTGGCACATGGTTTCTTG |
| | | | CATACGCTGGAAGCTTCATTGAAGGAGAAGGCCGTGACGCAGGAGCGGATTGACGAGGCTTGTCGTCGG |
| | | | GTATTGGAAGCCAAGTATAAGTTAGGATTGTTTGAAAATCCTTATAAGTATTGTGATACGCTTCGGGGA |
| | | | CGCAAGGAATTGTTTACGGAGGCGAATCGTAAAGCGGCACGTGAGATTGCGGCTGAAACGTTTGTGCTG |
| | | | TTGAAGAACGAGGGTAAGTTGTTGCCTTTGCAGAAAAAAGGACGCATTGCATTGATTGGGCCGATGGCT |
| | | | GATGCGCAGAACAATATGTGCGGCACGTGGAACATGGATTGTCAGACAGACCGTCATGTGACGATGTAC |
| | | | GAAGCTTTCCGTCGTGCGGTAGGTGATAAGGCTACGGTTTCTTATGCCAAGGGAAGTAATGTGTATTAT |
| | | | AGTGAGCATATTGAGAAAGGGGCGGTCGAACCTCGTCCGCTGACACGTGGCGATGACCGTCAGTTGCGG |
| | | | GCTGAGGCTTTGCGCGTGGCGGCTTCTGCCGATGTGATTGTGGCCGCATTAGGTGAGAGTGCTGAGATG |
| | | | AGCGGAGAGTCTTCTTCTCGTACAGATATTCAGATTCCGGATGCGCAGAAAGATTTGTTGAAGGCATTG |
| | | | ATAGCTACCGGAAAGCCGGTGGTACTGGCTTTGTTTACCGGTCGTCCGCTGGATTTATGCTGGGAGTCT |
| | | | GAGCATGTTCCGGCTATCCTGAACGTGTGGTTTGCCGGCAGTGAAGCGGGTGATGCCATTGCCGATGTG |
| | | | ATGTTTGGAGAAGTATCTCCTTCGGGTAAGCTGACTACGAGTTTCCCACGTGCGGTGGGACAGTTGCCG |
| | | | CTTTATTATAATCACCTGAATACGGGTCGTCCGGATACGGATGACACTACTTTCAATCGTTATGGCAGC |
| | | | AATTACATCGACCAGAGTAATGAACCGCTTTATCCTTTTGGCTATGGTTTGAGTTATACCACTTTCCGT |
| | | | TACGGTAATTTGCAGTTGAGTGCGGAGCGTATGGCCAAGGGTGGGCAGTTGAAGGTAACCGTGCCTGTA |
| | | | ACCAATTCCGGCGAGTGTGACGGAGTAGAGATTGTGCAGTTGTATCTTCACGATGTGTATGCAGAAATC |
| | | | TCCCGTCCGGTGAAGGAGCTGAAAGCTTTCCGCCGTGTGGCCCTTAAAAAGGGAGAGACACAGAATGTA |
| | | | GAGTTTGTACTCGATGAGGATGATTTGAAGTATTATAATTCTCGTCTGGAATATGGATATGAACCGGGA |
| | | | GAGTTTGAAGTGATGGTGGGTCCGGACAGCCGGAATGTGCAGCACGCGACTTTTGTGGCTGAA |

A gene variant is a permanent change in the DNA sequence that makes up a gene. Orthologs are two genes in two different species that share a common ancestor. In the disclosure, the term “ortholog” or “variant” is used interchangeably when referring to any of the 17 novel β-glucosidases comprising the nucleotide sequences of SEQ ID NOs: 2-18 listed in Table 1. For instance, the disclosure provides 17 novel β-glucosidase variants comprising the nucleotide sequences of SEQ ID NOs: 2-18, which are variants of β-glucosidase of SEQ ID NO: 1. These variants comprise changes in their nucleotide sequences which differentiate them from β-glucosidase of SEQ ID NO: 1. Similarly, the identified 17 novel β-glucosidase of SEQ ID NOs: 2-18 are orthologs of β-glucosidase of SEQ ID NO: 1 because sequence analysis show they are delineated from the same common ancestor.

In some aspects, the β-glucosidase is pBATS_0004 β-glucosidase. In some aspects, the β-glucosidase comprises SEQ ID NO: 1. In some aspects, the nucleotide sequence encoding the β-glucosidase comprises at least 80% sequence identity, at least 85% sequence identity, at least 90% sequence identity, at least 95% sequence identity, at least 98% sequence identity, at least 99% sequence identity, or 100% sequence identity to the nucleotide sequence of SEQ ID NO: 1.

In various aspects, the disclosure further provides 17 novel variants of β-glucosidase that exhibit superior properties (e.g., both pH stability and thermal stability) compared to the other known β-glucosidases described in the literature and/or available commercially. Such 17 novel variants of β-glucosidase are identified as follows: APC115038.102 (SEQ ID NO: 2), APC115043.102 (SEQ ID NO: 3), APC115044.102 (SEQ ID NO: 4), APC115045.102 (SEQ ID NO: 5), APC115068.102(SEQ ID NO: 6), APC115077.102 (SEQ ID NO: 7), APC115077.103 (SEQ ID NO: 8), APC115086.102 (SEQ ID NO: 9), CMR200017.102 (SEQ ID NO: 10), CMR200018.102 (SEQ ID NO: 11), CMR200027.102 (SEQ ID NO: 12), CMR200113.102 (SEQ ID NO: 13), CMR200122.102 (SEQ ID NO: 14), CMR200130.102 (SEQ ID NO: 15), CMR200135.102 (SEQ ID NO: 16), CMR200137.102 (SEQ ID NO: 17), and CMR200148.102 (SEQ ID NO: 18). In some aspects, the nucleotide sequence encoding the β-glucosidase comprises at least 80% sequence identity, at least 85% sequence identity, at least 90% sequence identity, at least 95% sequence identity, at least 98% sequence identity, at least 99% sequence identity, or 100% sequence identity to any one of the nucleotide sequences of SEQ ID NOs: 2-18.

### Other Components of the Expression Cassette

As described above, in aspects, the disclosure provides a biosensor and/or a biosensor expression cassette. The reporter enzyme and/or specifically the β-glucosidase is described above. Thus, in aspects, some of the other components of the biosensor and/or expression cassette are described as follows:

In some aspects, the biosensor and/or biosensor expression cassette of the disclosure (i.e., enzyme-linked and/or cell-based enzyme-linked) comprises a transcription factor. In some aspects, the biosensor and/or biosensor expression cassette of the disclosure (i.e., enzyme-linked and/or cell-based enzyme-linked) comprises a promoter. In some aspects, the biosensor and/or biosensor expression cassette of the disclosure (i.e., enzyme-linked and/or cell-based enzyme-linked) comprises a reporter. In some aspects, the biosensor and/or biosensor expression cassette of the disclosure (i.e., enzyme-linked and/or cell-based enzyme-linked) comprises a terminator. In some aspects, the biosensor and/or biosensor expression cassette of the disclosure (i.e., enzyme-linked and/or cell-based enzyme-linked) comprises a transcription factor, a promoter, a reporter, and a terminator to insulate the circuit.

### Transcription Factor (CatM)

In some aspects, the biosensor and/or biosensor expression cassette of the disclosure (i.e., enzyme-linked and/or cell-based enzyme-linked) comprises a nucleic acid comprising a nucleotide sequence encoding a transcription factor (TF). In some aspects, the transcription factor binds the product of the enzyme reaction and activates the transcription of the reporter.

In some aspects, the product of the enzymatic reaction feeds back on the transcription factor to produce a signal proportional to the product concentration and, therefore, to the enzyme activity.

TFs are proteins that can control the expression of genes by binding to specific DNA sequences. Some TFs are triggered after binding to a metabolite or external compound (known as allosteric transcription factors). Once activated, a conformational change in the TF makes itself release from or attach to the DNA sequence upstream of the target gene, thereby activating or repressing its expression. TFs can be assembled together with other DNA parts commonly used in synthetic biology, such as promoters, ribosome binding sites (RBSs), terminators, and reporter genes, to create TF-based biosensor circuits. These genetic devices can thus be used to sense and react to a range of intracellular or environmental ligand concentrations. In some aspects, the transcription factor drives the expression of a fluorescent protein reporter. In some aspects, the transcription factor is especially useful in biomanufacturing and clinical applications, for instance, to get a rapid readout of bioproduct formation.

In some aspects, the transcription factor is a CatM transcription factor. In some aspects, the CatM transcription factor activates the promoter for the reporter enzyme (e.g., without limitation, a β-glucosidase, e.g., without limitation, APC115086). In some aspects, the transcription factor (e.g., without limitation, a CatM transcription factor) activates the promoter for the reporter enzyme. In some aspects, the reporter enzyme is a β-glucosidase. In some aspects, the β-glucosidase is APC115086.

In some aspects, the method of disclosure utilizes a CatM transcription factor-based circuit to sense muconic acid. In some aspects, the disclosure provides additional biosensors (e.g., without limitation, a biosensor based on azelaic acid). For instance, a biosensor that utilizes AzerR as its transcriptional regulator, for instance, can respond to azelaic acid. In some aspects, the circuit of the disclosure enables the swap-in of other sensor cassettes (e.g., without limitation, transcription factor, promoter, and the like).

In some aspects, the nucleotide sequence encoding the CatM transcription factor comprises at least 80% sequence identity, at least 85% sequence identity, at least 90% sequence identity, at least 95% sequence identity, at least 98% sequence identity, at least 99% sequence identity, or 100% sequence identity to the nucleotide sequence of SEQ ID NO: 19. In some aspects, the nucleotide sequence encoding the CatM transcription factor comprises at least 80% sequence identity to the nucleotide sequence of SEQ ID NO: 19. In some aspects, the nucleotide sequence encoding the CatM transcription factor comprises the nucleotide sequence of SEQ ID NO: 19.

SEQ ID NO: 19: Nucleotide sequence of CatM transcription factor
ATGGAACTAAGACACCTCAGATATTTTGTGACCGTGGTTGAAGAGCAAAGCATTTCCAAAGCTGCTGAAA
AGTTGTGTATTGCCCAGCCGCCCCTCAGCCGACAAATTCAAAAACTCGAAGAAGAATTGGGAATTCAGCT
ATTTGAACGCGGCTTCAGACCGGCTAAAGTGACTGAAGCAGGCATGTTTTTTTATCAGCATGCTGTGCAG
ATTTTGACTCATACTGCACAAGCGTCCTCAATGGCAAAACGGATTGCAACGGTCAGTCAAACCTTGAGAA
TTGGTTACGTCAGCTCCTTACTGTATGGTTTGTTACCTGAAATTATTTATCTGTTTCGTCAACAAAATCC
TGAAATTCACATCGAACTCATCGAATGCGGCACCAAAGATCAAATTAATGCCCTTAAGCAGGGAAAAATC
GATCTGGGTTTTGGTCGGCTCAAAATTACCGATCCTGCAATTCGACGTATCGTGTTGCATAAAGAACAGC
TCAAACTTGCAATCCATAAGCATCATCACCTCAATCAGTTTGCAGCAACAGGGGTTCATCTCTCTCAAAT
TATTGATGAACCGATGCTGCTGTACCCAGTCTCTCAAAAGCCCAATTTTGCGACCTTTATTCAGTCACTC
TTTACCGAACTAGGCCTAGTACCATCCAAACTCACCGAAATTCGAGAAATTCAACTGGCACTCGGCTTGG
TGGCAGCAGGTGAAGGCGTCTGCATCGTACCGGCGTCTGCCATGGATATTGGGGTGAAGAATCTACTTTA
TATTCCAATTTTAGATGATGATGCCTATAGCCCAATTTCACTCGCGGTGCGAAATATGGACCACAGTAAT
TACATTCCTAAAATTCTCGCCTGTGTACAGGAGGTGTTTGCAACGCACCATATCAGGCCACTCATCGAAT
AA

### Promoter

In some aspects, the biosensor and/or biosensor expression cassette of the disclosure (i.e., enzyme-linked and/or cell-based enzyme-linked) comprises a nucleic acid comprising a nucleotide sequence encoding a promoter.

In some aspects, the promoter is a T7 promoter. In some aspects, the T7 promoter is a sequence of DNA 18 base pairs long up to the transcription start site at +1. In some aspects, the T7 promoter is recognized by T7 RNA polymerase. The T7 promoter is commonly used to regulate gene expression of recombinant proteins, which can be subsequently used for a variety of downstream research applications.

In some aspects, the nucleotide sequence encoding the T7 promoter comprises at least 80% sequence identity (e.g., at least 85% sequence identity, at least 90% sequence identity, at least 95% sequence identity, at least 98% sequence identity, at least 99% sequence identity, or 100% sequence identity) to the nucleotide sequence of SEQ ID NO: 20. In some aspects, the nucleotide sequence encoding the T7 promoter comprises at least 80% sequence identity to the nucleotide sequence of SEQ ID NO: 20. In some aspects, the nucleotide sequence encoding the T7 promoter comprises the nucleotide sequence of SEQ ID NO: 20.

SEQ ID NO: 20: Nucleotide sequence of T7 promoter
TAATACGACTCACTATAG

In some aspects, the promoter is a CatM promoter. In some aspects, the construct of the disclosure comprises a CatM promoter. In some aspects, the construct of the disclosure comprises an engineered CatM promoter. In some aspects, the engineered CatM promoter is distinguishable from the wild-type Acinetobacter baylyi ADP1 CatM promoter sequence. In some aspects, the engineered CatM promoter comprises a promoter that was previously modified from a wild-type Acinetobacter baylyi ADP1 sequence. In some aspects, the engineered catM promoter utilized in the disclosure has a nucleotide sequence of SEQ ID NO: 21. See “Acinetobacter sp. ADP1 ben operon and cat operon, complete sequence” (GenBank: AF009224.2).

In some aspects, the nucleotide sequence encoding the CatM promoter comprises at least 80% sequence identity (e.g., at least 85% sequence identity, at least 90% sequence identity, at least 95% sequence identity, at least 98% sequence identity, at least 99% sequence identity, or 100% sequence identity) to the nucleotide sequence of SEQ ID NO: 21.

In some aspects, the nucleotide sequence encoding the CatM promoter comprises at least 80% sequence identity to the nucleotide sequence of SEQ ID NO: 21. In some aspects, the nucleotide sequence encoding the CatM promoter comprises the nucleotide sequence of SEQ ID NO: 21.

SEQ ID NO: 21: Nucleotide sequence of CatM
promoter
TTTTCAATAAATACTATTTACATACCTTAAATTAATGTAATAATAAAAA
CCAACACCAATTTGGTATTTTTGCATACTAAAAAGGTATATAAAACCAA
TTAGGGCGTATAA

### T7 Terminator

In some aspects, the biosensor and/or biosensor expression cassette of the disclosure (i.e., enzyme-linked and/or cell-based enzyme-linked) comprises a nucleic acid comprising a nucleotide sequence encoding a terminator.

In some aspects, the terminator is a T7 terminator. In some aspects, the expression construct of the disclosure utilizes the canonical T7 terminator. Such a T7 terminator is commonly used for protein expression vectors where protein expression is regulated at the mRNA (message) level by an inducible T7 RNA polymerase.

In some aspects, the nucleotide sequence encoding the T7 terminator comprises at least 80% sequence identity (e.g., at least 85% sequence identity, at least 90% sequence identity, at least 95% sequence identity, at least 98% sequence identity, at least 99% sequence identity, or 100% sequence identity) to the nucleotide sequence of SEQ ID NO: 22.

In some aspects, the nucleotide sequence encoding the T7 terminator comprises at least 80% sequence identity to the nucleotide sequence of SEQ ID NO: 22. In some aspects, the nucleotide sequence encoding the T7 terminator comprises the nucleotide sequence of SEQ ID NO: 22.

SEQ ID NO: 22: Nucleotide sequence of T7
terminator
CTAGCATAACCCCTTGGGGCCTCTAAACGGGTCTTGAGGGGTTTTTTG

In some aspects, the method of the disclosure is capable of converting the pBTL2_CatM_C21 sensor by moving the engineered promoter and the catM gene into a commonly used protein expression vector and inserting a well-characterized, highly stable, and active β-glucosidase gene (pBATS_0004). See FIG. 2A.

In some aspects, the enzyme-linked biosensor expression cassette of the disclosure comprises a cassette that is inserted into a plasmid. In some aspects, the cassette that is inserted into the plasmid is further transformed into the target microbe.

In some aspects, the disclosure provides an expression cassette comprising a β-glucosidase reporter (APC115086), a CatM transcription factor, and a CatM promoter. In some aspects, the said expression cassette of the disclosure comprises a nucleotide sequence that comprises at least 80% sequence identity (e.g., at least 85% sequence identity, at least 90% sequence identity, at least 95% sequence identity, at least 98% sequence identity, at least 99% sequence identity, or 100% sequence identity) to the nucleotide sequence of SEQ ID NO: 23. In some aspects, the expression cassette of the disclosure comprises a nucleotide sequence that comprises at least 80% sequence identity to the nucleotide sequence of SEQ ID NO: 23. In some aspects, the nucleotide sequence encoding expression cassette comprising a β-glucosidase reporter (APC115086), a CatM transcription factor, and a CatM promoter comprises the nucleotide sequence of SEQ ID NO: 23.

SEQ ID NO: 23: Nucleotide sequence of a complete construct comprising a CatM
transcription factor, a CatM promoter, and a β-glucosidase reporter
(APC115086) (sensor cassette region)
TTATTGAAGCGAAAATAAGGCCTTATTCACATCACGGCTATCACCACCTATCATTACTTCAAAGTCACCC
GGTTCGCACACAAAATCCAGATTGTAATTGTAGAACTTCAACATATCAGCTGTTAACTTGAAAGACACTT
GTTTGGATTCACCGGCTTTCAAGAAGATTTTTTCAAAGCCTTTCAGTTCTTTCACCGGACGTGTTACACT
GCCTATAAGATCGCGGATATATAGCTGCACTACTTCCGAACCGTCATACTTACCTGTATTGGTTACCGTT
ACAGTTGCCATAATCTCTCCATTGATATTCATGGACGATTTATCCAATGTAATATCACTATAAGAGAAAG
TAGTATAGCTCAAGCCATATCCAAACGGATAAAGCGGTTCGTTATCTACATCCAGATAATTGCTACGGAA
CTTCTGGAACCAGGCCCCTTGAGGCAAAGGACGACCAGTATTCTTATGGTTATAGAACAAAGGAATCTGT
CCTACACTCTTCGGAAAAGTAGTAGTAAGTTTGCCACTCGGATTTACATTTCCAAACAGTACATCACCAA
TGGCAAGAGCAGCTTCACTACCACCAAACCACACATTCAGAATAGCAGGTACATTTTCCTGCTCCCAATT
CAATACTAACGGACGACCGGTAAACAACACCAATACCACCGGTTTGCCGGTTTTCAACAATTCCTGCAAA
AGTACACGTTGCGTATCCGGCATTTCGAGGTCTGTACGGCAACTACTTTCACCGCTCATCTCGGAAGACT
CACCCAAAGCAGCAACAATCACGTCAGACTTGGCAGCTACAGCAAGCGCCTCATCCAGCAGTTCCTTATC
TGTACGATTGTCACGATGCAGAGTACGGCCAAACATAGTAGCACGTTCTTCGTATTCGGCATCACTCATC
AGATTACTTCCTTTAGCCGTAAGAATTTTAGCCTTGCCTCCCACTACTTCTTTCAAGCCTTCAATCAAAG
AAGGATATTTGTTCATCACAGCGGCCACACTCCACGTGCCCGGCATATTGCTACGGCTATCGGCCAAAGG
ACCTACTACAGCAATGGTACCTTTCTTTGCCAGAGGGAGTACACTATTCTCATTCTTCAAGAGAACAAAG
CTTTCCGAAGCTGTCTTACGGGCTATAGCGCGGTGTTCTTTTGTAAAGATTTGTTTTTTAGGACGGGTTA
TATCACAATATTTATAGGGATTATCAAAAAGCCCCAGCTTATACTTAGCTTCCAGAATACGGCGACAAGC
AGCATCAACAGCCTTTACTGAAACTTTTCCTTCTTCCAGAGATTTTTTAAGTGTGCTTGTAAAAGCATCG
CTCACCATATCCATATCGACACCTGCATTCAGAGCCAGGGCTGCAACTGTTTGTGTATCACCCATACCAT
GATCGGTCATTTCAGTGATACCGGTATAGTCCGTCACAACGAACCCATCAAAATTCCACTGCTTACGAAG
TACATCGGTCATCAGCCACTTATTTCCGGTAGCCGGTACACCATCCACTTCATTGAATGAAGCCATCACA
CTACCCACACCTTCTTCCACGGCAGCCTGATAAGGTAACATATATTCGTTGAACATACGTTGATGACTCA
TATCCACTGTATTATAGTCGCGTCCGGCTTCTGATGCCCCATATAACGCAAAGTGCTTCACGCAAGCCAT
AATTTCATCATTACTACTCATATCTTTCCCTTGATAACCACGTACCATAGCACGCGCAATCTCCGCTCCC
AAGAAGGGATCTTCACCATTCCCTTCGGAAACTCGTCCCCAACGGGGATCACGGGAAACATCCACCATCG
GACTGAATGTCCAGCAAATACCATCAGCACTGGCTTCGATAGCAGCAATACGTGCAGATTCTTCAATAGC
TGTCATGTTCCAGGTACAGGATAATCCCAGAGGAATAGGAAATACCGTTTCGTATCCATGAATTACATCC
ATACCAAATAAAAGAGGAATACCCAGACGACTTTCTTCTACTGCCTGTTTCTGAACGTCACGAATACGCT
CCACGCCTTTCAAGTTAAAGAGTCCGCCCACTTCACCGGCACGGATACGCTTAGCCACATTACTACTCTT
GGCTTGTCCGGTGGTTATTTCACCCGTAACAGGCAAGTTCAACTGGCCGATTTTCTCTTCCAGAGTCATC
TTCTTCATCAGATCATCAATAAAGCGATCCATATCGACAGGAGATTTCATCTTTCCTGTGTGATTTTCAA
TAAATACTATTTACATACCTTAAATTAATGTAATAATAAAAACCAACACCAATTTGGTATTTTTGCATAC
TAAAAAGGTATATAAAACCAATTAGGGCGTATAAATGGAACTAAGACACCTCAGATATTTTGTGACCGTG
GTTGAAGAGCAAAGCATTTCCAAAGCTGCTGAAAAGTTGTGTATTGCCCAGCCGCCCCTCAGCCGACAAA
TTCAAAAACTCGAAGAAGAATTGGGAATTCAGCTATTTGAACGCGGCTTCAGACCGGCTAAAGTGACTGA
AGCAGGCATGTTTTTTTATCAGCATGCTGTGCAGATTTTGACTCATACTGCACAAGCGTCCTCAATGGCA
AAACGGATTGCAACGGTCAGTCAAACCTTGAGAATTGGTTACGTCAGCTCCTTACTGTATGGTTTGTTAC
CTGAAATTATTTATCTGTTTCGTCAACAAAATCCTGAAATTCACATCGAACTCATCGAATGCGGCACCAA
AGATCAAATTAATGCCCTTAAGCAGGGAAAAATCGATCTGGGTTTTGGTCGGCTCAAAATTACCGATCCT
GCAATTCGACGTATCGTGTTGCATAAAGAACAGCTCAAACTTGCAATCCATAAGCATCATCACCTCAATC
AGTTTGCAGCAACAGGGGTTCATCTCTCTCAAATTATTGATGAACCGATGCTGCTGTACCCAGTCTCTCA
AAAGCCCAATTTTGCGACCTTTATTCAGTCACTCTTTACCGAACTAGGCCTAGTACCATCCAAACTCACC
GAAATTCGAGAAATTCAACTGGCACTCGGCTTGGTGGCAGCAGGTGAAGGCGTCTGCATCGTACCGGCGT
CTGCCATGGATATTGGGGTGAAGAATCTACTTTATATTCCAATTTTAGATGATGATGCCTATAGCCCAAT
TTCACTCGCGGTGCGAAATATGGACCACAGTAATTACATTCCTAAAATTCTCGCCTGTGTACAGGAGGTG
TTTGCAACGCACCATATCAGGCCACTCATCGAATAA

In some aspects, the construct for a β-glucosidase-based muconate biosensor comprises a CatM transcription factor, a CatM promoter, and a β-glucosidase gene. In some aspects, the construct for a β-glucosidase-based muconate biosensor of the disclosure comprises a nucleotide sequence that comprises at least 80% sequence identity (e.g., at least 85% sequence identity, at least 90% sequence identity, at least 95% sequence identity, at least 98% sequence identity, at least 99% sequence identity, or 100% sequence identity) to the nucleotide sequence of SEQ ID NO: 24. In some aspects, the expression cassette of the disclosure comprises a nucleotide sequence that comprises at least 80% sequence identity to the nucleotide sequence of SEQ ID NO: 24.

In some aspects, the disclosure provides an expression cassette comprising a β-glucosidase reporter (APC115086), a CatM transcription factor, and a CatM promoter. In some aspects, the said expression cassette comprises a nucleotide sequence that comprises at least 80% sequence identity to the nucleotide sequence of SEQ ID NO: 24. In some aspects, the nucleotide sequence encoding expression cassette comprising a β-glucosidase reporter (APC115086), a CatM transcription factor, and a CatM promoter comprising the nucleotide sequence of SEQ ID NO: 24.

SEQ ID NO: 24: Nucleotide sequence of an expression cassette comprising a β-
glucosidase reporter (APC115086), a CatM transcription factor, and a catM
promoter.
TTATTGAAGCGAAAATAAGGCCTTATTCACATCACGGCTATCACCACCTATCATTACTTCAAAGTCACCC
GGTTCGCACACAAAATCCAGATTGTAATTGTAGAACTTCAACATATCAGCTGTTAACTTGAAAGACACTT
GTTTGGATTCACCGGCTTTCAAGAAGATTTTTTCAAAGCCTTTCAGTTCTTTCACCGGACGTGTTACACT
GCCTATAAGATCGCGGATATATAGCTGCACTACTTCCGAACCGTCATACTTACCTGTATTGGTTACCGTT
ACAGTTGCCATAATCTCTCCATTGATATTCATGGACGATTTATCCAATGTAATATCACTATAAGAGAAAG
TAGTATAGCTCAAGCCATATCCAAACGGATAAAGCGGTTCGTTATCTACATCCAGATAATTGCTACGGAA
CTTCTGGAACCAGGCCCCTTGAGGCAAAGGACGACCAGTATTCTTATGGTTATAGAACAAAGGAATCTGT
CCTACACTCTTCGGAAAAGTAGTAGTAAGTTTGCCACTCGGATTTACATTTCCAAACAGTACATCACCAA
TGGCAAGAGCAGCTTCACTACCACCAAACCACACATTCAGAATAGCAGGTACATTTTCCTGCTCCCAATT
CAATACTAACGGACGACCGGTAAACAACACCAATACCACCGGTTTGCCGGTTTTCAACAATTCCTGCAAA
AGTACACGTTGCGTATCCGGCATTTCGAGGTCTGTACGGCAACTACTTTCACCGCTCATCTCGGAAGACT
CACCCAAAGCAGCAACAATCACGTCAGACTTGGCAGCTACAGCAAGCGCCTCATCCAGCAGTTCCTTATC
TGTACGATTGTCACGATGCAGAGTACGGCCAAACATAGTAGCACGTTCTTCGTATTCGGCATCACTCATC
AGATTACTTCCTTTAGCCGTAAGAATTTTAGCCTTGCCTCCCACTACTTCTTTCAAGCCTTCAATCAAAG
AAGGATATTTGTTCATCACAGCGGCCACACTCCACGTGCCCGGCATATTGCTACGGCTATCGGCCAAAGG
ACCTACTACAGCAATGGTACCTTTCTTTGCCAGAGGGAGTACACTATTCTCATTCTTCAAGAGAACAAAG
CTTTCCGAAGCTGTCTTACGGGCTATAGCGCGGTGTTCTTTTGTAAAGATTTGTTTTTTAGGACGGGTTA
TATCACAATATTTATAGGGATTATCAAAAAGCCCCAGCTTATACTTAGCTTCCAGAATACGGCGACAAGC
AGCATCAACAGCCTTTACTGAAACTTTTCCTTCTTCCAGAGATTTTTTAAGTGTGCTTGTAAAAGCATCG
CTCACCATATCCATATCGACACCTGCATTCAGAGCCAGGGCTGCAACTGTTTGTGTATCACCCATACCAT
GATCGGTCATTTCAGTGATACCGGTATAGTCCGTCACAACGAACCCATCAAAATTCCACTGCTTACGAAG
TACATCGGTCATCAGCCACTTATTTCCGGTAGCCGGTACACCATCCACTTCATTGAATGAAGCCATCACA
CTACCCACACCTTCTTCCACGGCAGCCTGATAAGGTAACATATATTCGTTGAACATACGTTGATGACTCA
TATCCACTGTATTATAGTCGCGTCCGGCTTCTGATGCCCCATATAACGCAAAGTGCTTCACGCAAGCCAT
AATTTCATCATTACTACTCATATCTTTCCCTTGATAACCACGTACCATAGCACGCGCAATCTCCGCTCCC
AAGAAGGGATCTTCACCATTCCCTTCGGAAACTCGTCCCCAACGGGGATCACGGGAAACATCCACCATCG
GACTGAATGTCCAGCAAATACCATCAGCACTGGCTTCGATAGCAGCAATACGTGCAGATTCTTCAATAGC
TGTCATGTTCCAGGTACAGGATAATCCCAGAGGAATAGGAAATACCGTTTCGTATCCATGAATTACATCC
ATACCAAATAAAAGAGGAATACCCAGACGACTTTCTTCTACTGCCTGTTTCTGAACGTCACGAATACGCT
CCACGCCTTTCAAGTTAAAGAGTCCGCCCACTTCACCGGCACGGATACGCTTAGCCACATTACTACTCTT
GGCTTGTCCGGTGGTTATTTCACCCGTAACAGGCAAGTTCAACTGGCCGATTTTCTCTTCCAGAGTCATC
TTCTTCATCAGATCATCAATAAAGCGATCCATATCGACAGGAGATTTCATCTTTCCTGTGTGATTTTCAA
TAAATACTATTTACATACCTTAAATTAATGTAATAATAAAAACCAACACCAATTTGGTATTTTTGCATAC
TAAAAAGGTATATAAAACCAATTAGGGCGTATAAATGGAACTAAGACACCTCAGATATTTTGTGACCGTG
GTTGAAGAGCAAAGCATTTCCAAAGCTGCTGAAAAGTTGTGTATTGCCCAGCCGCCCCTCAGCCGACAAA
TTCAAAAACTCGAAGAAGAATTGGGAATTCAGCTATTTGAACGCGGCTTCAGACCGGCTAAAGTGACTGA
AGCAGGCATGTTTTTTTATCAGCATGCTGTGCAGATTTTGACTCATACTGCACAAGCGTCCTCAATGGCA
AAACGGATTGCAACGGTCAGTCAAACCTTGAGAATTGGTTACGTCAGCTCCTTACTGTATGGTTTGTTAC
CTGAAATTATTTATCTGTTTCGTCAACAAAATCCTGAAATTCACATCGAACTCATCGAATGCGGCACCAA
AGATCAAATTAATGCCCTTAAGCAGGGAAAAATCGATCTGGGTTTTGGTCGGCTCAAAATTACCGATCCT
GCAATTCGACGTATCGTGTTGCATAAAGAACAGCTCAAACTTGCAATCCATAAGCATCATCACCTCAATC
AGTTTGCAGCAACAGGGGTTCATCTCTCTCAAATTATTGATGAACCGATGCTGCTGTACCCAGTCTCTCA
AAAGCCCAATTTTGCGACCTTTATTCAGTCACTCTTTACCGAACTAGGCCTAGTACCATCCAAACTCACC
GAAATTCGAGAAATTCAACTGGCACTCGGCTTGGTGGCAGCAGGTGAAGGCGTCTGCATCGTACCGGCGT
CTGCCATGGATATTGGGGTGAAGAATCTACTTTATATTCCAATTTTAGATGATGATGCCTATAGCCCAAT
TTCACTCGCGGTGCGAAATATGGACCACAGTAATTACATTCCTAAAATTCTCGCCTGTGTACAGGAGGTG
TTTGCAACGCACCATATCAGGCCACTCATCGAATAACGATCTCGATCCCGCGAAATTAATACGACTCACT
ATAGGGGAATTGTGAGCGGATAACAATTCCCCTCTAGAAATAATTTTGTTTAACTTTAAGAAGGAGATAT
ACATATGCACCATCATCATCATCATTCTTCTGGTGTAGATCTGTGGTCTCATCCGCAGTTCGAAAAGGGT
ACCGAGAACCTGTACTTCCAATCCAATgccATGACCGTGAAAATTTCCCACACTGCCGACATTCAAGCCT
TCTTCAACCGGGTAGCTGGCCTGGACCATGCCGAAGGAAACCCGCGCTTCAAGCAGATCATTCTGCGCGT
GCTGCAAGACACCGCCCGCCTGATCGAAGACCTGGAGATTACCGAGGACGAGTTCTGGCACGCCGTCGAC
TACCTCAACCGCCTGGGCGGCCGTAACGAGGCAGGCCTGCTGGCTGCTGGCCTGGGTATCGAGCACTTCC
TCGACCTGCTGCAGGATGCCAAGGATGCCGAAGCCGGCCTTGGCGGCGGCACCCCGCGCACCATCGAAGG
CCCGTTGTACGTTGCCGGGGCGCCGCTGGCCCAGGGCGAAGCGCGCATGGACGACGGCACTGACCCAGGC
GTGGTGATGTTCCTTCAGGGCCAGGTGTTCGATGCCGACGGCAAGCCGTTGGCCGGTGCCACCGTCGACC
TGTGGCACGCCAATACCCAGGGCACCTATTCGTACTTCGATTCGACCCAGTCCGAGTTCAACCTGCGTCG
GCGTATCATCACCGATGCCGAGGGCCGCTACCGCGCGCGCTCGATCGTGCCGTCCGGGTATGGCTGCGAC
CCGCAGGGCCCAACCCAGGAATGCCTGGACCTGCTCGGCCGCCACGGCCAGCGCCCGGCGCACGTGCACT
TCTTCATCTCGGCACCGGGGCACCGCCACCTGACCACGCAGATCAACTTTGCTGGCGACAAGTACCTGTG
GGACGACTTTGCCTATGCCACCCGCGACGGGCTGATCGGCGAACTGCGTTTTGTCGAGGATGCGGCGGCG
GCGCGCGACCGCGGTGTGCAAGGCGAGCGCTTTGCCGAGCTGTCATTCGACTTCCGCTTGCAGGGTGCCA
AGTCGCCTGACGCCGAGGCGCGAAGCCATCGGCCGCGGGCGTTGCAGGAGGGCTGA

In some aspects, the construct of the disclosure comprises a minimal biosensor. In some aspects, the minimal biosensor comprises a transcription factor, a promoter, and a reporter. In some aspects, the DNA sequence is located in between the transcription factor and the reporter (e.g., a β-glucosidase). In some aspects, the activities of the promoter vary, giving rise to different expression levels of a given gene in the genome. The transcription of the transcription factor is driven by its own promoter. In some aspects, a ‘basal’ transcription of the transcription factor in the cell exists. In a ‘basal’ transcription, a dozen molecules are sufficient to bind to the analyte (e.g., a muconate) and bind strongly to the second promoter, thereby recruiting the transcription machinery to produce tens to hundreds to thousands of copies of mRNA of the reporter enzyme (e.g., β-glucosidase). The expression level of the mRNA for β-glucosidase is proportional to the level of analyte encountered by the system in a certain analyte concentration range. Surprisingly, the circuit was placed into E. coli retained the sensitivity, which is directly relevant in the biomanufacturing process (concentrations in the biomanufacturing process are in the optimal range of the sensor's sensitivity and linear range).

In some aspects, the enzyme-linked and/or cell-based enzyme-linked biosensor of the disclosure (e.g., without limitation, a that uses β-glucosidase as a reporter for gene expression) is accompanied by technical advantages. For instance, in various aspects of the disclosure, the use of the enzyme-linked and/or cell-based enzyme-linked biosensor of the disclosure results in signals recovery (e.g., signals that get detected and/or measured) that are about 10-to-1000-fold higher in fluorescence intensity than signals that are recovered from the transcription factor (FP)-based counterpart (i.e., fluorescent protein-based counterpart). For example, in various aspects, the signals that are recovered from the enzyme-linked and/or cell-based enzyme-linked biosensor of the disclosure, when carried out from equivalent microfluidic test volumes as the TF-based biosensor and/or whole-cell TF-based biosensor, are about 10 fold, about 20 fold, about 30 fold, about 40 fold, about 50 fold, about 60 fold, about 70 fold, about 80 fold, about 90 fold, about 100 fold, about 110 fold, about 120 fold, about 130 fold, about 140 fold, about 150 fold, about 160 fold, about 170 fold, about 180 fold, about 190 fold, about 200 fold, about 210 fold, about 220 fold, about 230 fold, about 240 fold, about 250 fold, about 260 fold, about 270 fold, about 280 fold, about 290 fold, about 300 fold, about 310 fold, about 320 fold, about 330 fold, about 340 fold, about 350 fold, about 360 fold, about 370 fold, about 380 fold, about 390 fold, about 400 fold, about 410 fold, about 420 fold, about 430 fold, about 440 fold, about 450 fold, about 460 fold, about 470 fold, about 480 fold, about 490 fold, about 500 fold, about 510 fold, about 520 fold, about 530 fold, about 540 fold, about 550 fold, about 560 fold, about 570 fold, about 580 fold, about 590 fold, about 600 fold, about 610 fold, about 620 fold, about 630 fold, about 640 fold, about 650 fold, about 660 fold, about 670 fold, about 680 fold, about 690 fold, about 700 fold, about 710 fold, about 720 fold, about 730 fold, about 740 fold, about 750 fold, about 760 fold, about 770 fold, about 780 fold, about 790 fold, about 800 fold, about 810 fold, about 820 fold, about 830 fold, about 840 fold, about 850 fold, about 860 fold, about 870 fold, about 880 fold, about 890 fold, about 900 fold, about 910 fold, about 920 fold, about 930 fold, about 940 fold, about 950 fold, about 960 fold, about 970 fold, about 980 fold, about 990 fold, or about 1000 fold higher in fluorescence intensity than signals recovered from the TF-based biosensor and/or whole-cell TF-based biosensor. A schematic illustration of the signal amplification is shown in FIGS. 3E-3F.

### Composition

In some aspects, the disclosure further provides a composition comprising the host cell of the disclosure and a diluent. In some aspects, the disclosure provides a composition comprising the host cell of the disclosure, wherein the host cell comprises the vector of the disclosure, wherein the vector comprises the expression cassette of the disclosure and a diluent.

In some aspects, the diluent utilized in the disclosure includes Luria-Bertani (LB) broth, which is used to grow E. coli sensor cells. In some aspects, Promega FastBreak Cell Lysis Reagent was used for cell lysis in accordance with the manufacturer's protocol. In some aspects, fluorescein di-β-D-glucopyranoside was utilized as a diluent in the disclosure.

### Methods

With respect to each of the methods provided herein, the disclosure contemplates that in addition to the method being for “detecting the presence of an analyte in a sample,” the disclosure also includes methods for “determining the amount of an analyte in the sample” using the disclosed enzyme-linked biosensor expression cassette.

In some aspects, the disclosure further provides a method of detecting the presence of an analyte in a sample comprising: (a) contacting the sample with the composition of the disclosure and a substrate of the reporter enzyme, and (b) detecting the expression of the reporter enzyme. In some aspects, the disclosure further provides a method of detecting the presence of an analyte in a sample comprising: (a) contacting the sample with the composition of the disclosure, wherein the composition comprises the host cell of the disclosure, wherein the host cell comprises the vector of the disclosure, wherein the vector comprises the expression cassette of the disclosure, and a diluent, and a substrate of the reporter enzyme, and (b) detecting the expression of the reporter enzyme.

In some aspects, the expression of an enzyme variant library in the same vector (e.g., without limitation, a pMCSG68 vector) as the biosensor does not need to take into consideration the plasmid copy number variations (e.g., whether it would be a biosensor plasmid or a plasmid with enzyme variants). In some aspects, the enzyme variants are expressed from a different plasmid.

In some aspects, the disclosure provides a method of detecting the presence of an analyte in a sample comprising: (a) contacting the sample with the expression cassette of the disclosure in a cell-free expression system environment, which thereby activates the transcription and translation of the reporter enzyme, and (b) detecting the expression of the reporter enzyme.

In some aspects, the disclosure provides a method of detecting the presence of an analyte in a sample comprising: (a) contacting the biosensor of the disclosure in a cell-free expression system environment, which thereby activates the transcription and translation of the reporter enzyme, and (b) detecting the expression of the reporter enzyme.

In some aspects, the detection of the expression of the reporter enzyme is carried out via an enzymatic reaction.

In some aspects, the analyte is cis, cis-muconate (CCM). Without wishing to be bound by any particular theory, it is believed that the method of detecting the presence of an analyte of the disclosure by contacting the biosensor in a cell-free expression system environment (e.g., without limitation, in an in vitro environment). Thus, the CCM binds to the CatM transcription factor. Next, CatM-CCM binds to the CatM-responsive promoter and is followed by CatM-CCM activating the transcription of the reporter coding sequence. Next, the reporter enzyme (e.g., without limitation, a β-glucosidase) is produced. Upon its production, the reporter enzyme (e.g., without limitation, a β-glucosidase) can be detected by adding a clear substrate that is converted by the enzyme into a fluorescent product. One of the advantages of the method described herein is that it is significantly more sensitive than other methods that utilize the expression of fluorescent protein as a reporter. Moreover, the method described herein produces a result wherein the CCM concentration is proportional to the expression of the reporter enzyme and the fluorescent signal observed once the fluorescent product is released.

In some aspects, the disclosure further provides a method of determining a concentration of an analyte present in a sample comprising: (a) contacting the sample with the composition of the disclosure; (b) detecting the expression of the reporter enzyme; (c) measuring the concentration of the reporter enzyme; and (d) comparing the concentration of the reporter enzyme to a control or standard to determine the concentration of the analyte present in the sample.

In some aspects, the disclosure further provides a method of determining a concentration of an analyte present in a sample comprising: (a) contacting the sample with the composition of the disclosure, wherein the composition comprises the host cell of the disclosure, wherein the host cell comprises the vector of the disclosure, wherein the vector comprises the expression cassette of the disclosure and a diluent; (b) detecting the expression of the reporter enzyme; (c) measuring the concentration of the reporter enzyme; and (d) comparing the concentration of the reporter enzyme to a control or standard to determine the concentration of the analyte present in the sample.

In some aspects, the method of determining the concentration of an analyte present in a sample comprises a standard curve. In some aspects, the standard curve may be established by running several reactions in parallel with varying concentrations of the analyte.

In some aspects, the amount of a product or bioproduct present in the sample can be quantified. In some aspects, the amount of a product or bioproduct present in the sample can be measured. In some aspects, the amount of a product or bioproduct present in the sample can be quantified or measured by comparing the amount to a standard curve. In some aspects, the standard curve may be generated using commercially available cis, cis-muconic acid for the muconate biosensors. In some aspects, the fluorescence of the reporter enzyme from the biosensors is measured with a fluorescent plate reader. In some aspects, the green fluorescence from the biosensors is measured either with a fluorescent plate reader or with a confocal microscope.

In some aspects, the disclosure further provides a method of monitoring product formation in a cell. In some aspects, the disclosure further provides a method of monitoring a product formation in a cell, wherein the product activates the transcription factor. In some aspects, the transcription factor drives the expression of the β-glucosidase reporter enzyme. In some aspects, the disclosure provides a method of monitoring a product formation in a cell, wherein the product activates the transcription factor, and the transcription factor drives the expression of the β-glucosidase reporter enzyme.

In some aspects, a method for producing the enzyme to be used as biosensors is provided herein. In aspects, the nucleic acids provided herein may be used in methods for the production of enzymes and enzyme cocktails through incorporation into cells, tissues, or organisms. In some aspects, a nucleic acid may be incorporated into a vector for expression in suitable host cells. In aspects, a vector may then be introduced into one or more host cells by any method known in the art. In aspects, a method to produce an encoded protein includes transforming a host cell with one or more recombinant nucleic acids (such as expression vectors) to form a recombinant cell. The term “transformation” is generally used herein to refer to any method by which an exogenous nucleic acid molecule (i.e., a recombinant nucleic acid molecule) can be inserted into a cell but can be used interchangeably with the term “transfection.”

In some aspects, the method of the disclosure detects CCM with at least about 10-fold greater sensitivity than a GFP biosensor. In some aspects, the method of disclosure is capable of measuring responses to extracellular CCM.

In some aspects, the method of the disclosure produces signals that are recovered from equivalent microfluidic test volumes that, when measured for the recovery of the signals, the fluorescence intensity of the signals is at least about 10-fold higher in fluorescence intensity than those found from the fluorescent protein-based counterparts. In some aspects, the method of disclosure results in yields with high sensitivity of product detection. In some aspects, the method of disclosure comprises an operational range even in a picoliter environment, e.g., microfluidic droplets. In some aspects, the method of the disclosure is capable of detecting microfluidic droplets.

Throughout this specification and claims, the word “comprise” or variations such as “comprises” or “comprising” will be understood to imply the inclusion of a stated integer or group of integers but not the exclusion of any other integer or group of integers.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

As used herein and in the appended claims, the singular forms “a,” “and,” and “the” include plural referents unless the context clearly dictates otherwise. Thus, for example, reference to “a β-glucosidase” includes a plurality of β-glucosidases and equivalents thereof known to those skilled in the art, and so forth. Similarly, reference to “a biosensor” includes a plurality of biosensors and equivalents thereof known to those skilled in the art, and so forth.

The term “about” signifies not more or less than 10 percent of the stipulated amount. Thus, an increase in the fluorescence intensity of the recovered signals by about 10-to-1000-fold (i.e., 10 is the lower value and 1000 is the higher value) may be interpreted as inclusive of 9-fold-to-1100.

As will be apparent to those of skill in the art upon reading this disclosure, each of the individual aspects described and illustrated herein has discrete components and features that may be readily separated from or combined with the features of any of the other several aspects without departing from the scope or spirit of the disclosure. Any recited method can be carried out in the order of events recited or in any other order which is logically possible. This is intended to provide support for all such combinations.

### Nucleotide Sequences of the Disclosure

In some aspects, the disclosure provides for any of the sequences (i.e., SEQ ID NOs: 1-24) provided herein, including the sequences set out herein and below, and a variant sequence having at least or about 70%, at least or about 75%, at least or about 80%, at least or about 85%, at least or about 90%, at least or about 91%, at least or about 92%, at least or about 93%, at least or about 94%, at least or about 95%, at least or about 96%, at least or about 97%, at least or about 98%, or at least or about 99% sequence identity thereto, or at least or about 40 mutations or substitutions, at least or about 30 mutations or substitutions, at least or about 20 mutations or substitutions, at least or about 10 mutations or substitutions, at least or about 9 mutations or substitutions, at least about 8 mutations or substitutions, at least or about 7 mutations or substitutions, at least or about 6 mutations or substitutions, at least or about 5 mutations or substitutions, at least or about 4 mutations or substitutions, at least or about 3 mutations or substitutions, at least or about 2 mutations or substitutions, or at least or about 1 mutation or substitution. In some aspects, a nucleotide or amino acid sequence of the disclosure comprises 100% identity to the disclosed sequence.

| SEQ ID NO: 1: Nucleotide sequence of the pBATS_0004 β-glucosidase; Glucosidase | |
|---|---|
| (APC115086_29_766) (2220 nt) | |
| 1 | ATGAAATCTC CTGTCGATAT GGATCGCTTT ATTGATGATC TGATGAAGAA |
| 51 | GATGACTCTG GAAGAGAAAA TCGGCCAGTT GAACTTGCCT GTTACGGGTG |
| 101 | AAATAACCAC CGGACAAGCC AAGAGTAGTA ATGTGGCTAA GCGTATCCGT |
| 151 | GCCGGTGAAG TGGGCGGACT CTTTAACTTG AAAGGCGTGG AGCGTATTCG |
| 201 | TGACGTTCAG AAACAGGCAG TAGAAGAAAG TCGTCTGGGT ATTCCTCTTT |
| 251 | TATTTGGTAT GGATGTAATT CATGGATACG AAACGGTATT TCCTATTCCT |
| 301 | CTGGGATTAT CCTGTACCTG GAACATGACA GCTATTGAAG AATCTGCACG |
| 351 | TATTGCTGCT ATCGAAGCCA GTGCTGATGG TATTTGCTGG ACATTCAGTC |
| 401 | CGATGGTGGA TGTTTCCCGT GATCCCCGTT GGGGACGAGT TTCCGAAGGG |
| 451 | AATGGTGAAG ATCCCTTCTT GGGAGCGGAG ATTGCGCGTG CTATGGTACG |
| 501 | TGGTTATCAA GGGAAAGATA TGAGTAGTAA TGATGAAATT ATGGCTTGCG |
| 551 | TGAAGCACTT TGCGTTATAT GGGGCATCAG AAGCCGGACG CGACTATAAT |
| 601 | ACAGTGGATA TGAGTCATCA ACGTATGTTC AACGAATATA TGTTACCTTA |
| 651 | TCAGGCTGCC GTGGAAGAAG GTGTGGGTAG TGTGATGGCT TCATTCAATG |
| 701 | AAGTGGATGG TGTACCGGCT ACCGGAAATA AGTGGCTGAT GACCGATGTA |
| 751 | CTTCGTAAGC AGTGGAATTT TGATGGGTTC GTTGTGACGG ACTATACCGG |
| 801 | TATCACTGAA ATGACCGATC ATGGTATGGG TGATACACAA ACAGTTGCAG |
| 851 | CCCTGGCTCT GAATGCAGGT GTCGATATGG ATATGGTGAG CGATGCTTTT |
| 901 | ACAAGCACAC TTAAAAAATC TCTGGAAGAA GGAAAAGTTT CAGTAAAGGC |
| 951 | TGTTGATGCT GCTTGTCGCC GTATTCTGGA AGCTAAGTAT AAGCTGGGGC |
| 1001 | TTTTTGATAA TCCCTATAAA TATTGTGATA TAACCCGTCC TAAAAAACAA |
| 1051 | ATCTTTACAA AAGAACACCG CGCTATAGCC CGTAAGACAG CTTCGGAAAG |
| 1101 | CTTTGTTCTC TTGAAGAATG AGAATAGTGT ACTCCCTCTG GCAAAGAAAG |
| 1151 | GTACCATTGC TGTAGTAGGT CCTTTGGCCG ATAGCCGTAG CAATATGCCG |
| 1201 | GGCACGTGGA GTGTGGCCGC TGTGATGAAC AAATATCCTT CTTTGATTGA |
| 1251 | AGGCTTGAAA GAAGTAGTGG GAGGCAAGGC TAAAATTCTT ACGGCTAAAG |
| 1301 | GAAGTAATCT GATGAGTGAT GCCGAATACG AAGAACGTGC TACTATGTTT |
| 1351 | GGCCGTACTC TGCATCGTGA CAATCGTACA GATAAGGAAC TGCTGGATGA |
| 1401 | GGCGCTTGCT GTAGCTGCCA AGTCTGACGT GATTGTTGCT GCTTTGGGTG |
| 1451 | AGTCTTCCGA GATGAGCGGT GAAAGTAGTT GCCGTACAGA CCTCGAAATG |
| 1501 | CCGGATACGC AACGTGTACT TTTGCAGGAA TTGTTGAAAA CCGGCAAACC |
| 1551 | GGTGGTATTG GTGTTGTTTA CCGGTCGTCC GTTAGTATTG AATTGGGAGC |
| 1601 | AGGAAAATGT ACCTGCTATT CTGAATGTGT GGTTTGGTGG TAGTGAAGCT |
| 1651 | GCTCTTGCCA TTGGTGATGT ACTGTTTGGA AATGTAAATC CGAGTGGCAA |
| 1701 | ACTTACTACT ACTTTTCCGA AGAGTGTAGG ACAGATTCCT TTGTTCTATA |
| 1751 | ACCATAAGAA TACTGGTCGT CCTTTGCCTC AAGGGGCCTG GTTCCAGAAG |
| 1801 | TTCCGTAGCA ATTATCTGGA TGTAGATAAC GAACCGCTTT ATCCGTTTGG |
| 1851 | ATATGGCTTG AGCTATACTA CITTCTCTTA TAGTGATATT ACATTGGATA |
| 1901 | AATCGTCCAT GAATATCAAT GGAGAGATTA TGGCAACTGT AACGGTAACC |
| 1951 | AATACAGGTA AGTATGACGG TTCGGAAGTA GTGCAGCTAT ATATCCGCGA |
| 2001 | TCTTATAGGC AGTGTAACAC GTCCGGTGAA AGAACTGAAA GGCTTTGAAA |
| 2051 | AAATCTTCTT GAAAGCCGGT GAATCCAAAC AAGTGTCTTT CAAGTTAACA |
| 2101 | GCTGATATGT TGAAGTTCTA CAATTACAAT CTGGATTTTG TGTGCGAACC |
| 2151 | GGGTGACTTT GAAGTAATGA TAGGTGGTGA TAGCCGTGAT GTGAATAAGG |
| 2201 | CCTTATTTTC GCTTCAATAA |

SEQ ID NO: 2: Nucleotide sequence of APC115038.102; APC115038.26-
783.P(785)..pMCSG68
AAGTCACCGCAAGACATGGATCGCTTCATCGACGCACTGATGAAGAAGATGACCGTGGAAGAGAAAATCG
GACAATTGAACCTACCCGTCACGGGAGACATCACCACGGGACAGGCCAAAAGTAGCGACGTGGCACAAAA
GATTGAAAAAGGATTGGTGGGCGGACTCTTCAACCTAAAAGGTGTAGACCGTATTCTTGAAGTGCAAAAG
CTGGCAGTAGAGAAATCACGCCTCGGTATTCCCCTGCTGTTCGGCATGGATGTGATACATGGCTACGAAA
CCATCTTCCCCATTCCATTGGGATTGTCCTGCACCTGGGATATGGCGGCTATCGAGAAATCCGCCCGTAT
TGCAGCCATCGAAGCAAGTGCCGATGGCATTTCCTGGACATTCAGTCCGATGGTAGACATCAGTCGCGAC
CCACGTTGGGGACGTGTCAGCGAGGGCTCGGGAGAAGATCCGTTTCTGGGTGGAGCTATCGCACAGGCAA
TGGTATACGGATACCAGGGTGCCAATCTGCAAGACCAATTGCACCGCAACGATGAAATCATGGCTTGCGT
AAAACACTTTGCATTGTATGGAGCCGGAGAAGCCGGACGCGACTATAATACAGTAGATATGAGCCGCAAC
CGGATGTTCAATGAATTCATGTACCCGTATGAGGCTGCCGTAGAGGCCGGAGTGGGTAGTGTGATGGCGT
CATTCAATGAAATAGACGGTATTCCCGCCACCGGAAACAAATGGCTGCTGAGCGATTTGCTGCGTGGCCA
GTGGGGCTTCGAAGGGTTTGTGGTAACGGACTTCACAGGCATTTCAGAGATGATAGAGCATGGTGTCGGC
GACTTGCAAACCGTCAGTGCACTCGCTCTTAATGCAGGGGTGGACATGGATATGGTAAGTGAGGGCTTCG
TCGGTACACTGATGAAATCAATTAAAGAAGGAAAAGTAAGAATGGGCACGTTGAATACAGCCTGCCGCCG
GATATTGGAAGCGAAATACAAGCTGGGACTGTTTGACAATCCTTATAAATACTGCGACGTGAACCGTCCG
AAGCGGGATATCTTCACAAAAGAGCATCGTGACGCCGCCCGCAAGATTGCCGGCGAAAGTTTTGTTCTTC
TGAAGAATGCCCCCGCCACCGCACAGCCACTCGCAGCTCATAGCTCGTCACCCGTAACTGCTTCCCCCGT
GCTTCCGTTGAAGAAACAAGGTACAGTTGCCGTCATCGGCCCTCTCGGAAATACCCGCAGCAACATGCCG
GGCACCTGGAGCGTAGCCGCACGCCTCAACGATTATCCTTCTTTATACGAAGGCTTGAAAGAAATGATGG
CAGGCAAGGTGAACATCACCTATGCCAAAGGTAGTAACCTCATCGGCGATGCAGCTTACGAAGAACGTGC
CACCATGTTCGGCCGTTCATTGAACCGCGATAATCGCACGGACCAGGAGTTACTGGACGAAGCACTGAAA
ATTGCAGCCGGCGCCGATGTTATCGTAGCTGCCCTGGGAGAATCTTCTGAAATGAGCGGTGAAAGTTCAA
GCCGCACCGAACTCGGCTTGCCCGATGTACAACATACCCTGTTGGAAGCCTTACTGAAAACGGGTAAACC
CGTAGTACTAACCCTCTTTACCGGTCGGCCGTTGACGCTGAACTGGGAACAGGAGCATGTACCTGCCATC
TTGAATGTATGGTTCGGAGGCAGTGAAGCGGCTTATGCCATTGGCGATGTTCTGTTCGGTGACGTCAATC
CGAGTGGAAAACTAACCATGACTTTCCCGAAGAATGTAGGCCAGATACCTTTGTTCTACAATCATAAGAA
TACCGGTCGGCCACTGGCGGCAGGCAAATGGTTCGAAAAGTTCCGTTCAAACTATCTGGATGTGGATAAC
GAACCGCTGTATCCCTTCGGTTATGGATTGTCGTATACCACTTTCCAGTACAGTGACATTGCATTGAGCA
CACCGACATTGGGAAAAGATGGTTCCGTTACAGCCGTAGTCACCGTCACCAATACTGGTAAACATGACGG
TGCGGAAGTAGTTCAACTCTATATCCGCGACCTCGTAGGAAGTATCACCCGCCCTGTACGCGAGTTGAAA
GGTTTCAATAAAATCTTCCTTCGCGCCGGAGAAAGCAAAACGGTATCATTCACTATCACGCGTGATCTGC
TTCGCTTCTATGATTACGACCTGAACTACGTAGCCGAACCGGGTGACTTTGACATCATGATCGGTGGAAA
CAGCCAGGCTGTGAAGACGGCGAAGTTGACACTT
SEQ ID NO: 3: Nucleotide sequence of APC115043.102; APC115043.26-
768.P(769)..pMCSG68
AAGAGTGGGGATGCGTCGATGAACAAATTTATTGATAAACTGATGGACAGGATGACCTTGGAAGAGAAGA
TTGGTCAGCTTAATCTTCCCAGCTCGGGAGATATAACCACCGGACAGGCACGCAGCAACAATATTGCAGA
CAAAATCAGAGCAGGTGCAGTGGGTGGCTTATTCAATATAAAAGGAGTTGAGAAGATACAGGAAGTACAA
CGTATTGCTGTAGAGGAGAGTCGCCTGAAAATTCCTTTACTCTTTGGCATGGATGTTATTCATGGGTATG
AAACTGTTTTCCCTATTCCTTTGGGTATGGCTGCCACATGGGATATGAAGGCTATAGAACAATCTGCTCG
TATAGCGGCGATAGAAGCCAGTGCCGATGGCATCTGCTGGACATTTAGTCCGATGGTTGATATCAGCCGT
GATCCACGTTGGGGACGTGTATCCGAAGGTAGCGGAGAAGATCCTTTTTTAGGTGGTGAAATTGCTAAGG
CGATGGTATATGGCTATCAGGGTAAAGGTGATAGCGCATATCGTGAAAAGACTAATATTATGGCTTGTGT
GAAGCACTATGCCTTGTATGGGGCAGCAGAAGCCGGTTTGGACTATAATACAACTGACATGAGCCGTATT
CGTATGTTTAATGAATATATGTATCCTTATCAGGCGGCTGTGGATGCGGGTGCCGGCAGTGTCATGTCTT
CTTTCAACGAGGTCGATGGAATTCCTGCAACAGCCAACAAATGGTTGATAACTGATGTCCTGCGTAAACA
GTGGGGATTCGGTGGTTTTGTCGTTACGGACTATACCGGTATCATGGAAATGGTAAATCATGGTATTGGA
GATATGCGAGAAGTCTCTGCCCGTGCTTTGAGTGCAGGAGTGGATATGGATATGGTGAGCGAAGGTTATC
TTTCTACACTTCAACAATCATTGAAGGAGGGTAAGATAACAGAGAAAGAGATAGATCAAGCTTGCCGTCG
TATTTTGGAGGCAAAATATAAGCTGGGATTATTTGATAATCCTTATAAGTATTGTGATACTGAACGTGCC
AAAACGGATATCTACACTGATGAACATCGGAGTATTGCACGCCGGATCTCTGCTGAAAGCTTTGTTCTTT
TAAAGAATGATAAACAGACACTGCCTATAAAGAAAAAAGGTAAGATTGCTGTAGTTGGGCCGTTGGCGAA
TACGAGTTCTAATATGCCCGGAACGTGGAGTGTAGCGGTCAATATGGAAGCTCCAGCTACGCTTGTGGAG
GGTTTGAAAGAAGTGGCAGGTGATAAAGTTGAAATTGTGTATGCTAAGGGTAGCCATCTGATGAGTGATG
CGGCTTATGAGGAACGTGCAACACTCTTTGGACGTACATTATACCGGGATAAGGAAAAACGTTCCGATAT
CCAGATGCTGAATGAAGCATTAAATGTTGCTCATGGTGCCGATGTTGTTGTTGCGGCATTAGGTGAATCT
TCTGAAATGAGTGGTGAATCGAGTAGTCGAACAGATTTGAATATTCCTGATGTTCAAAAAACATTATTGG
AAGAATTAGTGAAAACAGGTAAACCTGTCGTTCTGGTATTATTCACTGGGCGTCCGTTGACCCTGACATG
GGAAGACAAAAATGTATCTGCTATTCTGAATGTTTGGTTTGGAGGTACCGAAGCCGCTTATGCTATAGGA
GATGTCCTATTCGGAAATGTAAATCCTGGAGGTAAGCTGCCTGTAACATTTCCTCAGAATGTAGGGCAGA
TTCCTTTATTCTATAACCATAAAAATACTGGACGTCCGCTGGCTGAGGGCGGTTGGTTTGAGAAGTTCCG
GGCAAATTATCTGGATGTAACGAATGAACCTCTTTATCCATTTGGCTATGGACTAAGTTATGCACAATTT
GATTATAGCGATGTGAGATTAAGTACGGATCAAATAGACCGGAATGGCATGTTAACCGCAAGTGTGACTG
TAACCAATAACAGTGAGTGTGATGGAGATGAAATTGTTCAGTTGTATATTCGCGATTTGGTCGGTAGTGT
TACTCGTCCGGTGAAAGAATTGAAAGGATTTGAAAAAGTAACAATTAGAGCAGGGGAGTCAAAAGATATT
TCTTTTAAGATCACTCCGGAAATGCTTAAGTTCTACAATTCGGATATCCAGTTTGTGAATGAAGTTGGTG
AATTCGAAGTAATGATCGGAACGAACAGCAGGGATGTGAAAAAAGCAACGTTTAGCTTG
SEQ ID NO: 4: Nucleotide sequence of APC115044.102; APC115044.33-
744.P(748)..pMCSG68
GTAGAATCTCTCCTGTCTAAGATGACCCTTGAGGAGAAAATCGGTCAGATGAACCAGATTTCCTCTTACG
GTAATATCGAGGATATGAGTGCTTTGATTAAGAAAGGTGAAATCGGTTCCATCTTGAATGAGGTGGATCC
GGTGCGTATTAATGCGCTACAGCGCGTGGCAATGGAAGAATCCCGTTTGGGTATTCCTTTATTGATAGCG
CGTGATGTCATTCACGGGTTTAAAACAATTTTCCCTATTCCCTTGGGACAAGCGGCTTCGTTCAATCCGC
AGGTAGCGAAAGACGGTGCACGGATAGCAGCTATTGAAGCTTCGTCTGTAGGTATCCGGTGGACTTTTGC
GCCAATGATTGATATTGCCCGCGATCCTCGCTGGGGACGTATTGCCGAAGGGTGTGGTGAAGATACGTAC
CTTACTTCCGTAATGGGAGCAGCTATGGTAGAAGGTTTTCAGGGAGATTCGCTGAATAGTCCTACTTCAA
TTGCAGCTTGCCCTAAACATTTTGTAGGTTACGGTGCAGCCGAAGGAGGACGTGATTATAATTCCACGTT
CATTCCCGAACGTCGTCTGCGCAATGTTTATTTGCCACCTTTTGAAGCTGCCACCAAAGCGGGTGCAGCC
ACGTTTATGACTTCATTTAATGATAATGATGGAATCCCTTCTACCGGGAATGCTTTTATTTTGAAGAATG
TACTCCGTGACGAGTGGGGATTCGATGGTTTTGTTGTGACGGACTGGGCTTCTGCCAGCGAAATGATAAG
CCATGGTTTTGCCGCCGGTTCAAAAGAAGTGGCAATGAAATCTGTGAATGCAGGAGTAGATATGGAAATG
GTGAGTTACACTTTTGTGAAGGAACTGCCGGAATTAGTGAAAGAGGGAAAGGTGAAGGAAAGCACTATCG
ATGAGGCTGTTCGTAATATTTTGCGTATAAAGTATCGTTTAGGATTGTTTGATACACCTTATGTAGATGA
ACAACAAACATCTGTCATGTATGCTCCTTCTCATTTGGAAGCAGCTAAGCAAGCCGCTGTTGAATCGGCT
ATTCTGTTGAAGAATGATAAGGAAGTGTTGCCGTTACAGCCATCTGTGAAAACTGTTGCAGTGGTAGGAC
CTATGGCTAATGCACCTTATGAACAGTTAGGTACTTGGATATTTGATGGTGAGAAAGCTCGTACTCAGAC
TCCGTTGAACGCTATTAAAGAAATGGTTGGCGATAAAGTACAGGTGATTTATGAACCGGGACTAGCATAT
AGTCGTGAGAAAAATCCGGCAAGTGTGGCTAAAGCAGCTGCCGCCGCTGCACGTGCAGATGTCATTCTTG
CTTTTGTGGGTGAAGAATCTATTCTTTCGGGTGAAGCTCACTGTTTGGCTGATCTGGATTTGCAGGGTGA
TCAGGGAGCTTTGATTACAGCTTTGGCTAAGACGGGTAAACCTGTAGTGACTATTGTGATGGCGGGTCGT
CCGTTGACTATCGGTAAAGAAGTCGAAGAGTCGACTGCTGTTCTCTATTCATTCCATCCGGGCACAATGG
GCGGTCCTGCATTGGCTGATTTGCTTTGGGGGAAGGCTGTGCCGAGTGGAAAGGCGCCGGTCACTTTCCC
GAGGATGGTGGGACAAATTCCTGTGTACTACGCTCATAATAATACCGGACGTCCGGCTACACGGAATGAA
GTGTTGCTGAATGATATTGCTGTTGAGGCAGGACAGACTTCACTGGGCTGTACTTCCTTCTATATGGATG
CGGGTTTTGATCCCTTGTTTCCGTTTGGTTATGGCTTGTCGTACACCACATTTAAGTATAGCAACATCAA
ACTGGCGTCTGATGTACTGAAAAAAGATGATGTGCTGACAGTGACATTCGATCTGGAAAATACCGGGAAA
TATGAAGGAACGGAAGTAGCTCAATTGTATATACAAGATAAGATTGGTTCCGTGACTCGTCCGGTGAAAG
AACTGAAACGCTTCACTCGTGTGACATTGAAGCCGGGTGAGAAAAAAAGCGTTTCGTTTGAACTCCCTGT
TAGTGAACTTGCATTTTGGAACATAGATATGGCTAAAGTTGTGGAACCCGGAGACTTTGGGCTTTGGGTG
GCAACGGATAGTCAGTCCGGAGAAGAAGTTTTCTTC
SEQ ID NO: 5: Nucleotide sequence of APC115045.102; APC115045.26-
772.P(773)..pMCSG68
AAGTCTCCGCAGGACATGGATCGCTTCATCGATGCATTGATGAAGAAGATGACTGTAGAGGAAAAGATCG
GTCAGCTGAACCTACCCGTTTCCGGCGAGATCGTCACCGGGCAGGCACAAAACAGCGATGTGGCAAAAAA
GATTGAACAAGGGCTCGTGGGCGGACTCCTCAACCTGAAAGGGGTGGAGAAGATACGCGATGTACAAAAA
CTGGCCATAGAGAAGTCACGCCTGGGCATCCCCCTGATATTCGGCATGGACGTAGTGCATGGTTACGAAA
CCATTTTCCCTATTCCATTAGGCCTCTCCTGTTCCTGGGATATGGAAGCCATCAGGAAATCTGCCCGCGT
TGCAGCCATCGAGGCCAGTGCTGATGGTATTTCCTGGACATTCAGCCCGATGGTAGACATCAGCCGTGAT
CCGCGCTGGGGACGCGTCAGCGAGGGTAACGGCGAAGACCCATTCTTGGGTGGAGCCATCGCTAAAGCAA
TGGTATCGGGTTATCAGGGTATCGACCTCAACAACCAACTGAAGCGCAACGATGAAATTATGGCATGTGT
AAAGCACTTCGCACTGTATGGTGCCGGAGAAGCCGGACGTGATTACAATACCGTAGATATGAGTCGTAAC
CGTATGTTCAACGAATACATGTATCCCTACCAAGCTGCCGTAGATGCAGGTGTAGGCAGCGTAATGGCGT
CTTTCAACGAAATAGACGGCATACCAGCCACGGCCAATAAATGGCTGATGACCGACGTACTGCGCAAGCA
ATGGGGCTTCGACGGCTTTGTGGTGACAGACTTTACCGGTATCTCCGAAATGATAGCGCACGGCATCGGT
GACTTGCAGACTGTTTCCGCACGTGCACTCAATGCAGGCGTGGATATGGACATGGTAAGTGAAGGCTTCA
CGGGTACAATCAAGAAATCCATAGACGAAGGCAAGATCAGTATGGAAACCCTGGACAAAGCCTGTCGCCG
CATCCTTGAAGCCAAATACAAACTGGGATTATTCGACAATCCTTATAAGTACTGCGACCTGAAACGCCCG
AAGCGTGACATCTTCACCAAGGAACATCGCGACGCTGCTCGTAAGATTGCGGGAGAGAGCTTTGTACTCC
TGAAAAACGACAAGTCAGGTTCCTCTGCAAACCCAACACTTCCTTTGAAAAAAGAAGGTACGGTGGCTGT
CATCGGCCCACTGGCAAATACCCGCAGTAACATGCCGGGTACCTGGAGTGTAGCCGCACGCCTCAACGAC
TATCCTTCTGTGTACGAAGGATTGAAAGAGATGATGAAAGGCAAGGTAAACATCACTTATGCCAAAGGTA
GTAACCTCATCAGTGATGCAGCCTACGAAGAACGTGCCACAATGTTCGGCCGTTCATTAAATCGTGATAA
TCGTACAGACAAAGAGATGCTGGATGAGGCGCTGAAAGTGGCCGCTAATGCAGATGTAATAATAGCCGCA
TTGGGAGAATCATCTGAAATGAGTGGTGAAAGTTCAAGCCGCACTAACCTGGCTCTTCCCGATGTACAGC
GCACTCTATTGGAAGCTTTGCTGAAAACTGGAAAGCCTGTTGTACTGACGCTCTTTACAGGTCGCCCACT
AACGTTGACTTGGGAACAGGAGCATGTGCCCGCCATCCTGAATGTATGGTTCGGTGGAAGTGAGGCAGCA
TACGCCATTGGCGATGTATTGTTCGGCGATGTAAATCCCAGCGGCAAACTAACGATGACATTCCCCAAAA
ACGTAGGCCAAATACCTTTGTTTTACAATCATAAAAATACCGGTCGTCCTTTACTTGAAGGCAAATGGTT
CGAAAAATTCCGTAGTAATTACCTGGATGTAGACAACGATCCATTGTATCCATTCGGCTATGGTTTGTCG
TATACCAACTTTCAATACAGCGACATAACTCTGAGCGCCCCGACTATGGGACAGGATGGTTCTGTTACTG
CTATGGTCACGGTAACCAATACCGGTAAGTACGATGGTGCAGAAGTAGTGCAACTTTATATCCGTGACCT
TGTAGGAAGCATCACCCGTCCGGTAAAAGAACTGAAAGGGTTTGATAAAATTTTCCTCAAAGCGGGTGAA
AGTAAGACTGTATCTTTCAAAATCACTCCGGAATTACTGCGCTTCTACGACTATGAACTCAACTACGTAG
CCGAACCGGGAGACTTCGACATAATGATCGGGGGGAACAGCCAAAGTGTAAAAACGACTCATCTGAGTTT
SEQ ID NO: 6: Nucleotide sequence of APC115068.102; APC115068.26-
774.P(775)..pMCSG68
AAGTCCCCCCAAGACATGGACCGCTTCATCGATGCGCTGATGAAGAAAATGACTGTGGAAGAGAAAATCG
GACAGTTGAACCTACCCGTCACGGGAGACATCACCACAGGACAGGCCAAGAGCAGCGACGTAGCCGCAAA
GATTGAAAAAGGATTGGTAGGCGGACTCTTCAACCTGAAAGGGGTAGACCGCATTCTTGAAGTGCAAAAG
CTGGCAGTAGAGAAATCACGTCTCGGTATTCCCCTGTTATTCGGCATGGACGTGATACATGGATACGAAA
CCATCTTCCCCATCCCATTGGGGCTGTCCTGCACTTGGGATATGGCCGCCATCGAGAAGTCTGCCCGTAT
CGCAGCCATCGAAGCAAGTGCCGATGGCATCTCCTGGACATTCAGTCCGATGGTAGACATCAGCCGTGAT
CCACGTTGGGGACGTGTCAGCGAAGGTTCGGGAGAAGACCCTTTCCTGGGTGGAGCTATCGCACAGGCAA
TGGTATACGGATACCAGGGTGCCAATCTGCAAGACCAGTTGCGCCGTAATGATGAAATCATGGCCTGCGT
TAAACATTTCGCCCTGTATGGAGCCGGAGAGGCCGGACGCGATTATAACACAGTGGACATGAGCCGCAAC
CGGATGTTCAATGAATTTATGTATCCGTACGAAGCTGCCGTAGAGGCAGGTGTAGGTAGCGTAATGGCTT
CATTCAATGAAATAGACGGGATACCGGCTACCGGGAACAAATGGCTATTGAGCGACTTGCTGCGTGGCCA
ATGGGGGTTTGAAGGGTTTGTGGTAACAGACTTTACAGGTATTGCGGAGATGATAGAACATGGTGTCGGC
GACTTACAAACCGTCAGTGCACTTGCCCTGAATGCAGGTGTGGATATGGATATGGTAAGTGAAGGTTTTG
TCGGCACGCTGATGAAATCCATTAAAGAAGGAAAAGTGAGAATGGGTACGCTAAATACGGCTTGCCGCCG
GATATTGGAAGCAAAATATAAATTGGGCCTGTTCGACAATCCTTATAAATATTGTGATGTGAACCGTCCG
AAGCGGGACATCTTTACAAAAGAACATCGGGATGCCGCCCGTAAGATTGCCAGTGAAAGTTTTGTACTTT
TAAAGAACGCTCCCTTAGCAGCACAGAAAAATGCCGCCCCCGTGCTTCCATTAAAGAAGCAAGGCACCGT
TGCAGTAATCGGTCCTCTCGGCAATACGCGTAGCAATATGCCGGGCACTTGGAGTGTAGCTGCACGCCTC
AACGATTATCCTTCTTTGTACGAAGGACTGAAAGAGATGATGGCAGGCAAAGTCAACATCACCTACGCCA
AGGGCAGCAACCTTATCGGTGATGCTGCTTACGAAGAACGTGCCACCATGTTCGGTCGCTCACTGAACCG
CGACAACCGTACGGATCAGGAATTATTGGACGAAGCGCTGAAAGTGGCAGCCGGAGCCGATGTCATCGTA
GCCGCACTGGGGGAATCTTCTGAAATGAGTGGTGAAAGTTCAAGCCGCACAGAACTCGGCTTACCCGATG
TGCAGCATACTTTACTGGAAGCCTTACTAAAAACAGGCAAGCCTGTAGTACTTACTCTGTTTACCGGTCG
CCCGTTGACACTGAACTGGGAACAGGAACATGTACCTGCTATCCTCAATGTATGGTTCGGAGGTAGCGAG
GCAGCTTATGCCATTGGCGATGTATTGTTCGGCGACGTAAATCCAAGTGGAAAGCTGACGATGACGTTCC
CGAAGAATGTAGGCCAGATACCTTTGTTCTACAATCATAAGAATACCGGTCGCCCGTTGGCAGAAGGTAA
ATGGTTCGAAAAGTTCCGTTCAAATTATCTGGATGTGGATAATGAACCATTGTACCCCTTCGGTTATGGA
TTATCATATACCAACTTCCAGTATAGTGACATTGCACTGAGCACGCCTACACTGGGAAAAGACGGTTCTG
TTACCGCCGTAGTTACTGTAACCAATACGGGTAAATACGATGGTGCGGAAGTAGTACAACTCTATATCCG
TGATCTTGTAGGAAGCATCACCCGTCCGGTGCGCGAGCTGAAGGGGTTCAATAAGATCTTCCTTCGTGCC
GGAGAAAGTAAAACAGTATCATTCACCATCACGCGCGACCTGCTCCGGTTCTATGATTATGATATGAATT
ACGTAGCCGAACCCGGTGATTTCAATATTATGATCGGTGGAAACAGCCAGACGGTGAAGACGGCAAAATT
AACACTT
SEQ ID NO: 7: Nucleotide sequence of APC115077.102; APC115077.27-
740.P(800)..pMCSG68
CGGGAACAATCTTTCGATGAGGCATGGTTATTTCATCGTGGAGATATTGCCGAAGGAGAAAAGCAATCTT
TAGATGACTCACAATGGCGTCAGATAAATCTTCCTCATGATTGGAGTATTGAAGATATTCCTGGAACCAA
TTCTCCTTTTACAGCGGATGCTGCAACGGAAGTTGCAGGTGGTTTTACTGTAGGTGGTACGGGATGGTAT
AGAAAGCACTTCTACATAGATGCGGCTGAAAAAGGTAAATGTATTGCTGTCTCTTTCGATGGAATTTATA
TGAATGCAGATATCTGGGTGAATGATCGCCATGTAGCCAATCATGTTTATGGATATACTGCATTTGAACT
GGATATAACCGATTATGTACGTTTCGGAGCTGAAAATCTGATAGCTGTCCGTGTGAAGAATGAAGGTATG
AATTGCCGTTGGTATACAGGTTCGGGTATTTACAGGCATACTTTCTTGAAGATAACCAATCCGCTTCATT
TTGAAACTTGGGGAACGTTTGTCACGACTCCCGTTGCAACGGCGGATAAAGCAGAGGTACATGTACAGAG
TGTTCTGGCAAATACTGAAAAAGTAACCGGAAAAGTGATTCTGGAAACGCGGATTGTAGATAAGAATAAC
CATACTGTAGCTCGGAAAGAGCAACTGGTAACATTGGATAACAAAGAAAAAACAGAGGTTGGCCATGCGT
TGGAAGTGCTTGCTCCGCAATTATGGTCTATAGACAATCCTTACTTATATCAGGTTGTAAACCGTCTTCT
GCAAGATGATAAAGTTATAGATGAGGAATATATTTCAATAGGTATACGCAATATTGCATTTAGTGCGGAG
AATGGTTTCCAGCTGAATGGTAAATCCATGAAACTAAAAGGCGGATGTATCCATCATGACAATGGTCTTT
TGGGTGCAAAGGCTTTTGACCGGGCAGAGGAAAGGAAAATAGAACTACTGAAAGCGGCTGGTTTCAATGC
GCTGCGCTTGTCTCATAATCCTCCCTCAATCGCTTTACTCAATGCCTGCGACCGCTTAGGTATGCTGGTC
ATAGATGAGGCTTTTGATATGTGGCGCTATGGTCATTATCAGTATGATTATGCACAATACTTTGATAAAT
TGTGGAAAGAAGATTTGCATAGTATGGTTGCACGGGATAGGAATCATCCTAGTGTTATCATGTGGAGTAT
TGGTAATGAAATCAAGAACAAAGAAACTGCTGAAATTGTGGATATATGCAGGGAGTTGACAGGTTTTGTG
AAGACGCTTGATACAACGCGGCCTGTTACGGCGGGAGTTAATTCTATTGTTGATGCAACGGATGATTTTC
TGGCTCCTCTGGATGTTTGTGGTTATAATTACTGTTTAAACCGTTATGAATCGGATGCCAAACGTCATCC
GGACCGTATTATCTATGCTTCGGAGTCCTACGCATCCCAGGCTTATGATTATTGGAAAGGAGTAGAAGAT
CATTCATGGGTGATCGGTGATTTTATCTGGACTGCTTTTGACTATATTGGTGAGGCAAGTATCGGCTGGT
GTGGGTATCCGCTTGATAAACGTATTTTCCCTTGGAATCATGCCAATTGTGGTGATTTGAATCTTTCGGG
CGAACGTCGTCCCCAGTCCTATTTGCGTGAAACGTTATGGAGTGATGCACCGGTATCCCATATTGTTGTG
ACGCCTCCTGTTCCTTCTTTTCCTCTGAATCCGGATAAGGCGGATTGGAGTGTATGGGATTTTCCGGATG
TTGTGGATCATTGGAATTTCCCGGGATATGAGGGGAAAAAGATGACAGTATCTGTATACTCCAATTGTGA
ACAGGTTGAACTGTTCTTGAATGGGGAATCTTTAGGAAAACAAGAAAATACTGCCGATAAGAAAAATACG
CTTGTCTGGGAAGTACCTTATGCTCATGGAATATTGAAAGCCGTAAGTTATAATAAAGGCGGTGAAGTGG
GCACTGCAACGTTGGAAAGTGCTGGTAAGGTTGAAAAGATCAGATTATCTGCGGACAGAACGGAAATCGT
AGCTGATGGTAATGATCTAAGCTATATCACATTAGAATTGGTAGATAGTAAAGGCATTAGAAATCAGTTG
GCTGAAGAATTGGTAGCATTTTCTATAGAAGGAGATGCTACG
SEQ ID NO: 8: Nucleotide sequence of APC115077.103; APC115077.20-
800.P(800)..pMCSG68
GGGGAAAAAGATTCCACACTTCGGGAACAATCTTTCGATGAGGCATGGTTATTTCATCGTGGAGATATTG
CCGAAGGAGAAAAGCAATCTTTAGATGACTCACAATGGCGTCAGATAAATCTTCCTCATGATTGGAGTAT
TGAAGATATTCCTGGAACCAATTCTCCTTTTACAGCGGATGCTGCAACGGAAGTTGCAGGTGGTTTTACT
GTAGGTGGTACGGGATGGTATAGAAAGCACTTCTACATAGATGCGGCTGAAAAAGGTAAATGTATTGCTG
TCTCTTTCGATGGAATTTATATGAATGCAGATATCTGGGTGAATGATCGCCATGTAGCCAATCATGTTTA
TGGATATACTGCATTTGAACTGGATATAACCGATTATGTACGTTTCGGAGCTGAAAATCTGATAGCTGTC
CGTGTGAAGAATGAAGGTATGAATTGCCGTTGGTATACAGGTTCGGGTATTTACAGGCATACTTTCTTGA
AGATAACCAATCCGCTTCATTTTGAAACTTGGGGAACGTTTGTCACGACTCCCGTTGCAACGGCGGATAA
AGCAGAGGTACATGTACAGAGTGTTCTGGCAAATACTGAAAAAGTAACCGGAAAAGTGATTCTGGAAACG
CGGATTGTAGATAAGAATAACCATACTGTAGCTCGGAAAGAGCAACTGGTAACATTGGATAACAAAGAAA
AAACAGAGGTTGGCCATGCGTTGGAAGTGCTTGCTCCGCAATTATGGTCTATAGACAATCCTTACTTATA
TCAGGTTGTAAACCGTCTTCTGCAAGATGATAAAGTTATAGATGAGGAATATATTTCAATAGGTATACGC
AATATTGCATTTAGTGCGGAGAATGGTTTCCAGCTGAATGGTAAATCCATGAAACTAAAAGGCGGATGTA
TCCATCATGACAATGGTCTTTTGGGTGCAAAGGCTTTTGACCGGGCAGAGGAAAGGAAAATAGAACTACT
GAAAGCGGCTGGTTTCAATGCGCTGCGCTTGTCTCATAATCCTCCCTCAATCGCTTTACTCAATGCCTGC
GACCGCTTAGGTATGCTGGTCATAGATGAGGCTTTTGATATGTGGCGCTATGGTCATTATCAGTATGATT
ATGCACAATACTTTGATAAATTGTGGAAAGAAGATTTGCATAGTATGGTTGCACGGGATAGGAATCATCC
TAGTGTTATCATGTGGAGTATTGGTAATGAAATCAAGAACAAAGAAACTGCTGAAATTGTGGATATATGC
AGGGAGTTGACAGGTTTTGTGAAGACGCTTGATACAACGCGGCCTGTTACGGCGGGAGTTAATTCTATTG
TTGATGCAACGGATGATTTTCTGGCTCCTCTGGATGTTTGTGGTTATAATTACTGTTTAAACCGTTATGA
ATCGGATGCCAAACGTCATCCGGACCGTATTATCTATGCTTCGGAGTCCTACGCATCCCAGGCTTATGAT
TATTGGAAAGGAGTAGAAGATCATTCATGGGTGATCGGTGATTTTATCTGGACTGCTTTTGACTATATTG
GTGAGGCAAGTATCGGCTGGTGTGGGTATCCGCTTGATAAACGTATTTTCCCTTGGAATCATGCCAATTG
TGGTGATTTGAATCTTTCGGGCGAACGTCGTCCCCAGTCCTATTTGCGTGAAACGTTATGGAGTGATGCA
CCGGTATCCCATATTGTTGTGACGCCTCCTGTTCCTTCTTTTCCTCTGAATCCGGATAAGGCGGATTGGA
GTGTATGGGATTTTCCGGATGTTGTGGATCATTGGAATTTCCCGGGATATGAGGGGAAAAAGATGACAGT
ATCTGTATACTCCAATTGTGAACAGGTTGAACTGTTCTTGAATGGGGAATCTTTAGGAAAACAAGAAAAT
ACTGCCGATAAGAAAAATACGCTTGTCTGGGAAGTACCTTATGCTCATGGAATATTGAAAGCCGTAAGTT
ATAATAAAGGCGGTGAAGTGGGCACTGCAACGTTGGAAAGTGCTGGTAAGGTTGAAAAGATCAGATTATC
TGCGGACAGAACGGAAATCGTAGCTGATGGTAATGATCTAAGCTATATCACATTAGAATTGGTAGATAGT
AAAGGCATTAGAAATCAGTTGGCTGAAGAATTGGTAGCATTTTCTATAGAAGGAGATGCTACGATTGAAG
GAGTAGGTAATGCCAACCCTATGAGCATAGAAAGTTTCGTTGCTAATAGTCGGAAGACGTGGCGCGGAAG
TAACTTATTGGTTGTTCGTTCCGGGAAATCTTCAGGACGGATTATTGTAACAGCAAAGGTAAAGGCACTT
CCGGTTGCGAGTATTACTATAACTCAGAAAAAA
SEQ ID NO: 9: Nucleotide sequence of APC115086.102; APC115086.29-
766.P(766)..pMCSG68
AAATCTCCTGTCGATATGGATCGCTTTATTGATGATCTGATGAAGAAGATGACTCTGGAAGAGAAAATCG
GCCAGTTGAACTTGCCTGTTACGGGTGAAATAACCACCGGACAAGCCAAGAGTAGTAATGTGGCTAAGCG
TATCCGTGCCGGTGAAGTGGGCGGACTCTTTAACTTGAAAGGCGTGGAGCGTATTCGTGACGTTCAGAAA
CAGGCAGTAGAAGAAAGTCGTCTGGGTATTCCTCTTTTATTTGGTATGGATGTAATTCATGGATACGAAA
CGGTATTTCCTATTCCTCTGGGATTATCCTGTACCTGGAACATGACAGCTATTGAAGAATCTGCACGTAT
TGCTGCTATCGAAGCCAGTGCTGATGGTATTTGCTGGACATTCAGTCCGATGGTGGATGTTTCCCGTGAT
CCCCGTTGGGGACGAGTTTCCGAAGGGAATGGTGAAGATCCCTTCTTGGGAGCGGAGATTGCGCGTGCTA
TGGTACGTGGTTATCAAGGGAAAGATATGAGTAGTAATGATGAAATTATGGCTTGCGTGAAGCACTTTGC
GTTATATGGGGCATCAGAAGCCGGACGCGACTATAATACAGTGGATATGAGTCATCAACGTATGTTCAAC
GAATATATGTTACCTTATCAGGCTGCCGTGGAAGAAGGTGTGGGTAGTGTGATGGCTTCATTCAATGAAG
TGGATGGTGTACCGGCTACCGGAAATAAGTGGCTGATGACCGATGTACTTCGTAAGCAGTGGAATTTTGA
TGGGTTCGTTGTGACGGACTATACCGGTATCACTGAAATGACCGATCATGGTATGGGTGATACACAAACA
GTTGCAGCCCTGGCTCTGAATGCAGGTGTCGATATGGATATGGTGAGCGATGCTTTTACAAGCACACTTA
AAAAATCTCTGGAAGAAGGAAAAGTTTCAGTAAAGGCTGTTGATGCTGCTTGTCGCCGTATTCTGGAAGC
TAAGTATAAGCTGGGGCTTTTTGATAATCCCTATAAATATTGTGATATAACCCGTCCTAAAAAACAAATC
TTTACAAAAGAACACCGCGCTATAGCCCGTAAGACAGCTTCGGAAAGCTTTGTTCTCTTGAAGAATGAGA
ATAGTGTACTCCCTCTGGCAAAGAAAGGTACCATTGCTGTAGTAGGTCCTTTGGCCGATAGCCGTAGCAA
TATGCCGGGCACGTGGAGTGTGGCCGCTGTGATGAACAAATATCCTTCTTTGATTGAAGGCTTGAAAGAA
GTAGTGGGAGGCAAGGCTAAAATTCTTACGGCTAAAGGAAGTAATCTGATGAGTGATGCCGAATACGAAG
AACGTGCTACTATGTTTGGCCGTACTCTGCATCGTGACAATCGTACAGATAAGGAACTGCTGGATGAGGC
GCTTGCTGTAGCTGCCAAGTCTGACGTGATTGTTGCTGCTTTGGGTGAGTCTTCCGAGATGAGCGGTGAA
AGTAGTTGCCGTACAGACCTCGAAATGCCGGATACGCAACGTGTACTTTTGCAGGAATTGTTGAAAACCG
GCAAACCGGTGGTATTGGTGTTGTTTACCGGTCGTCCGTTAGTATTGAATTGGGAGCAGGAAAATGTACC
TGCTATTCTGAATGTGTGGTTTGGTGGTAGTGAAGCTGCTCTTGCCATTGGTGATGTACTGTTTGGAAAT
GTAAATCCGAGTGGCAAACTTACTACTACTTTTCCGAAGAGTGTAGGACAGATTCCTTTGTTCTATAACC
ATAAGAATACTGGTCGTCCTTTGCCTCAAGGGGCCTGGTTCCAGAAGTTCCGTAGCAATTATCTGGATGT
AGATAACGAACCGCTTTATCCGTTTGGATATGGCTTGAGCTATACTACTTTCTCTTATAGTGATATTACA
TTGGATAAATCGTCCATGAATATCAATGGAGAGATTATGGCAACTGTAACGGTAACCAATACAGGTAAGT
ATGACGGTTCGGAAGTAGTGCAGCTATATATCCGCGATCTTATAGGCAGTGTAACACGTCCGGTGAAAGA
ACTGAAAGGCTTTGAAAAAATCTTCTTGAAAGCCGGTGAATCCAAACAAGTGTCTTTCAAGTTAACAGCT
GATATGTTGAAGTTCTACAATTACAATCTGGATTTTGTGTGCGAACCGGGTGACTTTGAAGTAATGATAG
GTGGTGATAGCCGTGATGTGAATAAGGCCTTATTTTCGCTTCAA
SEQ ID NO: 10: Nucleotide sequence of CMR200017.102; CMR200017.21-
605.P(605)..pMCSG68
CAATGGAAACCGGCCGGAGATAGAATAAAGACAAAGTGGGCAGAACAGATCAATCCTTCCGATGTATTGC
CCGAGTATCCAAGGCCCATCATGCAGCGTAATGACTGGAAAAACCTGAATGGTTTGTGGGATTATGCTAT
TATTGATAAAGGTGGACGCATTCCAACGGATTTTGAAGGCCAAATTCTCGTACCTTTTGCTGTAGAATCG
TCTTTGTCCGGAGTAGGAAAAAGAGTGAACGAAAATCAGGAAGTAATCTATCAGCGGAGCTTTGAGATAC
CTTCAGCCTGGAGAGGAAAACAGGTTTTGCTACATTTTGGTGCCGTTGACTGGAAAACCGATGTATGGGT
GAACGATATTAAGGTTGGAAGTCATACCGGAGGATTTACTCCATTCTCCTTTGATATAACTCCTGCCTTG
TCGGCTAAAGGTAACAACCGTCTGGTTGTAAAGGTTTGGGACCCTACGGACAGAGGCCCTCAACCACGTG
GTAAGCAAGTCAGCAGACCGGAAGGTATCTGGTACACTCCTGTAACAGGTATCTGGCAAACTGTATGGCT
GGAACCTGTTGCTGGTAAACATATTGAGAATCTTCGTATTACTCCTGATATTGACCGTCATCTGTTAACG
GTAAAAGCTGAACTGAACACCAACAGCACATCAGACTTCGTGGAGGTGAATGTGTATGATGGTAATCAAT
TAATTGCTGCCGGTAAGAGTATTAATGGGGAACCTGTAGAAGTGGCAATGCCTGAAAATGCAAAACTGTG
GAGCCCTGATTCTCCTTTTCTCTATACTTTGAAAGTTACTTTAAAAGAGGGGAATAAGATTGTGGATAAG
GTGGATAGCTATGCGGCCATGCGTAAATATTCCACTCGCAGGGATGCCAATGGTATCGTACGTTTGGAAC
TGAATAATGAAGCGCTGTTCCAGTTTGGCCCGCTTGATCAAGGTTGGTGGCCTGACGGTCTGTATACGGC
TCCTACGGATGAAGCTTTGCTGTACGACATTCAGAAGACAAAAGATTTTGGTTATAATATGATCCGTAAA
CATATTAAAGTAGAGCCTGCCCGTTGGTATACATATTGCGACCAGCTTGGAATTATTGTGTGGCAAGACA
TGCCGAGTGGTGACCGCAACCCGCAATGGCAGAACCGGAAGTACTTTGATGGTACGGAAATGAAGCGTTC
AGCCGAATCAGAAGCTTATTATCGCAAAGAATGGAAAGAAATAATGGACTGTCTGTATTCTTATCCTTGC
ATTGGTACCTGGGTGCCATTTAATGAGGCTTGGGGACAGTTTAAGACCGTTGAAATTGCTGAATGGACGA
AACAATATGATCCGACCCGTTTGGTGAATCCAGCAAGTGGCGGTAATCATTATACTTGTGGTGATATGCT
TGACCTGCATAATTATCCGGCACCTGAGATGTACTTGTATGATGCTCAGCGTGCAACTGTTTTGGGTGAA
TACGGTGGTATCGGTCTTGTTCTGAAGGATCATATCTGGGAGCCGAACCGTAACTGGGGTTATGTTCAAT
TTAATTCTTCCAAAGAAGCTACGGATGAATATGTGAAGTATGCCGATATGCTGTATAAGATGGTAGACAG
AGGATTCTCCGCAGCTGTCTATACACAGACTACTGACGTGGAAGTGGAAGTGAATGGCCTGATGACCTAT
GACCGTAAGGTTATTAAACTGGATGAAAAGCGTGCTAAAGAAATAAATACACGTATCTGTAATTCGTTGA
AAAAG
SEQ ID NO: 11: Nucleotide sequence of CMR200018.102; CMR200018.20-
949.P(949)..pMCSG68
CAGACACTTCCGCAGACAGAGCGGCAATACCTCTCCGGCCACGGATGCGACGACACAGTAGAATGGGACT
TTTTCTGTACCGACGGACGTAACTCCGGTCGATGGACGAAAATAGGCGTCCCCTCTTGCTGGGAGTTGCA
GGGTTTTGGTACCTATCAGTATGGAATTAGTTTTTATGGTAAAGCCTTTCCCGAAGGCATTGCCGGTGAG
AAAGGAATGTATAAATATGAGTTTGAAGTTCCCGAGGAATTTCGTGGCAAGCAGGTCAGCCTTGTGTTCG
AAGCATCCATGACCGATACGGAAGTTAAGGTTAACGGACGTAAGGCAGGATCGAAACACCAGGGAGCCTT
CTATTGCTTTTCATATAATGTCACGGATTTACTGAAATATGGCAAGAAGAATCAGCTGGAAGTAACAGTT
TCCAAGGAGAGTGAGAATGCCAGTGTGAATCTTGCCGAACGGCGCGCCGATTATTGGAACTTTGGCGGTA
TCTTCCGCCCGGTATTTCTGGAAGTAAAACCTGCCGTCAATCTCCGTCATATTGCTATTGATGCACAAAT
GGACGGATCATTCCGTGCCAATTGCTACACGAATATCTCCGGTGACGGAATGAGTATCCGTGCACAGATT
TTGGACGGTAAAGGGAAGAAACTGGCAGATACCACCGTACCCCTAAAAGCCGGAAGCGACTGGACTACTT
TACAATTGAACGTTTCTGCCCCTGCCTTATGGACGGCAGAAACTCCGAATCTTTATAAAGCTCAATTTTC
ACTGTTGGATAAAGGAGGTAAAGTCCTGCATCATGAGACCGAGACATTCGGTTTCCGTACTATCGAAGTT
CGTGAAAGTGACGGATTGTACGTGAACGGGGTGCGTATCAACGTGCGTGGTGTCAACCGTCATAGTTTCC
GTCCCGAAAGCGGTCGTACCCTAAGTAAAGCGAAGAATATTGAAGATGTACTTCTGATGAAGGGCATGAA
TATGAATTCTGTCCGTCTGAGCCACTATCCGGCGGACCCGGAATTTCTGGAAGCATGCGACTCTCTTGGA
CTCTATGTTATGGATGAACTGGGTGGCTGGCATGGCAAGTACGACACCCCTACGGGAGTACGTCTGATTG
AAGGCATGATAGAACGTGATGTGAACCATCCGTCCATTATCTGGTGGAGCAATGGTAATGAAAAAGGCTG
GAACATTGAACTGGACGGAGAATTCCATAAATACGATCTGCAGAAACGCCCGGTCATCCATCCGCAAGGT
AACTTCTCCGGTTTCGAAACCATGCACTATCGTTCGTATGGAGAAAGCCAGAACTACATGCGCCTGCCGG
AAATCTTTATGCCTACTGAATTCCTGCATGGTTTGTACGACGGAGGTCATGGTGCCGGCCTGTATGATTA
CTGGGAAATGATGCGTAAACATCCGCGTTGTATCGGTGGTTTCCTGTGGGTATTGGCGGATGAAGGCGTG
AAGCGCGTGGATATGGACGGGTTCATAGACAATCAGGGAAATTTCGGAGCTGACGGAATTGTAGGCCCTC
ACCATGAAAAGGAAGGCAGCTATTACACTATCAAGCAGCTATGGAGCCCGGTGCAGGTTATGAATACCGC
TATCGACCGGAATTTCGACGGTAAACTCTCTGTGGAGAACCGTTATGATTATCTGAACCTGAACACCTGT
CGTTTTATCTGGCAGCAAGTGAAGTTCCCGTCGGTAACGGATGCTTCCAATACAACTACACGGATTCTGA
AACAAGGTGAAGTGCAAGGAAGCGATGTAGCAGCCCATGGAGTGGGAGTGGTGGATATCAAGACTTCTAT
TCTTCCCGAAGCGGATGCTCTTTTCCTGACAGTTATAGATAAATATGGGTATGAACTTTGGCGCTGGACT
TTCCCCGTAGATAAACTGAATCGGGAAACAGAACAGTTTTCTGCATCATCCGGCCGTGTATCCTATACGG
AAACAGAAAAAGGTATTACGGTAAAAGCAAACGGGCGTACTTTTGTCTTTTCAAAGAAAGACGGGCAGCT
GAAAGATGTATCCGTCAATAACCGTAAGATTAGTTTTGCTAACGGTCCCCGTTTTATCGGTGCACGTCGT
GCAGACCGTTCCCTAGATCAGTTCTATAATCATGATGACGAAAAAGCCAAGGCAAAGGACCGTACTTACA
GTGAATTTACCGATGCGGCAGTCTTCACGAAACTGGATGTGAAAGAAGAGGGGGGGAATCTGATCCTCAC
CGCTAATTATAAACTGGGTAATTTAGATAAAGCTCAGTGGACAATTCATCCGGACGGCATGGCTACTCTT
GATTATACCTACAACTTCTCCGGTGTGGTAGACCTGATGGGTATTTGCTTTGATTACCCTGAAGAACAAG
TGCTCAGCAAGCGTTGGTTGGGAGCAGGTCCGTATCGTGTATGGCAGAATCGTATTCATGGCACGCAGTA
TGATATCTGGGAGAATGATTATAACGATCCTATTCCGGGTGAGACATTCACCTATCCTGAATTCAAGGGA
TATTTTGGCAGTGTCTCTTGGATGAGTATTCGCACGAAAGAGGGAACCATCAGCCTGACGAATGAAACAC
CTGATTCCTATATCGGAGTATATCAACCCCGTGATGGTCGTGACCGGTTACTGTATACACTTCCCGAAAG
CGGAATTTCTGTTTTGAATGTAATTCCTCCGGTGCGTAATAAAGTAAATTCCACGGACTTGTGCGGTCCT
TCTTCACAACCAAAATGGGTGGATGGCTCGCAAACGGGACGCCTTGTTATCCGGTTTGAA
SEQ ID NO: 12: Nucleotide sequence of CMR200027.102; CMR200027.20-
824.P(824)..pMCSG68
CAGCGCAGTGAGTATCTACTTGAAAAGAACTGGAAGTTCATGAAGGGGGAAGCTCCGGAAGCCATGAAGC
CGGAATTTGACGACCGGAAGTGGGAAACCGTAACCGTGCCTCACGACTGGGCCATTTTTGGTCCCTTCGA
TCGCAGCAACGATTTGCAGGAAGTGGCGGTAACGCAGAACTTCGAGAAGAAAGCTTCCGTCAAGACCGGA
CGTACCGGTGGACTTCCTTATGTTGGCATCGGATGGTATCGTACTAGGTTCGATGCCCCCGTCAATCAAC
AGACGACACTTGTCTTTGATGGTGCCATGAGCGAAGCCCGTGTATATGTCAATGGACAAGAAGCATGCTT
CTGGCCATTTGGTTATAATTCTTTCCATTGTGATGTCACCGGACTTTTGAATAAAGACGGTAAAAACAAT
ACGCTTGCCGTGCGTTTGGAAAATAAACCACAATCTTCCCGTTGGTATCCTGGCGCAGGACTTTATCGCA
ATGTGCGTGTAGTGAGTACCGATAAAGTACATGTTCCTGTATGGGGTACTCAGCTGACTACTCCTCATGT
TTCTGATGAGTATGCTTCAGTACGTCTGTTGACCACTATTGCCAATGATGAAGAAAGAGATATCCGTATC
GTGACAGAGATAATCTCTCCCGATGGGAAAGTCGTTGCAACGAAGGATAATACCCGTAAGATTAATCATG
GTCAGCCTTTTGAACAAAACTTCCTGGTGAATGCTCCTTGCTTGTGGTCGCCGGAGACACCTTATTTATA
TAAAGCTGTTTCTAAAATCTATGCCGATGGCAAGCAAACGGATGAATACACTACTCGTTTCGGCATCCGC
AGCATAGAAATCATTGCCGACAAAGGATTTTTCCTGAACGGTAAGCATCGCAAGTTCCAGGGGGTGTGCA
ATCACCACGATCTTGGTCCGTTAGGCGCTGCCATCAATGTTGCTGCATTGCGCCGTCAACTTACGATGCT
GAAAGATATGGGTTGTGATGCCATCCGCACCGCTCACAATATGCCGGCACCGGAGTTAGTGCAACTTTGT
GATGAAATGGGTTTTATGATGATGCTGGAACCTTTCGACGAATGGGACATTGCCAAATGTGAGAATGGCT
ATCACCGTTATTTCAACGAGTGGGCAGAACGTGATATGATAAATATGTTGCATCAGTTCCGCAACAATCC
TTGTGTCGTAATGTGGAGTATCGGTAATGAAGTTCCTACCCAATGTAGTCCCGTAGGCTATAAAGTCGCT
TCTTTCTTGCAGGATATCTGTCATCGTGAAGATCCGACACGTCCTGTTACTTGCGGCATGGATCAGGTGA
CTTGTGTTCTTGCTAATGGTTTTGCCGCCATGATTGATGTGCCCGGTTTTAATTATCGCGCACACCGTTA
TCTGGAAGCTTATGAACTGTTGCCGCAGAATATAGTACTTGGTTCTGAAACATCCTCTACCGTTAGTTCT
CGTGGCGTATATAAATTTCCTGTAGAGAAACGCGGGGATGCGAAGTACGATGATCACCAGTCTTCCGGAT
ATGACTTGGAGCATTGTGCCTGGTCTAATGTTCCAGATGAAGATTTTGCTTTAGCGGATGATTATGACTG
GACTATCGGTCAATTCGTTTGGACAGGATTCGATTATCTGGGTGAGCCTTCTCCTTATGATACGGATGCA
TGGCCAAGTCATAGCTCTTTGTTTGGTATCATTGACCTTGCCAGTTTGCCAAAAGACCGCTACTATCTGT
ACCGTAGTCTTTGGAATAAGAATGTGAATACACTCCATATACTTCCTCACTGGACATGGCCGGGTAGGGA
AGGAGAGAATACTCCTGTCTTTGTTTACACAAACTATCCTGCTGCCGAACTTTTCGTTAATGGAAAAAGC
TATGGTAAACAGCATAAACTGACAGCCGAAGAGAGTAAAGCTATTCAGGACAAAGATACACTTGCCCTCC
AGCGTCGTTACCGCCTGATGTGGATGGACGTTCCTTATGAGCCGGGTGAAGTGAAAGTGGTGGCTTACGA
TGCTTCCGGCAAACCTGCTGAAGAAAAAGTAGTTCGTACTTCCGGCAAACCTCATCATCTGGAAGTCATT
GCTGACCGTGACCAACTCACTGCCGATGGTAAAGATTTGGCATACATCACTGTTCGTGTGGTTGATAAAG
ACGGAAACCTTTGTCCTGCTGATAATCGTCTTGTAAACTTTACGGTGAAAGGCGCGGGGCGTTATCGTGC
TGCCGCTAATGGAGATGCAACTTCACTTGATTTATTCCACTTGCCGAAGATGCCCGCTTTCAGTGGTCAG
CTGACAGCCATTGTTCAAATGACCGAACAGCCCGGTGAAATTATTTTCGAGGCTAAGGCTAAAGGGGTGA
AATCTGGTAAGCTTGTGCTGAGGTCTGTTAGAGAG
SEQ ID NO: 13: Nucleotide sequence of CMR200113.102; CMR200113.22-
415.P(415)..pMCSG68
GGTGAAAAGGCAGAAAAAATACAGGATTTTGCTGAGTTTATAACCATTCAGGGGCAAGACCTGATAAAAC
CTGATGGTACGAAACTCTTTATCATGGGTACCAATCTGGGCAATTGGCTGAATCCGGAAGGGTATATGTT
TAAGTTTAACAAAACGAATTCTCCCCGGTTTATCAATGAAATGTTCTGCCAATTGGTAGGACCCGACTTT
ACTGCTGAGTTTTGGAAAGCTTTCAAAGACAATTATATCATTCGTGAAGATATTCAGTTTATTAAGAATA
CAGGTGCGAATACCATTCGTCTTCCATTCCATTATAAGCTTTTCACGGATGAGGACTTTATGGGGTTGAC
TGCCGGTCAGGATGGTTTTGCCCGTGTAGACAGTGTTGTGGAATGGTGCCGTGAAGCCGATCTTTATCTG
ATTCTTGATATGCATGATGCTCCGGGTGGACAAACGGGTGATAATATAGATGATAGCTACGGATATCCTT
GGTTGTTTGAAAGTGAAGCCAGCCAGCAATTGTATTGCGATATCTGGCGCAAGATTGCAGACCGGTATAA
GAATGAACCGGTGATTCTCGGTTATGAGCTTTTCAATGAACCTATCGCTCCGTATTTTCCGAATATGGAA
GAATTGAACGGTAAACTGGAAGATATTTATAAGAAAGGGGTAGCTGCTATCCGCGAGGTGGACAATAACC
ATATTATTCTGTTGGGTGGCGCTCAGTGGAACGGTAACTTCAAGCCGTTCAAGGATTCTAAGTTTGATGA
TAAAATAATGTATACTTGCCATCGTTATGGAGGTGATCCTACTAAAGATGATATTCAAACTATAATAGAC
TTCCGCGACAGTGTGAACTTACCAATGTATATGGGTGAGATAGGACATAACACGGACGAATGGCAAGCTG
CTTTTTGCCAGACGATGCGTGAGAATAATATCGGTTATACCTTCTGGCCGTATAAGAAGATGGATGGTTC
CAGCTTTGTAGGTATTACTCCGCCGGAAAATTGGGCGAATATCCTTTATTTCTCCGAATCTCCACGCACA
TCTTATAAAGAAATCCGGGATGCCCGTCCCGACCAGATGATGGTACGCAAGGCAATGATGGATTTCATTG
AGGCTTGCAAACTGAAGAACTGTGTGGTGCAGGAAGGGTATATTCAGTCGTTAGGTATGAAA
SEQ ID NO: 14: Nucleotide sequence of CMR200122.102; CMR200122.20-
750.P(750)..pMCSG68
ACACAAGTGGCAAATAAAGGTAGCGATGCGGCAACCGAGAAAAAAGTAGAGTCTCTTTTATCCAGAATGA
CCCTTGAAGAGAAAATCGGTCAGATGAACCAGATTACCTCTTACGGGAATATTGAGGATATGAGTAGTTT
AATTAAGAAAGGTGAAGTCGGGTCTATCCTGAATGAGGTGGATCCGGTACGTATTAATGCGTTGCAACGC
GTAGCGATGGAGGAGTCCCGGTTGGGAATCCCTTTGTTGATAGCTCGCGATGTTATTCACGGGTTTAAAA
CCATTTTTCCCATCCCATTGGGACAAGCGGCTTCGTTCAATCCGCAGATTGCGAAAGACGGTGCACGGGT
AGCGGCTATTGAGGCTTCTTCCGTAGGTATCCGTTGGACTTTTGCACCGATGATCGACATTGCCCGTGAT
CCTCGCTGGGGGCGCATTGCCGAAGGATGTGGTGAAGACACTTACCTGACTTCTGTAATGGGAGCTGCCA
TGGTAGAAGGTTTTCAGGGAGATTCTTTGAATAGTCCCACTTCCATAGCTGCCTGTCCTAAACATTTTGT
GGGCTATGGTGCAGCTGAAGGCGGACGTGACTATAATTCGACATTTATTCCTGAACGTCGCCTGCGTAAT
GTTTACTTGCCACCGTTTGAAGCGGCAACGAAAGCGGGTGCAGCTACGTTTATGACTTCCTTTAATGATA
ATGATGGGATACCCTCTACCGGAAATGCTTTCATATTGAAAGATGTGCTTCGTGGCGAGTGGGGATTTGA
TGGTTTGGTAGTGACAGACTGGGCTTCTGCCAGCGAAATGATAAGTCATGGTTTTGCTGCCGATTCTAAA
GAGGTAGCCATGAAATCAGTGAATGCTGGGGTGGATATGGAAATGGTAAGTTATACCTTTGTAAAAGAAT
TGCCTGCATTGATAAAAGAAGGAAAGGTGAAAGAAAGCACCATTGATGAAGCCGTTCGTAATATATTGCG
CGTCAAGTATCGTCTGGGATTGTTTGATGTTCCTTATGTAGATGAAAAGCAACCCTCTGTCATGTATGAT
CCTTCTCATCTGAAAGTAGCTAAGCAGGCTGCTGTAGAATCGGCTATCCTGTTGAAGAATGATAAAGAAG
TACTGCCGTTACAGGAGTCTCTGAAAACCATTGCTGTGGTAGGACCTATGGCCAATGCGCCTTATGAACA
ATTGGGTACCTGGATCTTTGATGGTGAGAAAGCTCATACTCAGACACCACTGAATGCTATTAAGGAAATA
GTTGGCGACAAAGTACAGGTGATTTATGAACCCGGATTAGCTTATAGCCGTGAGAAAAATCCGGCAGGCG
TAGCAAAAGCTGCTGCTGTTGCTGCACGTGCAGATGTCATTCTTGCTTTTGTGGGTGAAGAAGCCATTCT
TTCGGGTGAAGCACACTGTCTGGCAGATTTGAATCTTCAGGGTGATCAAAGTGCTTTGATTACGGCTTTG
GCTAAGACAGGTAAACCTGTAGTAACCATTGTGATGGCAGGTCGTCCGTTGACTATCGGTCAGGAAGTGG
AAGAATCAACAGCTGTTCTTTATTCATTCCATCCGGGTACGATGGGTGGACCGGCATTGGCCGATCTGCT
GTGGGGTAAGGCGGTTCCAAGTGGAAAAACACCGGTTACTTTCCCGAAGATGGTAGGACAAATTCCGGTA
TATTATGCTCATAACAATACCGGGCGGCCGGCTACACGTAATGAGGTGTTGCTGGATGATATTGCTGTTG
AGGCTGGACAAACTTCATTGGGATGTACTTCTTTCTATATGGATGCCGGTTTTGATCCTTTATTCCCATT
TGGCTATGGCTTGTCGTATACAACGTTCAAGTATAGTAATGTCAAACTTTCATCAGCGTCATTGAAGAAA
GATGATGTATTGACTGTGACATTTGATCTGGAAAATACAGGTAAATATAAGGGGACGGAAGTTGCTCAAT
TGTATATACAAGATAAGGTTGGTTCTGTAACTCGTCCGGTGAAAGAACTGAAACGTTTTACTCGGGTAAC
CTTGAAACCGGGCGAGAAAAAGAATGTTTCGTTTGAACTACCCGTTAGTGAACTTGCATTTTGGAACATC
GATATGGTGAAAGTTGTGGAACCCGGAGACTTTGGACTTTGGGTGGCAACAGACAGCCAATCGGGAGAAG
AAGTTTTCTTTAAGGTGGTAGAT
SEQ ID NO: 15: Nucleotide sequence of CMR200130.102; CMR200130.32-
851.P(851)..pMCSG68
TCTGATTCAAATGTTGATTTCAATAAAGATTGGAAATTCGTACTGAAAGATTCTGCTCATTATTCATATA
CTTCTTATGTCCCTGGTGATGAATGGAAGAAAGTGAACCTGCCACACGACTGGAGTGTTGGTCTGCCTTA
CGACTCCATCTCTGGCGAAGGGTGTGTAGCTTTCCTTCAGGGAGGAATAGGATGGTATAGCAAATCATTT
CCCACAACAATCAGCGCAAATCAGAAATGCTATATAGTGTTCGATGGAGTATATAATAATTCTGAGTATT
GGATAAATGGCAAAAAACTTGGATATCATCTTTCGGGATATGCTCCTTTTTATTTTGATGTCACAGACTA
TCTCAATCCCAATGAGGATAACCGCATGACTGTAAGGGTCGACCACAGCCATTATGCCGACAGCAGATGG
TACACCGGTTCAGGTATATACAGGGATGTGAAAATGATTGTAACCGACAGACTGCATATTCCGGTTTGGG
GAACATTTGTCACTACTCCCGTGGTTACTGATAAATATGCTAAAGTAAACAACCAAATTACCGTGCGCAA
CAGTTACTCTGAACCCAGAACAGCTGTTGTTGAGATAGTGTATAAAGATAATAAAGGCAATATCGCAGCC
TTTGAGGTCTTCAGTATAAAACTGAATGCTGGTGAGGAGAAAATTATCGACATCGTATCGGAGATAAAAC
AGCCGGATTTGTGGAGCGTCGAGATACCAGTCCTCTATACAGCCGAGACCCGTATTAAGAATGGCGATGA
AGTCATTTCTGAAAACACTGTCAGGTTCGGTATACGAACATTCCACTTTGATGCAGACAAAGGTTTCTTC
CTTAACGGAAAAAATATGAAGATAAAAGGAGTATGCCTGCATCATGATGCCGGTATAGTTGGCACAGCAA
TGATACGCGATGTGTGGTACCGACGTCTGAAAACCCTTAAGGAAGGAGGATGTAACGCCATCCGCCTTTC
GCACAATCCGGGAGCGGATGAGTTTCTGTCTTTGTGCGATGAGATAGGTCTTCTGGTCCAGGAAGAGTTC
TTCGATGAGTGGGATTATCCCAAAGATAAAAGGCTCAATATGAAGGAAACGGTAGAAGACTATCCTACTC
ATGGTTATTGTGAGCATTTCCAGGAATGGGCTGAAAGGGATTTGAAAAACGTAATGAGGAGAAGCCGTAA
TCATGCCTGTATCTTCCAGTGGAGTATAGGTAATGAAATAGAATGGACTTATACCGGATGCCGTGAGGCA
ACAGGTTTCTTTGGAGCCGATTCCAACGGTAATTACTTCTGGAACCAGCCTCCATACTCTAAAGAAAAAA
TCAGAGAAATGTGGAAAATCCAGCCTAAACAAGCATACGACATTGGTCGTACAGCGCAAAAATTAGCAGC
ATGGACACGCCAGATGGATACTACACGAGTGGTTACCGCCAACTGCATCCTGCCTTCCATAAGTTTTGAG
ACAGGATATATCGATGCACTTGATGTGGCTGGTTTCAGCTACAGACGCGTGATGTATGATTATGCTAAGA
AGAATTATCCTGACAAACCTATAATGGGTACAGAAAATCTTGGTCAGTGGCACGAATGGAAGGCGGTGAT
TGAAAGAGATTTCGTTCCGGGTATGTTTATATGGACAGGAGTCGATTATCTGGGAGAAAGTGGAAGCCGC
CTTTCAAGATGGCCTCAAAAGTCAATAGGATGTGGTCTCCTGGATATGTGCGGCTATGTGAAGCCTTCGT
ACGACATGATGAAATCATTGTGGACTGACAAGCCTTTTATTGCTATATATTCACAGACTCCAGACAAATC
TTCGTATCTCCAGGTAAAAGATGGCTTTACTGATAAGAAAGGACATGAATGGGATAGAAGATTATGGGTT
TGGGATGATGTAAACTCTCACTGGAATTATCAGAAAGGTGACTCGGTAATAGTAGAAATATATTCCAATT
GTGATGAAGTGGAACTTTTCGTTAACGGCAAGTCGATGGGAAAGAAGTATATAGACGATTTTGAGGATCA
TATCTATAAATGGGCAGTTCAGTACAAGCCTGGCACTATTACCGCAAAAGGAAAAAATAAGTTAGGTAAT
ACCACTACAGCTATAAGGACTTCAGGCAAAGAACATTCGATATTGCTAGCGGTTGACAAACAAAGTATCG
CAGCAAATGGAAAGGATGTTCTGCATGTCACAGCCCAGCTTACAGACAAAAAAGGTAATCCTGTAAAGAC
AACAGAACAGATGCTTAAGTTCAACATCGATGGAGAGTACCGTCTGTTGGGTATAGACAATGGAAATGTA
AAGAACGTATCTCCATATCAAAGCAAGGAGATTATGACATATCAGGGAAGATGTATGCTGATGCTTCAGT
CAACAGAAAAAACATCGGTACTGAATATCAGTGCAGAAACAAGTGAATTACAGTCGAATAAACTAACAAT
TAATATAAAA
SEQ ID NO: 16: Nucleotide sequence of CMR200135.102; CMR200135.22-
812.P(812)..pMCSG68
CAGCGACATGAACAACTCTTGGAAACCGGCTGGAAATTCCACAAAGGAGAAACCAATGGAGCTGAAACTG
TTTCATTTAATGATTCTCAATGGGAATCTGTCTGTATTCCACACGACTGGGCCATTTATGGACCGTTTGA
CCGTAATAATGATTTACAAAATGTAGCCATTACTCAGAACTTGGAGAAACAGGCATCTGTCAAGACCGGA
CGTACCGGAGGACTTCCTTATGTGGGAGTAGGATGGTATCGCACCCGTTTCGATGCAGACCCTGACAAAA
AGACAACACTGGTTTTTGATGGAGCCATGAGTGAAGCCCGCGTGTATGTCAATGGAAAAGAAGCCTGCTT
CTGGCCTTTCGGTTACAATTCCTTCCATTGTGACATTACTGAGCTTCTGCACAAAGAAGGAAAAGACAAT
GTATTGGCTGTACGTCTGGAAAACCGTCCTCAATCTTCCCGCTGGTATCCGGGAGCCGGACTTTACCGGA
ATGTCCATCTGATTACTGCAGAAAAAATACATGTACCTGTATGGGGAACACAGGTTACCACCCCACACGT
AGCTAATGACTATGCTTCTGTTTGCCTTCGTACCTCTTTACAGAATGTGGGAAAAGAAGAAATTACCATA
GAAACAGAAATACTGGACCCGAACGGGAAAAAAGTTTCTTTCAAGAAGAACAGCGGACGCATCAATCACG
GGCAACCGTTTACACAAAATTTCATTGTGGAAAACCCGCAATTGTGGTCACCTGAAACACCGTTCTTATA
TCAGGCCGTATCTAAAATCTATGCCAACGGAAAACTTACAGATACTTATACCACCCGCTTTGGTATCCGT
TCCATCGAATTTGTAGCCGACAAGGGCTTTTTCCTGAACGGCCAGCACCGTAAATTCCAGGGGGTATGCA
ACCACCACGACTTAGGTCCTTTAGGAGCTGCCATCAACGTATCGGCTCTACGCCACCAGCTTACATTATT
AAAAGACATGGGCTGCGATGCCATTCGTACCGCACACAACATGCCGGCACCCGAGCTTGTCAGACTCTGC
GATGAAATGGGATTCATGATGATGATTGAGCCTTTCGATGAATGGGACATTGCCAAGTGTGAAAACGGAT
ACCACCGCTATTTCAACGAATGGGCCGAAAAAGACATGGTAAACATGCTACGGCAATACCGGAATAATCC
CTGTGTGGTGATGTGGAGTATCGGTAATGAAGTACCCACCCAATGCAGCAGTGAAGGATACAAAGTAGCC
AAGTTCCTGCAAGACATTTGCCATCGGGAAGACCCTACCCGTCCGGTTACCTGCGGCATGGACCAGGTTA
GTTGTGTACTCGACAACGGATTTGCGGCCATGCTCGACATTCCGGGATTCAATTATCGCGCACACCGCTA
TGAAGAAGCTTACCAACGCCTGCCTCAAAATCTTGTATTAGGCTCAGAAACCTCTTCTACCGTCAGTTCA
CGCGGTGTATACAAATTCCCGGCAGAGCGTAAAGCCGATGCAAAATACGAAGACCATCAGTCTTCTTCTT
ACGACTTGGAATACTGCTCCTGGTCTAACATTCCCGATATAGACTTTGCTCTGGCTGATGACCACCAATG
GACTTTGGGGCAGTTTGTCTGGACAGGTTTTGATTATCTGGGTGAACCCAGTCCATACGATACGGATGCA
TGGCCCAACCACAGCTCTATGTTCGGTATTATCGACCTGGCTTCCTTACCCAAAGACCGGTACTATTTAT
ACCGCAGCATATGGAACAAGCAAGCTGAAACACTTCATATTCTTCCTCATTGGAACTGGGAGGGCAGAGA
AGGAAAAGAAGTACCTGTATTCGTCTATACCAACTATCCGACAGCCGAACTTTTCATCAACGGAAAAAGT
TATGGGAAACAGACGAAGAACAACCAAAGCGTAGAGAACCGTTACCGCCTGATGTGGCACAACGCCATTT
ACGAACCGGGAGAAGTAAAAGTCGTGGCATACGATGAACACGGTACGGCTAAAGCAGAAAAGATAATCCG
CACGGCAGGCAAACCTCACCATATTGAATTGGTTTCTTCACGCCAGTCGCTCACAGCCGATGGAAAAGAT
TTGGCTTACGTAACCGTACGTGTTGTGGACAAAGACGGAAATCTCTGCCCCACAGATATGCGCTTGGTGA
AATTTAAAGTAAAAGGAGCTGGAAGCTACAAAGCCTCAGCCAATGGAGATCCAACTTGTCTGGATTTGTT
CCACCTGCCTCAGATGCACGCCTTCAACGGCATGCTGACTGCAATTGTGCAATCAGGAAAAGAAGCAGGT
ACCCTTGAGTTACAAGTCACCGCAAAAGGGCTGAAATCAGGAAAGATACAAATCGAAGTAAAA
SEQ ID NO: 17: Nucleotide sequence of CMR200137.102; CMR200137.19-
931.P(931)..pMCSG68
CAGACTGATAAGATTGACCTGGCCGGCTCGTGGACATTTTCTACGGACAGCATGGACTGGAGCCGGGTGA
TTGAACTGCCGGGTTCAATGGCTTCCAATGGTTTTGGGGAAGATATTGCCGTGGGTACTGATTGGACGGG
CGGTATTGTGGATTCTTCTTATTTCTTTAAACCTTCGTATGCCAAATACCGTGAGGCAGGAAATATCAAG
GTACCTTTCTGGCTTCAGCCGGTAAAATATTACAAGGGTAAGGCGTGGTATCAGAAAGAGGTGGTGATTC
CGGACAGTTGGGAAGGAAAGGACATTTCTCTCTTTTTGGAACGATGCCATTGGGAGAGCCGTTTGTATAT
AGACGGAAAGGAAATCGGCATGCAAAATGCTTTGGGGGCGCCCCATCGTTATGACCTGACAGGCAAGCTT
TCAGCAGGGAAACATGTGTTGATGCTGTGTGTAGACAATCGGGTGAAAAACATTGATCCGGGGGAGAACT
CACATAGTATTTCCGACCATACACAAGGAAACTGGAACGGGGTGGTAGGCGATATGTTCCTGGAAGTAAA
GCCGGAAGTGAATGTGTCTTCCGTCAAGATTATGCCGGAGCGTCTGGCTAAGAAAGTCAGTGTGTCGGCT
TCCTTGATGAACCGTTATGAAAAAGATGCCAATGTGGTACTGGAGATGACGGTAGGTAATGAAAAAGTAC
AGCAACAATGTACGTTGAAGCCGGGCGAAAATCAAGTGATGATGTCGCTGGCCATGAAGGGAGACATTAA
GTGCTGGGATGAGTTTTCTCCATCCTTATATGATTTGAAGCTGAGTGTGAAGGATGCGGATAGCGGTGAA
ACGGATGTCTATGCGGAACGTTTTGGTTTCCGTGATGTGAAGGTGAAAGACGGCAAACTCACCATCAACG
ACCGCCGTTTGTTCCTGCGTGGTACGCTGGATTGTGCCGTATTTCCGAAGACCGGTTTCCCGCCCACGGA
TGTAGAATCCTGGAAAAAGATTTATACCACCTGTCGGCAGCACGGACTGAACCATGTGCGCTTCCATTCC
TGGTGTCCGCCCGAAGCTGCTTTTGCAGCTGCCGATGGGATGGGTATGTACCTGGAGATAGAATGTTCTT
CCTGGGCTAACCAGTCGACTACCATTGGCGATGGAGGCGATCTGGACCGCTTTATCTGGGAGGAAAGTGA
ACGCATCGTCCGTGAGTTTGGTAACCATCCTTCTTTCTGCATGATGATGTACGGTAACGAACCGGCTGGT
GAGGGAAGTAATGCCTATCTGACTAATTTTGTTACTACCTGGAAAGAGCGCGATGCCCGCCGTTTATATT
GTTCGGGTGCCGGATGGCCCAATTTGCCGGTTAACGACTTCTTGAGCGATTCCAATCCTCGTATTCAGGC
GTGGGGACAAGGTGTGAAGAGTATTATCAACGCACAGGCTCCGCGTACCGACTATGACTGGTCAGAATAC
ATCGGACGTTTCCAGCAGCCGATGGTGAGCCACGAAATCGGGCAGTGGTGTGTATATCCCAACTTCAAGG
AAATGGCCAAATACGACGGGGTGATGCGCCCGCGTAATTTTGAGATATTCCAGGAAACACTGGCTGAAAA
CGGTATGGCACATTTGGCTGACAGCTTCCTGCTGGCTTCCGGAAAATTGCAGGCGTTGTGTTATAAGGCC
GATATCGAAGCTGCTTTGCGTACAAAAGACTTCGGTGGATTCCAGTTACTGGGCTTGTCTGATTTCCCGG
GGCAGGGTACGGCTTTGGTAGGAGTGCTCGATGCGTTCTGGGAAGAAAAAGGCTACATCCGTCCGGAAGA
ATACCGTCGTTTCTGTAATAGTACGGTACCATTACTGCGCTTGCCGAAGTTGATTTATACCAACCAGGAA
ACGGTGAAAGGAAGTCTGGAAGTGGCACATTTCGGAGCTGCTCCGCTGGAGGTGACTTCTACTGTCTGGA
CCCTGAAAACAAAAGAAGGAAAGACAATTGCTTCGGGCACGCTGGCACACCAGCCGGTAGGTATCGGCAA
TTGTATTCCGTTGGGGCAGCTGGAGATTCCATTGGATAAGGTGGACGTCCCTTCATGTCTGACACTGGAA
GCTACATTGGGAGATTACGCCAACAGCTGGCACATCTGGGTATATCCTGCTGCGGTACAGAAAGTAGCTG
ATGAAGCACAATTGCTGATGACCGACCGTCTGGATGCAAAAGCTTTGCAACGTCTTCAGGAAGGTGGCAA
CGTACTGCTTTCTTTACGGAAAGGCTCCTTGCCTGCCGAAGCGGGAGGCGAAGTAGTGATAGGTTTCTCT
AGCATCTTCTGGAACACGGCCTGGACGCTGGGACAAGCACCGCACACACTGGGTATCCTGTGTAACCCCG
CTCATCCGGCACTTTCAGAGTTCCCTACAGAGTATTACAGTGATTATCAGTGGTGGGATGCCATGAGCCA
TTCCGGTGCCATCGAAGTGGTCAAGATTGATAAAAACTTGCAGCCGATTGTACGAGTTATCGACGACTGG
TTTACGAACCGTCCGCTGGCTTTGTTGTTCGAAGTGAAGGTGGGTAAGGGTAAATTGCTTGTGTCAGGAA
TTGATTTCTGGCAGGATATGGACAAGCGTACGGAAGCCCGTCAGTTACTCTACAGCTTGAAGAAATATAT
GTGCGGTAATCGCTTCAATCCCTCTTCTGAAGTCGATGCGAAAGATTTAAGTATTTTGTTTTCCATTAAA
AATCAAAAA
SEQ ID NO: 18: Nucleotide sequence of CMR200148.102; CMR200148.28-
761.P(761)..pMCSG68
AAGGATGCGGAGATGGACCGCTTTATCAGTGACCTGATGGGAAGGATGACCTTGCAGGAAAAGTTAGGAC
AGTTGAATCTGCCGGCTGGGAATGACCTGGTGTCGGGAGCAGTGAAGAACAGCAAGATGGCAGAAGCTAT
CCGAGCTGGTGAGGTCGGCGGCTTTTTCAATGTGAAGGGAGTGGATAAGATTTACCAGATGCAGCGTATG
GCGGTGGAGGAAACTCGTCTGGGAATTCCTTTGATAGTGGGTGCCGATGTGATTCACGGGTACGAAACAA
TCTTCCCGATTCCGTTGGCCCTGTCTTGTAGCTGGGATACGGCGGCGGTGACACGTATGGCACGTATTTC
TGCCACGGAAGCCAGTGCCGATGGAATCAGCTGGACCTTCAGTCCGATGGTAGACATCTGTCGGGATGCC
CGCTGGGGACGTATTGCAGAAGGAAGTGGAGAGGACCCGTACCTCGGGGCGTTGATGGCTGGAGCCTATG
TGCGCGGTTATCAGGGTGACGGCATGAAGCAGAACAATGAAATCATGGCCTGTGTGAAGCACTTTGCGCT
GTATGGAGCTTCGGAATCGGGACGTGACTACAATTCGGTGGATATGAGTCGAAACCTGATGTATAATGTG
TACCTGGCTCCTTATAAAGGGGCGGTGGAAGCCGGAGTGGGTTCGGTGATGAGCTCGTTCAATACCATCA
ACGGGGTACCTGCTACAGCTGACAAATGGCTGCTGACGGATTTGCTCCGCAATGAGTGGGGGTTCACGGG
GTTTGTGGTGACCGACTACAATTCGATTGGTGAGATGAAGACTCATGGGGTGGCCGACTTGAAGGAGGCT
TCTGCACGGGCGTTGAATGCAGGAACGGACATGGATATGGTGGCACATGGTTTCTTGCATACGCTGGAAG
CTTCATTGAAGGAGAAGGCCGTGACGCAGGAGCGGATTGACGAGGCTTGTCGTCGGGTATTGGAAGCCAA
GTATAAGTTAGGATTGTTTGAAAATCCTTATAAGTATTGTGATACGCTTCGGGGACGCAAGGAATTGTTT
ACGGAGGCGAATCGTAAAGCGGCACGTGAGATTGCGGCTGAAACGTTTGTGCTGTTGAAGAACGAGGGTA
AGTTGTTGCCTTTGCAGAAAAAAGGACGCATTGCATTGATTGGGCCGATGGCTGATGCGCAGAACAATAT
GTGCGGCACGTGGAACATGGATTGTCAGACAGACCGTCATGTGACGATGTACGAAGCTTTCCGTCGTGCG
GTAGGTGATAAGGCTACGGTTTCTTATGCCAAGGGAAGTAATGTGTATTATAGTGAGCATATTGAGAAAG
GGGCGGTCGAACCTCGTCCGCTGACACGTGGCGATGACCGTCAGTTGCGGGCTGAGGCTTTGCGCGTGGC
GGCTTCTGCCGATGTGATTGTGGCCGCATTAGGTGAGAGTGCTGAGATGAGCGGAGAGTCTTCTTCTCGT
ACAGATATTCAGATTCCGGATGCGCAGAAAGATTTGTTGAAGGCATTGATAGCTACCGGAAAGCCGGTGG
TACTGGCTTTGTTTACCGGTCGTCCGCTGGATTTATGCTGGGAGTCTGAGCATGTTCCGGCTATCCTGAA
CGTGTGGTTTGCCGGCAGTGAAGCGGGTGATGCCATTGCCGATGTGATGTTTGGAGAAGTATCTCCTTCG
GGTAAGCTGACTACGAGTTTCCCACGTGCGGTGGGACAGTTGCCGCTTTATTATAATCACCTGAATACGG
GTCGTCCGGATACGGATGACACTACTTTCAATCGTTATGGCAGCAATTACATCGACCAGAGTAATGAACC
GCTTTATCCTTTTGGCTATGGTTTGAGTTATACCACTTTCCGTTACGGTAATTTGCAGTTGAGTGCGGAG
CGTATGGCCAAGGGTGGGCAGTTGAAGGTAACCGTGCCTGTAACCAATTCCGGCGAGTGTGACGGAGTAG
AGATTGTGCAGTTGTATCTTCACGATGTGTATGCAGAAATCTCCCGTCCGGTGAAGGAGCTGAAAGCTTT
CCGCCGTGTGGCCCTTAAAAAGGGAGAGACACAGAATGTAGAGTTTGTACTCGATGAGGATGATTTGAAG
TATTATAATTCTCGTCTGGAATATGGATATGAACCGGGAGAGTTTGAAGTGATGGTGGGTCCGGACAGCC
GGAATGTGCAGCACGCGACTTTTGTGGCTGAA
SEQ ID NO: 19: Nucleotide sequence of CatM transcription factor
ATGGAACTAAGACACCTCAGATATTTTGTGACCGTGGTTGAAGAGCAAAGCATTTCCAAAGCTGCTGAAA
AGTTGTGTATTGCCCAGCCGCCCCTCAGCCGACAAATTCAAAAACTCGAAGAAGAATTGGGAATTCAGCT
ATTTGAACGCGGCTTCAGACCGGCTAAAGTGACTGAAGCAGGCATGTTTTTTTATCAGCATGCTGTGCAG
ATTTTGACTCATACTGCACAAGCGTCCTCAATGGCAAAACGGATTGCAACGGTCAGTCAAACCTTGAGAA
TTGGTTACGTCAGCTCCTTACTGTATGGTTTGTTACCTGAAATTATTTATCTGTTTCGTCAACAAAATCC
TGAAATTCACATCGAACTCATCGAATGCGGCACCAAAGATCAAATTAATGCCCTTAAGCAGGGAAAAATC
GATCTGGGTTTTGGTCGGCTCAAAATTACCGATCCTGCAATTCGACGTATCGTGTTGCATAAAGAACAGC
TCAAACTTGCAATCCATAAGCATCATCACCTCAATCAGTTTGCAGCAACAGGGGTTCATCTCTCTCAAAT
TATTGATGAACCGATGCTGCTGTACCCAGTCTCTCAAAAGCCCAATTTTGCGACCTTTATTCAGTCACTC
TTTACCGAACTAGGCCTAGTACCATCCAAACTCACCGAAATTCGAGAAATTCAACTGGCACTCGGCTTGG
TGGCAGCAGGTGAAGGCGTCTGCATCGTACCGGCGTCTGCCATGGATATTGGGGTGAAGAATCTACTTTA
TATTCCAATTTTAGATGATGATGCCTATAGCCCAATTTCACTCGCGGTGCGAAATATGGACCACAGTAAT
TACATTCCTAAAATTCTCGCCTGTGTACAGGAGGTGTTTGCAACGCACCATATCAGGCCACTCATCGAAT
AA
SEQ ID NO: 20: Nucleotide sequence of T7 promoter
TAATACGACTCACTATAG
SEQ ID NO: 21: Nucleotide sequence of CatM promoter
TTTTCAATAAATACTATTTACATACCTTAAATTAATGTAATAATAAAAACCAACACCAATTTGGTATTTT
TGCATACTAAAAAGGTATATAAAACCAATTAGGGCGTATAA
SEQ ID NO: 22: Nucleotide sequence of T7 terminator
CTAGCATAACCCCTTGGGGCCTCTAAACGGGTCTTGAGGGGTTTTTTG
SEQ ID NO: 23: Nucleotide sequence of a complete construct comprising a CatM
transcription factor, a CatM promoter, and a β-glucosidase reporter (APC115086)
(sensor cassette region)
TTATTGAAGCGAAAATAAGGCCTTATTCACATCACGGCTATCACCACCTATCATTACTTCAAAGTCACCC
GGTTCGCACACAAAATCCAGATTGTAATTGTAGAACTTCAACATATCAGCTGTTAACTTGAAAGACACTT
GTTTGGATTCACCGGCTTTCAAGAAGATTTTTTCAAAGCCTTTCAGTTCTTTCACCGGACGTGTTACACT
GCCTATAAGATCGCGGATATATAGCTGCACTACTTCCGAACCGTCATACTTACCTGTATTGGTTACCGTT
ACAGTTGCCATAATCTCTCCATTGATATTCATGGACGATTTATCCAATGTAATATCACTATAAGAGAAAG
TAGTATAGCTCAAGCCATATCCAAACGGATAAAGCGGTTCGTTATCTACATCCAGATAATTGCTACGGAA
CTTCTGGAACCAGGCCCCTTGAGGCAAAGGACGACCAGTATTCTTATGGTTATAGAACAAAGGAATCTGT
CCTACACTCTTCGGAAAAGTAGTAGTAAGTTTGCCACTCGGATTTACATTTCCAAACAGTACATCACCAA
TGGCAAGAGCAGCTTCACTACCACCAAACCACACATTCAGAATAGCAGGTACATTTTCCTGCTCCCAATT
CAATACTAACGGACGACCGGTAAACAACACCAATACCACCGGTTTGCCGGTTTTCAACAATTCCTGCAAA
AGTACACGTTGCGTATCCGGCATTTCGAGGTCTGTACGGCAACTACTTTCACCGCTCATCTCGGAAGACT
CACCCAAAGCAGCAACAATCACGTCAGACTTGGCAGCTACAGCAAGCGCCTCATCCAGCAGTTCCTTATC
TGTACGATTGTCACGATGCAGAGTACGGCCAAACATAGTAGCACGTTCTTCGTATTCGGCATCACTCATC
AGATTACTTCCTTTAGCCGTAAGAATTTTAGCCTTGCCTCCCACTACTTCTTTCAAGCCTTCAATCAAAG
AAGGATATTTGTTCATCACAGCGGCCACACTCCACGTGCCCGGCATATTGCTACGGCTATCGGCCAAAGG
ACCTACTACAGCAATGGTACCTTTCTTTGCCAGAGGGAGTACACTATTCTCATTCTTCAAGAGAACAAAG
CTTTCCGAAGCTGTCTTACGGGCTATAGCGCGGTGTTCTTTTGTAAAGATTTGTTTTTTAGGACGGGTTA
TATCACAATATTTATAGGGATTATCAAAAAGCCCCAGCTTATACTTAGCTTCCAGAATACGGCGACAAGC
AGCATCAACAGCCTTTACTGAAACTTTTCCTTCTTCCAGAGATTTTTTAAGTGTGCTTGTAAAAGCATCG
CTCACCATATCCATATCGACACCTGCATTCAGAGCCAGGGCTGCAACTGTTTGTGTATCACCCATACCAT
GATCGGTCATTTCAGTGATACCGGTATAGTCCGTCACAACGAACCCATCAAAATTCCACTGCTTACGAAG
TACATCGGTCATCAGCCACTTATTTCCGGTAGCCGGTACACCATCCACTTCATTGAATGAAGCCATCACA
CTACCCACACCTTCTTCCACGGCAGCCTGATAAGGTAACATATATTCGTTGAACATACGTTGATGACTCA
TATCCACTGTATTATAGTCGCGTCCGGCTTCTGATGCCCCATATAACGCAAAGTGCTTCACGCAAGCCAT
AATTTCATCATTACTACTCATATCTTTCCCTTGATAACCACGTACCATAGCACGCGCAATCTCCGCTCCC
AAGAAGGGATCTTCACCATTCCCTTCGGAAACTCGTCCCCAACGGGGATCACGGGAAACATCCACCATCG
GACTGAATGTCCAGCAAATACCATCAGCACTGGCTTCGATAGCAGCAATACGTGCAGATTCTTCAATAGC
TGTCATGTTCCAGGTACAGGATAATCCCAGAGGAATAGGAAATACCGTTTCGTATCCATGAATTACATCC
ATACCAAATAAAAGAGGAATACCCAGACGACTTTCTTCTACTGCCTGTTTCTGAACGTCACGAATACGCT
CCACGCCTTTCAAGTTAAAGAGTCCGCCCACTTCACCGGCACGGATACGCTTAGCCACATTACTACTCTT
GGCTTGTCCGGTGGTTATTTCACCCGTAACAGGCAAGTTCAACTGGCCGATTTTCTCTTCCAGAGTCATC
TTCTTCATCAGATCATCAATAAAGCGATCCATATCGACAGGAGATTTCATCTTTCCTGTGTGATTTTCAA
TAAATACTATTTACATACCTTAAATTAATGTAATAATAAAAACCAACACCAATTTGGTATTTTTGCATAC
TAAAAAGGTATATAAAACCAATTAGGGCGTATAAATGGAACTAAGACACCTCAGATATTTTGTGACCGTG
GTTGAAGAGCAAAGCATTTCCAAAGCTGCTGAAAAGTTGTGTATTGCCCAGCCGCCCCTCAGCCGACAAA
TTCAAAAACTCGAAGAAGAATTGGGAATTCAGCTATTTGAACGCGGCTTCAGACCGGCTAAAGTGACTGA
AGCAGGCATGTTTTTTTATCAGCATGCTGTGCAGATTTTGACTCATACTGCACAAGCGTCCTCAATGGCA
AAACGGATTGCAACGGTCAGTCAAACCTTGAGAATTGGTTACGTCAGCTCCTTACTGTATGGTTTGTTAC
CTGAAATTATTTATCTGTTTCGTCAACAAAATCCTGAAATTCACATCGAACTCATCGAATGCGGCACCAA
AGATCAAATTAATGCCCTTAAGCAGGGAAAAATCGATCTGGGTTTTGGTCGGCTCAAAATTACCGATCCT
GCAATTCGACGTATCGTGTTGCATAAAGAACAGCTCAAACTTGCAATCCATAAGCATCATCACCTCAATC
AGTTTGCAGCAACAGGGGTTCATCTCTCTCAAATTATTGATGAACCGATGCTGCTGTACCCAGTCTCTCA
AAAGCCCAATTTTGCGACCTTTATTCAGTCACTCTTTACCGAACTAGGCCTAGTACCATCCAAACTCACC
GAAATTCGAGAAATTCAACTGGCACTCGGCTTGGTGGCAGCAGGTGAAGGCGTCTGCATCGTACCGGCGT
CTGCCATGGATATTGGGGTGAAGAATCTACTTTATATTCCAATTTTAGATGATGATGCCTATAGCCCAAT
TTCACTCGCGGTGCGAAATATGGACCACAGTAATTACATTCCTAAAATTCTCGCCTGTGTACAGGAGGTG
TTTGCAACGCACCATATCAGGCCACTCATCGAATAA
SEQ ID NO: 24: Nucleotide sequence of an expression cassette comprising a β-
glucosidase reporter (APC115086), a CatM transcription factor, and a catM
promoter.
TTATTGAAGCGAAAATAAGGCCTTATTCACATCACGGCTATCACCACCTATCATTACTTCAAAGTCACCC
GGTTCGCACACAAAATCCAGATTGTAATTGTAGAACTTCAACATATCAGCTGTTAACTTGAAAGACACTT
GTTTGGATTCACCGGCTTTCAAGAAGATTTTTTCAAAGCCTTTCAGTTCTTTCACCGGACGTGTTACACT
GCCTATAAGATCGCGGATATATAGCTGCACTACTTCCGAACCGTCATACTTACCTGTATTGGTTACCGTT
ACAGTTGCCATAATCTCTCCATTGATATTCATGGACGATTTATCCAATGTAATATCACTATAAGAGAAAG
TAGTATAGCTCAAGCCATATCCAAACGGATAAAGCGGTTCGTTATCTACATCCAGATAATTGCTACGGAA
CTTCTGGAACCAGGCCCCTTGAGGCAAAGGACGACCAGTATTCTTATGGTTATAGAACAAAGGAATCTGT
CCTACACTCTTCGGAAAAGTAGTAGTAAGTTTGCCACTCGGATTTACATTTCCAAACAGTACATCACCAA
TGGCAAGAGCAGCTTCACTACCACCAAACCACACATTCAGAATAGCAGGTACATTTTCCTGCTCCCAATT
CAATACTAACGGACGACCGGTAAACAACACCAATACCACCGGTTTGCCGGTTTTCAACAATTCCTGCAAA
AGTACACGTTGCGTATCCGGCATTTCGAGGTCTGTACGGCAACTACTTTCACCGCTCATCTCGGAAGACT
CACCCAAAGCAGCAACAATCACGTCAGACTTGGCAGCTACAGCAAGCGCCTCATCCAGCAGTTCCTTATC
TGTACGATTGTCACGATGCAGAGTACGGCCAAACATAGTAGCACGTTCTTCGTATTCGGCATCACTCATC
AGATTACTTCCTTTAGCCGTAAGAATTTTAGCCTTGCCTCCCACTACTTCTTTCAAGCCTTCAATCAAAG
AAGGATATTTGTTCATCACAGCGGCCACACTCCACGTGCCCGGCATATTGCTACGGCTATCGGCCAAAGG
ACCTACTACAGCAATGGTACCTTTCTTTGCCAGAGGGAGTACACTATTCTCATTCTTCAAGAGAACAAAG
CTTTCCGAAGCTGTCTTACGGGCTATAGCGCGGTGTTCTTTTGTAAAGATTTGTTTTTTAGGACGGGTTA
TATCACAATATTTATAGGGATTATCAAAAAGCCCCAGCTTATACTTAGCTTCCAGAATACGGCGACAAGC
AGCATCAACAGCCTTTACTGAAACTTTTCCTTCTTCCAGAGATTTTTTAAGTGTGCTTGTAAAAGCATCG
CTCACCATATCCATATCGACACCTGCATTCAGAGCCAGGGCTGCAACTGTTTGTGTATCACCCATACCAT
GATCGGTCATTTCAGTGATACCGGTATAGTCCGTCACAACGAACCCATCAAAATTCCACTGCTTACGAAG
TACATCGGTCATCAGCCACTTATTTCCGGTAGCCGGTACACCATCCACTTCATTGAATGAAGCCATCACA
CTACCCACACCTTCTTCCACGGCAGCCTGATAAGGTAACATATATTCGTTGAACATACGTTGATGACTCA
TATCCACTGTATTATAGTCGCGTCCGGCTTCTGATGCCCCATATAACGCAAAGTGCTTCACGCAAGCCAT
AATTTCATCATTACTACTCATATCTTTCCCTTGATAACCACGTACCATAGCACGCGCAATCTCCGCTCCC
AAGAAGGGATCTTCACCATTCCCTTCGGAAACTCGTCCCCAACGGGGATCACGGGAAACATCCACCATCG
GACTGAATGTCCAGCAAATACCATCAGCACTGGCTTCGATAGCAGCAATACGTGCAGATTCTTCAATAGC
TGTCATGTTCCAGGTACAGGATAATCCCAGAGGAATAGGAAATACCGTTTCGTATCCATGAATTACATCC
ATACCAAATAAAAGAGGAATACCCAGACGACTTTCTTCTACTGCCTGTTTCTGAACGTCACGAATACGCT
CCACGCCTTTCAAGTTAAAGAGTCCGCCCACTTCACCGGCACGGATACGCTTAGCCACATTACTACTCTT
GGCTTGTCCGGTGGTTATTTCACCCGTAACAGGCAAGTTCAACTGGCCGATTTTCTCTTCCAGAGTCATC
TTCTTCATCAGATCATCAATAAAGCGATCCATATCGACAGGAGATTTCATCTTTCCTGTGTGATTTTCAA
TAAATACTATTTACATACCTTAAATTAATGTAATAATAAAAACCAACACCAATTTGGTATTTTTGCATAC
TAAAAAGGTATATAAAACCAATTAGGGCGTATAAATGGAACTAAGACACCTCAGATATTTTGTGACCGTG
GTTGAAGAGCAAAGCATTTCCAAAGCTGCTGAAAAGTTGTGTATTGCCCAGCCGCCCCTCAGCCGACAAA
TTCAAAAACTCGAAGAAGAATTGGGAATTCAGCTATTTGAACGCGGCTTCAGACCGGCTAAAGTGACTGA
AGCAGGCATGTTTTTTTATCAGCATGCTGTGCAGATTTTGACTCATACTGCACAAGCGTCCTCAATGGCA
AAACGGATTGCAACGGTCAGTCAAACCTTGAGAATTGGTTACGTCAGCTCCTTACTGTATGGTTTGTTAC
CTGAAATTATTTATCTGTTTCGTCAACAAAATCCTGAAATTCACATCGAACTCATCGAATGCGGCACCAA
AGATCAAATTAATGCCCTTAAGCAGGGAAAAATCGATCTGGGTTTTGGTCGGCTCAAAATTACCGATCCT
GCAATTCGACGTATCGTGTTGCATAAAGAACAGCTCAAACTTGCAATCCATAAGCATCATCACCTCAATC
AGTTTGCAGCAACAGGGGTTCATCTCTCTCAAATTATTGATGAACCGATGCTGCTGTACCCAGTCTCTCA
AAAGCCCAATTTTGCGACCTTTATTCAGTCACTCTTTACCGAACTAGGCCTAGTACCATCCAAACTCACC
GAAATTCGAGAAATTCAACTGGCACTCGGCTTGGTGGCAGCAGGTGAAGGCGTCTGCATCGTACCGGCGT
CTGCCATGGATATTGGGGTGAAGAATCTACTTTATATTCCAATTTTAGATGATGATGCCTATAGCCCAAT
TTCACTCGCGGTGCGAAATATGGACCACAGTAATTACATTCCTAAAATTCTCGCCTGTGTACAGGAGGTG
TTTGCAACGCACCATATCAGGCCACTCATCGAATAACGATCTCGATCCCGCGAAATTAATACGACTCACT
ATAGGGGAATTGTGAGCGGATAACAATTCCCCTCTAGAAATAATTTTGTTTAACTTTAAGAAGGAGATAT
ACATATGCACCATCATCATCATCATTCTTCTGGTGTAGATCTGTGGTCTCATCCGCAGTTCGAAAAGGGT
ACCGAGAACCTGTACTTCCAATCCAATgccATGACCGTGAAAATTTCCCACACTGCCGACATTCAAGCCT
TCTTCAACCGGGTAGCTGGCCTGGACCATGCCGAAGGAAACCCGCGCTTCAAGCAGATCATTCTGCGCGT
GCTGCAAGACACCGCCCGCCTGATCGAAGACCTGGAGATTACCGAGGACGAGTTCTGGCACGCCGTCGAC
TACCTCAACCGCCTGGGCGGCCGTAACGAGGCAGGCCTGCTGGCTGCTGGCCTGGGTATCGAGCACTTCC
TCGACCTGCTGCAGGATGCCAAGGATGCCGAAGCCGGCCTTGGCGGCGGCACCCCGCGCACCATCGAAGG
CCCGTTGTACGTTGCCGGGGCGCCGCTGGCCCAGGGCGAAGCGCGCATGGACGACGGCACTGACCCAGGC
GTGGTGATGTTCCTTCAGGGCCAGGTGTTCGATGCCGACGGCAAGCCGTTGGCCGGTGCCACCGTCGACC
TGTGGCACGCCAATACCCAGGGCACCTATTCGTACTTCGATTCGACCCAGTCCGAGTTCAACCTGCGTCG
GCGTATCATCACCGATGCCGAGGGCCGCTACCGCGCGCGCTCGATCGTGCCGTCCGGGTATGGCTGCGAC
CCGCAGGGCCCAACCCAGGAATGCCTGGACCTGCTCGGCCGCCACGGCCAGCGCCCGGCGCACGTGCACT
TCTTCATCTCGGCACCGGGGCACCGCCACCTGACCACGCAGATCAACTTTGCTGGCGACAAGTACCTGTG
GGACGACTTTGCCTATGCCACCCGCGACGGGCTGATCGGCGAACTGCGTTTTGTCGAGGATGCGGCGGCG
GCGCGCGACCGCGGTGTGCAAGGCGAGCGCTTTGCCGAGCTGTCATTCGACTTCCGCTTGCAGGGTGCCA
AGTCGCCTGACGCCGAGGCGCGAAGCCATCGGCCGCGGGCGTTGCAGGAGGGCTGA

### EXAMPLES

The examples are offered for illustrative purposes only and are not intended to limit the scope of the present invention in any way.

The following examples describe representative materials and methods for generating the constructs and biosensors of the disclosure, further characterize the various features of the constructs and biosensors of the disclosure, and disclose additional materials and methods for the identification of stable and highly active β-glucosidases and characterize their physical and functional features to demonstrate their roles in various applications including for the biosensors as disclosed herein.

### Example 1—Construction and Characterization of Enzyme-Linked Biosensors

The main objective of this Example is to engineer, design, and construct a novel enzyme-linked biosensor that is able to withstand harsh conditions and has potential applications in microfluidics. This Example will also characterize the enzyme-linked biosensor produced by comparing the fluorescence intensity of the signals recovered from the enzyme-linked biosensor and compared to the signal produced from the transcription factor (TF)-based biosensor with FP reporter.

It was hypothesized herein that a novel mechanism may lead to the construct of a novel enzyme-based linked biosensor, also referred to as an enzyme-linked biosensor, an enzyme-based biosensor, or the like. The construction of an enzyme-based biosensor may be feasible by modifying the TF-based biosensor systems and utilizing the enzyme-linked-reporter systems. Significant research and experimentation were carried out and described in the disclosure herein.

Two TF-based sensors, pBTL2_CatM_C21 and pBTL2_CatM_C2, were designed and optimized previously as described in Shin et al. “Tackling the Catch-22 situation of optimizing a sensor and a transporter system in a whole-cell microbial biosensor design for an anthropogenic small molecule.” ACS Synthetic Biology 11.12 (2022): 3996-4008 to monitor the intracellular product formation of cis, cis-muconic acid (CCM) in an engineered Pseudomonas putida KT2440 strain was adopted. Both biosensors were shown to be successful in selecting top CCM producers from engineered or variants obtained from adaptive laboratory evolution of P. putida KT2440 strains. As CCM is produced by the engineered P. putida, carrying the plasmid-based biosensor, the expression of the fluorescent protein reporter is proportional to the intracellular CCM concentration and, therefore, it is important to tune the biosensor such that its dynamic range aligns with the range of intracellular CCM concentration in the surveyed organism.

### Construction of an Enzyme-Linked Biosensor

The objective of this experiment was to design and construct an enzyme-linked biosensor from an existing traditional TF-based biosensor comprising a GFP fluorescent protein reporter. The objectives were to replace the CatM-promoter with the protein expression vector, pMCSG68, upstream of the T7 promoter. Then, the reporter enzyme GFP of the previous construct was replaced with a β glucosidase reporter enzyme.

### Experiments

In order to construct the enzyme-linked biosensor, the pBTL2_CatM_C21 sensor was converted by moving the engineered promoter and catM transcription factor into pMCSG68, a commonly used protein expression vector.

### Selection of β-Glucosidase Gene

Next, the objective of this experiment was to screen and identify variants of β-glucosidase that would be optimal as a reporter enzyme. The specific β-glucosidase variant would fit the profile of being a well-characterized, highly stable, and active β-glucosidase gene.

Through significant research and experimentation, a β-glucosidase gene known as “pBATS_0004” was identified. The β-glucosidase gene (pBATS_0004) performed well in both microfluidic and plate-reader experiments (data not shown). The pBATS_0004 was specifically selected as the β-glucosidase enzyme to be used as a reporter enzyme for the construction of the enzyme-linked biosensor. Preliminary experiments suggested that pBATS_0004 would likely prove to provide an ideal readout in ultra-high-throughput screens. Since pBATS_0004 had already been shown to perform significantly well in both microfluidic and plate-reader experiments, it was reasonable to continue using pBATS_0004 as the lead candidate and for providing an ideal readout in ultra-high-throughput screens.

The β-glucosidase gene (pBATS_0004) was subcloned into the pMCSG68 construct. The β-glucosidase gene (pBATS_0004) has also been selected for later optimization and/or identification of additional variants.

In summary, this experiment converts the construct from the previous transcription factor (TF)-based biosensor that was linked to a fluorescent protein as its reporter, specifically, a green fluorescent protein reporter (GFP), into an enzyme-linked biosensor of the disclosure. A schematic representation of the construction of the enzyme-linked biosensor of the disclosure is shown in FIG. 2A.

FIG. 2A depicts a schematic representation for constructing the enzyme-linked biosensor (or “enzyme-based biosensor”) of the disclosure (FIG. 2A) and an example of a mechanism of the biosensor of the disclosure (FIG. 2B). The native catM and promoter region were previously inserted into the pBTL-2 vector and optimized for Pseudomonas putida response. The transcription factor (TF) and promoter region were transferred along with a β-glucosidase reporter and cloned into the pMCSG68 vector. In order to increase the sensitivity of product detection and operational range in a picoliter setting, a β-glucosidase enzyme has been inserted into the biosensor. Thus, this enzyme-linked reporter provides a much greatly enhanced signal and a much more stable biosensor. Moreover, the construct and/or the biosensor, as described herein, allow for a significantly enhanced signal and a significantly more stable biosensor compared to a counterpart without the addition of the enzyme-linked reporter. Additionally, in some aspects, the new biosensor can accept genes for protein expression.

Moreover, an example of a mechanism of the biosensor of the disclosure is shown in FIG. 2A is further depicted in FIG. 2B.

FIG. 2B depicts an example of a mechanism of the biosensor of the disclosure shown in FIG. 2A. In the example, a library of catA genes is tested for activities. The expression of the CatA enzymes is induced with a separate inducible promoter (T7) in the biosensor cell (e.g., E. coli BL21(DE3)), the enzyme is produced within hours, the substrate (catechol in this example) is added externally, taken up by the cells, converted into the product (cis-cis-muconate in this example), which in turn detected by the product sensor circuit (TF and associated promoter region; catM and promoter region in this example). The expressed enzyme activity is proportional to the production of the reporter β-glucosidase enzyme and the observed fluorescent signal. The design can be easily adapted for studying other systems by simply replacing the product sensor portion (labeled with a rectangle) sensing the given product produced by the cloned enzyme (Enzyme E1).

FIGS. 3A-3H depict experimental results comparing the sensitivity of product detection and operational range between the enzyme-linked biosensor of the disclosure and the traditional biosensor that uses a fluorescent protein (e.g., GFP) reporter. FIG. 3A shows the results from an experiment that compares the biosensor having the enzyme-linked reporter as provided in the disclosure and the traditional biosensor using GFP. The results show a significantly better detection by the enzyme-linked reporter (labeled enzyme-linked reporter) than by the GFP reporter (labeled as sfGFP reporter). FIG. 3B depicts a schematic representation of the major differences between the two biosensors. The enzyme-linked reporter construct of the disclosure has a gene for expressing β-glucosidase (top), whereas the traditional biosensor has a gene for expressing GFP (bottom). FIGS. 3C-3D compare the Relative Fluorescence Units (RFU) vs. Time between the biosensor with GFP (FIG. 3C) and biosensor with β-glucosidase (APC115086, the β-glucosidase variant with SEQ ID NO: 9.) (FIG. 3D). The results show that the biosensor with the β-glucosidase reporter has a slightly narrower dynamic range (e.g., without limitation, a linear detection between 4-14 mM for cis, cis-muconate compared to 2.5-20 mM for the sfGFP-based reporter), but the signal-to-noise measured is substantially higher favoring the β-glucosidase reporter (FIG. 3A). The signal amplification is due to the fact that even when similar number of sfGFP or β-glucosidase are produced in the presence of cis, cis-muconate, one enzyme (β-glucosidase) is capable of turning hundreds of substrate molecules into fluorescent products (FIG. 3E) versus measuring the production of the fluorescent sfGFP molecule via fluorescence (FIG. 3F). Since the enzyme-linked biosensor circuit is inserted into a vector (e.g., pMCSG68) that is normally used for protein expression in E. coli, the overall design enables for the screening of the activities of a given target enzyme with the following criteria: a) the substrate of the target enzyme can be introduced into E. coli cells, and b) the corresponding TF and promoter region is identified and cloned into the biosensor region (FIG. 3G and FIG. 3H). In the specific example in FIG. 3H, a library of catA genes, is evaluated using this approach. The expression of the CatA enzymes is induced with a separate inducible promoter (e.g., a T7 promoter) in the biosensor cell (e.g., E. coli BL21(DE3)), the enzyme is produced within hours, the substrate (e.g., catechol) is added externally, taken up by the cells, converted into the product (e.g., cis, cis-muconate), which in turn detected by the product sensor circuit (e.g., TF and associated promoter region, e.g., catM and promoter region). The expressed enzyme activity is proportional to the production of the reporter β-glucosidase enzyme and the observed fluorescent signal. The design can be easily adapted for the study of other systems by simply replacing the product sensor portion (labeled with a rectangle) sensing the given product produced by the target enzyme (Enzyme E1). Almost all biomanufacturing applications rely on enzymes that have been optimized for increased performance (e.g., without limitation, activity, thermotolerance, chemical tolerance, etc.).

FIGS. 4A-4B show a schematic representation of the chemical reaction of the biosensor having the enzyme-linked reporter as provided in the disclosure (FIG. 4A) and the intensity of the fluorescence being detected when using the enzyme-linked reporter of the disclosure (FIG. 4B). FIG. 4A shows the detection of the expressed β-glucosidase by the addition of a lysis buffer to permeabilize the sensor cell such that the clear non-fluorescent substrate (fluorescein quenched by two glucose moieties), fluorescein di-β-D-glucopyranoside, can enter the cell and the glucose molecules cleaved off by the β-glucosidase reporter enzyme to release the highly fluorescent fluorescein product. FIG. 4B shows the reporter activity when the sensor cells are incubated with cis, cis-muconate for more than 4 hours, followed by the addition of a lysis buffer and the di-β-D-glucopyranoside substrate. The release of fluorescein is monitored via a fluorescent plate reader. While the signal developed after one hour is sufficient to calculate the cis, cis-muconate concentration in the solution, the figure shows that the signal develops over time linearly, producing even more intense fluorescence.

In summary, this Example includes experiments for the construction of the enzyme-linked biosensor from the TF-based biosensor followed by experiments to show that the enzyme-linked biosensor allows for signal recovery that were about 10-1000-fold higher in fluorescence intensity than those recovered from the TF-based biosensor. The construction scheme involved replacing the fluorescent protein (e.g., a GFP) in the TF-based biosensor with an enzyme (e.g., a β-glucosidase) that catabolizes a commercially available fluorescent substrate. The signals that are recovered from the enzyme-linked biosensor, and when carried out from equivalent microfluidic test volumes, are about 10-1000-fold higher in fluorescence intensity than those recovered from the TF-based biosensor or, also referred to as, herein, a fluorescent protein (FP)-based biosensor. The difference between the signals that are recovered from the TF-based biosensor and the enzyme-linked biosensor is shown in FIGS. 5A-5B.

FIGS. 5A-5B illustrate the difference between the signals that are recovered from the fluorescent protein (FP)-based biosensor (FIG. 5A) and the enzyme-linked biosensor of the disclosure (FIG. 5B). Equivalent microfluidic test volumes were used between the FP-based biosensor that is linked to GFP fluorescent protein (FIG. 5A) and the enzyme-linked biosensor of the disclosure that uses β-glucosidase as a reporter for gene expression (FIG. 5B). FIG. 5 shows that the signals recovered from equivalent microfluidic test volumes for the enzyme-linked biosensor of the disclosure that uses β-glucosidase as a reporter for gene expression (FIG. 5B) are about 10-1000-fold higher in fluorescence intensity than the signals recovered from the traditional FP-based biosensor that is linked to GFP fluorescent protein (FIG. 5A). Fluorescent signal is observed where the sensor cells are located in the microfluidic droplet (FIG. 5A), while the fluorescent product generated by the enzyme-linked biosensor after the addition of a lysis buffer and the clear substrate occupies the entire droplet with more intense fluorescent signal, providing a signal with higher signal-to-noise ratio required for downstream droplet manipulations (e.g., droplet sorting to identify droplets with the highest cis, cis-muconate concentration). Bar scale=10 μm.

This study described the construction and development of the enzyme-linked biosensor of the disclosure. Experiments were carried out to compare the signals that were recovered from equivalent microfluidic test volumes of the enzyme-linked sensor and the TF-based biosensor. The results showed that the signals recovered are, unexpectedly, 100-1000 fold higher in fluorescence intensity for the enzyme-linked biosensor than for the TF-based and/or fluorescent protein (FP)-based biosensor. Such sensitivity makes the enzyme-linked sensor of the disclosure to be potentially useful for HTP applications.

### Example 2—Cell-Based Enzyme-Linked Biosensor

This Example describes the features of the cell-based enzyme-linked biosensor of the disclosure. Specifically, the results are compared to those of the biosensors that were using fluorescent protein as a reporter.

This Example seeks to construct and test the properties, features, and characteristics of the cell-based enzyme-linked biosensor because, at least in part, the previous results shown in Example 1 strongly suggested that a cell-based enzyme-linked biosensor would also perform significantly better than FP-based reporter counterparts. The application of the cell-based enzyme-linked biosensor has several advantages. There is no need to optimize the sensor's dynamic range since the solution with the analyte can be diluted prior to detection if too concentrated. Moreover, the assay is easily automated and can provide rapid monitoring of any analytes with the corresponding transcription factor, operator, and promoter sequences driving the transcription of the enzyme reporter.

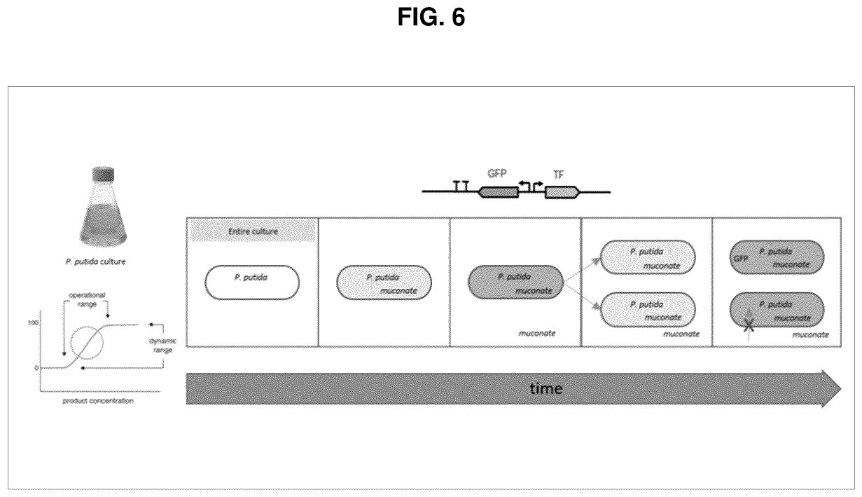

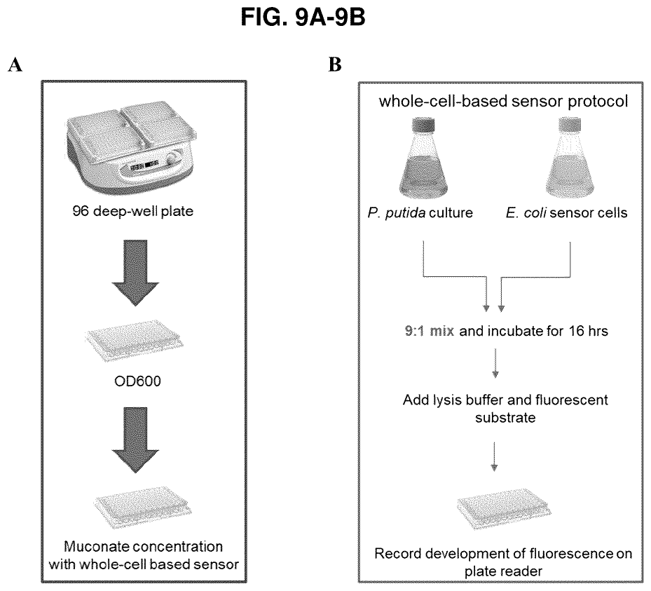

FIG. 6 shows the application of cell-based enzyme-linked biosensors of the disclosure. FIG. 6 shows the application of the FP-based biosensor (cis, cis-muconate sensor) when inserted into the engineered Pseudomonas putida cells producing the product of interest (cis, cis-muconate, CCM). The cells are secreting CCM into the media. The sensor is activated only by the CCM produced inside the cell since P. putida cannot take up external CCM. This is in contrast to the E. coli-based sensor shown in FIGS. 9A-9C, where the E. coli enzyme-based sensor is deployed to measure CCM produced by the engineered P. putida cells. E. coli can take up the CCM present in the media and activate the enzyme-based biosensor. After a 4-hour incubation, the produced reporter is measured by the addition of lysis buffer and clear substrate to produce the fluorescent product occupying the entire well content.

In order to characterize the enzyme-linked CCM biosensor, a cell-based system that can be used to detect bioproducts in broth has been developed. As opposed to the engineered CCM-producing P. putida KT2440 strains. Escherichia coli cells are capable of taking up extracellular CCM, providing an ideal chassis for cell-based biosensor development. The addition of CCM to the growth media of E. coli and their incubation overnight resulted in the expression of a detectable level of fluorescent protein or reporter enzyme that is proportional to the extracellular CCM concentration.

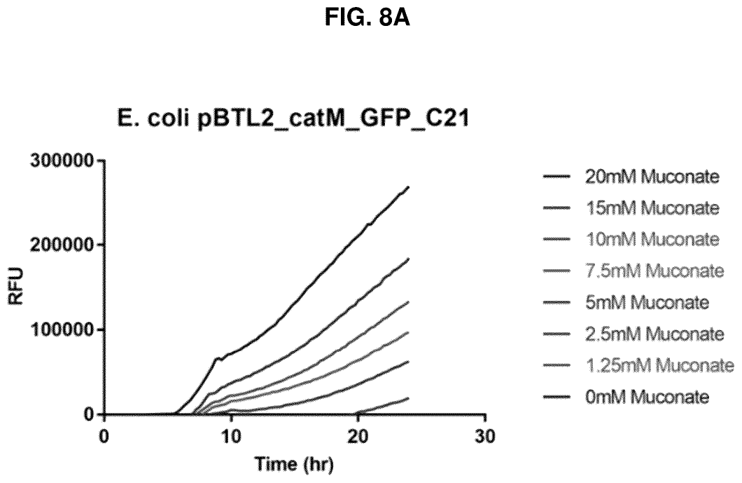

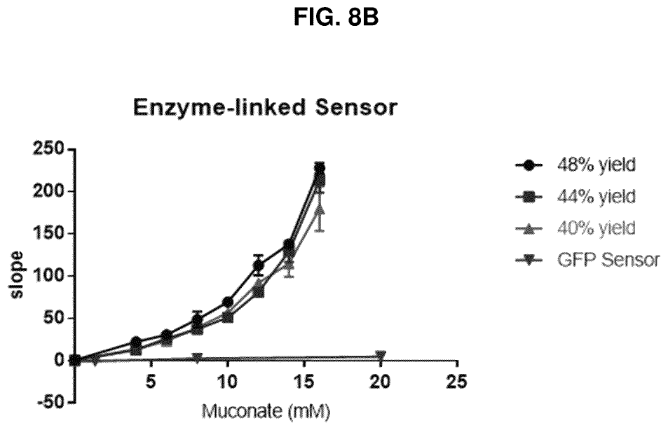

The results obtained showed the performance of the pBTL2_CatM_C21 (green fluorescent protein reporter) and the pBATS_0004 (enzyme-linked reporter) sensors after transforming them into E. coli cells were compared. The results show that a high concentration of CCM in the media did not affect cell growth, and a linear response was observed when CCM concentration and the amount of GFP produced were compared (FIG. 8A).

FIG. 8A depicts the characterization of a cell-based biosensor where the pBTL2_catM_GFP sensor was transformed into E. coli cells and the response to cis, cis-muconate (CCM) in the extracellular medium was measured to evaluate the effects of the increase in CCM concentration on the ability of the bacterial cell to produce a green fluorescent protein (GFP). An E. coli cell that had been engineered into a cell-based biosensor takes up extracellular cis, cis-muconate (CCM) from the broth, which leads the cell-based biosensor to express either a fluorescent protein (if engineered as a TF-based biosensor that is linked to a fluorescent protein) or a reporter enzyme (if engineered as an enzyme-linked reporter) that is proportional with the extracellular CCM concentration. The performance of pBTL2_CatM_C21 (green fluorescent protein reporter) and pBATS_0004 (enzyme-linked reporter) sensors were compared after transforming the respective constructs into E. coli cells. The results in FIG. 8A shows that a linear response was observed when CCM concentration and the amount of GFP produced were compared. The results for the pBATS_0004 (enzyme-linked reporter) sensor were not shown in FIG. 8A.

Thus, to successfully produce the cell-based enzyme-linked biosensor of the disclosure, the cells must be able to detect cis, cis-muconate (CCM) in broth. Escherichia coli cells are capable of taking up extracellular CCM, providing an ideal chassis for cell-based biosensor development. Simply adding CCM to the growth media of E. coli overnight incubation results in the expression of a detectable level of fluorescent protein or reporter enzyme that is proportional to the extracellular CCM concentration. The performance of the pBTL2_CatM_C21 (green fluorescent protein reporter) and the pBATS_0004 (enzyme-linked reporter) sensors were compared after transforming them into E. coli cells. The results showed that the high concentration of CCM in the media did not affect cell growth, and a linear response was observed when CCM concentration and the amount of GFP produced were compared.

When the enzyme-linked and GFP-based sensors were compared, the enzyme-linked variant was 100-1000-fold more sensitive, providing a robust signal up to 15 mM external CCM concentration (FIG. 8B).

FIG. 8B shows a comparison of the traditional fluorescent protein-based (e.g., GFP-based) biosensor and the enzyme-linked biosensor of the disclosure. The experiment is set up as follows: sensor cells (a: E. coli with GFP reporter or b: E. coli with β-glucosidase reporter) are incubated with different concentrations of CCM for at least 4 hours (overnight is more convenient for the sfGFP sensor to get a good signal). The evolution of sfGFP production is measured for the sfGFP reporter by correlating the slope observed in FIG. 8A versus CCM concentration. For the β-glucosidase reporter, the P. putida culture (with CCM in the medium) is mixed with the E. coli enzyme reporter, incubated for at least 4 hours, and a cocktail of lysis buffer and clear substrate is added. As the clear substrate is converted into the fluorescent product (fluorescein), the fluorescence increases over time. The slope of this fluorescence evolution is correlated with CCM concentration to get the slope vs. CCM graph. The pBTL2_catM_GFP and pBATS_0004 sensors were transformed into E. coli cells and their responses to the extracellular CCM were quantified for either GFP or β-glucosidase production, respectively. The results show that the cell-based enzyme-linked biosensor (i.e., cells transformed with pBATS_0004) produced significantly stronger signals for detection compared to those the weaker signals produced by the traditional TF-cell-based biosensor that is linked to a GFP protein (i.e., cells transformed with pBTL2_catM_GFP). The figure also shows the sensor response in the presence of different glucose concentrations in the media, mimicking bioreactor conditions where a mixture of glucose and CCM might be present. The sensor cells were not affected by varying glucose concentrations in the medium.

Some advantages of cell-based enzyme-linked biosensors observed from the disclosure were as follows. There was no need to optimize the sensor's dynamic range since the solution with the analyte could be diluted prior to detection if too concentrated. This was in contrast to when intracellular CCM was measured in the producing cell. Moreover, the assay was easily automated and could provide rapid monitoring of any analytes with the corresponding transcription factor, operator, and promoter sequences driving the transcription of the enzyme reporter.

Some applications of the cell-based enzyme-linked biosensor workflow observed from the disclosure are included as follows. A culture of E. coli cells carrying the pBATS_0004 sensor is mixed with engineered P. putida broth and incubated overnight to induce the reporter enzyme production. The enzyme level is measured in a functional assay.

An enzyme-linked sensor that is about 100-1000 fold more sensitive than fluorescent protein-based variants has been successfully developed and provided as part of the disclosure. The enzyme-linked sensors have shown to be ideally suited for high-throughput (HTP) applications.

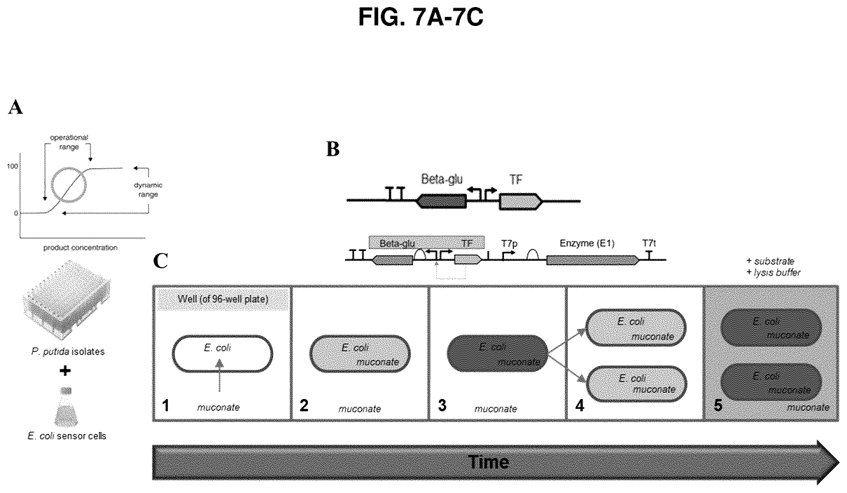

FIGS. 7A-7C depict an application of the cell-based enzyme-linked biosensor in which the muconate (product) is sensed by a cell-based sensor with an enzyme (β-glucosidase) reporter. The biosensor cells are added to the medium and incubated, followed by the addition of a cocktail of lysis buffer and clear substrate. The addition of the low concentration of lysis buffer does not completely destroy the cells but permeabilizes them to allow the enzyme reporter substrate and the product to diffuse freely into and out of the cell. The amount of fluorescent product (fluorescein), as measured by the fluorescence, is proportional to the CCM concentration in the medium. FIG. 7A depicts Pseudomonas putida isolates in a 96-well plate (e.g., 96 different isolates or 30 isolates in triplicates, etc.), and the E. coli depicted are sensor cells in regular LB medium, in which the cells reach late exponential phase growth. The cells do not express the β-glucosidase under these conditions (no CCM added). FIG. 7B depicts two constructs in which the upper construct is displaying the minimal biosensor design with the transcription factor (TF, catM for CCM sensing), a promoter region, and the β-glucosidase reporter gene in the arrangement normally found in transcription regulation circuits in bacteria. The bottom construct in FIG. 7B is a configuration where the biosensor can be coupled to the evaluation of a library of enzyme variants that produce the said product sensed by the sensor using the pMCSG68 plasmid. FIG. 7C depicts the mechanism of the E. coli cell-based CCM sensor. E. coli sensor cells are added to the media containing CCM (bioproduct). The cell can take up the muconate (panel 1), which can bind to the TF (catM) and activate the transcription of the reporter enzyme (β-glucosidase). The cells are incubated for at least 4 hours (panel 2, panel 3). The sensor cells will be dividing during this time, leading to possible differing levels of enzyme reporter on the cell-to-cell basis (panel 4). The differences are averaged out when the lysis buffer and substrate are added, resulting in the conversion of the clear substrate into a fluorescent product.

FIGS. 9A-9B depict a workflow of conducting a screening experiment of P. putida isolates for CCM production using the whole cell-based enzyme-linked biosensor of the disclosure. FIG. 9A depicts a workflow of using a 96-deep-well plate to grow P. putida cells to produce CCM. At the end of the production phase, the OD₆₀₀ is measured to gauge cell densities. The muconate concentration is measured next with a whole-cell-based biosensor (E. co/i-based). FIG. 9B shows a culture of E. coli cells carrying the pBATS_0004 sensors incubated with engineered P. putida broth overnight to induce the reporter enzyme production. The enzyme level is measured in a functional assay in which the lysis buffer and substrates are added, and the fluorescence is monitored.

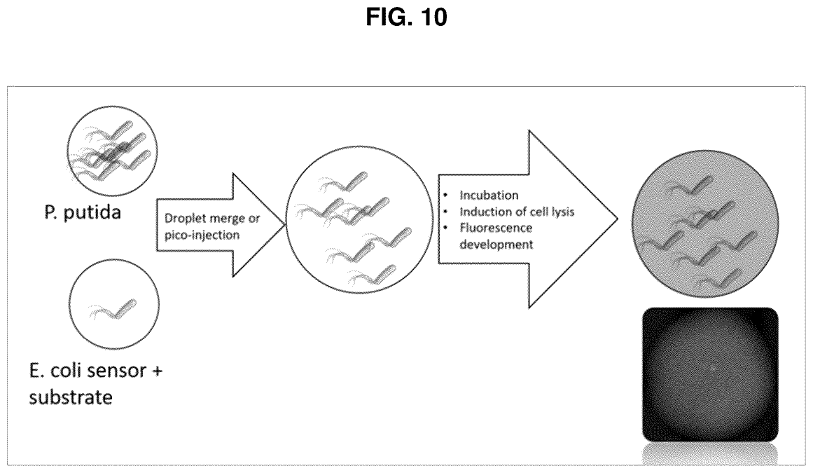

FIG. 10 shows the application of the enzyme-based reporter for the detection of products in a microfluidic setting. The level of CCM in the droplet produced by the engineered P. putida is detected by picoinjection of the E. coli enzyme-based reporter biosensor. The CCM is taken up by the E. coli cells, and β-glucosidase is produced proportional to the CCM level. After at least 4 hours of incubation, there are two ways to initiate the β-glucosidase detection (reporter readout): a) by picoinjection of the substrate and lysis buffer, or b) the substrate is already present, and the E. coli cells are permeabilized by external stimuli to make the substrate accessible to the enzyme.

### Example 3—Identification and Characterization of Stable and Highly Active, β-Glucosidases

The main objective of this Example is to identify novel β-glucosidases that can be used in a wide variety of industrial applications, e.g., without limitation, to be used as a biosensor reporter that is compatible with high-throughput (HTP) assays and for microfluidics. The novel β-glucosidases should remain active under physiologically relevant conditions, such as temperatures up to 46° C. and a pH range of 6-8. Exhibiting such characteristics would make these enzymes strong candidates for enzyme reporter applications.

### Identification of B-Glucosidases

Multiple variants of β-glucosidases were identified using known methods to identify sequences that are homologous to β-glucosidase from gut microbes. A computational screen with a glycosyl-hydrolase specific Position Specific Scoring Matrix (PSSM) was used to identify CAZymes from the selected set of gut microbial genomes. pBATS_0004 β-glucosidase was one of the CAZymes identified along with other variants based on sequence homology. Bioinformatics methodologies were used to predict the 3-dimensional structure of the identified β-glucosidases. β-glucosidases that were predicted to exhibit the desired properties, e.g., optimal for protein expression, were cloned into the pMCSG68 expression vector. The variants of β-glucosidase were expressed in E. coli, and the variants of β-glucosidase were purified. A selected number of variants/orthologs of β-glucosidase were purified and were characterized using basic enzymology and biochemical methodologies. Many variants/orthologs of β-glucosidase were found in the gut microbiota. Gut microbes were used for the identification and cloning of CAZymes. Some of these may have hundreds of CAZymes, while some pathogenic microbes have only a few.

### Identification of Novel B-Glucosidases

Novel variants of nucleic acids that encode the β-glucosidase enzyme that exhibit superior properties are nucleic acids comprising the nucleotide sequences of SEQ ID NOs: 2-18, as shown in Table 1.

The β-glucosidase enzymes, as listed in Table 1, i.e., with SEQ ID NOs: 2-18, are expressed in bacteria and transported into the periplasm as the final destination. Each full sequence encodes for a signal peptide that directs it to the periplasm, where it would be cleaved off. Thus, the DNA sequences, as listed in Table 1, do not include the portion of the DNA sequence that encodes the signal peptide from the genes, thereby tricking the bacteria, and the enzymes are expressed in the cytosol. The approach described herein allows for about a 10-1000-fold increase in protein expression level. Thus, the characterization of the β-glucosidases of SEQ ID NOs: 2-18 described in the disclosure are from the truncated sequences, i.e., without the portion that encodes the signal peptide. These sequences are expressed by adding the ‘ATG’ (bacterial start codon) as an artificial ‘M’ as the first residue. For experiments that require swapping out APC115086.102 with any of the sequences in the sensor, the ‘ATG’ could be added to the sequences of SEQ ID NOs: 2-18.

As an example, clone APC115038.102 (APC115038.26-783.P(785) . . . pMCSG68), i.e., SEQ ID NO: 2, denotes a sequence where the first 25 amino acids were not included in the clone. Thus, this strategy indirectly instructs the E. coli to express the enzyme in the cytosol as opposed to the natural route of directing it to the periplasm.

A representative map of the construct for a β-glucosidase-based muconate biosensor is depicted in FIG. 19. The nucleotide sequence of the β-glucosidase-based muconate biosensor construct is provided as SEQ ID NO: 24.

FIG. 19 shows a map of the β-glucosidase-based muconate sensor. The pMCSG68 protein expression vector, as described in Eschenfeldt et al., “New LIC vectors for production of proteins from genes containing rare codons.” Journal of Structural and Functional Genomics 14 (2013):135-144, was further modified to incorporate a sensor circuit. The plasmid carries an ampicillin resistance gene for selection and maintenance in E. coli. The vector was used for protein expression, where the DNA sequence encoding the protein of interest was inserted downstream of the ‘TEV-site’. Protein expression was initiated by high-level transcription of the T7-inducible system. RNA copies were generated from the T7-promoter to the T7-terminator region. A strong ribosomal binding site (RBS) ensured that the mRNA copies were efficiently used by the ribosomes to produce large quantities of the target protein. The disclosure introduced the sensor circuit into a ‘silent’ region of the vector upstream of the T7 promoter. The transcription of the reporter enzyme gene, therefore, was solely induced by the transcription factor binding to the upstream promoter region. Basal transcription of the reporter circuit was very low. In order to produce the enzyme efficiently, a relatively strong ribosome binding site was introduced upstream of the reporter enzyme gene. Due to the high copy number of this plasmid in E. coli, efficient expression of the reporter enzyme was possible. The nucleotide sequence of the β-glucosidase-based muconate biosensor construct is provided as SEQ ID NO: 24.

The CatM-promoter-GFP reporter circuit in Pseudomonas putida KT2440 cells was previously developed. The circuit was not previously tested in E. coli. The use of the CatM-promoter-β-glucosidase for the monitoring of muconate concentration was the only system that was reduced to practice in the disclosure. However, the plasmid construct and the methods provided in the disclosure could be used for the monitoring of other analytes for which a transcription factor-based system exists. At least more than 20 systems have been reported to date.

It is noted that the muconate sensor has additional importance in biomanufacturing since muconate is produced by bacteria and turned into adipic acid, which is a nylon precursor with the potential to replace oil-based nylon production.

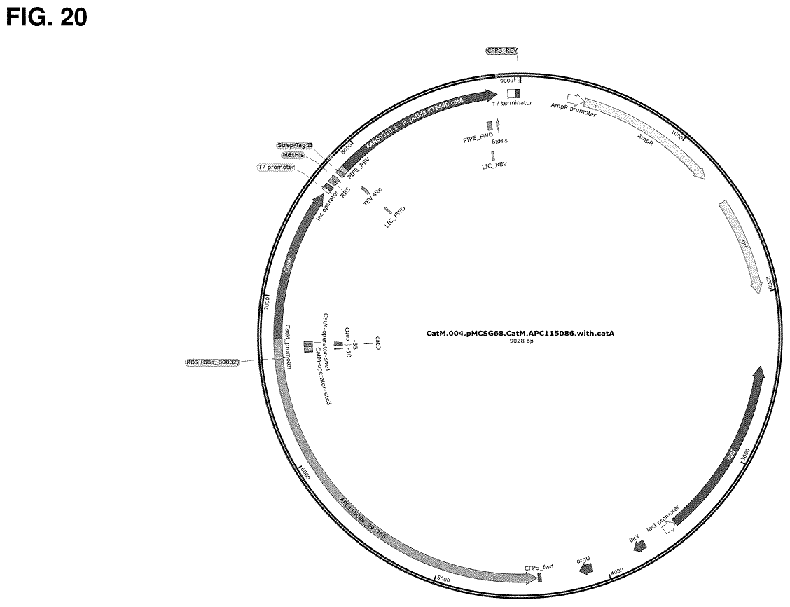

A representative map of the construct for a β-glucosidase-based muconate sensor, along with the coding sequence of P. putida KT2440 CatA enzyme, is depicted in FIG. 20.

FIG. 20 shows a schematic map of the β-glucosidase-based muconate sensor along with the coding sequence of P. putida KT2440 CatA enzyme. The catA gene is in the protein expression region, i.e., a T7 promoter-driven, of the vector. It is hypothesized that the vector depicted herein, which has not yet been constructed, could be used for the engineering of CatA enzyme variants with higher efficiencies (higher catalytic rate (kcat), more optimal Km values, or a combination of the two) in the following manner. The vector would be introduced into E. coli BL21(DE3) host tailored for high-level protein expression. The protein expression (CatA expression) can be induced by the addition of IPTG to the media when cells are grown in the exponential phase. The IPTG induces the expression of T7 polymerase encoded on the E. coli BL21(DE3) genome. The elevated T7 polymerase expression, in turn, induces the transcription of the T7p-T7t portion of the plasmid, generating large copies of mRNA. The E. coli BL21(DE3) cells are ‘tricked’ and will allocate up to 40-50% of resources to producing the foreign protein (CatA in this case). This, in turn, would allow for a simple screening of enzyme activities of introduced variants. The reaction converting catechol to cis, cis-muconate can be followed by the addition of catechol. The catechol would enter the cells and get converted to muconate by the CatA enzyme. The novelty is in the notion that the plasmid encodes for an enzyme-linked muconate sensor. The muconate turns on the production of the reporter enzyme, i.e., β-glucosidase. A researcher could simply monitor the level of β-glucosidase via a fluorescent signal. It is envisaged that the cloning of 96 variants of CatA and their screening is performed in a 96-cell well plate format. The cells would be grown in the plate, the expression of CatA enzyme would be induced with IPTG when the cells reach a certain cell density (e.g., OD₆₀₀=0.4-0.6), the enzyme variants would be expressed within 4-16 h (based on induction temperature, i.e., 37-18° C., respectively). The enzyme substrate could be added to the cultures (e.g., enzyme substrate of catechol in the present example), and the level of produced enzyme reporter (after 4-16 h incubation) could be measured after the addition of fluorogenic substrate by monitoring the evolution of the fluorescent product.

The system could be used for the optimization of not only a single enzyme but also a pathway because the plasmid is designed for the expression of multiple pathway components (i.e., which had been demonstrated with up to 6 genes).

Multiple experiments were performed to test the thermostability and pH tolerance of each of the variants of β-glucosidase.

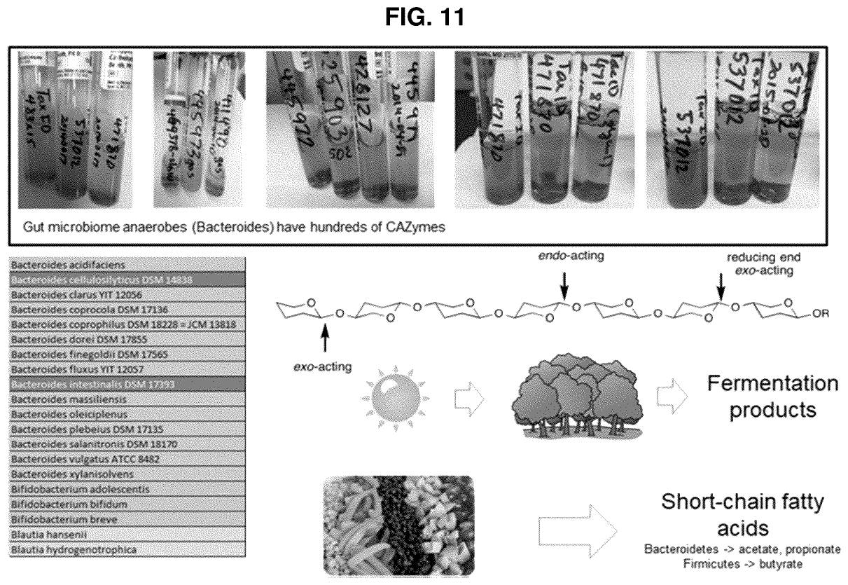

FIG. 11 depicts a list of gut microbiota that encode a large repertoire of Carbohydrate-Active enZymes (CAZymes). Several gut microorganisms were cultivated to extract genomic DNA for the cloning, expression, and characterization of exo-acting CAZymes (mostly β-glucosidases and β-galactosidases). The table lists the gut microbes used for the selection of CAZymes. Cellulose is the most abundant biopolymer on Earth, produced mostly by plants via photosynthesis and by microorganisms via alternative pathways. The long chains of cellulose are broken down via enzymes acting on non-reducing and reducing ends of the polymer or endo-acting enzymes, cleaving the long chain into smaller products. The gut microbes use cellulose and other sugar polymers as carbon sources to convert them into energy while secreting short-chain fatty acids that are important to the gut health. Since these gut microbes solely rely on the degradation of complex polysaccharides, their genome encodes for a plethora of CAZymes, providing an opportunity to identify enzymes with superior activities.

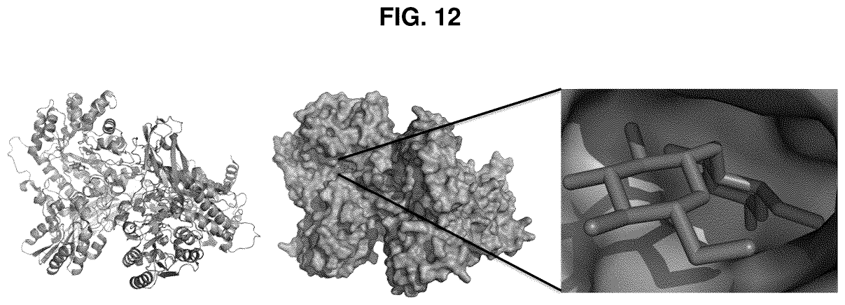

FIG. 12 depicts the structure of one of the β-glucosidases. The functional protein is a homodimer where residues from both subunits contribute to the enzyme activity. This figure shows how the terminal glucosyl moiety of a maltose molecule fits into the active site of the β-glucosidase protein.

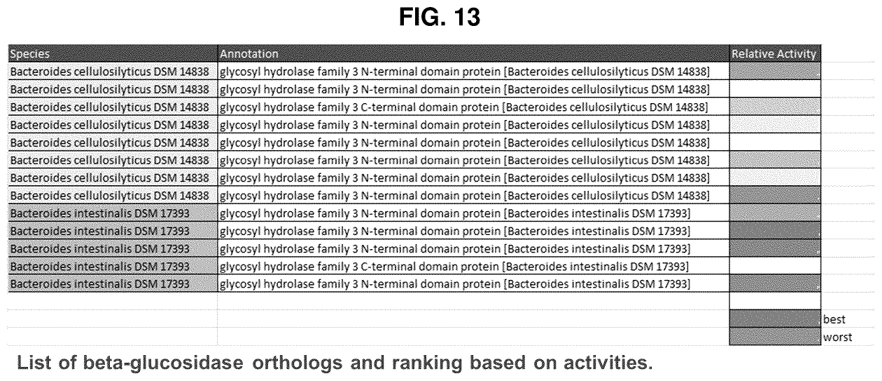

FIG. 13 depicts a list of β-glucosidase orthologs and a survey of β-glucosidase activities. Some gut microbes have many enzymes in their genomes that are annotated as β-glucosidases. The orthologs were cloned, expressed, purified, and characterized. A wide range of activities were identified, suggesting that the different orthologs have preferences for slightly different terminal sugars.

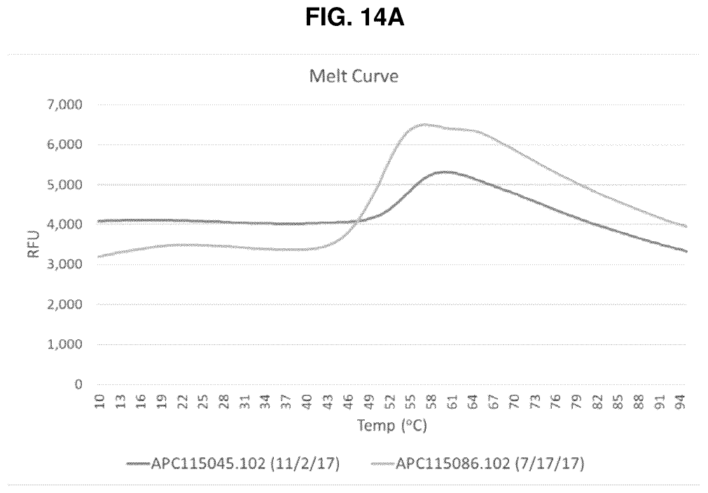

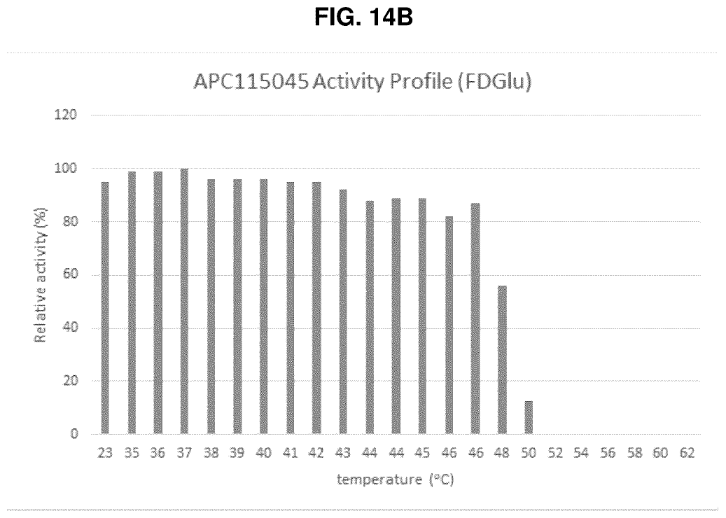

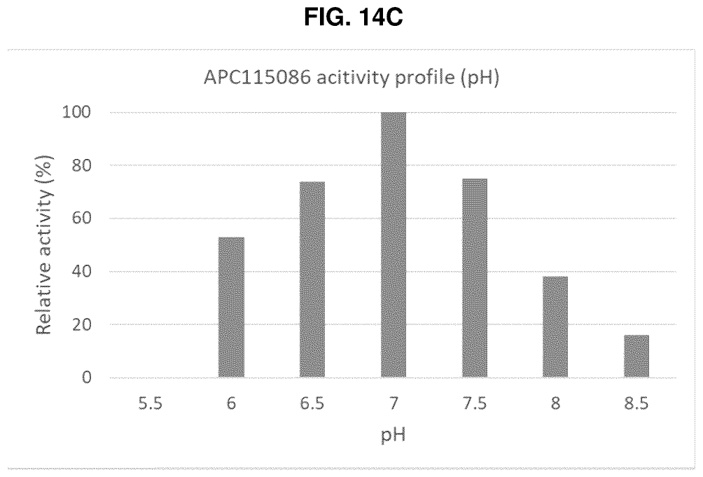

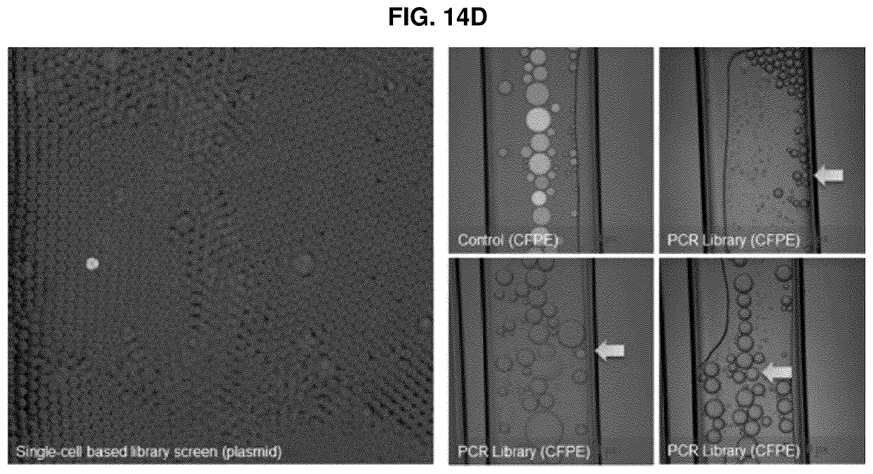

FIGS. 14A-14D depict various characteristics of two β-glucosidase variants. Two candidate β-glucosidases, APC115045 and APC115086, from a collection of more than 40 enzymes were selected for further analysis. The enzymes were tested for melting temperature (FIG. 14A), activity profile over various temperatures, e.g., a temperature optimum (FIG. 14B), activity profile over various pH conditions, e.g., a pH optimum (FIG. 14C), and for compatibility in a microfluidic setting (FIG. 14D). FIG. 14A shows the two β-glucosidases tested for melting temperature, indicating the relative stability of the proteins. FIG. 14B shows the two β-glucosidases tested for temperature optimum. FIG. 14B shows the activity profile of the leading candidate of β-glucosidase variants, APC115045. Aliquots of APC115045 were incubated in various temperatures ranging from 23° C. to 50° C. for 5 min, followed by cooling down and an analysis of their relative activities. The results show that the enzyme retains most of its activity up to 46° C. The enzyme is not particularly thermotolerant since gut microbes do not experience extreme temperatures, and enzymes are evolved to display maximum activities around the normal body temperature (37° C.). APC115045 is the β-glucosidase variant/ortholog with SEQ ID NO: 5. FIG. 14C shows the two β-glucosidases tested for pH optimum. FIG. 14C shows the activity profile of the leading candidate of β-glucosidase variants, APC115086. Aliquots of APC115086 were incubated at pH 5.5, pH 6.0, pH 6.5, pH 7.0, pH 7.5, pH 8.0, and pH 8.5 for 10 min, followed by an analysis of their relative activities under those pH conditions. The results show that the enzyme activity is maximal around a neutral pH, retaining at least 40% activity between pH 6 and pH 8, suggesting an adaptation to the human gut environment. APC115086 is the β-glucosidase variant/ortholog with SEQ ID NO: 9. FIG. 14D shows the two β-glucosidases tested for compatibility in a microfluidic setting. The β-glucosidases were tested for enzyme activities in a droplet microfluidic setting. This is an important aspect for microfluidic applications where the surfactants that are used to stabilize droplets might interfere with some enzyme activities. The β-glucosidase is active in droplets using cell-based and cell-free systems.

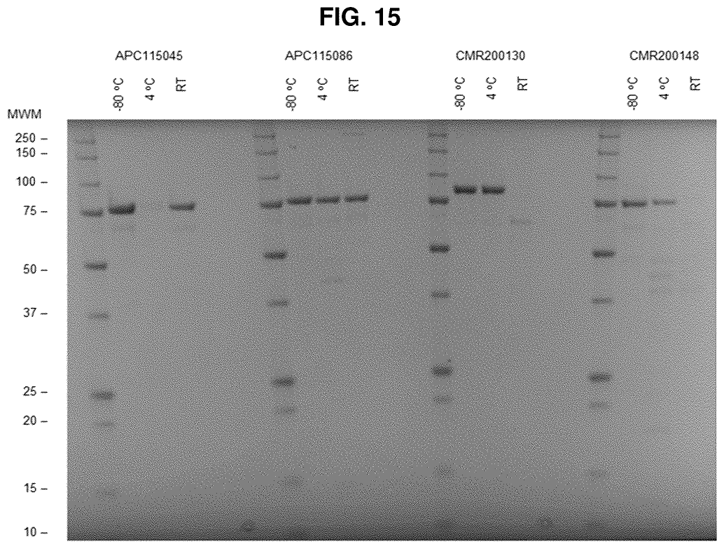

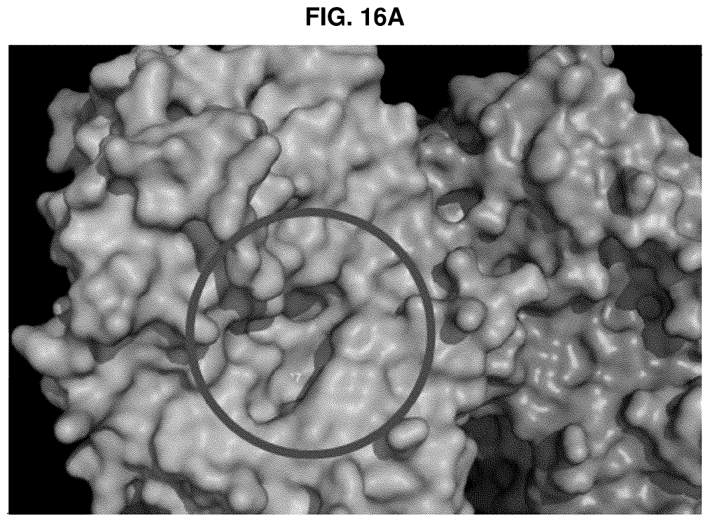

FIG. 15 depicts enzyme stability measurement of four variants of β-glucosidases with each being stored at −80° C., 4° C., and at room temperature for about 5 years. The results from the SDS-PAGE analysis show that the APC115045 and APC115086 enzymes remained stable for a significant amount of time at room temperature, confirming that these enzymes in the biosensor will be stable in a kit. In some instances, the thermostability of the β-glucosidases makes them highly desirable in bioproduction, and in the production of other analytes and products.

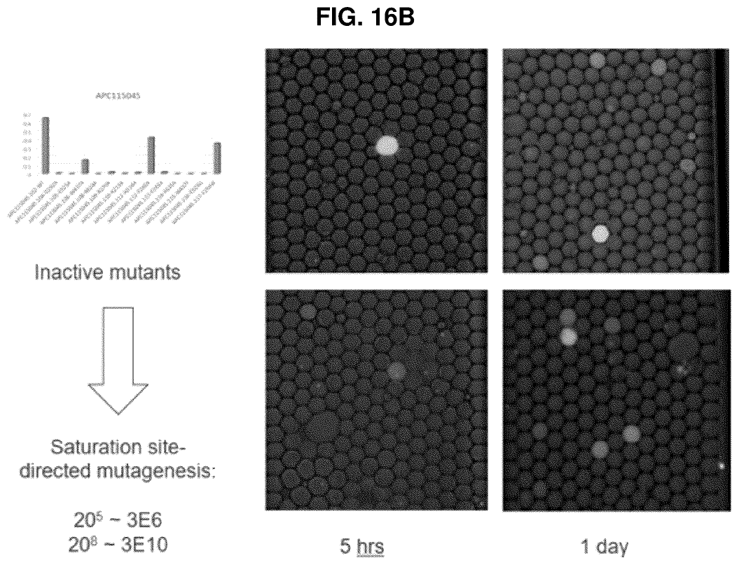

FIGS. 16A-16B depict the characterization of a Bacteroides intestinalis β-glucosidase. The structure of the APC115045 enzyme (FIG. 16A) was determined and used to design mutant libraries around the active site. The mutants were tested in microfluidic droplet assays (FIG. 16B). FIG. 16A shows a structure of the APC115045 variant of β-glucosidase. The active form is a homodimer with both subunits contributing to the activity (residues part of the active site). Residues in the vicinity of the active site were selected and mutated to alanine. FIG. 16B shows activity profiles of the wild-type and mutant enzymes. Most mutations destroyed activities as expected. The relative activities observed in the microfluidic droplets followed those observed in traditional plate-based assays.

The experiments described thus far have identified APC115045.102 and APC115086.102 as the top two leading candidates for β-glucosidase. Next, biochemistry methodologies were carried out to determine the enzyme kinetics of APC115045.102 and APC115086.102.

### Enzyme Kinetics of APC115045.102 and APC115086.102

Biochemistry methodologies were carried out to determine the enzyme kinetics of APC115045.102 and APC115086.102. See Lehninger, A. L.; Nelson, D. L.; Cox, M. M. (2008). Principles of Biochemistry (5th ed.). New York, NY: W.H. Freeman and Company.

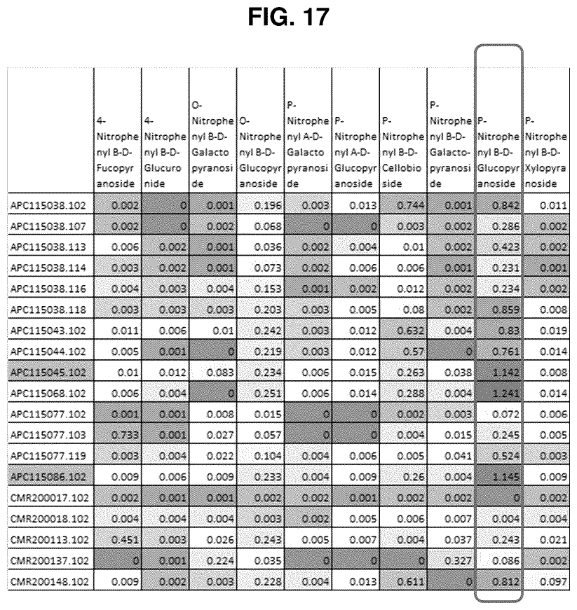

The enzymes were screened against a panel of substrates (fucopyranoside, glucuronide, galactopyranoside, and xylopyranoside). The best β-glucosidases were used for further studies and sensor reporter development. The sugar hydrolase activities of selected β-glucosidase candidate enzymes are shown in FIG. 17.

FIG. 17 depicts a table of sugar hydrolase activities of selected β-glucosidase candidate enzymes. FIG. 17 shows comparisons of 19 candidates/variants/orthologs of β-glucosidase, each undergoing enzymatic assays using a panel of ten substrates. The numbers depict the relative activities. The results show that APC115045.102 and APC115086.102 performed well under p-Nitrophenyl-β-D-glucopyranoside. These enzymes did not show activities against fucopyranoside, glucuronide, galactopyranoside, and xylopyranoside substrates, while others showed preferences for some of these substrates. APC115045.102 and APC115086 did not cleave substrates efficiently when a hydroxyl group was in close proximity to the cleavage site. These results suggest that APC115045.102 and APC115086 are specific β-glucosidases and may be good candidates for reporter development. APC115045.102 is the β-glucosidase variant/ortholog with SEQ ID NO: 5. APC115086.102 is the β-glucosidase variant/ortholog with SEQ ID NO: 9.

### Km (the Michaelis Constant)

Km (also known as the Michaelis constant) is the substrate concentration at which the reaction rate is 50% of the Vmax (i.e., the maximum rate of the reaction when all the enzyme's active sites are saturated with substrate). Km is a measure of the affinity an enzyme has for its substrate, as the lower the value of Km, the more efficient the enzyme is at carrying out its function at a lower substrate concentration. The Km for APC115045.102 and APC115086.102 were determined to be 1.01±0.14 mM and 0.63±0.03 mM, respectively. Thus, APC115086.102 is more efficient than APC115045.102 in carrying out its function at lower substrate concentrations.

Catalytic Constant (kcat)

The catalytic constant (k_{cat}) or turnover number is the number of enzymatic reactions a single saturated enzyme molecule can catalyze per unit of time. The k_{cat} for APC115045.102 and APC115086.102 were determined to be 346±52 s⁻¹ and 142±4 s⁻¹, respectively. Thus, APC115045.102 is more catalytically active than APC115086.102 in turning over more molecules per second.

### Catalytic Efficiency (Kcat/KM)

The kcat/KM and the catalytic efficiency are frequently used to compare the catalytic effectiveness of enzymes. The kcat/KM for APC115045.102 and APC115086.102 were determined to be 341±3 1/mMs⁻¹ and 224±7 1/mMs⁻¹, respectively. Thus, APC115045.102 has higher catalytic efficiency than APC115086.102.

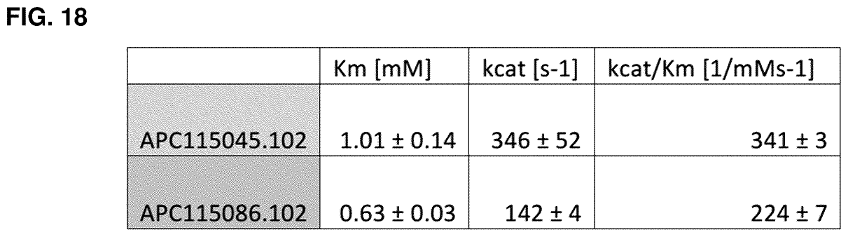

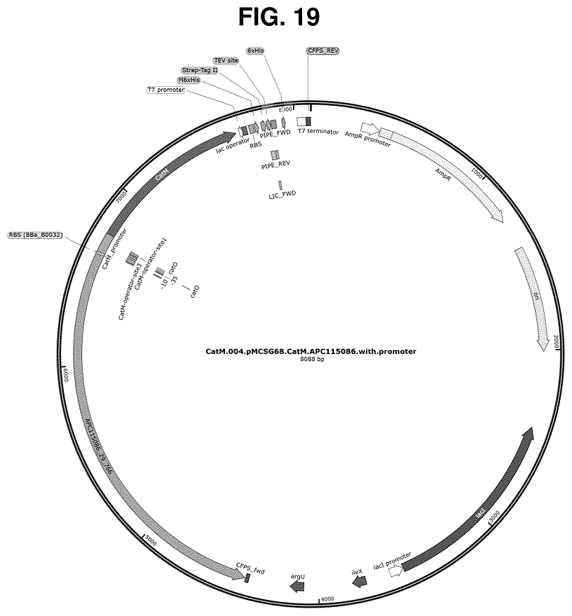

The enzyme kinetics results for the two lead candidates of β-glucosidase APC115045.102 and APC115086.102 are summarized in FIG. 18.

FIG. 18 provides a table of enzyme kinetics results from two β-glucosidase variants, i.e., APC115045.102 and APC115086.102. The APC115086.102 enzyme has a lower Km value, reaching half-max reaction velocity at lower substrate concentration than the APC115045.102 enzyme, albeit with lower catalytic efficiency (kcat). Overall, the two enzymes are very similar. For reporter development, the APC115086.102 enzyme was selected.

The foregoing description is given for clearness of understanding only, and no unnecessary limitations should be understood therefrom, as modifications within the scope of the invention may be apparent to those having ordinary skill in the art.

Throughout this specification and the claims which follow, unless the context requires otherwise, the word “comprise” and variations such as “comprises” and “comprising” will be understood to imply the inclusion of a stated integer or step or group of integers or steps but not the exclusion of any other integer or step or group of integers or steps.

It should be understood that while various embodiments of the specification are presented using “comprising” language, under various circumstances, a related aspect may also be described using “consisting of” or “consisting essentially of” language. The disclosure contemplates embodiments described as “comprising” a feature to include embodiments that “consist of” or “consist essentially of” the feature. The use of any and all examples or exemplary language (e.g., “such as”) provided herein is intended merely to better illuminate the disclosure and does not pose a limitation on the scope of the disclosure unless otherwise claimed. No language in the specification should be construed as indicating any non-claimed element as essential to the practice of the disclosure.

It should also be understood that when describing a range of values, the disclosure contemplates individual values found within the range. In any of the ranges described herein, the endpoints of the range are included in the range. However, the description also contemplates the same ranges in which the lower and/or the higher endpoint is excluded.

As will be apparent to those of skill in the art upon reading this disclosure, each of the individual aspects described and illustrated herein has discrete components and features that may be readily separated from or combined with the features of any of the other several aspects. Any recited method can be carried out in the order of steps recited or in any other order which is logically possible. This is intended to provide support for all such combinations.

Preferred embodiments of this disclosure are described herein. Variations of these embodiments may become apparent to those of ordinary skill in the art upon reading the foregoing description. This disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context. Also, only such limitations that are described herein as critical to the disclosure should be viewed as such; variations of the disclosure lacking limitations that have not been described herein as critical are intended as aspects of the disclosure.

Throughout the specification, where compositions are described as including components or materials, it is contemplated that the compositions can also consist essentially of, or consist of, any combination of the recited components or materials unless described otherwise. Likewise, where methods are described as including particular steps, it is contemplated that the methods can also consist essentially of, or consist of, any combination of the recited steps unless described otherwise. The products and methods illustratively disclosed herein suitably may be practiced in the absence of any element or step that is not specifically disclosed herein.

The practice of a method disclosed herein and individual steps thereof can be performed manually and/or with the aid of automation provided by electronic equipment. Although processes have been described with reference to particular embodiments, a person of ordinary skill in the art will readily appreciate that other ways of performing the acts associated with the methods may be used. For example, the order of various steps may be changed without departing from the scope or spirit of the method unless described otherwise. In addition, some of the individual steps can be combined, omitted, or further subdivided into additional steps.

All patents, publications, and references cited herein are hereby fully incorporated by reference. In case of conflict between the present disclosure and incorporated patents, publications, and references, the present disclosure should control.

1. A thermostable enzyme-linked biosensor expression cassette comprising a nucleic acid comprising a nucleotide sequence encoding a β-glucosidase reporter, wherein the nucleotide sequence comprises at least 80%, at least 85%, at least 90%, or at least 95% sequence identity to the nucleotide sequence of any one of SEQ ID NOs: 1-18.

2. The expression cassette of claim 1, wherein the nucleic acid further comprises a nucleotide sequence encoding (i) a transcription factor, (ii) a promoter, (iii) a terminator, and (iv) a cloning site for a gene of interest.

3. The expression cassette of claim 2, wherein the cloning site for the gene of interest is between the promoter and the terminator.

4. The expression cassette of claim 2, wherein the gene of interest is a nucleotide sequence encoding an enzyme.

5. The expression cassette of claim 4, wherein the enzyme produces a product or analyte that binds the transcription factor and activates transcription of the β-glucosidase reporter, producing a signal proportional to the product.

6. The expression cassette of claim 2, wherein the transcription factor is a CatM transcription factor.

7. The expression cassette of claim 6, wherein the nucleotide sequence encoding the CatM transcription factor comprises at least 80%, at least 85%, at least 90%, or at least 95% sequence identity to the nucleotide sequence of SEQ ID NO: 19.

8. The expression cassette of claim 6, wherein the nucleotide sequence encoding the CatM transcription factor comprises the nucleotide sequence of SEQ ID NO: 19.

9. The expression cassette of claim 2, wherein the promoter is a T7 promoter.

10. The expression cassette of claim 9, wherein the nucleotide sequence encoding the T7 promoter comprises at least 80%, at least 85%, at least 90%, or at least 95% sequence identity to the nucleotide sequence of SEQ ID NO: 20.

11. The expression cassette of claim 9, wherein the nucleotide sequence encoding the T7 promoter comprises the nucleotide sequence of SEQ ID NO: 20.

12. The expression cassette of claim 2, wherein the promoter is a CatM promoter.

13. The expression cassette of claim 12, wherein the nucleotide sequence encoding the CatM promoter comprises at least 80%, at least 85%, at least 90%, or at least 95% sequence identity to the nucleotide sequence of SEQ ID NO: 21.

14. The expression cassette of claim 12, wherein the nucleotide sequence encoding the CatM promoter comprises the nucleotide sequence of SEQ ID NO: 21.

15. The expression cassette of claim 2, wherein the terminator is a T7 terminator.

16. The expression cassette of claim 15, wherein the nucleotide sequence encoding the T7 terminator comprises at least 80%, at least 85%, at least 90%, or at least 95% sequence identity to the nucleotide sequence of SEQ ID NO: 22.

17. The expression cassette of claim 15, wherein the nucleotide sequence encoding the T7 terminator comprises the nucleotide sequence of SEQ ID NO: 22.

18. The expression cassette of claim 1, wherein the nucleotide sequence comprises at least 80%, at least 85%, at least 90%, or at least 95% sequence identity to the nucleotide sequence of SEQ ID NO: 23.

19. The expression cassette of claim 1, wherein the nucleotide sequence comprises at least 80%, at least 85%, at least 90%, or at least 95% sequence identity to the nucleotide sequence of SEQ ID NO: 24.

20. A vector comprising the expression cassette of claim 1.

21. A host cell comprising the vector of claim 20.

22. The host cell of claim 21, wherein the host cell is an Escherichia coli BL21(DE3) cell.

23. A composition comprising the host cell of claim 21 and a diluent.

24. A method of detecting a product or analyte present in a sample comprising:
(a) contacting the sample with the expression cassette of claim 1 and a substrate of the β-glucosidase reporter, and
(b) detecting expression of the β-glucosidase reporter in the sample.

25. A method of determining a concentration of a product or analyte present in a sample comprising:
(a) contacting the sample with the expression cassette of claim 1;
(b) detecting expression of the β-glucosidase reporter in the sample;
(c) measuring the concentration of the β-glucosidase reporter; and
(d) comparing the concentration of the β-glucosidase reporter to a control or standard to determine the concentration of the product or analyte present in the sample.

26. The method of claim 24, wherein the analyte is muconate.

27. The method of claim 24, wherein the expression cassette is in a vector.

28. The method of claim 27, wherein the vector is in a host cell.

29. The method of claim 25, wherein the analyte is muconate.
